(19) United States
(12) Patent Application Publication
(10) Pub. No.: US 2026/0215417 A1
(43) Pub. Date: Jul. 30, 2026

(54) INSECT CONTROL NANOBODIES AND USES THEREOF

(71) Applicant: IBI-Ag Innovative Bio Insecticides Ltd., Ness Ziona (IL)

(72) Inventors: Rony OREN-BENAROYA, Givat Brenner (IL); Amir AYALI, Sde Warburg (IL); Jessica MONSERRATE, Chapel Hill, NC (US); Jeroen VAN RIE, Merelbeke (BE)

(73) Assignee: IBI-Ag Innovative Bio Insecticides Ltd., Ness Ziona (IL)

(21) Appl. No.: 19/640,450

(22) Filed: Apr. 7, 2026

Related U.S. Application Data
(62) Division of application No. 17/775,898, filed on Nov. 11, 2020, now Pat. No. 12,622,431.
(60) Provisional application No. 62/933,533, filed on Nov. 11, 2019.

(51) Int. Cl. A01N 25/28; A01N 63/50; C07K 16/18; C07K 16/40

(52) U.S. Cl. CPC A01N 25/28; A01N 63/50; C07K 16/18; C07K 16/40; C07K 2317/22

(57) ABSTRACT

Insect control nanobodies are provided. Accordingly there is provided a nanobody which specifically binds to an insect polypeptide selected from the group consisting of: a polypeptide comprising a chitin binding domain (CBD), V-ATPase subunit c, trehalase, cytochrome p450 monooxygenase, chitin deacetylase, chitin synthase and NPC1 sterol transporter, wherein binding of the nanobody to the insect polypeptide confers an insect control activity to the nanobody. Also provided are polynucleotides encoding the nanobody, host cells expressing the nanobody and methods of using it.

### RELATED APPLICATIONS

This application is a Division of U.S. patent application Ser. No. 17/775,898 filed on
May 11, 2022, which is a National Phase of PCT Patent Application No. PCT/IL2020/051170 having the International Filing Date of Nov. 11, 2020, which claims the benefit of priority under 35 USC § 119 (e) of U.S. Provisional Patent Application No. 62/933,533 filed on
Nov. 11, 2019. The contents of the above applications are all incorporated by reference as if fully set forth herein in their entirety.

### SEQUENCE LISTING STATEMENT

The XML file, entitled 106851SequenceListing.xml, created on Apr. 5, 2026, comprising 764,907 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to insect control nanobodies and uses thereof.

Management of insect pests in the past 70 years has been achieved mainly through application of synthetic pesticides. Since the discovery of the insecticidal properties of DDT by Paul Müller in 1939, hundreds of insecticidal compounds have been developed, accompanied by a relatively steady increase in insecticide use. Most of the currently used chemical insecticides have a high potential for damaging the ecosystem, they are toxic to humans directly or through the food chain and their use is also impeded by development of genetic insect resistance. The most prominent biological solutions currently in use include beneficial organisms or natural enemies that reduce harm caused by insects, pheromones that act as bait or harm reproduction, release of sterile males, spraying with Bacillus thuringiensis toxin or genetically modified crops containing a gene for Bacillus thuringiensis toxin that is lethal to the insects but not to humans. However, these biological solutions also have disadvantage, including low efficiency, danger of violating the natural ecological balance and development genetic insect resistance (e.g. in the case of the use of Bacillus toxin).

Nanobodies, also known as VHH antibodies, are single domain antibodies which practically contain the heavy chain of an antibody (HCAb) and completely lack the light chain. They were discovered in the blood of camels by Raymond Hamers who was credited with this discovery in 1989 at Vrije Universiteit Brussel. Nanobodies are the smallest available intact antigen binding fragment (Cortez-Retamozo et al., 2004; Revets et al., 2005) with a size of approximately 15 kDa. The nanobodies have significant advantages including high production yield in a broad variety of expression systems, their minimal size allows high accessibility to their epitopes, high physical-chemical stability, reversible refolding and high solubility in aqueous solutions, highly homogenous showing no signs of spontaneous dimerization and ability to specifically recognize unique epitopes with sub-nanomolar affinities. The use of nanobodies as insecticides has been previously suggested (see e.g. EP Patent Application Publication Nos: EP3415010 and EP2609116; US Patent Publication No: U.S. Pat. No. 9,516,879; US Patent Application Publication No: U.S. Pat. No. 9,803,003B2; and International Patent Application Publication No. WO2014191146).

### SUMMARY OF THE INVENTION

According to an aspect of some embodiments of the present invention there is provided a nanobody which specifically binds to an insect polypeptide selected from the group consisting of:
(i) a polypeptide comprising a chitin binding domain (CBD), wherein the nanobody binds the CBD;
(ii) V-ATPase subunit c, wherein the nanobody comprises complementarity determining regions (CDRs) as set forth in SEQ ID NOs: 167-169; 171 and 168-169; 174 and 168-169; 167, 178 and 169; or 180-182 arranged in a sequential order from N to C on the nanobody;
(iii) trehalase;
(iv) cytochrome p450 monooxygenase;
(v) chitin deacetylase;
(vi) chitin synthase; and
(vii) NPC1 sterol transporter, wherein binding of the nanobody to the insect polypeptide confers an insect control activity to the nanobody.

According to an aspect of some embodiments of the present invention there is provided a composition comprising a nanobody which specifically binds to an insect polypeptide selected from the group consisting of:
(i) a polypeptide comprising a chitin binding domain (CBD), wherein the nanobody binds the CBD;
(ii) V-ATPase subunit c, wherein the nanobody comprises complementarity determining regions (CDRs) as set forth in SEQ ID NOs: 167-169; 171 and 168-169; 174 and 168-169; 167, 178 and 169; 180-182; 187-189; or 191-193 arranged in a sequential order from N to C on the nanobody;
(iii) trehalase;
(iv) cytochrome p450 monooxygenase;
(v) chitin deacetylase;
(vi) chitin synthase; and
(vii) NPC1 sterol transporter, and a toxin moiety having an insect control activity.

According to some embodiments of the invention, the binding of the nanobody to the insect polypeptide confers an insect control activity to the nanobody.

According to some embodiments of the invention, the nanobody downregulates activity of the insect polypeptide.

According to some embodiments of the invention, the nanobody or the composition being formulated for delivery by spraying, irrigation and/or fumigation.

According to some embodiments of the invention, the CBD is type 2 chitin-binding domain (ChtBD2)

According to some embodiments of the invention, the CBD comprises an amino acid sequence selected form the group consisting of SEQ ID NO: 3-7.

According to some embodiments of the invention, the nanobody specifically binds the CBD and comprises complementarity determining regions (CDRs) as set forth in SEQ ID NOs: 15-17; 19-21; 23-25; or 15 and 28-29 arranged in a sequential order from N to C on the nanobody.

According to some embodiments of the invention, the nanobody specifically binds the CBD and comprises complementarity determining regions (CDRs) as set forth in SEQ ID NOs: 15-17; 19-21; 23-25; 15 and 28-29; 31-33; 35-37; 39-41; 43-45; 47-49; 51-53; 55-57; 59-61; 63-65; 67-69; 71-73; 75-77; 79-81; 83-85; 87-89; 91, 80 and 93; 95-97; 99-101; 103-105; 107-109; 111-113; 115-117; 119-121; 123-125; 127-129; 131-133; 135-137; 139-141; 143-145; 147-149; 151-153; 15 and 156-157; 159-161; or 163-165 arranged in a sequential order from N to C on the nanobody.

According to some embodiments of the invention, the nanobody specifically binds the V-ATPase subunit c and comprises complementarity determining regions (CDRs) as set forth in SEQ ID NOs: 167-169; 171 and 168-169; 174 and 168-169; 167, 178 and 169; or 180-182 arranged in a sequential order from N to C on the nanobody.

According to some embodiments of the invention, the nanobody specifically binds the trehalase and comprises complementarity determining regions (CDRs) as set forth in SEQ ID NOs: 195-197; 199 and 196-197; 201-203; 201 and 205-206; 208-210; 212 and 209-210; 215-217; 219-221; 223-225; 227-229; 231, 228 and 232; 234-236; 238-240; 242-244; 246-248; 250-252; 254-256; 258-260; 588-590; 5889-589 and 592; or 595, 589 and 592 arranged in a sequential order from N to C on the nanobody.

According to some embodiments of the invention, the nanobody specifically binds the trehalase and comprises complementarity determining regions (CDRs) as set forth in SEQ ID NOs: 195-197; 199 and 196-197; 201-203; 201 and 205-206; 208-210; 212 and 209-210; 215-217; 219-221; 223-225; 227-229; 231, 228 and 232; 234-236; 238-240; 242-244; 246-248; 250-252; 254-256; or 258-260 arranged in a sequential order from N to C on the nanobody.

According to some embodiments of the invention, the nanobody specifically binds the trehalase and comprises complementarity determining regions (CDRs) as set forth in SEQ ID NOs: 201 and 205-206; 208-210; 212 and 209-210; 223-225; 238-240; 242-244; 246-248; 588-590; or 595, 589 and 592 arranged in a sequential order from N to C on the nanobody.

According to some embodiments of the invention, the nanobody specifically binds the cytochrome p450 monooxygenase and comprises complementarity determining regions (CDRs) as set forth in SEQ ID NOs: 262-264; 262-263 and 266; 262, 270 and 264; 274-276; 280 and 275-276; 282 and 275-276; 282, 275 and 285; 287, 220 and 289; 291, 220 and 289; 239-294 and 289; 296-298; 300-302; 304-306; 308-310; 312-314; or 316-318 arranged in a sequential order from N to C on the nanobody.

According to some embodiments of the invention, the nanobody specifically binds the cytochrome p450 monooxygenase and comprises complementarity determining regions (CDRs) as set forth in SEQ ID NOs: 262-264; 287, 220 and 289; 296-298; 300-302; 304-306; or 312-314 arranged in a sequential order from N to C on the nanobody.

According to some embodiments of the invention, the nanobody specifically binds the chitin deacetylase and comprises complementarity determining regions (CDRs) as set forth in SEQ ID NOs: 320-322; 320 and 324-325; 327, 321 and 328; 320-321 and 332; 208 and 336-337; 320, 324 and 339; 343-345; 347-349; 351-353; 351, 347 and 356; 358-360; 358-359 and 362; 365, 359 and 366; 368-370; 372 and 369-370; 374, 369 and 375; 377-379; 381-383; 385 and 382-383; 387-389; 387, 391 and 389; 394-396; 398-400; 402-404; or 406-408 arranged in a sequential order from N to C on the nanobody.

According to some embodiments of the invention, the nanobody specifically binds the chitin deacetylase and comprises complementarity determining regions (CDRs) as set forth in SEQ ID NOs: 320-322; 320 and 324-325; 372 and 369-370; 374, 369 and 375; 377-379; 387-389; or 398-400 arranged in a sequential order from N to C on the nanobody.

According to some embodiments of the invention, the nanobody specifically binds the chitin synthase and comprises complementarity determining regions (CDRs) as set forth in SEQ ID NOs: 412-414; 418-420; 422-424; 426-428; 426-427 and 430; 432-434; 436, 140 and 437; 439-441; 443-445; 447-449; 356 and 451-452; 454-456; 458-460; 462-464; 466-468; 470-472; 474-476; 478-480; 482, 479 and 483; 485-487; 489-491; 493-495; 426 and 497-498; 500-502; 504-506; or 508-510 arranged in a sequential order from N to C on the nanobody.

According to some embodiments of the invention, the nanobody specifically binds the chitin synthase and comprises complementarity determining regions (CDRs) as set forth in SEQ ID NOs: 412-414; 418-420; 426-428; 432-434; 443-445; 447-449; 466-468; 482, 479 and 483; 426 and 497-498; or 504-506 arranged in a sequential order from N to C on the nanobody.

According to some embodiments of the invention, the nanobody specifically binds the NPC1 sterol transporter and comprises complementarity determining regions (CDRs) as set forth in SEQ ID NOs: 512-514; 517 and 513-514; 521-523; 521-522 and 526; 531-533; 539-541; 485 and 540-541; 545 and 540-451; 547-549; 552-554; 556-558; 561-563; 565-567; 569-571; 573-575; 485 and 577-578; 580-582; or 584-586 arranged in a sequential order from N to C on the nanobody.

According to some embodiments of the invention, the nanobody specifically binds the NPC1 sterol transporter and comprises complementarity determining regions (CDRs) as set forth in SEQ ID NOs: 517 and 513-514; 485 and 540-541; 545 and 540-541; 556-558; 565-567; 573-575; 485 and 577-578; or 580-582 arranged in a sequential order from N to C on the nanobody.

According to an aspect of some embodiments of the present invention there is provided a polynucleotide encoding the nanobody or the composition.

According to an aspect of some embodiments of the present invention there is provided a nucleic acid construct comprising the polynucleotide and a cis-acting regulatory element for directing expression of the polynucleotide.

According to an aspect of some embodiments of the present invention there is provided a host cell comprising the nanobody or the composition or a polynucleotide or a nucleic acid construct encoding it.

According to an aspect of some embodiments of the present invention there is provided a method of producing an insect control nanobody, the method comprising expressing in a host cell the polynucleotide or the nucleic acid construct.

According to some embodiments of the invention, the method comprising isolating the nanobody.

According to an aspect of some embodiments of the present invention there is provided a method of insect control, the method comprising contacting the insect with the nanobody or the composition, a polynucleotide or a nucleic acid construct encoding same or a host cell expressing same.

According to some embodiments of the invention, the contacting comprises applying the nanobody or the composition directly to the insect.

According to some embodiments of the invention, the contacting comprises applying the nanobody or the composition to an organism or a surface, which may be in contact with the insect.

According to some embodiments of the invention, the nanobody or the composition is formulated as a liquid formulation.

According to some embodiments of the invention, the nanobody or the composition is formulated as a dry formulation.

According to an aspect of some embodiments of the present invention there is provided a plant comprising the nanobody or the composition or a polynucleotide or a nucleic acid construct encoding it.

According to some embodiments of the invention, the plant being a transgenic plant.

According to an aspect of some embodiments of the present invention there is provided a commodity product comprising the nanobody or the composition.

According to some embodiments of the invention, the commodity product is produced from the plant.

According to an aspect of some embodiments of the present invention there is provided a method of producing a nanobody, the method comprising immunizing a camelid with a recombinant or purified insect polypeptide selected from the group consisting of:
(i) a polypeptide comprising a chitin binding domain (CBD);
(ii) V-ATPase subunit c;
(iii) trehalase;
(iv) cytochrome p450 monooxygenase;
(v) chitin deacetylase;
(vi) chitin synthase; and
(vii) NPC1 sterol transporter,
wherein purity of the insect polypeptide in an insect polypeptide preparation is at least 80%.

According to some embodiments of the invention, the method comprising isolating the antibody following the immunizing.

According to some embodiments of the invention, the camelid is a llama.

According to some embodiments of the invention, the insect is selected from the group consisting of moth, stinkbug, hopper, beetle, aphid and honeybee.

According to some embodiments of the invention, the insect is a moth.

According to some embodiments of the invention, the moth is selected from the group consisting of Helicoverpa armigera and Spodoptera frugiperda.

According to some embodiments of the invention, the moth is Helicoverpa armigera.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph demonstrating specific binding of the generated anti-CBD nanobodies referred to herein as CB20501, CB20801, CB20901 and CB201101 to the target CBD antigen, as determined by ELISA. Each experiment was performed five times; data is presented as average±SE.

FIG. 2 is a graph demonstrating stability of the generated anti-CBD nanobodies CB20501, CB20801, CB20901 and CB201101 following 1 hour incubation with larvae gut juice harvested from H. armigera larvae guts as compared to PBS control, as determined by ELISA. Each experiment was performed three times; data is presented as average±SE.

FIG. 3 is a graph demonstrating stability of the generated anti-CBD nanobodies CB20501, CB20801, CB20901 and CB201101 following 1-3 hours incubation in field like conditions as compared to room temperature (RT) control, as determined by ELISA. Each treatment included 5 repetitions; data is presented as average±SE.

FIGS. 4A-C demonstrate the effect of the anti-CBD nanobodies CB20501, CB20801, CB20901 and CB20110 on H. armigera body length (FIG. 4A), weight (FIG. 4B) and mortality (FIG. 4C) following feeding with single dose of 40 μg of the indicated nanobody. The measurements were taken on day 7 and on day 7 or 14 for mortality. Each experiment included 12 larvae and had 3-5 repetitions with error bars indicating the standard error deviations. Shown the percentage of length of the larvae fed with the indicated nanobody relative to the length of the PBS fed larvae control (FIG. 4A), percentages of weight of the larvae fed with the indicated nanobody relative to the weight of the PBS fed larvae control (FIG. 4B) and the percentages of dead larvae following 7 and 14 days (FIG. 4C). Statistically significant differences are indicated by a (P>0.05) and b (P>0.01).

FIGS. 5A-C demonstrate presence of the generated anti-CBD nanobody CB20901 in insect gut following incubation with artificial food containing a fluorescently Cy3 labeled CB20901. FIG. 5A demonstrates fluorescently labeled nanobody tracked in larvae food following 48 hours and 6 days of incubation with larvae. The upper panel shows bright field images and the lower panel shows images reflecting the Cy3 signal of food fed to larvae. In addition, the yellow dashed line represents two different areas, the right side of the dashed line signals intact food, while the left side of the dashed line signals larvae feces. Differences in fluorescents intensity between food and feces represent uptake by larvae's gut. FIG. 5B demonstrates presence of the Cy5-labeled CB20901 nanobody throughout the intestinal track of live larvae following 48 hours of incubation with food containing the Cy3-labeled nanobody or following 36 hours of incubation with food containing the Cy3-labeled nanobody followed by 12 hours incubation with food not containing the nanobody, as indicated. FIG. 5C demonstrates presence of the Cy5-labeled CB20901 nanobody in larvae gut and specifically the peritrophic membrane following 36 hours of incubation with food containing the Cy3-labeled nanobody followed by 12 hours incubation with food not containing the nanobody as compared to larvae fed with food not containing the nanobody for 48 hours, as indicated. The upper panel shows bright field images and the lower panel shows images reflecting the Cy3 signal.

FIG. 6 is a graph demonstrating specific binding of the generated anti-ATPase V subunit c (referred to herein as “VAT”) nanobodies referred to herein as VAT0101A, VAT0101B, VAT0101C, VAT0101D, VAT0101E, VAT0101F, and VAT0101G to the target VAT antigen, as determined by ELISA. Each experiment was performed five times; data is presented as average±SE.

FIG. 7 is a graph demonstrating stability of the generated anti-VAT nanobodies VAT0101A and VAT0101G following 1 hour incubation with larvae gut juice harvested from H. armigera larvae guts as compared to PBS control, as determined by ELISA. Each experiment was performed three times; data is presented as average±SE.

FIG. 8 is a graph demonstrating stability of the generated anti-VAT nanobodies VAT0101A, VAT0101B, VAT0101C, VAT0101D, VAT0101E, VAT0101F, and VAT0101G following 1-3 hours incubation in field like conditions as compared to room temperature (RT) control, as determined by ELISA. Each treatment included three repetitions; data is presented as average±SE.

FIGS. 9A-C demonstrate the effect of the anti-VAT nanobodies VAT0101A, VAT0101B, VAT0101C, VAT0101D, VAT0101E, VAT0101F, VAT0101G and VAT0201 on H. armigera body length (FIG. 9A), weight (FIG. 9B) and mortality (FIG. 9C) following feeding with single dose of 40 μg of the indicated nanobody. The measurements were taken on day 7 or 14, as indicated. Each experiment included 12 larvae and had 3-5 repetitions with error bars indicating the standard error deviations. Shown the percentage of length of the larvae fed with the indicated nanobody relative to the length of the PBS fed larvae control (FIG. 9A), percentages of weight of the larvae fed with the indicated nanobody relative to the weight of the PBS fed larvae control (FIG. 9B) and the percentages of dead larvae following 7 and 14 days (FIG. 9C). Statistically significant differences are indicated by a (P>0.05) and b (P>0.01).

FIG. 10 is a graph demonstrating specific binding of the generated anti-trehalase (referred to herein as “TRH”) nanobodies, referred to herein as TRH1A, TRH12, TRH13 and TRH14, to the target TRH antigen, as determined by ELISA. Each experiment was performed five times; data is presented as average±SE.

FIGS. 11A-B are graphs demonstrating stability of the generated anti-TRH nanobodies referred to herein as TRH1A, TRH1D, TRH3B, TRH5A and TRH5C, following 6 hours or overnight incubation in field-like conditions (FIG. 11A), and following 6 hours or overnight incubation at 50° C. (FIG. 11B), as compared to room temperature (RT) control, as determined by ELISA. Each treatment included three repetitions; data is presented as average±SE.

FIG. 12A demonstrates the effect of the anti-TRH nanobodies TRH1A, TRH1D, TRH3B, TRH5A, TRH5B, TRH5C and TRH8 on mortality of H. armigera fed with artificial food containing the indicated nanobody at a final concentration of 0.4 mg/ml on days 0 and 7. The measurements were taken on days 7 and 14, as indicated. Each experiment included 12 larvae and had 3 repetitions, with error bars indicating the standard error. Statistically significant differences are indicated by a (P>0.05).

FIG. 12B demonstrates the effect of the anti-TRH3B nanobody on mortality of H. armigera fed with cotton leave discs applied with nanobody concentrations of 0.4 or 1 mg/ml, on day 0. Larvae were moved on days 4 and 7 to fresh leave discs treated with similar concentration of the indicated nanobody. The mortality measurements were taken on days 4, 7 and 10, as indicated. Each experiment included 12 larvae and had 2 repetitions with error bars indicating the standard error.

FIG. 13 is a graph demonstrating specific binding of the generated anti-cytochrome p450 monooxygenase (referred to herein as “P450”) nanobodies, referred to herein as P45001A, P45003A, P45004, P45005 and P45006, to the target P450 antigen, as determined by ELISA. Each experiment was performed five times; data is presented as average±SE.

FIG. 14 demonstrates the effect of the generated anti-P450 nanobody referred to herein as P45008 on mortality of H. armigera fed with an artificial diet containing the indicated nanobody at a final concentration of 0.4 mg/ml on days 0 and 7. The measurements were taken on days 7 and 14, as indicated. Each experiment included 12 larvae and had 3 repetitions with error bars indicating the standard error.

FIG. 15 is a graph demonstrating specific binding of the generated anti-chitin deacetylase (referred to herein as “CDA”) nanobodies, referred to herein as CDA01A, CDA05, CDA07A and CDA10, to the target CDA antigen, as determined by ELISA. Each experiment was performed five times; data is presented as average±SE.

FIGS. 16A-B are graphs demonstrating stability of the generated CDA7A nanobody following 6 hours or overnight incubation in field-like conditions (FIG. 16A), and following 6 hours or overnight incubation at 50° C. (FIG. 16B), as compared to room temperature (RT) control, as determined by ELISA. Each treatment included three repetitions; data is presented as average±SE.

FIG. 17A demonstrates the effect of the anti-CDA nanobodies referred to herein as CDA1B, CDA4B, CDA4C and CDA7A on mortality of H. armigera fed with an artificial diet containing the indicated nanobody at a final concentration of 0.4 mg/ml on days 0 and 7. The measurements were taken on day 7 and 14, as indicated. Each experiment included 12 larvae and had 3 repetitions with error bars indicating the standard error. Statistically significant differences are indicated by a (P>0.05).

FIG. 17B demonstrates the effect of the CDA7A nanobody on mortality of H. armigera fed with cotton leave discs applied with increasing nanobody concentrations (0.2, 0.4, 1, 2 or 4 mg/ml, as indicated) on day 0. Larvae were moved on days 4 and 7 to fresh leave discs treated with similar concentrations of the indicated nanobody. The mortality measurements were taken on day 4, 7 and 10, as indicated. Each experiment included 12 larvae and had 2 repetitions with error bars indicating the standard error.

FIG. 18 is a graph demonstrating specific binding of the generated anti-chitin synthase (referred to herein as “CHS”) nanobodies, referred to herein as CHS01A, CHS02A, CHS08, CHS13, CHS18, CHS22 and CHS24, to the target CHS antigen, as determined by ELISA. Each experiment was performed five times; data is presented as average±SE.

FIGS. 19A-B are graphs demonstrating stability of the generated anti-CHS nanobody referred to herein as CHS3A following 6 hours or overnight incubation in field-like conditions (FIG. 19A), and following 6 hours or overnight incubation at 50° C. (FIG. 19B), as compared to room temperature (RT) control, as determined by ELISA. Each treatment included three repetitions; data is presented as average±SE.

FIG. 20A demonstrates the effect of the generated anti-CHS nanobodies referred to herein as CHS3A, CHS4, and CHS7 on mortality of H. armigera fed with an artificial diet containing the indicated nanobody at a final concentration of 0.4 mg/ml on day 0 and 7. The measurements were taken on day 7 and 14, as indicated. Each experiment included 12 larvae and had 3 repetitions with error bars indicating the standard error. Statistically significant differences are indicated by a (P>0.05).

FIG. 20B demonstrates the effect of the CHS3A nanobody on mortality of H. armigera fed with cotton leave discs applied with increasing nanobody concentration (0.2, 0.4, 1, 2 and 4 mg/ml, as indicated) on day 0. Larvae were moved on days 4 and 7 to fresh leave discs treated with similar concentrations of the indicated nanobody. The mortality measurements were taken on days 4, 7 and 10, as indicated. Each experiment included 12 larvae and had 3 repetitions with error bars indicating the standard error. Statistically significant differences are indicated by a (P>0.05).

FIG. 21 is a graph demonstrating specific binding of the generated anti-NPC1 sterol transporter (referred to herein as “NPC”) nanobodies, referred to herein as NPC01C01, NPC04C01, NPC04D01, NPC07A01, NPC0901, NPC01101 and NPC01201, to the target NPC antigen, as determined by ELISA. Each experiment was performed five times; data is presented as average±SE.

FIG. 22 demonstrates the effect of the generated anti-NPC nanobodies referred to herein as NPC4C and NPC4D on mortality of H. armigera fed with an artificial diet containing the indicted nanobody at a final concentration of 0.4 mg/ml on days 0 and 7. The measurements were taken on days 7 or 14, as indicated. Each experiment included 12 larvae and had 3 repetitions with error bars indicating the standard error.

### DESCRIPTION OF SPECIFIC EMBODIMENTS OF THE INVENTION

The present invention, in some embodiments thereof, relates to insect control nanobodies and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Management of insect pests in the past 70 years has been achieved mainly through application of synthetic pesticides, most of them have a high potential for damaging the ecosystem, they are toxic to humans directly or through the food chain and their use is also impeded by development of genetic insect resistance. The most prominent biological solutions currently in use also have many disadvantages, including low efficiency, danger of violating the natural ecological balance and development genetic insect resistance.

Whilst reducing the present invention to practice, the present inventors have now developed nanobodies targeting several insect polypeptides having insect control activities. Consequently, specific embodiments of the present teachings suggest their use as insecticides.

Thus, according to a first aspect of the present invention, there is provided a nanobody which specifically binds to an insect polypeptide selected from the group consisting of:
(i) a polypeptide comprising a chitin binding domain (CBD), wherein said nanobody binds said CBD;
(ii) V-ATPase subunit c, wherein said nanobody comprises complementarity determining regions (CDRs) as set forth in SEQ ID NOs: 167-169; 171 and 168-169; 174 and 168-169; 167, 178 and 169; or 180-182 arranged in a sequential order from N to C on said nanobody;
(iii) trehalase;
(iv) cytochrome p450 monooxygenase;
(v) chitin deacetylase;
(vi) chitin synthase; and
(vii) NPC1 sterol transporter,
wherein binding of said nanobody to said insect polypeptide confers an insect control activity to said nanobody.

According to an additional or an alternative aspect of the present invention, there is provided a composition comprising a nanobody, which specifically binds to an insect polypeptide selected from the group consisting of:
(i) a polypeptide comprising a chitin binding domain (CBD), wherein said nanobody binds said CBD;
(ii) V-ATPase subunit c, wherein said nanobody comprises complementarity determining regions (CDRs) as set forth in SEQ ID NOs: 167-169; 171 and 168-169; 174 and 168-169; 167, 178 and 169; 180-182; 187-189; or 191-193 arranged in a sequential order from N to C on said nanobody;
(iii) trehalase;
(iv) cytochrome p450 monooxygenase;
(v) chitin deacetylase;
(vi) chitin synthase; and
(vii) NPC1 sterol transporter,
and a toxin moiety having an insect control activity.

As used herein, the term “nanobody” refers to a single-domain antigen binding fragment.

According to a specific embodiment, the nanobody is a single variable domain derived from naturally occurring heavy chain of an antibody. Nanobodies are usually derived from heavy chain only antibodies (devoid of light chains) seen in camelids (Hamers-Casterman et al., 1993, Nature 363:446-448; Desmyter et al., 1996, Nat. Struct. Biol. 803-811) and consequently are often referred to as VHH antibody or VHH sequence. Camelids comprise old world camelids (Camelus bactrianus and Camelus dromedarius) and new world camelids (for example, Lama paccos, Lama glama, Lama guanicoe and Lama vicugna). Non-limiting examples of camelids include dromedary camels, Bactrian camels, wild Bactrian camels, llamas, alpacas, vicuñas, and guanacos. According to specific embodiments, the camelid is a llama.

NANOBODY® and NANOBODIES® are registered trademarks of Ablynx NV (Belgium).

For a further description of VHH's or Nanobodies, reference is made to the book “Single domain antibodies,” Methods in Molecular Biology, Eds. Saerens and Muyldermans, 2012, Vol. 911, in particular to the Chapter by Vincke and Muyldermans (2012), as well as to a non-limiting list of patent applications, which are mentioned as general background art, and include: WO 94/04678, WO 95/04079, WO 96/34103 of the Vrije Universiteit Brussel; WO 94/25591, WO 99/37681, WO 00/40968, WO 00/43507, WO 00/65057, WO 01/40310, WO 01/44301, EP 1 134 231 and WO 02/48193 of Unilever; WO 97/49805, WO 01/21817, WO 03/035694, WO 03/054016 and WO 03/055527 of the Vlaams Instituut voor Biotechnologie (VIB); WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As will be known by the person skilled in the art, the nanobodies are particularly characterized by the presence of one or more Camelidae “hallmark residues” in one or more of the framework sequences (according to Kabat numbering), as described, for example, in WO 08/020079, on page 75, Table A-3, incorporated herein by reference.

According to a specific embodiment, the nanobody refers to an intact molecule (i.e. comprising 4 frameworks regions and 3 complementarity-determining regions) or a functional fragment thereof capable of binding to an epitope of the antigen to which the intact molecule binds.

As used herein, the term “epitope” refers to any antigenic determinant on an antigen to which the paratope of a nanobody binds. Epitopic determinants usually consist of chemically active surface groupings of molecules such as amino acids or carbohydrate side chains and usually have specific three dimensional structural characteristics, as well as specific charge characteristics.

According to specific embodiments, the nanobody is a whole or intact nanobody.

According to specific embodiments, the nanobody is a nanobody fragment.

According to specific embodiments, the size of the nanobody is 5-30 kDa, 10-30 Kda or 10-20 Kda.

According to specific embodiments, the size of the nanobody is about 15 kDa.

The term “nanobody” also encompasses natural or synthetic analogs, homologous, mutants and variants of a nanobody.

Generally, intact nanobodies comprise three complementarity-determining region (CDRs) (CDR1; CDR2; and CDR3).

As used herein, the terms “complementarity-determining region” or “CDR” are used interchangeably to refer to the antigen binding regions found within the variable region of the heavy chain polypeptide. The identity of the amino acid residues in a particular nanobody that make up a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), as applied to VHH domains from Camelids in the article of Riechmann and Muyldermans (1999), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www (dot) bioinf-org (dot) uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “CDRs” may refer to CDRs defined by any approach known in the art, including combinations of approaches.

According to specific embodiments, the CDR is as defined by Kabat et al. as applied to VHH domains from Camelids in the article of Riechmann and Muyldermans (1999).

The nanobody may be mono-specific (capable of recognizing one epitope or protein), bi-specific (capable of binding two epitopes or proteins) or multi-specific (capable of recognizing multiple epitopes or proteins).

According to specific embodiments, the nanobody is a mono-specific nanobody.

According to specific embodiments, the nanobody is a multi-specific e.g. bi-specific, tri-specific, tetra-specific.

According to specific embodiments, the nanobody is a bi-specific nanobody. Methods of generating bi-specific nanobodies are known in the art and disclosed e.g., in Deffar K, Shi H, Li L, Wang X, Zhu X (2009) Afr J Biotechnol 8(12):2645-2652); and Zhu, Y. et al. (2017). Scientific reports, 7(1), 2602; the contents of which are fully incorporated herein by reference.

The nanobodies disclosed herein specifically bind an insect polypeptide described herein. Preferably, the nanobody specifically binds at least one epitope of an insect polypeptide described herein.

Assays for testing binding are well known in the art and include, but not limited to ELISA, radioimmunoassays (RIA), flow cytometry, BiaCore, bio-layer interferometry Blitz® assay, HPLC.

According to specific embodiments, the nanobody binds the insect polypeptide with a Kd≤10⁻⁶ M, ≤10⁻⁷ M, ≤10⁻⁸ M, ≤10⁻⁹ M, each possibility represents a separate embodiment of the present invention.

According to specific embodiments, the nanobody is capable of binding to the insect polypeptide under open field or controlled conditions, e.g., greenhouse.

The term “insect” is used herein in the broad popular sense and includes all species of the superphylum Panarthropoda (classification Systema Naturae, Brands, S. J. (comp.) 1989-2005. Systema Naturae 2000. Amsterdam, The Netherlands, [www(dot)sn2000(dot)taxonomy(dot)nl/]), including the phyla Arthropoda, Tardigrada and Onychophora; and includes all the different phases of the life cycle, such as, but not limited to eggs, larvae, nymphs, pupae and adults. According to specific embodiments, the insect belongs to the phylum Arthropoda (including, but not limited to the orders Archaeognatha, Thysanura, Paleoptera and Neoptera, also ticks, mites and spiders), even more preferably to the epiclass Hexapoda, most preferably to the class Insecta. According to specific embodiments, the insect belongs to the order Lepidoptera. Non-limiting examples of insects include bedbugs, house flies, moths, beetles, grasshoppers, caterpillars, aphids, mosquitos, fleas, horseflies, hornets, cockroaches and ants, such as, but not limited to:
from the order Lepidoptera, for example: Acleris spp., Adoxophyes spp., Agrotis spp., Alabama argillacea, Amyelois spp., Anticarsia gemmatalis, Archips spp., Argyrotaenia spp., Autographa spp., Busseola fusca, Cadra cautella, Carposina nipponensis, Chilo suppressalis, Chilo spp., Choristoneura conflictana, Choristoneura fumiferana, Choristoneura occidentalis, Choristoneura rosaceana, Choristoneura spp., Clysia ambiguella, Cnaphalocrocis spp., Cnephasia spp., Cochylis spp., Coleophora spp., Crocidolomia binotalis, Cryptophlebia leucotreta, Cydalima perspectalis, Cydia inopinata, Cydia spp., Diatraea spp., Diparopsis castanea, Earias spp., Ephestia spp., Eucosma spp., Eupoecilia ambiguella, Euproctis spp., Euxoa spp., Grapholita prunivora, Grapholita spp., Hedya nubiferanal, Helicoverpa armigera, Helicoverpa zea, Helicoverpa spp., Heliothis spp., Hellula undalis, Hyphantria cunea, Keiferia lycopersicella, Leucoptera scitella, Lithocolletis spp., Lobesia botrana, Lymantria spp., Lyonetia spp., Malacosoma spp., Mamestra brassicae, Manduca sexta, Numonia pyrivorella, Operophtera spp., Opogona sacchari, Ostrinia nubilalis, Pammene spp., Pandemis spp., Panolis flammea, Paysandisia archon, Pectinophora gossypiella, Phthorimaea operculella, Phyllonorycter spp., Pieris rapae, Pieris spp., Platynota rostrana, Plutella xylostella, Prays spp., Scirpophaga spp., Sesamia spp., Sesia spp., Sparganothis spp., Spodoptera dolichos, Spodoptera eridania, Spodoptera exigua, Spodoptera frugiperda, Spodoptera littoralis, Spodoptera litura, Spodoptera spp., Synanthedon spp., Tecia solanivora, Thaumatotibia leucotreta, Thaumetopoea processionea, Thaumetopoea spp., Tortrix spp., Trichoplusia ni, and Yponomeuta spp.; from the order Coleoptera, for example, Agrilus anxius, Agrilus planipennis, Agriotes spp., Anomala orientalis, Anoplophora chinensis, Anoplophora glabripennis, Anoplophora spp., Anthonomus bisignifer, Anthonomus eugenii, Anthonomus grandis, Anthonomus quadrigibbus, Anthonomus signatus, Anthonomus spp., Apriona spp., Arrhenodes minutus, Atomaria linearis, Chaetocnema tibialis, Conotrachelus nenuphar, Cosmopolites spp., Curculio spp., Dendroctonus micans, Dendrolimus sibiricus, Dermestes spp., Diabrotica virgifera, Diabrotica virgifera zeae, Diabrotica virgifera, Diabrotica balteata, Diabrotica barberi, Diabrotica undecimpunctata howardi, Diabrotica undecimpunctata tenella, Diabrotica undecimpunctata, Diabrotica undecimpunctata, Diabrotica spp., Epilachna varivestis, Epilachna spp., Epitrix cucumeris, Eremnus cerealis, Eremnus spp., Gonipterus scutellatus, Ips amitinus, Ips cembrae, Ips duplicatus, Ips sexdentatus, Ips typographus, Ips spp., Leptinotarsa decemlineata, Leptinotarsa juncta, Leptinotarsa texana, Lissorhoptrus spp., Listronotus bonariensis, Melolontha spp., Monochamus spp., Naupactus leucoloma, Oryzaephilus spp., Otiorhynchus spp., Phlyctinus spp., Pissodes nemorensis, Pissodes strobi, Pissodes terminalis, Pissodes spp., Popilia japonica, Popilia spp., Premnotrypes spp., Pseudopityophthorus minutissimus, Pseudopityophthorus pruinosus, Psylliodes spp., Rhizopertha spp., Rhynchophorus ferrugineus, Rhynchophorus palmarum, Scarabaeidae family spp., Scolytidae family spp., Sitophilus spp., Sitotroga spp., Sternochetus mangiferae, Tenebrio spp., Tribolium castaneum, Tribolium spp, and Trogoderma spp.;
from the order Orthoptera, for example, Gryllotalpa spp., Locusta spp., and Schistocerca spp.;
from the order Blattaria, from example, Blatta spp., Blattella spp., Periplaneta spp., and Leucophaea maderae,
from the order Isoptera, for example, Coptotermes spp, and Reticulitermes spp.;
from the order Psocoptera, for example, Liposcelis spp.;
from the order Phthiraptera, suborder Anoplura, for example, Haematopinus spp., Linognathus spp., and Pediculus spp., and Trichodectes spp.;
from the order Phthiraptera, suborder Ischnocera, for example, Damalinia spp.;
from the order Thysanoptera, for example, Frankliniella occidentalis, Frankliniella platensis, Frankliniella spp., Hercinothrips spp., Taeniothrips spp., Thrips palmi, Thrips tabaci, Scirtothrips aurantii, Scirtothrips citri, Scirtothrips dorsalis, and Scirtothrips spp.;
from the order Hemiptera, suborder Heteroptera, for example, Cimex spp., Distantiella theobroma, Dysdercus spp., Euschistus spp., Eurygaster spp., Leptocorisa spp., Nezara spp., Piesma spp., Rhodnius spp., Sahlbergella singularis, Scotinophara spp., Triatoma spp., Miridae family spp, such as Lygus hesperus and Lygus lineoloris, Lygaeidae family spp, such as Blissus leucopterus, and Pentatomidae family spp.;
from the order Hemiptera, suborder Sternorrhyncha, for example, Aleurocanthus spiniferus, Aleurocanthus woglumi, Aleurocanthus spp., Aleurothrixus floccosus, Aleyrodes brassicae, Aonidella citrina, Aonidiella spp., Aphididae family spp., Acyrthosiphon spp., Aphis fabae, Aphis glycines, Aphis gossypii, Aphis spp., Aspidiotus spp., Bemisia tabaci, Ceroplastes spp., Chrysomphalus aonidium, Chrysomphalus dictyospermi, Coccus hesperidum, Daktulosphaira vitifoliae, Diaphorina citri, Eriosoma larigerum, Gascardia spp., Lacanium corni, Lepidosaphes spp., Lopholeucaspis japonica, Macrosiphus spp., Margarodes prieskaensis, Margarodes vitis, Margarodes vredendalensis, Myzus persicae, Myzus spp., Parasaissetia nigra, Pemphigus spp., Phylloxera spp., Planococcus spp., Pseudaulacaspis spp., Pseudococcus spp., Psylla spp., Pulvinaria aethiopica, Quadraspidiotus spp., Rhopalosiphum spp., Ripersiella hibisci, Saissetia spp., Schizaphis spp., Sitobion spp., Toxoptera citricida, Trialeurodes vaporariorum, Trioza erytreae, and Unaspis citri;
from the order Hemiptera, suborder Auchenorrhyncha, for example, Circulifer haematoceps, Circulifer tenellus, Draeculacephala minerva, Empoasca spp., Erythroneura spp., Graphocephala atropunctata, Hishimonus phycitis, Myndus crudus, Laodelphax spp., Nephotettix spp., Nilaparvata spp., Scaphoideus luteolus, Scaphoideus spp., and Xyphon fulgida;
from the order Hymenoptera, for example, Acromyrmex, Atta spp., Cephus spp., Diprionidae family spp, such as Diprion spp, and Gilpinia polytoma, Hoplocampa spp., Lasius spp., Monomorium pharaonis, Neodiprion spp., Formicidae family spp. such as Solenopsis spp., and Vespa spp.;
from the order Diptera, for example, Aedes albopictus, Aedes cinereus, Aedes polynesiensis, Aedes spp., Amauromyza maculosa, Anastrepha fraterculus, Anastrepha ludens, Anastrepha obliqua, Anastrepha suspensa, Anastrepha spp., Anopheles gambiae, Anopheles spp., Aschistonyx eppoi, Atherigona soccata, Bactrocera spp., Bibio hortulanus, Calliphora erythrocephala, Cephalcia lariciphila, Ceratitis rosa, Ceratitis spp., Chrysomyia spp., Culex spp., Cuterebra spp., Dacus spp., Drosophila melanogaster, Dryocosmus kuriphilus, Euphranta canadensis, Euphranta japonica, Fannia spp., Gastrophilus spp., Gilpinia hercyniae, Glossina spp., Hypoderma spp., Hippobosca spp., Liriomyza bryoniae, Liriomyza huidobrensis, Liriomyza sativae, Liriomyza trifolii, Liriomyza spp., Lucilia spp., Melanagromyza spp., Musca spp., Oestrus spp., Orseolia spp., Oscinella frit, Pardalaspis cyanescens, Pardalaspis quinaria, Pegomyia hyoscyami, Phorbia spp., Rhagoletis pomonella, Rhagoletis spp., Sciara spp., Stomoxys spp., Tabanus spp., and Tipula spp.;
from the order Siphonaptera, for example, Ceratophyllus spp, and Xenopsylla cheopis; and
from the infraclass Thysanura, order Zygentoma, for example, Lepisma saccharina.

According to specific embodiments, the insect is considered as a pest. As used herein, the term “pest” refers to an agricultural pest organisms, including but not limited to aphids, grasshoppers, caterpillars, beetles, moths, stinkbugs, thrips, white flied, household pest organisms, such as cockroaches, ants, wasps, flies, house crickets, bed bugs, wood worms, mealworm beetles, earwigs, silverfish, termites, blood-feeding pest insects such as mosquitos, fleas and lice etc.

According to specific embodiments, the insect is an agricultural pest organism.

According to specific embodiments, the insect is selected from the group consisting of moth, stinkbug, hopper, beetle, aphid and honeybee.

According to specific embodiments, the insect is a moth.

According to specific embodiments, the insect is a Noctuid.

Non-limiting Examples of moths include Helicoverpa armigera, Cydia pomonella and Spodoptera frugiperda.

According to specific embodiments, the moth is Helicoverpa armigera and/or, Spodoptera frugiperda.

According to specific embodiments, the moth is Helicoverpa armigera.

According to specific embodiments, the insect is selected from the group consisting of Helicoverpa armigera, Spodoptera frugiperda, Nezara viridula, Nilaraparvata lugens, Aphis gossypii, Cydia pomonella, Leaf Feeding beetles (e.g. Leptinotarsa decemlineata or Phaedon cochleariae;) and honeybee.

According to specific embodiments, the nanobody binds an insect polypeptide selected from the group consisting of:
(i) a polypeptide comprising a chitin binding domain (CBD), wherein said nanobody binds said CBD;
(ii) V-ATPase subunit c, wherein said nanobody comprises complementarity determining regions (CDRs) as set forth in SEQ ID NOs: 167-169; 171 and 168-169; 174 and 168-169; 167, 178 and 169; 180-182; 187-189; or 191-193 arranged in a sequential order from N to C on said nanobody;
(iii) trehalase;
(iv) cytochrome p450 monooxygenase;
(v) chitin deacetylase;
(vi) chitin synthase; and
(vii) NPC1 sterol transporter.

As used herein, the term “specifically bind” refers to the ability of the nanobody to bind a target insect polypeptide in a physiological environment e.g., in the insect under physiological conditions at a higher affinity compared to other polypeptides in said environment.

According to specific embodiments, the nanobody binds an insect polypeptide with no cross reactivity with non-insect (e.g. plant, human) polypeptides.

According to specific embodiments, the nanobody specifically binds one of the insect polypeptides (i)-(vii) with no cross reactivity with the other insect polypeptides.

According to specific embodiments, the nanobody binds at least two of the insect polypeptides (i)-(vii).e.g., by way of multi-specificities such as a bi-specific nanobody.

According to specific embodiments, the nanobody is a nanobody combination comprising at least two nanobodies each binding distinct insect polypeptides selected from the group consisting of insect polypeptides (i)-(vii).

According to specific embodiments, the nanobody or the nanobody combination binds (i)+(ii), (i)+(iii), (i)+(iv), (i)+(v), (i)+(vi), (i)+(vii), (ii)+(iii), (ii)+(iv), (ii)+(v), (ii)+(vi), (ii)+(vii), (iii)+(iv), (iii)+(v), (iii)+(vi), (iii)+(vii), (iv)+(v), (iv)+(vi), (iv)+(vii), (v)+(vi), (v)+(vii), (vi)+(vii).

According to specific embodiments, the nanobody binds a polypeptide comprising a chitin binding domain (CBD), wherein said nanobody binds said CBD.

As used herein the term “chitin binding domain (CBD)” refers to an insect amino acid domain capable of binding an insect chitin (a linear polysaccharide consisting of (1->4)-beta-linked D-glucosamine residues, most of which are N-acetylated), or to antigenic fragments thereof capable of eliciting a specific immune response against said fragment. Non-limiting examples of CBDs include type 2 chitin-binding domain (ChtBD2) and Rebers and Riddiform consensus sequence. According to other specific embodiments, the CBD comprises the Rebers and Riddiford consensus sequence.

As used herein the term “Rebers and Riddiford consensus sequence” refers to a consensus sequence as described by Pfam database entry pfam00379 (see Rebers and Riddiford, 1988; Anderson, 2010; Karouzou et al., 2007; Willis, 2010, the content of which are incorporated herein by reference in their entirety).

According to specific embodiments, the CBD is type 2 chitin-binding domain (ChtBD2). As used herein, the term “type 2 chitin-binding domain (ChtBD2)”, also referred to as the peritrophin A domain refers to a an amino acid sequence having six cysteines that form three disulphide bridges. Such CBDs are known in the art and disclosed e.g. in Tetreau, Guillaume, et al. Insect biochemistry and molecular biology 62 (2015): 127-141). According to specific embodiments, the ChtBD2 has a consensus sequence of: CX11-30CX5-6CX9-24CX12-17CX6-12C (SEQ ID NO: 409).

According to specific embodiments, the ChtBD2 comprises an amino acid sequence having at least 60%, at least 70%, at least 75%, at least 80%, at least 85%, at least 90%, at least 95%, at least 96%, at least 97%, at least 98%, at least 99%, or 100% identity to an amino acid sequence selected form the group consisting of SEQ ID NO: 3-7.

According to specific embodiments, the ChtBD2 comprises an amino acid sequence having at least 60%, at least 70%, at least 75%, at least 80%, at least 85%, at least 90%, at least 95%, at least 96%, at least 97%, at least 98%, at least 99%, or 100% identity to an amino acid sequence provided in GeneBank Accession No XP_021181774 (SEQ ID NO: 410).

Sequence identity or homology can be determined using any protein or nucleic acid sequence alignment algorithm such as Blast, ClustalW, and MUSCLE.

According to specific embodiments, the ChtBD2 comprises an amino acid sequence selected form the group consisting of SEQ ID NO: 3-7.

Non-limiting examples of polypeptides comprising ChtBD2 include Peritrophic Matrix Proteins (PMPs) such as TcPMP5-B, HaIIM86; and Cuticle Proteins Analogous to Peritrophins (CPAPs) such as BmCPAP3-A1, BmCPAP3-A2, BmCPAP3-B, BmCPAP3-C, BmCPAP3-D1 and BmCPAP3-D2.

According to specific embodiments, the polypeptide comprising ChtBD2 is a PMP protein.

According to specific embodiments, the polypeptide comprising ChtBD2 is TcPMP5-B (PMP5-B of Tribolium castaneum).

According to specific embodiments, the polypeptide comprising ChtBD2 comprises an amino acid sequence selected from the group consisting of SEQ ID NO: 1-2.

According to specific embodiments, the polypeptide comprising ChtBD2 consists of an amino acid sequence selected from the group consisting of SEQ ID NO: 1-2.

Non-limiting examples of nanobodies specifically binding ChtBD2 and their respective CDRs are shown in Tables 2A-B hereinbelow.

According to specific embodiments, the nanobody specifically binds ChtBD2 and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 15-17; 19-21; 23-25; 15 and 28-29; 31-33; 35-37; 39-41; 43-45; 47-49; 51-53; 55-57; 59-61; 63-65; 67-69; 71-73; 75-77; 79-81; 83-85; 87-89; 91, 80 and 93; 95-97; 99-101; 103-105; 107-109; 111-113; 115-117; 119-121; 123-125; 127-129; 131-133; 135-137; 139-141; 143-145; 147-149; 151-153; 15 and 156-157; 159-161; or 163-165 arranged in a sequential order from N to C on said nanobody.

According to specific embodiments, the nanobody specifically binds ChtBD2 and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 15-17; 19-21; 23-25; or 15 and 28-29 arranged in a sequential order from N to C on said nanobody.

According to specific embodiments, the nanobody specifically binds ChtBD2 and comprises CDRs as set forth in SEQ ID NOs: 15-17; 19-21; 23-25; 15 and 28-29; 31-33; 35-37; 39-41; 43-45; 47-49; 51-53; 55-57; 59-61; 63-65; 67-69; 71-73; 75-77; 79-81; 83-85; 87-89; 91, 80 and 93; 95-97; 99-101; 103-105; 107-109; 111-113; 115-117; 119-121; 123-125; 127-129; 131-133; 135-137; 139-141; 143-145; 147-149; 151-153; 15 and 156-157; 159-161; or 163-165 arranged in a sequential order from N to C on said nanobody.

According to specific embodiments, the nanobody specifically binds ChtBD2 and comprises CDRs as set forth in SEQ ID NOs: 15-17; 19-21; 23-25; or 15 and 28-29 arranged in a sequential order from N to C on said nanobody.

According to specific embodiments, the nanobody specifically binds ChtBD2 and comprises CDRs as set forth in SEQ ID NOs: 15-17 arranged in a sequential order from N to Con said nanobody.

According to specific embodiments, the nanobody specifically binds ChtBD2 and comprises CDRs as set forth in SEQ ID NOs: 19-21 arranged in a sequential order from N to C on said nanobody.

According to specific embodiments, the nanobody specifically binds ChtBD2 and comprises CDRs as set forth in SEQ ID NOs: 23-25 arranged in a sequential order from N to C on said nanobody.

According to specific embodiments, the nanobody specifically binds ChtBD2 and comprises CDRs as set forth in SEQ ID NOs: 15 and 28-29 arranged in a sequential order from N to C on said nanobody.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158 and 162 each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 14, 18, 22 and 26.

According to specific embodiments, the nanobody comprises an amino acid sequence selected from the group consisting of SEQ ID NOs: 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158 and 162.

According to specific embodiments, the nanobody comprises an amino acid sequence selected from the group consisting of SEQ ID NOs: 14, 18, 22 and 26.

According to specific embodiments, the nanobody comprises SEQ ID NO: 14.

According to specific embodiments, the nanobody comprises SEQ ID NO: 18.

According to specific embodiments, the nanobody comprises SEQ ID NO: 22.

According to specific embodiments, the nanobody comprises SEQ ID NO: 26.

According to specific embodiments, the nanobody consists of an amino acid sequence selected from the group consisting of SEQ ID NOs: 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138, 142, 146, 150, 154, 158 and 162.

According to specific embodiments, the nanobody consists of SEQ ID NOs: 14, 18, 22 or 26.

According to specific embodiments, the nanobody consists of SEQ ID NO: 14.

According to specific embodiments, the nanobody consists of SEQ ID NO: 18.

According to specific embodiments, the nanobody consists of SEQ ID NO: 22.

According to specific embodiments, the nanobody consists of SEQ ID NO: 26.

According to specific embodiments, the nanobody binds a V-ATPase subunit c.

Non-limiting examples of nanobodies specifically binding V-ATPase subunit c and their respective CDRs are shown in Tables 3A-B hereinbelow.

According to specific embodiments, the nanobody binds a V-ATPase subunit C, wherein said nanobody comprises complementarity determining regions (CDRs) as set forth in SEQ ID NOs: 167-169; 171 and 168-169; 174 and 168-169; 167, 178 and 169; 180-182; 187-189; or 191-193 arranged in a sequential order from N to C on said nanobody.

According to specific embodiments, the nanobody binds a V-ATPase subunit C, wherein said nanobody comprises complementarity determining regions (CDRs) as set forth in SEQ ID NOs: 167-169; 171 and 168-169; 174 and 168-169; 167, 178 and 169; or 180-182 arranged in a sequential order from N to C on said nanobody.

According to specific embodiments, the nanobody binds a V-ATPase subunit c, wherein said nanobody comprises complementarity determining regions (CDRs) as set forth in SEQ ID NOs: 167-169; or 167, 178 and 169 arranged in a sequential order from N to C on said nanobody.

According to specific embodiments, the nanobody binds a V-ATPase subunit C, wherein said nanobody comprises complementarity determining regions (CDRs) as set forth in SEQ ID NOs: 167-169 arranged in a sequential order from N to C on said nanobody.

According to specific embodiments, the nanobody binds a V-ATPase subunit C, wherein said nanobody comprises complementarity determining regions (CDRs) as set forth in SEQ ID NOs: 171 and 168-169 arranged in a sequential order from N to C on said nanobody.

According to specific embodiments, the nanobody binds a V-ATPase subunit c, wherein said nanobody comprises complementarity determining regions (CDRs) as set forth in SEQ ID NOs: 174 and 168-169 arranged in a sequential order from N to C on said nanobody.

According to specific embodiments, the nanobody binds a V-ATPase subunit c, wherein said nanobody comprises complementarity determining regions (CDRs) as set forth in SEQ ID NOs: 167, 178 and 169 arranged in a sequential order from N to C on said nanobody.

According to specific embodiments, the nanobody binds a V-ATPase subunit c, wherein said nanobody comprises complementarity determining regions (CDRs) as set forth in SEQ ID NOs: 180-182 arranged in a sequential order from N to C on said nanobody.

As used herein, the term “V-ATPase subunit c” refer to the amino acid sequence of the insect enzyme Pfam PF03223, an enzyme that catalyzes proton electrochemical potential gradient, or to antigenic fragments thereof capable of eliciting a specific immune response against said fragment.

According to specific embodiments, the V-ATPase subunit c is the Helicoverpa armigera V-ATPase subunit c, such as provided in GeneBank Accession No. XP_021198264.

According to specific embodiments, the V-ATPase subunit c comprises SEQ ID NO: 8.

According to specific embodiments, the V-ATPase subunit c consists of SEQ ID NO: 8.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 166, 170, 172, 173, 175, 176, 177, 179, 183, 185, 186 and 190, each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 166, 170, 172, 173, 175, 176, 177 and 179.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 176 and 177.

According to specific embodiments, the nanobody comprises an amino acid sequence selected from the group consisting of SEQ ID NOs: 166, 170, 172, 173, 175, 176, 177, 179, 183, 185, 186 and 190.

According to specific embodiments, the nanobody comprises an amino acid sequence selected from the group consisting of SEQ ID NOs: 166, 170, 172, 173, 175, 176, 177 and 179.

According to specific embodiments, the nanobody comprises an amino acid sequence selected from the group consisting of SEQ ID NOs: 176 and 177.

According to specific embodiments, the nanobody comprises SEQ ID NO: 166.

According to specific embodiments, the nanobody comprises SEQ ID NO: 170.

According to specific embodiments, the nanobody comprises SEQ ID NO: 172.

According to specific embodiments, the nanobody comprises SEQ ID NO: 173.

According to specific embodiments, the nanobody comprises SEQ ID NO: 175.

According to specific embodiments, the nanobody comprises SEQ ID NO: 176.

According to specific embodiments, the nanobody comprises SEQ ID NO: 177.

According to specific embodiments, the nanobody comprises SEQ ID NO: 179.

According to specific embodiments, the nanobody consists of an amino acid sequence selected from the group consisting of SEQ ID NOs: 166, 170, 172, 173, 175, 176, 177, 179, 183, 185, 186 and 190.

According to specific embodiments, the nanobody consists of an amino acid sequence selected from the group consisting of SEQ ID NOs: 166, 170, 172, 173, 175, 176, 177 and 179.

According to specific embodiments, the nanobody consists of an amino acid sequence selected from the group consisting of SEQ ID NOs: 176 and 177.

According to specific embodiments, the nanobody consists of SEQ ID NO: 166.

According to specific embodiments, the nanobody consists of SEQ ID NO: 170.

According to specific embodiments, the nanobody consists of SEQ ID NO: 172.

According to specific embodiments, the nanobody consists of SEQ ID NO: 173.

According to specific embodiments, the nanobody consists of SEQ ID NO: 175.

According to specific embodiments, the nanobody consists of SEQ ID NO: 176.

According to specific embodiments, the nanobody consists of SEQ ID NO: 177.

According to specific embodiments, the nanobody consists of SEQ ID NO: 179.

According to specific embodiments, the nanobody binds a trehalase.

As used herein, the term “trehalase” refer to the amino acid sequence of the insect enzyme E.C. No. 3.2.1.2, an enzyme which catalyzes the conversion of trehalose to glucose, or to antigenic fragments thereof capable of eliciting a specific immune response against said fragment.

According to specific embodiments, the trehalase is the Helicoverpa armigera trehalase, such as provided in GeneBank Accession No. AJK29979.

According to specific embodiments, the trehalase comprises SEQ ID NO: 10.

According to specific embodiments, the trehalase consists of SEQ ID NO: 10.

Non-limiting examples of nanobodies specifically binding trehalase and their respective CDRs are shown in Table 4 hereinbelow.

According to specific embodiments, the nanobody specifically binds trehalase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 195-197; 199 and 196-197; 201-203; 201 and 205-206; 208-210; 212 and 209-210; 215-217; 219-221; 223-225; 227-229; 231, 228 and 232; 234-236; 238-240; 242-244; 246-248; 250-252; 254-256; 258-260; 588-590; 5889-589 and 592; or 595, 589 and 592 arranged in a sequential order from N to C on said nanobody.

According to specific embodiments, the nanobody specifically binds trehalase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 195-197; 199 and 196-197; 201-203; 201 and 205-206; 208-210; 212 and 209-210; 215-217; 219-221; 223-225; 227-229; 231, 228 and 232; 234-236; 238-240; 242-244; 246-248; 250-252; 254-256; or 258-260 arranged in a sequential order from N to C on said nanobody.

According to specific embodiments, the nanobody specifically binds trehalase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 201 and 205-206; 208-210; 212 and 209-210; 223-225; 238-240; 242-244; 246-248; 588-590; or 595, 589 and 592 arranged in a sequential order from N to C on said nanobody.

According to specific embodiments, the nanobody specifically binds trehalase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 201 and 205-206; 208-210; 212 and 209-210; 223-225; 588-590; or 595, 589 and 592; arranged in a sequential order from N to C on said nanobody.

According to specific embodiments, the nanobody specifically binds trehalase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 201 and 205-206; 208-210; 223-225; or 588-590; arranged in a sequential order from N to C on said nanobody.

According to specific embodiments, the nanobody specifically binds trehalase and comprises CDRs as set forth in SEQ ID NOs: 195-197; 199 and 196-197; 201-203; 201 and 205-206; 208-210; 212 and 209-210; 215-217; 219-221; 223-225; 227-229; 231, 228 and 232; 234-236; 238-240; 242-244; 246-248; 250-252; 254-256; 258-260; 588-590; 5889-589 and 592; or 595, 589 and 592 arranged in a sequential order from N to C on said nanobody.

According to specific embodiments, the nanobody specifically binds trehalase and comprises CDRs as set forth in SEQ ID NOs: 195-197; 199 and 196-197; 201-203; 201 and 205-206; 208-210; 212 and 209-210; 215-217; 219-221; 223-225; 227-229; 231, 228 and 232; 234-236; 238-240; 242-244; 246-248; 250-252; 254-256; or 258-260 arranged in a sequential order from N to C on said nanobody.

According to specific embodiments, the nanobody specifically binds trehalase and comprises CDRs as set forth in SEQ ID NOs: 201 and 205-206; 208-210; 212 and 209-210; 223-225; 238-240; 242-244; 246-248; 588-590; or 595, 589 and 592 arranged in a sequential order from N to C on said nanobody.

According to specific embodiments, the nanobody specifically binds trehalase and comprises CDRs as set forth in SEQ ID NOs: 201 and 205-206; 208-210; 212 and 209-210; 223-225; 588-590; or 595, 589 and 592 arranged in a sequential order from N to C on said nanobody.

According to specific embodiments, the nanobody specifically binds trehalase and comprises CDRs as set forth in SEQ ID NOs: 201 and 205-206; 208-210; 223-225; or 588-590; arranged in a sequential order from N to C on said nanobody.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 194, 198, 200, 204, 207, 211, 213, 214, 218, 222, 226, 230, 233, 237, 241, 245, 249, 253, 257, 587, 591, 593, 594 and 596 each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 194, 198, 200, 204, 207, 211, 213, 214, 218, 222, 226, 230, 233, 237, 241, 245, 249, 253 and 257, each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 204, 207, 211, 213, 222, 237, 241, 245, 587 and 594.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 204, 207, 211, 213, 222, 587 and 594.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 204, 207, 222 and 587.

According to specific embodiments, the nanobody comprises an amino acid sequence selected from the group consisting of SEQ ID NOs: 194, 198, 200, 204, 207, 211, 213, 214, 218, 222, 226, 230, 233, 237, 241, 245, 249, 253, 257, 587, 591, 593, 594 and 596. According to specific embodiments, the nanobody comprises an amino acid sequence selected from the group consisting of SEQ ID NOs: 194, 198, 200, 204, 207, 211, 213, 214, 218, 222, 226, 230, 233, 237, 241, 245, 249, 253 and 257.

According to specific embodiments, the nanobody comprises an amino acid sequence selected from the group consisting of SEQ ID NOs: 204, 207, 211, 213, 222, 237, 241, 245, 587 and 594.

According to specific embodiments, the nanobody comprises an amino acid sequence selected from the group consisting of SEQ ID NOs: 204, 207, 211, 213, 222, 587 and 594.

According to specific embodiments, the nanobody comprises an amino acid sequence selected from the group consisting of SEQ ID NOs: 204, 207, 222 and 587.

According to specific embodiments, the nanobody comprises SEQ ID NO: 204.

According to specific embodiments, the nanobody comprises SEQ ID NO: 207.

According to specific embodiments, the nanobody comprises SEQ ID NO: 211.

According to specific embodiments, the nanobody comprises SEQ ID NO: 213.

According to specific embodiments, the nanobody comprises SEQ ID NO: 222.

According to specific embodiments, the nanobody comprises SEQ ID NO: 587.

According to specific embodiments, the nanobody comprises SEQ ID NO: 594.

According to specific embodiments, the nanobody consists of an amino acid sequence selected from the group consisting of SEQ ID NOs: 194, 198, 200, 204, 207, 211, 213, 214, 218, 222, 226, 230, 233, 237, 241, 245, 249, 253, 257, 587, 591, 593, 594 and 596.

According to specific embodiments, the nanobody consists of an amino acid sequence selected from the group consisting of SEQ ID NOs: 194, 198, 200, 204, 207, 211, 213, 214, 218, 222, 226, 230, 233, 237, 241, 245, 249, 253 and 257.

According to specific embodiments, the nanobody consists of an amino acid sequence selected from the group consisting of SEQ ID NOs: 204, 207, 211, 213, 222, 237, 241, 245, 587 and 594.

According to specific embodiments, the nanobody consists of an amino acid sequence selected from the group consisting of SEQ ID NOs: 204, 207, 211, 213, 222, 587 and 594.

According to specific embodiments, the nanobody consists of an amino acid sequence selected from the group consisting of SEQ ID NOs: 204, 207, 222 and 587.

According to specific embodiments, the nanobody consists of SEQ ID NO: 204.

According to specific embodiments, the nanobody consists of SEQ ID NO: 207.

According to specific embodiments, the nanobody consists of SEQ ID NO: 211.

According to specific embodiments, the nanobody consists of SEQ ID NO: 213.

According to specific embodiments, the nanobody consists of SEQ ID NO: 222.

According to specific embodiments, the nanobody consists of SEQ ID NO: 587.

According to specific embodiments, the nanobody consists of SEQ ID NO: 594.

According to specific embodiments, the nanobody binds a cytochrome p450 monooxygenase.

As used herein, the term “cytochrome p450 monooxygenase”, refer to the amino acid sequence of the insect enzyme E.C. No. 1.14, an enzyme containing heme as a cofactor that incorporates one hydroxyl group into a substrate leading to reduction of two atoms of dioxygen to one hydroxyl group and one H₂O molecule by the concomitant oxidation of NAD (P) H, or to antigenic fragments thereof capable of eliciting a specific immune response against said fragment.

According to specific embodiments, the cytochrome p450 monooxygenase is the Helicoverpa armigera cytochrome p450 monooxygenase, such as provided in GeneBank Accession No. AKS48889.

According to specific embodiments, the cytochrome p450 monooxygenase comprises SEQ ID NO: 11.

According to specific embodiments, the cytochrome p450 monooxygenase consists of SEQ ID NO: 11.

Non-limiting examples of nanobodies specifically binding cytochrome p450 monooxygenase and their respective CDRs are shown in Table 5 hereinbelow.

According to specific embodiments, the nanobody specifically binds cytochrome p450 monooxygenase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 262-264; 262-263 and 266; 262, 270 and 264; 274-276; 280 and 275-276; 282 and 275-276; 282, 275 and 285; 287, 220 and 289; 291, 220 and 289; 239-294 and 289; 296-298; 300-302; 304-306; 308-310; 312-314; or 316-318 arranged in a sequential order from N to C on said nanobody.

According to specific embodiments, the nanobody specifically binds cytochrome p450 monooxygenase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 262-264; 287, 220 and 289; 296-298; 300-302; 304-306; or 312-314 arranged in a sequential order from N to C on said nanobody.

According to specific embodiments, the nanobody specifically binds cytochrome p450 monooxygenase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 300-302; or 312-314 arranged in a sequential order from N to C on said nanobody.

According to specific embodiments, the nanobody specifically binds cytochrome p450 monooxygenase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 312-314 arranged in a sequential order from N to C on said nanobody.

According to specific embodiments, the nanobody specifically binds cytochrome p450 monooxygenase and comprises CDRs as set forth in SEQ ID NOs: 262-264; 262-263 and 266; 262, 270 and 264; 274-276; 280 and 275-276; 282 and 275-276; 282, 275 and 285; 287, 220 and 289; 291, 220 and 289; 239-294 and 289; 296-298; 300-302; 304-306; 308-310; 312-314; or 316-318 arranged in a sequential order from N to C on said nanobody.

According to specific embodiments, the nanobody specifically binds cytochrome p450 monooxygenase and comprises CDRs as set forth in SEQ ID NOs: 262-264; 287, 220 and 289; 296-298; 300-302; 304-306; or 312-314 arranged in a sequential order from N to C on said nanobody.

According to specific embodiments, the nanobody specifically binds cytochrome p450 monooxygenase and comprises CDRs as set forth in SEQ ID NOs: 300-302; or 312-314 arranged in a sequential order from N to C on said nanobody.

According to specific embodiments, the nanobody specifically binds cytochrome p450 monooxygenase and comprises CDRs as set forth in SEQ ID NOs: 312-314 arranged in a sequential order from N to C on said nanobody.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 261, 265, 267, 268, 269, 271, 272, 273, 277, 278, 279, 281, 283, 284, 286, 29, 292, 295, 299, 303, 307, 311 and 315, each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 261, 286, 295, 299, 303 and 311.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 299 and 311.

According to specific embodiments, the nanobody comprises an amino acid sequence selected from the group consisting of SEQ ID NOs: 261, 265, 267, 268, 269, 271, 272, 273, 277, 278, 279, 281, 283, 284, 286, 29, 292, 295, 299, 303, 307, 311 and 315.

According to specific embodiments, the nanobody comprises an amino acid sequence selected from the group consisting of SEQ ID NOs: 261, 286, 295, 299, 303 and 311.

According to specific embodiments, the nanobody comprises an amino acid sequence selected from the group consisting of SEQ ID NOs: 299 and 311.

According to specific embodiments, the nanobody comprises SEQ ID NO: 311.

According to specific embodiments, the nanobody comprises SEQ ID NO: 299.

According to specific embodiments, the nanobody consists of an amino acid sequence selected from the group consisting of SEQ ID NOs: 261, 265, 267, 268, 269, 271, 272, 273, 277, 278, 279, 281, 283, 284, 286, 29, 292, 295, 299, 303, 307, 311 and 315.

According to specific embodiments, the nanobody consists of an amino acid sequence selected from the group consisting of SEQ ID NOs: 261, 286, 295, 299, 303 and 311.

According to specific embodiments, the nanobody consists of an amino acid sequence selected from the group consisting of SEQ ID NOs: 299 and 311.

According to specific embodiments, the nanobody consists of SEQ ID NO: 311.

According to specific embodiments, the nanobody consists of SEQ ID NO: 299.

According to specific embodiments, the nanobody binds a chitin deacetylase.

As used herein, the term “chitin deacetylase” refers to the amino acid sequence of the insect enzyme E.C. No. 3.5.1.41, an enzyme that catalyzes the conversion of chitin and H₂O to chitosan and acetate, or to antigenic fragments thereof capable of eliciting a specific immune response against said fragment.

According to specific embodiments, the chitin deacetylase is the Helicoverpa armigera chitin deacetylase, such as provided in GeneBank Accession No. AJA30435.

According to specific embodiments, the chitin deacetylase comprises SEQ ID NO: 9.

According to specific embodiments, the chitin deacetylase consists of SEQ ID NO: 9.

Non-limiting examples of nanobodies specifically binding chitin deacetylase and their respective CDRs are shown in Table 6 hereinbelow.

According to specific embodiments, the nanobody specifically binds chitin deacetylase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 320-322; 320 and 324-325; 327, 321 and 328; 320-321 and 332; 208 and 336-337; 320, 324 and 339; 343-345; 347-349; 351-353; 351, 347 and 356; 358-360; 358-359 and 362; 365, 359 and 366; 368-370; 372 and 369-370; 374, 369 and 375; 377-379; 381-383; 385 and 382-383; 387-389; 387, 391 and 389; 394-396; 398-400; 402-404; or 406-408 arranged in a sequential order from N to C on said nanobody.

According to specific embodiments, the nanobody specifically binds chitin deacetylase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 320-322; 320 and 324-325; 372 and 369-370; 374, 369 and 375; 377-379; 387-389; or 398-400 arranged in a sequential order from N to C on said nanobody.

According to specific embodiments, the nanobody specifically binds chitin deacetylase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 320 and 324-325; 372 and 369-370; 374, 369 and 375; or 387-389 arranged in a sequential order from N to C on said nanobody.

According to specific embodiments, the nanobody specifically binds chitin deacetylase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 320 and 324-325; 374, 369 and 375; or 387-389 arranged in a sequential order from N to C on said nanobody.

According to specific embodiments, the nanobody specifically binds chitin deacetylase and comprises CDRs as set forth in SEQ ID NOs: 320-322; 320 and 324-325; 327, 321 and 328; 320-321 and 332; 208 and 336-337; 320, 324 and 339; 343-345; 347-349; 351-353; 351, 347 and 356; 358-360; 358-359 and 362; 365, 359 and 366; 368-370; 372 and 369-370; 374, 369 and 375; 377-379; 381-383; 385 and 382-383; 387-389; 387, 391 and 389; 394-396; 398-400; 402-404; or 406-408 arranged in a sequential order from N to C on said nanobody.

According to specific embodiments, the nanobody specifically binds chitin deacetylase and comprises CDRs as set forth in SEQ ID NOs: 320-322; 320 and 324-325; 372 and 369-370; 374, 369 and 375; 377-379; 387-389; or 398-400 arranged in a sequential order from N to C on said nanobody.

According to specific embodiments, the nanobody specifically binds chitin deacetylase and comprises CDRs as set forth in SEQ ID NOs: 320 and 324-325; 372 and 369-370; 374, 369 and 375; or 387-389 arranged in a sequential order from N to C on said nanobody.

According to specific embodiments, the nanobody specifically binds chitin deacetylase and comprises CDRs as set forth in SEQ ID NOs: 320 and 324-325; 374, 369 and 375; or 387-389 arranged in a sequential order from N to C on said nanobody.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 319, 323, 326, 329, 331, 333, 334, 338, 340, 341, 342, 346, 350, 354, 355, 357, 361, 363, 364, 367, 371, 373, 376, 380, 384, 386, 390, 392, 393, 397, 401 and 405, each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 319, 323, 371, 373, 376, 386 and 397.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 323, 371, 373 and 386.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 323, 373 and 386.

According to specific embodiments, the nanobody comprises an amino acid sequence selected from the group consisting of SEQ ID NOs: 319, 323, 326, 329, 331, 333, 334, 338, 340, 341, 342, 346, 350, 354, 355, 357, 361, 363, 364, 367, 371, 373, 376, 380, 384, 386, 390, 392, 393, 397, 401 and 405.

According to specific embodiments, the nanobody comprises an amino acid sequence selected from the group consisting of SEQ ID NOs: 319, 323, 371, 373, 376, 386 and 397.

According to specific embodiments, the nanobody comprises an amino acid sequence selected from the group consisting of SEQ ID NOs: 323, 371, 373 and 386.

According to specific embodiments, the nanobody comprises an amino acid sequence selected from the group consisting of SEQ ID NOs: 323, 373 and 386.

According to specific embodiments, the nanobody comprises SEQ ID NO: 323.

According to specific embodiments, the nanobody comprises SEQ ID NO: 371 According to specific embodiments, the nanobody comprises SEQ ID NO: 373.

According to specific embodiments, the nanobody comprises SEQ ID NO: 386.

According to specific embodiments, the nanobody consists of an amino acid sequence selected from the group consisting of SEQ ID NOs: 319, 323, 326, 329, 331, 333, 334, 338, 340, 341, 342, 346, 350, 354, 355, 357, 361, 363, 364, 367, 371, 373, 376, 380, 384, 386, 390, 392, 393, 397, 401 and 405.

According to specific embodiments, the nanobody consists of an amino acid sequence selected from the group consisting of SEQ ID NOs: 319, 323, 371, 373, 376, 386 and 397.

According to specific embodiments, the nanobody consists of an amino acid sequence selected from the group consisting of SEQ ID NOs: 323, 371, 373 and 386.

According to specific embodiments, the nanobody consists of SEQ ID NO: 323.

According to specific embodiments, the nanobody consists of SEQ ID NO: 371 According to specific embodiments, the nanobody consists of SEQ ID NO: 373.

According to specific embodiments, the nanobody consists of SEQ ID NO: 386.

According to specific embodiments, the nanobody binds a chitin synthase.

As used herein, the term “chitin synthase” refers to the amino acid sequence of the insect enzyme E.C. No. EC 2.4.1.16, an enzyme which catalyzes the conversion of UDP-N-acetyl-D-glucosamine and [1,4-(N-acetyl-beta-D-glucosaminyl)]n to UDP+[1,4-(N-acetyl-beta-D-glucosaminyl)]n₊1, or to antigenic fragments thereof capable of eliciting a specific immune response against said fragment.

According to specific embodiments, the chitin synthase is the Helicoverpa armigera chitin synthase, such as provided in GeneBank Accession No. AKZ08594.

According to specific embodiments, the chitin synthase comprises SEQ ID NO: 12.

According to specific embodiments, the chitin synthase consists of an amino acid sequence selected from the group consisting of SEQ ID NO: 12.

Non-limiting examples of nanobodies specifically binding chitin synthase and their respective CDRs are shown in Table 7 hereinbelow.

According to specific embodiments, the nanobody specifically binds chitin synthase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 412-414; 418-420; 422-424; 426-428; 426-427 and 430; 432-434; 436, 140 and 437; 439-441; 443-445; 447-449; 356 and 451-452; 454-456; 458-460; 462-464; 466-468; 470-472; 474-476; 478-480; 482, 479 and 483; 485-487; 489-491; 493-495; 426 and 497-498; 500-502; 504-506; or 508-510 arranged in a sequential order from N to C on said nanobody.

According to specific embodiments, the nanobody specifically binds chitin synthase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 412-414; 418-420; 426-428; 432-434; 443-445; 447-449; 466-468; 482, 479 and 483; 426 and 497-498; or 504-506 arranged in a sequential order from N to C on said nanobody.

According to specific embodiments, the nanobody specifically binds chitin synthase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 426-428; 432-434; or 443-445 arranged in a sequential order from N to C on said nanobody.

According to specific embodiments, the nanobody specifically binds chitin synthase and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 426-428; or 443-445 arranged in a sequential order from N to C on said nanobody.

According to specific embodiments, the nanobody specifically binds chitin synthase and comprises CDRs as set forth in SEQ ID NOs: 412-414; 418-420; 422-424; 426-428; 426-427 and 430; 432-434; 436, 140 and 437; 439-441; 443-445; 447-449; 356 and 451-452; 454-456; 458-460; 462-464; 466-468; 470-472; 474-476; 478-480; 482, 479 and 483; 485-487; 489-491; 493-495; 426 and 497-498; 500-502; 504-506; or 508-510 arranged in a sequential order from N to C on said nanobody.

According to specific embodiments, the nanobody specifically binds chitin synthase and comprises CDRs as set forth in SEQ ID NOs: 412-414; 418-420; 426-428; 432-434; 443-445; 447-449; 466-468; 482, 479 and 483; 426 and 497-498; or 504-506 arranged in a sequential order from N to C on said nanobody.

According to specific embodiments, the nanobody specifically binds chitin synthase and comprises CDRs as set forth in SEQ ID NOs: 426-428; 432-434; or 443-445 arranged in a sequential order from N to C on said nanobody.

According to specific embodiments, the nanobody specifically binds chitin synthase and comprises CDRs as set forth in SEQ ID NOs: 426-428; or 443-445 arranged in a sequential order from N to C on said nanobody.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 411, 415, 416, 417, 421, 425, 429, 431, 435, 438, 442, 446, 450, 453, 457, 461, 465, 469, 473, 477, 481, 484, 488, 492, 496, 499, 503 and 507, each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 411, 417, 425, 431, 442, 446, 465, 481, 496 and 503.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 425, 431 and 442.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 425 and 442.

According to specific embodiments, the nanobody comprises an amino acid sequence selected from the group consisting of SEQ ID NOs: 411, 415, 416, 417, 421, 425, 429, 431, 435, 438, 442, 446, 450, 453, 457, 461, 465, 469, 473, 477, 481, 484, 488, 492, 496, 499, 503 and 507.

According to specific embodiments, the nanobody comprises an amino acid sequence selected from the group consisting of SEQ ID NOs: 411, 417, 425, 431, 442, 446, 465, 481, 496 and 503.

According to specific embodiments, the nanobody comprises an amino acid sequence selected from the group consisting of SEQ ID NOs: 425, 431 and 442.

According to specific embodiments, the nanobody comprises an amino acid sequence selected from the group consisting of SEQ ID NOs: 425 and 442.

According to specific embodiments, the nanobody comprises SEQ ID NO: 425.

According to specific embodiments, the nanobody comprises SEQ ID NO: 431.

According to specific embodiments, the nanobody comprises SEQ ID NO: 442.

According to specific embodiments, the nanobody consists of an amino acid sequence selected from the group consisting of SEQ ID NOs: 411, 415, 416, 417, 421, 425, 429, 431, 435, 438, 442, 446, 450, 453, 457, 461, 465, 469, 473, 477, 481, 484, 488, 492, 496, 499, 503 and 507.

According to specific embodiments, the nanobody consists of an amino acid sequence selected from the group consisting of SEQ ID NOs: 411, 417, 425, 431, 442, 446, 465, 481, 496 and 503.

According to specific embodiments, the nanobody consists of an amino acid sequence selected from the group consisting of SEQ ID NOs: 425, 431 and 442.

According to specific embodiments, the nanobody consists of an amino acid sequence selected from the group consisting of SEQ ID NOs: 425 and 442.

According to specific embodiments, the nanobody consists of SEQ ID NO: 425.

According to specific embodiments, the nanobody consists of SEQ ID NO: 431.

According to specific embodiments, the nanobody consists of SEQ ID NO: 442.

According to specific embodiments, the nanobody binds a NPC1 sterol transporter.

As used herein, the term “NPC1 sterol transporter” refers to the amino acid sequence of the insect protein encoded by the NPCI gene (Gene ID: 4864, or to antigenic fragments thereof capable of eliciting a specific immune response against said fragment.

According to specific embodiments, the NPC1 sterol transporter is the Helicoverpa armigera NPC1 sterol transporter, such as provided in GeneBank Accession No. XP_021186115.

According to specific embodiments, the NPC1 sterol transporter comprises SEQ ID NO: 13.

According to specific embodiments, the NPC1 sterol transporter consists of SEQ ID NO: 13.

Non-limiting examples of nanobodies specifically binding NPC1 sterol transporter and their respective CDRs are shown in Table 8 hereinbelow.

According to specific embodiments, the nanobody specifically binds NPC1 sterol transporter and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 512-514; 517 and 513-514; 521-523; 521-522 and 526; 531-533; 539-541; 485 and 540-541; 545 and 540-451; 547-549; 552-554; 556-558; 561-563; 565-567; 569-571; 573-575; 485 and 577-578; 580-582; or 584-586 arranged in a sequential order from N to C on said nanobody.

According to specific embodiments, the nanobody specifically binds NPC1 sterol transporter and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 517 and 513-514; 485 and 540-541; 545 and 540-541; 556-558; 565-567; 573-575; 485 and 577-578; or 580-582 arranged in a sequential order from N to C on said nanobody.

According to specific embodiments, the nanobody specifically binds NPC1 sterol transporter and comprises CDRs having an amino acid sequence having at least 70%, at least 75%, at least 80%, at least 85%, at least 90%, at least 95%, at least 96%, at least 97%, at least 98%, at least 99%, or 100% identity to amino acid sequences as set forth in SEQ ID NOs: 485 and 540-541; or 545 and 540-541 arranged in a sequential order from N to C on said nanobody.

According to specific embodiments, the nanobody specifically binds NPC1 sterol transporter e and comprises CDRs as set forth in SEQ ID NOs: 512-514; 517 and 513-514; 521-523; 521-522 and 526; 531-533; 539-541; 485 and 540-541; 545 and 540-451; 547-549; 552-554; 556-558; 561-563; 565-567; 569-571; 573-575; 485 and 577-578; 580-582; or 584-586 arranged in a sequential order from N to C on said nanobody.

According to specific embodiments, the nanobody specifically binds NPC1 sterol transporter and comprises CDRs as set forth in SEQ ID NOs: 517 and 513-514; 485 and 540-541; 545 and 540-541; 556-558; 565-567; 573-575; 485 and 577-578; or 580-582 arranged in a sequential order from N to C on said nanobody.

According to specific embodiments, the nanobody specifically binds NPC1 sterol transporter and comprises CDRs as set forth in SEQ ID NOs: 485 and 540-541; or 545 and 540-541 arranged in a sequential order from N to C on said nanobody.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 511, 515, 516, 518, 519, 520, 524, 525, 527, 528, 529, 530, 534, 535, 536, 537, 538, 542, 543, 544, 546, 550, 551, 555, 559, 560, 564, 568, 572, 576, 579 and 583, each possibility represents a separate embodiment of the present invention.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 516, 543, 544, 555, 564, 572, 576 and 579.

According to specific embodiments, the nanobody comprises an amino acid sequence having at least 70%, at least 75%, at least 80%, at least 85%, at least 90%, at least 95%, at least 96%, at least 97%, at least 98%, at least 99% or 100% identity to an amino acid sequence selected from the group consisting of SEQ ID NOs: 543 and 544.

According to specific embodiments, the nanobody comprises an amino acid sequence selected from the group consisting of SEQ ID NOs: 511, 515, 516, 518, 519, 520, 524, 525, 527, 528, 529, 530, 534, 535, 536, 537, 538, 542, 543, 544, 546, 550, 551, 555, 559, 560, 564, 568, 572, 576, 579 and 583.

According to specific embodiments, the nanobody comprises an amino acid sequence selected from the group consisting of SEQ ID NOs: 516, 543, 544, 555, 564, 572, 576 and 579.

According to specific embodiments, the nanobody comprises an amino acid sequence selected from the group consisting of SEQ ID NOs: 543 and 544.

According to specific embodiments, the nanobody comprises SEQ ID NO: 543.

According to specific embodiments, the nanobody comprises SEQ ID NO: 544.

According to specific embodiments, the nanobody consists of an amino acid sequence selected from the group consisting of SEQ ID NOs: 511, 515, 516, 518, 519, 520, 524, 525, 527, 528, 529, 530, 534, 535, 536, 537, 538, 542, 543, 544, 546, 550, 551, 555, 559, 560, 564, 568, 572, 576, 579 and 583.

According to specific embodiments, the nanobody consists of an amino acid sequence selected from the group consisting of SEQ ID NOs: 516, 543, 544, 555, 564, 572, 576 and 579.

According to specific embodiments, the nanobody consists of an amino acid sequence selected from the group consisting of SEQ ID NOs: 543 and 544.

According to specific embodiments, the nanobody consists of SEQ ID NO: 543.

According to specific embodiments, the nanobody consists of SEQ ID NO: 544.

It should be noted that the nanobodies of some embodiments of the present invention in their broadest sense are not limited to a specific biological source or to a specific method of preparation. For example, nanobodies, can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a mammalian species, such as from a human being, or by expression of a nucleic acid encoding such a camelized VH domain; (5) by “camelization” of a “domain antibody” or “Dab,” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing. A further description of nanobodies, including humanization and/or camelization of nanobodies, can be found, e.g., in WO 08/101985 and WO 08/142164, as well as further herein. A recently reported fully in vitro platform based on yeast surface display for nanobody discovery is disclosed in McMahon, Conor, et al. “Nature structural & molecular biology 25.3 (2018): 289.

According to specific embodiments, the nanobody is “camelized.” For example, “camelization” can be performed by providing a nucleotide sequence that encodes a naturally occurring VH domain and then changing, in a manner known per se, one or more codons in the nucleotide sequence in such a way that the new nucleotide sequence encodes a “camelized” nanobody, respectively. This nucleic acid can then be expressed in a manner known per se, so as to provide the desired nanobody.

Other suitable methods and techniques for obtaining the nanobody and/or nucleic acid sequence encoding same, starting from naturally occurring VH sequences or preferably VHH sequences, will be clear from the skilled person, and may, for example, comprise combining one or more parts of one or more naturally occurring VH sequences (such as one or more FR sequences and/or CDR sequences), one or more parts of one or more naturally occurring VHH sequences (such as one or more FR sequences or CDR sequences), and/or one or more synthetic or semi-synthetic sequences, in a suitable manner, so as to provide the nanobody or a nucleic acid sequence encoding same.

A specific method of generating nanobodies is described herein.

According to an aspect of the present invention there is provided a method of producing a nanobody, the method comprising immunizing a camelid with a recombinant or purified insect polypeptide selected from the group consisting of:
(i) a polypeptide comprising a chitin binding domain (CBD);
(ii) V-ATPase subunit c;
(iii) trehalase;
(iv) cytochrome p450 monooxygenase;
(v) chitin deacetylase;
(vi) chitin synthase; and
(vii) NPC1 sterol transporter,
wherein purity of said insect polypeptide in an insect polypeptide preparation is at least 80%.

As used herein, the phrase “purified insect polypeptide” refers to a polypeptide purified from an insect such that its purity compared to other polypeptides present in the protein preparation is at least 80%.

According to specific embodiments, the purity of the insect polypeptide in the purified insect protein preparation is at least 85%, at least 90%, at least 95%, at least 96%, at least 97%, at least 98%, at least 99% or 100%, each possibility represents a separate embodiment of the present invention.

According to specific embodiments, the method further comprising isolating the nanobody. Methods of isolating nanobodies are well known and are disclosed e.g. in Pardon et al. Nat Protoc. 2014 March; 9(3): 674-693, the contents of which are fully incorporated herein by reference, and in the Examples section that follows.

Once nanobodies are obtained, the binding and/or the biological activity (e.g. insect control activity) of the nanobody can be assayed either in vivo or in vitro. Such methods are known in the art and are further disclosed hereinabove and below.

Thus, according to specific embodiments, the method further comprises selecting a nanobody demonstrating an insect control activity in a biological assay. Such assays are known in the art and are further described hereinbelow.

According to specific embodiments, the nanobody is a naked nanobody.

As used herein, the tern “naked nanobody” refers to a nanobody which does not comprise a heterologous effector moiety e.g. toxin moiety, detectable moiety.

According to specific embodiments, the nanobody comprises a heterologous effector moiety e.g. toxin moiety, detectable moiety. The effector moiety can be proteinaceous or non-proteinaceous (e.g. small molecule chemical compounds); the latter generally being generated using functional groups on the nanobody and on the conjugate partner.

Thus, for example, various types of detectable or reporter moieties may be conjugated to the nanobody of the invention. These include, but not are limited to, a radioactive isotope (such as iodine), a phosphorescent chemical, a chemiluminescent chemical, a fluorescent chemical or

polypeptide (e.g. phycoerythrin (PE), fluorescein isothiocyanate (FITC), Cy-chrome, rhodamine, green fluorescent protein (GFP), blue fluorescent protein (BFP), Texas red, Cy5, PE-Cy5, and the like), an enzyme (e.g. e.g., horseradish peroxidase (HPR), beta-galactosidase, and alkaline phosphatase (AP), an affinity tag [e.g. an antigen identifiable by a corresponding antibody (e.g., digoxigenin (DIG) which is identified by an anti-DIG antibody) or a molecule having a high affinity towards the tag (e.g., streptavidin and biotin)], and molecules (contrast agents) detectable by Positron Emission Tomography (PET) or Magnetic Resonance Imaging (MRI).

According to specific embodiments, the nanobody comprises a toxin.

As used herein, the term “toxin” or “toxin moiety” refers to a compound having an insect control activity (as defined hereinbelow) including, but not limited to, a polypeptide, a polynucleotide, a small molecule, etc.

Non-limiting Examples of toxin moieties include δ-endotoxins [such as Cry1A, Cry1B, Cry1C, Cry1D, Cry1E, Cry1F, Cry1G, Cry1H, Cry1I, Cry1J, Cry1K, Cry2A, Cry7B, Cry8D, Cry9A, Cry9B, Cry9C, Cry9D, Cry9E, Cry15A, Cry22A, Cry32A, Cry51A, Cyt1A (Crickmore et al., 1998; van Frankenhuyzen, 2009], colicins (such as colicin E1, colicin la, colicin A, colicin N), actinoporins (such as equinatoxin II, sticholysin II, fragaceatoxin C), ClyA family toxins (such as cytolysin A, non-haemolytic tripartite enterotoxin, haemolysin BL), haemolysins (such as α-haemolysin, γ-haemolysin, leukocidins, nectrotic enteritis toxin B, 8-toxin, Vibrio cholerae cytolysin, Vibrio vulnificus haemolysin), aerolysin family toxins (such as aerolysin, a-toxin, hydralysin, ε-toxin, enterotoxin, haemolytic lectin, kysenin), cholesterol-dependent cytolysins (such as perfringolysin, suilysin, intermedilysin, listeriolysin O, lectinolysin, anthrolysin, streptolysin), membrane attack complex components/perforins (such as Plu-MACPF, Bth-MACPF), repeats-in-toxins (such as HlyA, bifunctional haemolysin-adenylyl cyclase toxin, MARTX) (Dal Peraro and van der Goot, 2016), spider toxins, scorpion toxins, patatin, a Bacillus thuringiensis insecticidal protein, a Xenorhabdus insecticidal protein, a Photorhabdus insecticidal protein, a Bacillus laterosporous insecticidal protein, a Bacillus bombysepticus insecticidal protein, a Bacillus sphaericus insecticidal protein, and insect-controlling double-stranded RNAs

The effector moiety e.g. toxin moiety, detectable moiety may be attached or conjugated to the nanobody of the invention in various ways, depending on the context, application and purpose.

The effector moiety may be coupled directly or indirectly (e.g. when comprised in a carrier) to the nanobody. The coupling can be a covalent or non-covalent binding.

When the effector moiety is a polypeptide, the immunoconjugate may be produced by recombinant means. For example, the nucleic acid sequence encoding a toxin or a fluorescent protein may be ligated in-frame with the nucleic acid sequence encoding the nanobody and be expressed in a host cell to produce a recombinant conjugated antibody. Alternatively, the effector moiety may be chemically synthesized by, for example, the stepwise addition of one or more amino acid residues in defined order such as solid phase peptide synthetic techniques.

An effector moiety may also be attached to the nanobody using standard chemical synthesis techniques widely practiced in the art [see e.g., worldwideweb (dot) chemistry (dot) org/portal/Chemistry)], such as using any suitable chemical linkage, direct or indirect, as via a peptide bond (when the effector moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling is provided in details in U.S. Pat. Nos. 3,940,475, 4,289,747, and 4,376,110.

Exemplary methods for conjugating peptide moieties to the nanobody include, but not limited to SPDP conjugation, Glutaraldehyde conjugation and Carbodiimide conjugation.

The nanobody can also be attached to particles or carriers, which comprise the effector moiety. Methods of covalently binding a nanobody to an encapsulating particle are known in the art and disclosed for example in U.S. Pat. Nos. 5,171,578, 5,204,096 and 5,258,499.

Any of the polypeptides (e.g. nanobodies and proteinaceous compositions) described herein can be encoded from a polynucleotide. These polynucleotides can be used per se or in the recombinant production of the polypeptides disclosed herein.

Thus, according to an aspect of the present invention there is provided a polynucleotide encoding the nanobody or the composition comprising the nanobody and the toxin.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein, which, encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a polynucleotide which encodes a polypeptide is implicit in a described sequence with respect to the expression product.

According to specific embodiments, the nucleic acid sequences disclosed herein are codon optimized for e.g. mammalian or plan expression.

Methods of codon optimization are known in the art and disclosed e.g. in the Codon Usage Database through the NIAS (National Institute of Agrobiological Sciences) DNA bank in Japan (www (dot) kazusa (dot) or (dot) jp/codon/); International Patent Application on. 93/07278; and Grote et al. Nucleic Acid Res. Nucleic Acids Res. (2005) July 1; 33 (Web Server issue): W526-W531).

Thus, some embodiments of the invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

To express an exogenous polypeptide in a cell, a polynucleotide sequence encoding the polypeptide is preferably ligated into a nucleic acid construct suitable for expression in the cell. Such a nucleic acid construct includes a promoter sequence for directing transcription of the polynucleotide sequence in the cell in a constitutive or inducible manner.

Hence, according to an aspect of the present invention there is provided a nucleic acid construct comprising the polynucleotide and a cis-acting regulatory element for directing expression of said polynucleotide.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the polypeptide from a cell in which it is placed. According to specific embodiments, the signal sequence is the native signal sequence of the polypeptide (e.g. nanobody) of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], pancreas-specific promoters [Edlunch et al. (1985) Science 230:912-916] or mammary gland-specific promoters such as the milk whey promoter (U.S. Pat. No. 4,873,316 and European Application Publication No. 264,166).

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843], include AlcR/AlcA (ethanol inducible); GR fusions, GVG, and pOp/LhGR (dexamethasone inducible); XVE/OlexA (beta-estradiol inducible); or heat shock promoters, e.g., soybean hsp17.5-E or hsp17.3-B [Gurley et al. (1986) Mol. Cell. Biol. 6:559-565] can be used.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long terminal repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference. Enhancers specific for distinct neuronal cell types that can be included in AAV expression vectors to gain specificity without a Cre-driver line have also been described in the arts and described e.g. in Hrvatin et al. (doi: www (dot) doi (dot) org/10.1101/570895), which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such, no general description of selection consideration is provided herein.

The cells may be transformed stably or transiently with the nucleic acid constructs of some embodiments of the invention. In stable transformation, the nucleic acid molecule of some embodiments of the invention is integrated into the cell genome and as such it represents a stable and inherited trait. In transient transformation, the nucleic acid molecule is expressed by the cell transformed but it is not integrated into the genome and as such, it represents a transient trait.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In cases where plant expression vectors are used, the constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, yield or toxicity of the expressed peptide. For example, the expression of a fusion protein or a cleavable fusion protein comprising the polypeptide of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the polypeptide and the heterologous protein, the polypeptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

The present invention also contemplates cells comprising the polypeptides, polynucleotides and nucleic acid constructs described herein.

Thus, according to an aspect of the present invention there is provided a host cell comprising the nanobody or the composition comprising the nanobody and the toxin or a polynucleotide or a nucleic acid construct encoding same.

As mentioned hereinabove, a variety of prokaryotic or eukaryotic cells can be used as host-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

According to specific embodiments, the cell is a mammalian cell.

According to specific embodiments, the cell is a camelid cell.

Suitable mammalian cells include primary cells and immortalized cell lines.

According to other specific embodiments, the mammalian cell is an immortalized cell line.

Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), HEK293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, NSO, Sp2/0, BHK, Namalwa, and the like.

According to specific embodiments, the cell is E. coli e.g. SHuffle T7 Express & BL21.

According to specific embodiments, the cell is a plant cell.

According to an aspect of the present invention there is provided a method of producing an insect control nanobody, the method comprising expressing in a host cell the polynucleotide or the nucleic acid construct disclosed herein.

According to specific embodiments, the method further comprising isolating the nanobody.

Isolation or recovery of any of the recombinant polypeptides (e.g. nanobody) may be effected by any method known in the art. According to specific embodiments, recovery or isolation of the recombinant polypeptide is effected following an appropriate time in culture. The phrase “recovering the recombinant polypeptide” or “isolating the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, mix mode chromatography, metal affinity chromatography, Lectins affinity chromatography chromatofocusing and differential solubilization.

According to specific embodiments, following synthesis and purification, the binding and/or the insect control activity of the nanobody can be assayed either in vivo or in vitro. Such methods are known in the art and are further disclosed hereinabove and below.

The compositions disclosed herein (e.g. the nanobodies, the composition comprising the nanobody and the toxin, polynucleotides and nucleic acid constructs encoding same and host cells expressing same) may be formulated in an appropriate means such as lyophilized, freeze-dried, desiccated, or in an aqueous carrier, medium or suitable diluent, such as saline or other buffer. According to specific embodiments, the formulated compositions may be in the form of a dust or granular material, powder, gel, cream, paste, pellet, tablet or a suspension in oil (vegetable or mineral), or water or oil/water emulsions, capsule suspension, emulsifiable concentrate, or as a wettable powder, wettable granules, water dispersible granules, aerosols, foam, slurries or flowable concentrates.

According to specific embodiments, the composition is formulated as a liquid concentrate, dry powder, tablet, capsule suspension, slurry or “wet cake”, which can be suitably diluted, dispersed, suspended, emulsified or otherwise suitably reconstituted by the end user prior to final use.

According to specific embodiments, the composition is formulated as a liquid formulation.

According to specific embodiments, the composition is formulated as a dry formulation.

According to specific embodiments, the composition is formulated for delivery by spraying, irrigation and/or fumigation.

According to specific embodiments, the compositions disclosed herein (e.g. the nanobodies, the composition comprising the nanobody and the toxin, polynucleotides and nucleic acid constructs encoding same and host cells expressing same) are stable, both during storage and during utilization, meaning that the integrity of the composition is maintained under storage and/or utilization conditions of the composition, which may include elevated temperatures, freeze-thaw cycles, changes in pH or in ionic strength, UV-irradiation, presence of harmful chemicals and the like.

According to specific embodiments, the integrity and activity of the composition is maintained under storage and/or utilization conditions of the composition, which may include elevated temperatures, freeze-thaw cycles, changes in pH or in ionic strength, UV-irradiation, presence of harmful chemicals and the like.

According to specific embodiments, the integrity and activity of the composition is maintained under open field or controlled conditions, e.g., greenhouse.

It should be noted that the compositions disclosed herein can be formulated with various carriers designed to increase e.g. delivery, stability, permeability and the like.

A “carrier”, as used herein, means any solid, semi-solid or liquid carrier in or on (to) which a compound (e.g. nanobody and/or toxin, composition comprising same) can be suitably incorporated, included, immobilized, adsorbed, absorbed, bound, encapsulated, embedded, attached, or comprised. Non-limiting examples of such carriers include nanocapsules, microcapsules, nanospheres, microspheres, nanoparticles, microparticles, liposomes, vesicles, beads, a gel, weak ionic resin particles, liposomes, cochleate delivery vehicles, small granules, granulates, nano-tubes, bucky-balls, water droplets that are part of an water-in-oil emulsion, oil droplets that are part of an oil-in-water emulsion, organic materials such as cork, wood or other plant-derived materials (e.g. in the form of seed shells, wood chips, pulp, spheres, beads, sheets or any other suitable form), paper or cardboard, inorganic materials such as talc, clay, microcrystalline cellulose, silica, alumina, silicates and zeolites, or even microbial cells (such as yeast cells) or suitable fractions or fragments thereof.

According to specific embodiments, the carriers are such that they have immediate or gradual or slow release characteristics, for example over several minutes, several hours, several days or several weeks. Also, the carriers may be made of materials (e.g. polymers) that rupture or slowly degrade (for example, due to prolonged exposure to high or low temperature, sunlight, high or low humidity or other environmental factors or conditions) over time (e.g. over minutes, hours, days or weeks) and so release the compound (e.g. nanobody and/or toxin, composition comprising same) from the carrier. According to specific embodiments, the carrier is coupled, bound, linked or otherwise attached to or associated with the compound. According to specific embodiments, the carrier is covalently coupled to the compound.

The compositions disclosed herein (e.g. the nanobodies, the composition comprising the nanobody and the toxin, polynucleotides and nucleic acid constructs encoding same and host cells expressing same) may be formulated in a composition such as an agrochemical or insecticidal composition where is mixed with suitable physiologically acceptable carriers or excipients.

Herein the term “active ingredient” refers to the nanobodies, the composition comprising the nanobody and the toxin, thepolynucleotides and nucleic acid constructs encoding same and the host cells expressing same accountable for the biological effect.

An “agrochemical formulation” as used herein means a composition for agricultural use, comprising one or more of the active ingredients described with other chemical components such as agriculturally acceptable carriers and excipients.

Hereinafter, the phrases “physiologically acceptable carrier” refer to a carrier or a diluent that does not cause significant irritation to an organism and does not abrogate the biological activity and properties of the administered compound. An adjuvant is included under these phrases.

According to specific embodiments, the composition is formulated with an agriculturally acceptable carrier. Suitable agricultural carriers can be solid or liquid and are well known in the art. The term “agriculturally acceptable carrier” covers all adjuvants, e.g., inert components, dispersants, surfactants, tackifiers, binders, etc. that are ordinarily used in insecticide formulation technology and are well known to the skilled artisan.

Herein the term “excipient” refers to an inert substance added to a composition to further facilitate administration of an active ingredient. Examples, without limitation, of excipients include calcium carbonate, calcium phosphate, various sugars and types of starch, cellulose derivatives, surfactant, gelatin, vegetable oils, polyethylene glycols, wetting agents, spreading agents, buffering agents, acidifiers and the like.

According to specific embodiments, the compositions disclosed herein (e.g. the nanobodies, the composition comprising the nanobody and the toxin, polynucleotides and nucleic acid constructs encoding same and host cells expressing same) may be the only active substance in the composition.

According to other specific embodiments, the composition comprises one or more additional active agents in addition to the compositions disclosed herein (e.g. the nanobodies, the composition comprising the nanobody and the toxin, polynucleotides and nucleic acid constructs encoding it and host cells expressing it). Non-limiting examples of such agents include herbicides, insecticides, plant growth regulators, safeners and the like.

According to specific embodiments, the composition disclosed herein and the additional active agent are in a co-formulation.

According to specific embodiments, the composition disclosed herein and the additional active agent are in separate containers.

According to other specific embodiments, the composition may comprise an insect attractant. The attractant may be a pheromone, such as a male or female pheromone for example. As an example, the pheromones referred to in the book “Insect Pheromones and their use in Pest Management” (Howse et al, Chapman and Hall, 1998) may be used in the invention.

The attractant may be present in the formulation or it may be applied separately from the formulation, to ensure that the insects are attracted to the site where the formulation is applied.

The nanobody and the compositions comprising the nanobody and the toxin of some embodiments of the invention are endowed with an insect control activity.

As used herein, the term “insect control” refers to preventing, inhibiting or reducing the ability of an insect to feed, grow, move, spread, develop, survive, and/or reproduce, and/or to limit insect-related damage, which may be manifested by e.g. killing of the insect, decreasing insect survival or longevity, decreasing insect's fecundity and/or fertility, decreasing or arresting insect's feeding, decreasing or arresting insect's growth, decreasing or arresting insect's development, decreasing or arresting insect's mobility and/or preventing infestation by an insect. According to specific embodiments, the insect control activity is manifested by killing of the insect.

Methods of determining insect control activity are well known to the skilled in the art and are also disclosed in the Examples section which follows and include, but are not limited to in-vitro growing a larva in the presence of the nanobody or the composition and determining mortality, weight, length, pupation and adult emerged timing as compared to same in the absence of the nanobody or the composition.

The nanobody disclosed herein may have an insect control activity by itself or it may exert its insect control activity by delivering a toxin to an insect.

Thus, according to specific embodiments, binding of the nanobody to the insect polypeptide confers an insect control activity to the nanobody.

According to specific embodiments, the nanobody downregulates activity of the insect polypeptide it binds.

As used herein, “downregulates activity” refers to a decrease of at least 5% in biological function of the insect polypeptide in the presence of the nanobody in comparison to it in the absence of the nanobody, as determined by a method suitable for determining activity of the insect polypeptide. Thus, for example determining the activity of a polypeptide comprising CBD may be effected by e.g. ELISA, Western blot analysis, immunoprecipitation or flow cytometry; determining the activity of V-ATPase subunit c may be effected by e.g. ATPase activity assays and/or ATP dependent proton transporter assays, determining activity of trehalase may be effected by e.g. ferric-ferrocyanide reaction for determining reducing sugars or quantitation of released glucose using glucose oxidase-peroxidase following incubation with trehalase; determining activity of cytochrome p450 monooxygenase may be effected by e.g. measurements of the oxidation of drugs or surrogate compounds, heme assay or the P450-Glo luminescent assay; determining the activity of chitin deacetylase may be effected by e.g. radiometric assay using as substrate partially O-hydroxyethylated chitin (glycol chitin), radiolabeled in N-acetyl groups or the Bergmeyer enzymatic assay determining acetate released by the action of chitin deacetylase on various chitinous substrates; determining the activity of chitin synthase may be effected by e.g. a radioactive assay using [14C] UDP-N-acetyl-D-glucosamine (GlcNAc) as a substrate followed by quantization of insoluble 14C-labeled chitin after acid precipitation; and determining the activity of NPC1 sterol transporter may be effected by e.g. cholesterol absorption assay, sterol quantitation assay.

According to other specific embodiments the decrease is by at least a 10%, at least 20%, at least 30%, at least 40%, at least 50%, at least 60%, at least 70%, at least 80%, at least 90%, at least 95%, at least 99% or 100% as compared to same in the absence of the nanobody, as may be determined by e.g. any of the methods described hereinabove.

According to additional or alternative embodiments, the nanobody is a targeting agent, which serves to provide specific delivery of e.g. a toxin having an insect control activity to the insect.

Non-limiting examples of toxins and methods of coupling them to the nanobody are further described hereinabove.

As the nanobodies and compositions comprising the nanobody and the toxin of some embodiments of the invention are endowed with insect control activity, the present invention also encompasses methods of insect control.

Thus, according to an aspect of the present invention there is provided a method of insect control, the method comprising contacting the insect with the nanobody or the composition comprising the nanobody and the toxin, a polynucleotide or a nucleic acid construct encoding same or a host cell expressing same.

According to specific embodiments, the contacting comprises applying the nanobody or the composition comprising the nanobody and the toxin the directly to the insect.

According to specific embodiments, the contacting comprises applying the nanobody or the composition comprising the nanobody and the toxin to an organism or a surface, which may be in contact with said insect.

According to another aspect of the present invention, there is provided a method of preventing insect infestation of a commodity product, the method comprising adding to the product the nanobody or the composition comprising the nanobody and the toxin.

According to another aspect of the present invention, there is provided a method of preventing insect infestation of a commodity product, the method comprising packaging the product in a packaging material comprising the nanobody or the composition comprising the nanobody and the toxin.

The contacting or the adding may be effected using any suitable method known in the art, including, but not limited to spraying (including high volume (HV), low volume (LV) and ultra low volume (ULV) spraying), atomizing, brushing, dressing, dripping, coating, dipping, immersing, submerging, encrusting, spreading, foaming, fogging, injecting, adding to a culture, irrigating, applying as small droplets, a mist or an aerosol, recombinantly expressing the nucleic acid construct in a cell of an organism (as further disclosed hereinabove).

According to specific embodiments, contacting or adding is effected by spraying, irrigating and/or fumigation.

According to specific embodiments, contacting or adding is effected by introducing the polynucleotide or the nucleic acid construct into a cell of the organism.

According to specific embodiments, the organism is a plant.

As used herein, the term “surface” refers to any object, which may be in contact with an insect. Non-limiting surfaces include nets (e.g. mosquito nets), a light source, a colored object, a shape or silhouette that stand out of a contrasting background greenhouses, outdoor camping facilities, soil and the like.

According to specific embodiments, the commodity product is produced from a plant.

The term “plant” as used herein encompasses whole plants, a grafted plant, ancestors and progeny of the plants and plant parts, including seeds, shoots, stems, roots (including tubers), rootstock, scion, fruits, vegetables, flowers and plant cells, tissues and organs. The plant may be in any form including suspension cultures, embryos, meristematic regions, callus tissue, leaves, gametophytes, sporophytes, pollen, and microspores.

According to specific embodiments, the plant is a crop. “Crop” as used herein means a plant species or variety that is grown to be harvested as food, livestock fodder, fuel raw material, or for any other economic purpose. As a non-limiting example, the crops can be maize, cereals, such as wheat, rye, barley and oats, sorghum, rice, sugar beet and fodder beet, fruit, such as pome fruit (e.g., apples and pears), citrus fruit (e.g., oranges, lemons, limes, grapefruit, or mandarins), stone fruit (e.g., peaches, nectarines or plums), nuts (e.g., almonds or walnuts), soft fruit (e.g., cherries, strawberries, blackberries or raspberries), the plantain family or grapevines, leguminous crops, such as beans, lentils, peas and soya, oil crops, such as sunflower, safflower, rapeseed, canola, castor or olives, cucurbits, such as cucumbers, melons or pumpkins, fiber plants, such as cotton, flax or hemp, fuel crops, such as sugarcane, miscanthus or switchgrass, vegetables, such as potatoes, tomatoes, peppers, lettuce, spinach, onions, carrots, eggplants, asparagus or cabbage, ornamentals, such as flowers (e.g., petunias, pelargoniums, roses, tulips, lilies, or chrysanthemums), shrubs, broad-leaved trees (e.g., poplars or willows) and evergreens (e.g., conifers), grasses, such as lawn, turf or forage grass or other useful plants, such as coffee, tea, tobacco, hops, pepper, rubber or latex plants.

According to specific embodiments, the plant may be selected from the group consisting of maize, soybean, alfalfa, cotton, sunflower, Brassica oil seeds such as Brassica napus (e.g. canola, rape-seed), Brassica rapa, Brassica juncea (e.g. (field) mustard) and Brassica carinata, Arecaceae sp. (e.g. oilpalm, coconut), rice, wheat, sugar beet, sugar cane, oats, rye, barley, millet and sorghum, triticale, flax, nuts, grapes and vine and various fruit and vegetables from various botanic taxa, e.g. Rosaceae sp. (e.g. pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g. olive tree), Actinidaceae sp., Lauraceae sp. (e.g. avocado, cinnamon, camphor), Musaceae sp. (e.g. banana trees and plantations), Rubiaceae sp. (e.g. coffee), Theaceae sp. (e.g. tea), Sterculiceae sp., Rutaceae sp. (e.g. lemons, oranges, mandarins and grapefruit); Solanaceae sp. (e.g. tomatoes, potatoes, peppers, capsicum, aubergines, tobacco), Liliaceae sp., Compositae sp. (e.g. lettuce, artichokes and chicory—including root chicory, endive or common chicory), Umbelliferae sp. (e.g. carrots, parsley, celery and celeriac), Cucurbitaceae sp. (e.g. cucumbers—including gherkins, pumpkins, watermelons, calabashes and melons), Alliaceae sp. (e.g. leeks and onions), Cruciferae sp. (e.g. white cabbage, red cabbage, broccoli, cauliflower, Brussels sprouts, pak choi, kohlrabi, radishes, horseradish, cress and Chinese cabbage), Leguminosae sp. (e.g. peanuts, peas, lentils and beans—e.g. common beans and broad beans), Chenopodiaceae sp. (e.g. Swiss chard, fodder beet, spinach, beetroot), Linaceae sp. (e.g. hemp), Cannabeacea sp. (e.g. cannabis), Malvaceae sp. (e.g. okra, cocoa), Papaveraceae (e.g. poppy), Asparagaceae (e.g. asparagus); useful plants and ornamental plants in the garden and woods including turf, lawn, grass and Stevia rebaudiana, and genetically modified types of these plants.

According to specific embodiments, the plant may be a harvestable part of the plant selected from the group consisting of a fruit, a flower, a nut, a vegetable, a fruit or vegetable with inedible peel, such as avocados, bananas, plantains, lemons, grapefruits, melons, oranges, pineapples, kiwi fruits, guavas, mandarins, mangoes and pumpkin.

According to specific embodiments, the plant is forest plant, such as described in e.g. www(dot)fao(dot)org/3/10640e/i0640e13.pdf.

According to specific embodiments, the plant is an ornamental plant.

According to specific embodiments, the plant may be a cut flower of ornamental plants, preferably selected from Alstroemeria, carnation, Chrysanthemum, Freesia, Gerbera, Gladiolus, baby's breath (Gypsophila spp.), Helianthus, Hydrangea, Lilium, Lisianthus, roses and summer flowers.

According to specific embodiments, the plant is a cut grass or wood.

According to specific embodiments, the plant is cotton.

According to specific embodiments, the plant is a transgenic plant.

According to specific embodiments, the plant is a transgenic plant recombinantly expressing the nanobody or the composition comprising the nanobody and the toxin.

Methods of generating a transgenic plant are well known in the art and are also described hereinabove.

Following, the present invention also encompasses products comprising the nanobody or the composition comprising the nanobody and the toxin, polynucleotides or nucleic acid constructs encoding same or host cells expressing same.

According to specific embodiments, such products are more resistant to insect infestation or damage as compared to products not comprising the nanobody or the composition comprising the nanobody and the toxin, polynucleotides or nucleic acid constructs encoding same or host cells expressing same.

Thus, according to an aspect of the present invention, there is provided a plant comprising the nanobody or the composition comprising the nanobody and the toxin, or a polynucleotide or a nucleic acid construct encoding same.

According to another aspect of the present invention, there is provided a commodity product comprising the nanobody or the composition comprising the nanobody and the toxin.

According to another aspect of the present invention, there is provided a surface covered with the nanobody or the composition comprising the nanobody and the toxin.

According to another aspect of the present invention, there is provided a packaged product comprising a commodity product contained within a packaging material comprising the nanobody or the composition comprising the nanobody and the toxin.

Tables 1-8 hereinbelow list polypeptides and nanobodies that can be used with specific embodiments of the present invention.

TABLE 1
Target proteins Amino Acid Sequences

| SEQ | | |
|---|---|---|
| ID | | |
| NO: | Target | Amino Acid Sequence |
| 1 | A polypeptide | MGSSHHHHHHSSGLVPRGSHMGDRGISEPGNDQGNDNDSN |
| | comprising | DNNSSNEQGGVCNCNPEEAPAICASPGSEGVLVAHENCEK |
| | Chitin binding | YYICNHGRPVVASCSGNLLFNPYTNECGWPRDVDCGDRI |
| | domain CBD- | EPGCTGCNDNNNNDDDDSDCDGDDPVPPPADNDDSESADI |
| | HaPMP5B1 | DDLPPPGDDASVRPPVDEGTCNCNPEQAPSICAEDDSDGVL |
| | (The CBDs are | VAHEDCNKFYKCHNGKPVALYCPGNLLYNPNTEQCDW |
| | marked in | PEKVDCGDRVIPDPEDNTVGGNNDGEDDSEGVLVAHENC |
| | bold, SEQ ID | NQFYKCSGGKPVALLCPGNLLFNPNTDQCDWPWEVDC |
| | Nos: 3-7) | GDRIIPDPDRTHCGSHCSTHCSTHCGSLLRLPLRLHCGSHCG |
| | | SHCCTNTATNRRRNMQLQSWSTFHLCSRRLLIAHEDCNKF |
| | | YICDHGKPVALSCPGNLLYNPYTEKCDWPENVECGDRAP |
| | | DPDASQAPAICADSGSEGVLVAHENCDQYYICDGGRPVA |
| | | RPCQGGLLYNPLTQYCDGQEMSTAVTGLSLMTAPVIPEM |
| | | RPDCAVSQTPKEAW |
| 2 | A polypeptide | MGSSHHHHHHSSGLVPRGSHMDRVIPDPEDNTVGGNNDGE |
| | comprising | DDSEGVLVAHENCNQFYKCSGGKPVALLCPGNLLFNPN |
| | Chitin binding | TDQCDWPWEVDCGDRIIPDPDRTHCGSHCSTHCSTHCGSL |
| | domain CBD- | LRLPLRLHCGSHCGSHCCTNTATNRRRNMQLQSWSTFHLC |
| | HaPMP5B2 | SRRLLIAHEDCNKFYICDHGKPVALSCPGNLLYNPYTEKC |
| | (The CBDs are | DWPENVECGDRAPDPDASQAPA |
| | marked in | |
| | bold, SEQ ID | |
| | Nos: 5 and 7) | |
| 8 | V-ATPase sub | MHHHHHHMSEYWVISAPGDKTCQQTWDTLNNATKSGNLS |
| | unit c | ANYKFPIPDLKVGTLDQLVGLSDDLGKLDTFVESVTRKVAQ |
| | | YLGEVLEDQRDKLHENLMANNSDMPSYLTRFQWDMAKYP |
| | | IKQSLRNIADIISKQVGQIDSDLKQKSAAYNALKGNLQNLEK |
| | | KQTGSLLTRNLADLVKREHFILDSEYLTTLLVIVPKSMFND |
| | | WTANYEKITDMIVPRSSQLIHQDNDYGLFNVTLFKKVVEEF |
| | | KHHARERKFVVREFSYNEADMAAARTRSPSSSPTRRSSSILR |
| | | KFINFLGPLVRWLKVNFSECFCAWIHVKALRVFVESVLRYG |
| | | LPVNFQAVVMVPSRKNTKKLREVLQTLYAHLDHSAHQHTS |
| | | SAQDNAELAGLGFGSSEYFPYVFYKINVDMLDKN |
| 9 | Chitin | METRVKRQEEDGGDEVNAEQLCDGRPADEYFRLTTEGDCR |
| | deacetylase | DVVRCTRSGLKQITCPSGLAFDLDKQTCDWKGKVTNCDKL |
| | | EKPRKVLPILKTDEPICPEGKLACGSGDCIEKELFCNGKPDC |
| | | KDESDENACTVDVDPNRAPDCDPNQCALPDCFCSADGTRIP |
| | | GGIEVNQVPQMITITFNGAVNVDNIDLYEQIFNGNRHNPNG |
| | | CQIRGTFFVSHKYTNYAAVQELHRKGHEISVFSITHKDDPQ |
| | | YWSSGSYDDWLAEMAGARLIVERFANITDSSIIGVRAPYLR |
| | | VGGNKQFEMMADQYFVYDASITAPLGRVPIWPYTLYFRMP |
| | | HKCNGNAHNCPSRSHPVWEMVMNELDRRDDPTFDESLPG |
| | | CHVVDSCSNIQTGEQFARLLRHNSNRHYSTNRAPLGFHFHA |
| | | SWLKSKKEFRDELIKFIEEMLEKNDVYFTSLIQVIQWMQNP |
| | | TELTSLRDFQEWKQDKCDVKGQPFCSLPNACPLTTRELPGE |
| | | TLRLFTCMECPNNYPWILDPTGEGFNVK |
| 10 | Trehalase | MDLPLTCTKPVYCNSNLLHQIQMARLYNDSKTFVDLQMNF |
| | | DENKTLTDFETFFNLHNKNPTKEQLMBFVNEYFSNDNELEP |
| | | WQPKDFSDNPAFLAKIKDDALREFGKGINNIWPLLARKVKA |
| | | EVFQKPDQFSLVPLTHGFIIPGGRFKEIYYWDTFWIIEGLLIS |
| | | GMQETAKGMIENLIELLNLFGHIPNGSRGYYQQRSQPPMLN |
| | | AMVATYYMYTKDLEFLRNNIAYLEKELDFWMDNRVVSVN |
| | | RGGKNYTLLRYYAPSKGPRPESYYEDYSNTEGFSEEDSTNF |
| | | CIDIKSAAESGWDFSTRWFLMPDGSNNGTLTDLHTRYIIPVD |
| | | LNAIFAGAAQYVSNFHALLKNPQKAARYGQLAQTWRDNIQ |
| | | AVLWNDQDAMWYDFNIRDNLHRRYYYSSNAAPLWQNAV |
| | | NPDFLKLNADRILKAITESGGVDFPGGVPTSLIRSGEQWDFP |
| | | NVWPPEVSIEVAAIENIGTPEAITLAQEVAQTFVRSCHWGFQ |
| | | KYKQMFEKYDAETPGRFGGGGEYNVQFGFGWSNGVVLEF |
| | | LNKYGSQLTADDSNNTNNS |
| 11 | cytochrome | MRTFNYWKKRNVRGPEPVVFFGNLKDSALRKKNMGVVM |
| | p450 | EELYNMFPEEKVIGIYRMTSPCLLVRDLDVIKHIMIKDFEVF |
| | monooxygenase | SDRGVEFSKEGLGSNLFHADGETWRALGNRFTPIFTSGKLK |
| | | NMFYLMHEGADNFIDHVSAECEKNQEFEVHSLLQTYTMSTI |
| | | AACAFGISYDSIGDKVKALDIVDKIISEPSYAIELDMMYPGL |
| | | LSKLNLSIFPTVVKNFFKSLVDNIVAQRNGKPSGRNDFMDLI |
| | | LELRQLGEVTSNKYGSSASSLEITDEVICAQAFVFYIAGYET |
| | | SATTMAYMIYQLALNPDIQNKLIAEVDEVLKANDGKVTYD |
| | | TVKEMKYLNKAFDETLRMYSIVEPLQRKATRDYKIPGTDV |
| | | VIEKDTIVLISPRGIHYDPKYYDNPKQFNPDRFDAEEVGKRH |
| | | PCAYLPFGLGQRNCIGMRFGRLQSLLCITKILSKFRIEPSKNT |
| | | DRNLQVEPHRGLIGPKGGIRVNAIPRKLVS |
| 12 | Chitin | NCYFHGTVPDYLYFESPPVFTLSDFISRQMAWICRTFGLNEK |
| | synthase | LFVMPMYNGLLIDQSMALNRKRNDQRDVKTEDLAEIEKEK |
| | | GDEYYETISVHTDNTGSSPKAIKSSDQITRIYACATMWHETK |
| | | DEMMEFLKSILRLDEDQCARRVAQKYLRVVDPDYYEFETH |
| | | IFLDDAFEISDHSDDDSQVNRFVKLLVDTIDEAASEVHQTNI |
| | | RDVHVLPSRSSFNGTADILDRKEVMAENTYLLTLDGDIDFQ |
| | | PHAVRLLIDLMKKNKNLGAACGRIHPVGSGPMVWYQMFE |
| | | YAIGHWLQKATEHMIGCVLCSPGCFSLFRGKALMDDNVM |
| | | KKYTLRSDEARHYVHTIRGRSMVMYAITATWLSCRILSCLR |
| | | CYTHCPEGFNEFYNRRRWVPSTIANIMDLLADCKHTIKIND |
| | | NISSPYIA |
| 13 | NPC1-sterol | DSFQTKYFQYLNRYLNIGPPVYFVVTEGLNYSDMDTQNMI |
| | transporter | CGTRFCRPDSLSMQLYAAYRNPNETYIAQPPNSWLDDYFD |
| | | WSALPNCCKYFPSNSSFCPNDRGAPCKACGIALEGDEQRPN |
| | | STEFERYVPFFLQDIPDTSGSGCVKGGHAAYGQAVNYKMF |
| | | NKTQAHVGATYYQGYHTVLKTSLDYYSALKGAREVAANL |
| | | TETLNRNLKHQLNGTTINVFPYSVFYVFYEQYLTMWPDTL |

TABLE 2A
Amino acid sequences of anti-CBD Nanobodies

| | Nanobody | |
|---|---|---|
| SEQ ID NO: | name | Amino Acid Sequence |
| 14 (The CDRs | CB20501 | MQVQLQESGGGLVQAGDSLRLSCAASG |
| are marked | | RSFSIYTMGWFRQAPGNEREFVTAISP |
| in bold, SEQ | | SGGSTHYADFVKGRFTISRDNTKNTMY |
| ID Nos: | | LQMNSLKPEDTAVYFCASTSSQHYEDT |
| 15-17) | | EESYKYWGQGTQVTVSSAAAYPYDVPD |
| | | YGSHHHHHH |
| 18 (The CDRs | CB20801 | MQVQLQESGGGLVEAAGSLRLSCAALG |
| are marked | | SNLRINTMGWYRQAPGKQRELVATITN |
| in bold, SEQ | | GGRKNYADSVKGRFTIFRGNANTVYLQ |
| ID Nos: | | MNSLKPEDTAVYYCNAGLLDPPYSAPG |
| 19-21) | | DYWGEGTQVTVSSAAAYPYDVPDYGSH |
| | | HHHHH |
| 22 (The CDRs | CB20901 | MQVQLQESGGGLVQAGGSLTLSCAASG |
| are marked | | RSFSTYAMGWSRQAPGKEREFLAGISR |
| in bold, SEQ | | GGGTTVYADSVKGRFTISRDNVKNTVY |
| ID Nos: | | LQMNSLKPEDAAVYYCAAALRPFDGSG |
| 23-25) | | ERRYDYWGQGTQVTVSSAAAYPYDVPD |
| | | YGSHHHHHH |
| 26 (The CDRs | CB201101 | MQVQLQESGGGLVQAGDSLRLSCAASG |
| are marked | | RSFSIYTMGWFRQAPGKEREFVAAISP |
| in bold, SEQ | | SGVSTDYADSVRGRFTISRDNAKNTMY |
| ID Nos: 15, | | LQMNSLKPEDTAVYYCAAGGRHYTRHP |
| 28-29) | | YDYDYWGQGTQVTVSSAAAYPYDVPDY |
| | | GSHHHHHH |

TABLE 2B
Amino acid sequences of anti-CBD Nanobodies

| | Nanobody | |
|---|---|---|
| SEQ ID NO: | name | Amino Acid Sequence |
| 30 (The CDRs are | CB10101 | MQVQLQESGGGLVQTGGSLRLSCAASGRSF |
| marked in bold, | | SSYSMGWFRQGPGKGREWVADINESGSSTS |
| SEQ ID Nos: 31-33) | | YYDPVKGRFTISRDNSKNTVYLQMNDLKPE |
| | | DTADYYCAALVTGGDTDLGEWDFWGPGTQV |
| | | TVSSAAAYPYDVPDYGSHHHHHH |
| 34 (The CDRs are | CB10201 | MQVQLQESGGGLVQAGDSLKLSCAASGGTF |
| marked in bold, | | SSYVMGWFRQAPGKEREFVARIGVSEGYLY |
| SEQ ID Nos: 35-37) | | YADSVKDRFTISRDSAKNTGYLQMNALKPE |
| | | DTAVYYCAAGPRRYWTREPDAYDYWGQGTQ |
| | | VTVSSAAAYPYDVPDYGSHHHHHH |
| 38 (The CDRs are | CB10301 | MQVQLQESGGGLVQAGGSLKLSCGASGRTF |
| marked in bold, | | STNAMGWFRQAPGKEREFVATISAGGSLTY |
| SEQ ID Nos: 39-41) | | YADSVKGRFTISRDNAKNTVYLRMNSLKPD |
| | | DTAVYYCAADQDSGRLPLINSGYEYWGQGT |
| | | QVTVSSAAAYPYDVPDYGSHHHHHH |
| 42 (The CDRs are | CB10401 | MQVQLQESGGGLVQAGASLRLSCAASGRTF |
| marked in bold, | | SSYGTAMGWFRQAPGKEREFVAAILWTGSS |
| SEQ ID Nos: 43-45) | | SYDSVKGRFTISRDNAKNTVYLQMISLNPE |
| | | DTAVYYCAARSRYTGSYYEESTYNYWGQGT |
| | | QVTVSSAAAYPYDVPDYGSHHHHHH |
| 46 (The CDRs are | CB10501 | MQVQLQESGGGVVQPGGSLRLSCAASGRDF |
| marked in bold, | | SNYNMAWFRQAPGKERDVVATIRRSGDITS |
| SEQ ID Nos: 47-49) | | YTDSVKGRFTISRDNAKNTVYLQMNSLKPE |
| | | DTAAYYCAARTGSFLTVLITTPGNYNYWGQ |
| | | GTQVTVSSAAAYPYDVPDYGSHHHHHH |
| 50 (The CDRs are | CB10701 | MQVQLQESGGGLVQAGDSLRLSCAASGRTF |
| marked in bold, | | SAFRMAWFRQAPGKERALVADISRLSTRTY |
| SEQ ID Nos: 51-53) | | YADSVKGRFTISRDNAKNTVYLQMNSLKPE |
| | | DTAVYYCAADLEGVGPMWEYWVQGTQVIVS |
| | | SAAAYPYDVPDYGSHHHHHH |
| 54 (The CDRs are | CB10801 | MQVQLQESGGGSVQAGNSLRLSCTYSGRTF |
| marked in bold, | | STRAMGWFRQAPGKERELVAGIGWNGATQY |
| SEQ ID Nos: 55-57) | | YADSVKGRFTISRDSASNTVALQMNSLEPE |
| | | DTAVYYCAAHGREYVTPSYNNYDYWGQGTQ |
| | | VTVSSAAAYPYDVPDYGSHHHHHH |
| 58 (The CDRs are | CB101001 | MQVQLQESGGGLTQAGDSLRLSCAASGRSF |
| marked in bold, | | VRYTTGWFRQAPGKEREFVASISWSRGSTY |
| SEQ ID Nos: 59-61) | | YADSVKGRFTISKDNAENTVYLQMNSPEPE |
| | | DTAVYYCAGNSRGATTFAQYYDDWGQGTQV |
| | | TVSSAAAYPYDVPDYGSHHHHHH |
| 62 (The CDRs are | CB101101 | MQVQLQESGGGLVQAGGSLRLSCAASGRTF |
| marked in bold, | | SLTRMGWFRQAPGKEREIVAHIMRSSDSTF |
| SEQ ID Nos: 63-65) | | YGDSVKGRFTISRDNAKNTVYLQMNRLNPE |
| | | DTAVYYCAAAQWAGYDYWGQGTQVTVSSAA |
| | | AYPYDVPDYOSHHHHHH |
| 66 (The CDRs are | CB101201 | MQVQLQESGGGLVPAGGSLRLSCAASGRTF |
| marked in bold, | | TSSTMAWFRQVPGKEREFVAAISPRGLSQD |
| SEQ ID Nos: 67-69) | | YGHSVKGRETISRDNAENTVYLQMNSLKSE |
| | | DTALYYCAATSGSYSSSRNDYYYWGQGTQV |
| | | TVSSAAAYPYDVPDYGSHHHHHH |
| 70 (The CDRs are | CB101301 | MQVQLQESGGGLVQPGGSLRLSCAASGFTL |
| marked in bold, | | SNYRMAWFRQGLGKEREFVAHIMRNSDTTW |
| SEQ ID Nos: 71-73) | | YTESVKGRFTISRDNSKNTVYLQMNSLKPE |
| | | DTAVYYCAASNAGTFDYWGQGTQVTVSSAA |
| | | AYPYDVPDYGSHHHHHH |
| 74 (The CDRs are | CB101401 | MQVQLQESGGGLVQPGGSLRLSCASSGANI |
| marked in bold, | | RLYGMAWYRQPPGEERELVASITVGGSITY |
| SEQ ID Nos: 75-77) | | AESVKARFTISRDNARDMVFLQMNSLKPED |
| | | TAVYYCNAMNPWYYWAWGQGTQVTVSSAAA |
| | | YPYDVPDYGSHHHHHH |
| 78 (The CDRs are | CB101601 | MQVQLQESGGGLVQAGGSLRLSCAASGGTL |
| marked in bold, | | SSYDMGWFRQAPGKGRDFVAGIDWSGGSTN |
| SEQ ID Nos: 79-81) | | YERSVKGRFTITRDNAKNTVLLQMNSLKPE |
| | | DTAVYYCAAARANSDLGIYDYWGQGTQVTV |
| | | SSAAAYPYDVPDYGSHHHHHH |
| 82 (The CDRs are | CB101701 | MQVQLQESGGGLVEAGGSLGLACTTSGIIF |
| marked in bold, | | SRNDMGWFRQAPGEQRTAVATITRSSSTNY |
| SEQ ID Nos: 83-85) | | AGSVKGRFTISRDNAENTVYLQMSNLKPED |
| | | TAVYYCATIPTATQPYVYWGQGTQVTVSSA |
| | | AAYPYDVPDYGSHHHHHH |
| 86 (The CDRs are | CB101801 | MQVQLQESGGGLVQTGGSLRLSCAASGRSF |
| marked in bold, | | SSYSMGWFRQGPGKGREWVADINESGTTTN |
| SEQ ID Nos: 87-89) | | YWDPVKGRFIISRDNAQNMVYLQMNSLKPE |
| | | DTAVYYCAAGPRTRWTREPDAYDYWGQGTQ |
| | | VTVSSAAAYPYDVPDYGSHHHHHH |
| 90 (The CDRs are | CB102001 | MQVQLQESGGGLVQAGGSLRLSCVASGRTF |
| marked in bold, | | STYDMGWFRQAPGKGREFVAGIDWSGGSTN |
| SEQ ID Nos: 91, | | YVNFVKGRFTISRDNAKNTMYLQMNSLKPE |
| 80, 93) | | DTAVYYCAAAVGDSEMATYDYWGQGTQVTV |
| | | SSAAAYPYDVPDYGSHHHHHH |
| 94 (The CDRs are | CB102901 | MQVQLQESGGGLVQVGGSLRLSCVASEPTF |
| marked in bold, | | RPNRMGWFRQAPGKERELVAHIMWSSGSTW |
| SEQ ID Nos: | | YGDSVKGRFTISRDNAKNTVILQMNSLNPE |
| 95-97) | | DTAVYYCAAAQRAGYDYWGQGTQVTVSSAA |
| | | AYPYDVPDYGSHHHHHH |
| 98 (The CDRs are | CB103001 | MQVQLQESGGGLVQAGGSLRLSCAASGRSL |
| marked in bold, | | SSYSMGWIRQAPGKERDFVTAIRWSGGSTY |
| SEQ ID Nos: | | YADSVKGRFTISRDIAKNAVYLQMNSLKTE |
| 99-101) | | DTAVYYCAARLGGRSWDAGDYQYWGQGTQV |
| | | TVSSAAAYPYDVPDYGSHHHHHH |
| 102 (The CDRs are | CB103101 | MQVQLQESGGGSVQSGGSLFLSCAASGFVF |
| marked in bold, | | ETSPMTWVRQAPGQGVRWVGSITTDGRRAD |
| SEQ ID Nos: | | YEDAVKGRFTISRDNVKNMLYLEMNNLKPE |
| 103-105) | | DTAMYFCRESRDLNAVTRGTQVTVSSAAAY |
| | | PYDVPDYGSHHHHHH |
| 106 (The CDRs are | CB103301 | MQVQLQESGGGLVQTGDSLRLSCAVSGRTG |
| marked in bold, | | SINRMGWFRQAPGKEREIVSHIFWSNVGTW |
| SEQ ID Nos: | | SAESVKGRFIISRDNAKNTVYLQMNSLKPE |
| 107-109) | | DTAVYSCAAATGSAYNYWVPSRGDPGHVSS |
| | | AAAYPYDVPDYGSHHHHHH |
| 110 (The CDRs are | CB20401 | MQVQLQESGGGQVQAGDSLRLSCVTSGMSF |
| marked in bold, | | STSAMGWYRQASGKQREWVAIIREDSTTNY |
| SEQ ID Nos: | | SSFAKGRFTISRDNTNKTVYLLMNSLEPDD |
| 111-113) | | TAVYYCRTYTGGYWGQGTQVTVSSAAAYPY |
| | | DVPDYGSHHHHHH |
| 114 (The CDRs are | CB20601 | MQVQLQESGGGLVQAGGSLRLSCAASGRTF |
| marked in bold, | | SSYAMGWFRQAPGKEREFVAGISWSGRSTY |
| SEQ ID Nos: | | YADSVKGRFTISRDDAKNTVYLQMNSLKPE |
| 115-117) | | DTAVYYCAAPDTAAQFTTPLYEYAYWGQGT |
| | | QVTVSSAAAYPYDVPDYGSHHHHHH |
| 118 (The CDRs are | CB201001 | MQVQLQESGGGLVQAGDSLRLACASSSRTF |
| marked in bold, | | STYTMGWFRQTPGRERDFVAAISPSGATAD |
| SEQ ID Nos: | | YADSVKGRFTISRDNAKNTLYLQMDSLKPE |
| 119-121) | | DTAVYYCAARYLSWSRMNHEYPYWGQGTQV |
| | | TVSSAAAYPYDVPDYGSHHHHHH |
| 122 (The CDRs are | CB201201 | MQVQLQESGGGLVQAGDSLRLSCAASERTF |
| marked in bold, | | SSYVMGWFRQAPRKEREFVAAMTWSGSSRI |
| SEQ ID Nos: | | YYADSVKGRFTISRDNAKNTAYLQMNSLKP |
| 123-125) | | EDTAVYYCAAKDAYGGISFRPNTYHSWGQG |
| | | TQVTVSSAAAYPYDVPDYGSHHHHHH |
| 126 (The CDRs are | CB201301 | MQVQLQESGGGLVQPGGSLRLSCAALGNIV |
| marked in bold, | | NINNMGWYRQAPGGQRELVATITRGAIKNY |
| SEQ ID Nos: | | ADSVKGRFTIFRGNANTVYLQMNSLKPEDT |
| 127-129) | | AVYYCVADSSWGQGTQVTVSSAAAYPYDVP |
| | | DYGSHHHHHH |
| 130 (The CDRs are | CB201401 | MQVQLQESGGGVVQAGGFLRLSCAASGLIF |
| marked in bold, | | DDTAIGWFRQAPGKEREFVAAVSPSGVSTD |
| SEQ ID Nos: | | YTDSVKGRFTISRDNAKKTVFLQMSRLKPE |
| 131-133) | | DTAVYYCAARLRHYSNDQHEYDSWGQGTQV |
| | | TVSSAAAYPYDVPDYGSHHHHHH |
| 134 (The CDRs are | CB201501 | MQVQLQESGGGLVQAGGSLRLSCAASGIVF |
| marked in bold, | | SITTMGWYRQAPGKQRELVATIASGVRADY |
| SEQ ID Nos: | | ADSVKGRFTISRDNGKNTVYLQMNSLKPED |
| 135-137) | | TAVYYCNANRFSLGNYWGQGTQVTVSSAAA |
| | | YPYDVPDYGSHHHHHH |
| 138 (The CDRs are | CB201601 | MQVQLQESGGGLVQTGDSLRLSCAASGGTF |
| marked in bold, | | SAYTMGWFRQAPGKEREFVAAISRSGSSTH |
| SEQ ID Nos: | | YANSVKGHFTISRDNAKKTVYLQMNSLKPE |
| 139-141) | | DTARYYCAGERTGHFTDLYYEYDYWGQGTQ |
| | | VTVSSAAAYPYDVPDYGSHHHHHH |
| 142 (The CDRs are | CB201701 | MQVQLQESGGGLVQAGGSLRLSCAAPGSNF |
| marked in bold, | | RINTMGWYRQAPGKQRELVATIANSGRINS |
| SEQ ID Nos: | | ANSVKGRFTIFRGNANTVYLQMNSLKPEDT |
| 143-145) | | AVYYCNADVVESSNYQTLNYWGQGTQVTVS |
| | | SAAAYPYDVPDYGSHHHHHH |
| 146 (The CDRs are | CB201801 | MQVQLQESGGGLVQAGGSLRLSCVASRASG |
| marked in bold, | | SIFGAQTMAWYRQASGERRELVATITSSGS |
| SEQ ID Nos: | | TNYADSVKGRFTISRDNAKSTMFLQMNNLK |
| 147-149) | | PEDTAVYYCNVGFRSRYSYDSSVWGEGTQV |
| | | TVSSAAAYPYDVPDYGSHHHHHH |
| 150 (The CDRs are | CB201901 | MQVQLQESGGGLVQAGGSLRLSCAASGRTF |
| marked in bold, | | STSNMGWFRQAPGKEREFVGAISPSGRSTD |
| SEQ ID Nos: | | YASSVEGRFTISRDNPKNTMYLQMNSLKPE |
| 151-153) | | DTAVYYCAARRSPSYTRVGDEYDYWGQGTQ |
| | | VTVSSAAAYPYDVPDYGSHHHHHH |
| 154 (The CDRs are | CB202201 | MQVQLQESGGGLVQAGDSLRLSCAASGRSF |
| marked in bold, | | SIYTMGWFRQAPGKEREFVSLIMRSGGIIY |
| SEQ ID Nos: 15, | | ADFVKGRFTISRDNAKNLVYLQMNSLKPED |
| 156-157) | | TAVYYCAAGGSTNSRAYNYYKLSLAYDYWG |
| | | QGTQVTVSSAAAYPYDVPDYGSHHHHHH |
| 158 (The CDRs are | CB202501 | MQVQLQESGGGLVQAGDSLRLSCAASGGTF |
| marked in bold, | | STYTMGWFRQAPGKEREFVAAISRTGRGTD |
| SEQ ID Nos: | | YADSVKGRFTISRDNVKNTVSLQMNSLKPE |
| 159-161) | | DTAVYYCAERSSVHYSGIAADYDYWSQGTQ |
| | | VTVSSAAAYPYDVPDYGSHHHHHH |
| 162 (The CDRs are | CB202901 | MQVQLQESGGGLVQPGGSLRLSCAASGRNL |
| marked in bold, | | SRSAMGWFRQNPGEEREFVAAIDWWGDSTY |
| SEQ ID Nos: | | YGDSVTGRFTISRDNAKNTLYLQMNSLKPE |
| 163-165) | | DTAVYYCAAGLRPFDGSWERRYDYWGQGTQ |
| | | VTVSSAAAYPYDVPDYGSHHHHHH |

TABLE 3A
Amino acid sequences of anti-V-ATPase subunit Nanobodies

| | Nanobody | |
|---|---|---|
| SEQ ID NO: | name | Amino Acid Sequence |
| 166 (The CDRs are | VAT0101A | QVQLQESGGGLVQAGGSLRLSCAASGSTF |
| marked in bold, SEQ ID | | SGYIMSWFRQPPGKERELVAAITYVGSTY |
| Nos: 167-169) | | VEDSVKGRFTISRDNAKNTVSLQMNSLKP |
| | | EDTAVYYCRAREGEQLDFWGQGTQVTVSS |
| | | AAAYPYDVPDYGSHHHHHH |
| 170 (The CDRs are | VAT0101B | QVQLQESGGGLVQAGGSLRLSCAASGSTL |
| marked in bold, SEQ ID | | SGYIMSWFRQPPGKERELVAAITYVGSTY |
| Nos: 171,168-169) | | VEDSVKGRFTISRDNAKNTVSLQMNSLKP |
| | | EDTAVYYCRAREGEQLDFWGQGTQVTVSS |
| | | AAAYPYDVPDYGSHHHHHH |
| 172 (The CDRs are | VAT0101C | QVQLQESGGGLVQSGDSLRLSCAASGSTF |
| marked in bold, SEQ ID | | SGYIMSWFRQPPGKERELVAAITYVGSTY |
| Nos: 167-169) | | VEDSVKGRFTISRDNAKNTVSLQMNSLKP |
| | | EDTAVYYCRAREGEQLDFWGQGTQVTVSS |
| | | AAAYPYDVPDYGSHHHHHH |
| 173 (The CDRs are | VAT0101D | QVQLQESGGGLVQPGGSLRLSCVASGSTF |
| marked in bold, SEQ ID | | SGYIMSWFRQPPGKERELVAAITYVGSTY |
| Nos: 174, 168-169) | | VEDSVKGRFTISRDNAKNTVSLQMNSLKP |
| | | EDTAVYYCRAREGEQLDFWGQGTQVTVSS |
| | | AAAYPYDVPDYGSHHHHHH |
| 175 (The CDRs are | VAT0101E | QVQLQESGGGLVQAGDSLRLSCAASGSTF |
| marked in bold, SEQ ID | | SGYIMSWFRQPPGKERELVAAITYVGSTY |
| Nos: 167-169) | | VEDSVKGRFTISRDNAKNTVSLQMNSLKP |
| | | EDTAVYYCRAREGEQLDFWGQGTQVTVSS |
| | | AAAYPYDVPDYGSHHHHHH |
| 176 (The CDRs are | VAT0101F | QVQLQESGGGTVQAGGSLRLSCAASGSTF |
| marked in bold, SEQ ID | | SGYIMSWFRQPPGKERELVAAITYVGSTY |
| Nos: 167-169) | | VEDSVKGRFTISRDNAKNTVSLQMNSLKP |
| | | EDTAVYYCRAREGEQLDFWGQGTQVTVSS |
| | | AAAYPYDVPDYGSHHHHHH |
| 177 (The CDRs are | VATO101G | QVQLQESGGGLVQAGGSLRLSCAASGSTF |
| marked in bold, SEQ ID | | SGYIMSWFRQPPGKERELVAAITYVGSTW |
| Nos: 167, 178, 169) | | YQDSVKGRFTISRDNAKNTVSLQMNSLKP |
| | | EDTAVYYCRAREGEQLDFWGQGTQVTVSS |
| | | AAAYPYDVPDYGSHHHHHH |
| 179 (The CDRs are | VAT0201 | QVQLQESGGGTVQPGGSLKLSCAASGNLN |
| marked in bold, SEQ ID | | YINVWSWYRQAPGKQRERVAGIATGGGRI |
| Nos: 180-182) | | SYSESVKGRFIISRDDATNTVSLQMSGLT |
| | | PEDTAVYYCNAFGSDPDFSDYKHDYWGQG |
| | | TQVTVSSAAAYPYDVPDYGSHHHHHH |

TABLE 3B
Amino acid sequences of anti-V-ATPase subunit Nanobodies

| | Nanobody | |
|---|---|---|
| SEQ ID NO: | name | Amino Acid Sequence |
| 183 (The CDRs are | VAT101H01 | QVQLQESGGGLVQTGGSLRLSCAASGST |
| marked in bold, SEQ ID | | FSGYIMSWFRQPPGKERELVAAITYVGS |
| Nos: 167-169) | | TYVEDSVKGRFTISRDNAKNTVSLQMNS |
| | | LKPEDTAVYYCRAREGEQLDFWGQGTQV |
| | | TVSSAAAYPYDVPDYGSHHHHHH |
| 185 (The CDRs are | VAT101I01 | QVQLQESGGGLVQAGGSLRLSCAASGST |
| marked in bold, SEQ ID | | FSGYIMSWFRQPPGKERELVAAITYVGS |
| Nos: 167-169) | | TYVEDSVKGRFTISRDNVKNTVSLQMNS |
| | | LKPEDTAVYYCRAREGEQLDFWGQGTQV |
| | | TVSSAAAYPYDVPDYGSHHHHHH |
| 186 (The CDRs are | VAT10301 | QVQLQESGGGLVQPGGSLRLSCAASGTI |
| marked in bold, SEQ ID | | FSAKALGWHRQAPGKQREVVAGITSGGS |
| Nos: 187-189) | | TNYADSVKGRFTISRDNAKNTVYLQMNS |
| | | VKFEDTAVYYCVLYDLIKDRTYWGQGTQ |
| | | VTVSSAAAYPYDVPDYGSHHHHHH |
| 190 (The CDRs are | VAT10401 | QVQLQESGGGLVQPGGSLRLSCATSTSI |
| marked in bold, SEQ ID | | FSINVMDWYRQAPGKQRELVAGITSGDN |
| Nos: 191-193) | | TNYADSVKGRFTISRDNANNTVWLQMNS |
| | | LKPEDTAVYYCRGRVYNGGWYDYWGQGT |
| | | QVTVSSAAAYPYDVPDYGSHHHHHH |

TABLE 4
Amino acid sequences of anti-trehalase Nanobodies

| | Nanobody | |
|---|---|---|
| SEQ ID NO: | name | Amino Acid Sequence |
| 587 (The CDRs are | TRH1a | QVQLQESGGGLVQAGGSLRLSCATSGRAF |
| marked in bold, SEQ ID | | TNYHMGWFRQTPGKEREFVALISWSGDST |
| Nos: 588-590) | | RYADSVKGRFTISRDNAKRTVALQMDSLK |
| | | PEDSAVYYCASRLVGQSQYEVWGQGTQVT |
| | | VSSAAAYPYDVPDYGSHHHHHH |
| 591 (The CDRs are | TRH1b | QVQLQESGGGLVQAGGSLRLSCAASGRAF |
| marked in bold, SEQ ID | | TNYHMGWFRQTPGKEREFVALISWSGDST |
| Nos: 588-589 and 592) | | RYADSVKGRFTISRDNAKRTVALQMDSLK |
| | | PEDSAVYYCASRLVGQSQYEIWGQGTQVT |
| | | VSSAAAYPYDVPDYGSHHHHHH |
| 593 (The CDRs are | TRH1c | QVQLQESGGGLVQAGGSLRLSCAASGRAF |
| marked in bold, SEQ ID | | TNYHMGWFRQTPGKEREFVALISWSGDST |
| Nos: 588-590) | | RYADSVKGRFTISRDNAKRTVALQMDSLK |
| | | PEDSAVYYCASRLVGQSQYEVWGQGTQVT |
| | | VSSAAAYPYDVPDYGSHHHHHH |
| 594 (The CDRs are | TRH1d | QVQLQESGGGLVQAGGSLRLSCAASTRAF |
| marked in bold, SEQ ID | | TNYHMGWFRQTPGKEREFVALISWSGDST |
| Nos: 595, 589 and 592) | | RYADSVKGRFTISRDNAKRTVALQMDSLK |
| | | PEDSAVYYCASRLVGQSQYEIWGQGTQVT |
| | | VSSAAAYPYDVPDYGSHHHHHH |
| 596 (The CDRs are | TRH1e | QVQLQESGGGLVQTGGSLRLSCAASGRAF |
| marked in bold, SEQ ID | | TNYHMGWFRQTPGKEREFVALISWSGDST |
| Nos: 588-590) | | RYADSVKGRFTISRDNAKRTVALQMDSLK |
| | | PEDSAVYYCASRLVGQSQYEVWGQGTQVT |
| | | VSSAAAYPYDVPDYGSHHHHHH |
| 194 (The CDRs are | TRH2a | QVQLQESGGGLVQAGGSLRLACAASERTF |
| marked in bold, SEQ ID | | SSYVMGWFRQGPGKEREFVAAISWSGGAR |
| Nos: 195-197) | | YYADSVKGRFTISRDNAKNTVYLQMSSLK |
| | | PEDTAVYVCAARRTYSPRTLEYDFWGQGT |
| | | QVTVSSAAAYPYDVPDYGSHHHHHH |
| 198 (The CDRs are | TRH2b | QVQLQESGGGLVQAGGSLRLSCAASGDTF |
| marked in bold, SEQ ID | | STYVMGWFRQGPGKEREFVAAISWSGGAR |
| Nos: 199, 196-197) ’ | | YYADSVKGRFTISRDNAKNTVYLQMSSLK |
| | | PEDTAVYVCAARRTYSPRTLEYDFWGQGT |
| | | QVTVSSAAAYPYDVPDYGSHHHHHH |
| 200 (The CDRs are | TRH3a | QVQLQESGGGLVQPGGSLRLSCAASGFTF |
| marked in bold, SEQ ID | | SDYVMSWVRQAPGKGFEWVSIINTDGIGS |
| Nos: 201-203) | | RYADSVMGRFTISRDNEKKMMYLQMNSLK |
| | | PEDTAMYYCARGNAALTVIRGRPWGQGTQ |
| | | VTVSSAAAYPYDVPDYGSHHHHHH |
| 204 (The CDRs are | TRH3b | QVQLQESGGGLVQPGGSLRLSCAASGFTF |
| marked in bold, SEQ ID | | SDYVMSWVRQAPGKGFEWVSIINTDGNGS |
| Nos: 201, 205-206) | | RYADSVMGRFTISRDNDKKMMYLQMNSLK |
| | | PEDTAMYYCAKGNAALSLIRGRPWGQGTQ |
| | | VTVSSAAAYPYDVPDYGSHHHHHH |
| 207 (The CDRs are | TRH5a | QVQLQESGGGLVQPGGSLRLSCAASGRTF |
| marked in bold, SEQ ID | | STYAMGWFRQAHGKEREFVAAISWGRGNT |
| Nos: 208-210) | | YYGDSLKGRFTISRDNAKNTVYLQMDSLK |
| | | PEDTAVYYCAADPGRAYVSGNYYSAATYD |
| | | YWGQGTQVTVSSAAAYPYDVPDYGSHHHH |
| | | HH |
| 211 (The CDRs are | TRH5b | QVQLQESGGGLVQAGGSLRLSCAASGRTF |
| marked in bold, SEQ ID | | SSYAMGWFRRFPGKEREFVAAISWGRGNT |
| Nos: 212, 209-210) | | YYGDSLKGRFTISRDNAKNTVYLQMDSLK |
| | | PEDTAVYYCAADPGRAYVSGNYYSAATYD |
| | | YWGQGTQVTVSSAAAYPYDVPDYGSHHHH |
| | | HH |
| 213 (The CDRs are | TRH5c | QVQLQESGGGLVQAGGSLRLSCAASGRTF |
| marked in bold, SEQ ID | | STYAMGWFRQAHGKEREFVAAISWGRGNT |
| Nos: 208-210) | | YYGDSLKGRFTISRDNAKNTVYLQMDSLK |
| | | PEDTAVYYCAADPGRAYVSGNYYSAATYD |
| | | YWGQGTQVTVSSAAAYPYDVPDYGSHHHH |
| | | HH |
| 214 (The CDRs are | TRH6 | QVQLQESGGGLVQAGGSLRLSCAASGGSF |
| marked in bold, SEQ ID | | STIPMGWFRQAPEMERGFVAAISYRGTYT |
| Nos: 215-217) | | YYTNSVKGRFTISRDNAKNTVYLQMNSLK |
| | | PEDTAVYYCAAGSPGISAYWGDLSNWKNW |
| | | GQGTQVTVSSAAAYPYDVPDYGSHHHHHH |
| 218 (The CDRs are | TRH7 | QVQLQESGGGVVQAGGSLRLSCVASGRSF |
| marked in bold, SEQ ID | | SSHAMGWFRQATGKEREFVATISWNSGST |
| Nos: 219-221) | | FYADSLRGRFTISRDNAKNTLYLQMNSLT |
| | | AEDTAVYYCAAASGRGITASAFRYDVWGQ |
| | | GTQVTVSSAAAYPYDVPDYGSHHHHHH |
| 222 (The CDRs are | TRH8 | QVQLQESGGGLVQAGDSLRLSCAASGRTF |
| marked in bold, SEQ ID | | SSYTMGWFRQAPGKEREFVAAIRWSGSIT |
| Nos: 223-225) | | YYSDSVKGRFTISRDNAKNTVYLQMNSLK |
| | | PEDTAVYYCAADPGRIFGSGSYYGNRNTY |
| | | DYWGQGTQVTVSSAAAYPYDVPDYGSHHH |
| | | HHH |
| 226 (The CDRs are | TRH9a | QVQLQESGGGLVQAGGSLRLSCAVSERTF |
| marked in bold, SEQ ID | | RTYTMAWFRQAPGKEREFVAAIRWNGDST |
| Nos: 227-229) | | YYADSVKGRFTISRDNAKNTMYLQMNSLK |
| | | PEDTAVYNCAARAPSGGYYYPNALSEYNY |
| | | WGQGTQVTVSSAAAYPYDVPDYGSHHHHH |
| | | H |
| 230 (The CDRs are | TRH9b | QVQLQESGGGLVQPGGSLRLSCAASERTF |
| marked in bold, SEQ ID | | RTYTMGWFRQAPGKEREFVAAIRWNGDST |
| Nos: 231,228,232) | | YYADSVKGRFTISRDNAKNTMYLQMNSLK |
| | | PEDTAVYSCAARAYSIGYYYPNALSEYNY |
| | | WGQGTQVTVSSAAAYPYDVPDYGSHHHHH |
| | | H |
| 233 (The CDRs are | TRH11 | QVQLQESGGGLVQAGDSLRLSCAASGVTI |
| marked in bold, SEQ ID | | SRYTMGWFRQAPGKEREFVGLIRWSNGNT |
| Nos: 234-236) | | YYTDSVKGRFTISRDNAKNTVYLQMNSLK |
| | | PEDTAVYYCATLRSGYVGSAYSQQAYDYW |
| | | GQGTQVTVSSAAAYPYDVPDYGSHHHHHH |
| 237 (The CDRs are | TRH12 | QVQLQESGGGLVQAGGSLRLSCAASTSTV |
| marked in bold, SEQ ID | | SDYHMGWFRQGPGKEREFVAAISWSGDST |
| Nos: 238-240) | | DYADSVKARFTISRDNAKKTMSLQMNSLK |
| | | PEDTAAYYCAARRVFKATWDYWGQGTQVT |
| | | VSSAAAYPYDVPDYGSHHHHHH |
| 241 (The CDRs are | TRH13 | QVQLQESGGGLVQAGGSLRLTCAATVRIF |
| marked in bold, SEQ ID | | SSYTMGWFRQSPGKEREFVGAITWSGGNT |
| Nos: 242-244) | | YYADSVKGRFTISRDNAKNTVYLQMNSLK |
| | | SEDTAVYYCAADPGRSYVLSRYYDQASYD |
| | | YWGQGTQVTVSSAAAYPYDVPDYGSHHHH |
| | | HH |
| 245 (The CDRs are | TRH14 | QVQLQESGGGLVQAGDSLRLSCAASGRPF |
| marked in bold, SEQ ID | | SSYTMGWFRQAPGKEREFVAAISWSGGTT |
| Nos: 246-248) | | YYTDSVQGRFTISRDNAKNTVYLQMNRLK |
| | | PEDTAVYYCATDVGRIYGGGSLYSSAFSY |
| | | DYWGQGTQVTVSSAAAYPYDVPDYGSHHH |
| | | HHH |
| 249 (The CDRs are | TRH17 | QVQLQESGGGLVQAGASLRLSCAASGGTF |
| marked in bold, SEQ ID | | SDYVMGWFRQPPGKEREFVASINYGGDKI |
| Nos: 250-252) | | SYADSLEGRFTILRDNTKDTTSLQMNTLK |
| | | PDDTAVYYCAAKWGYKTGPTYSYWGQGTQ |
| | | VTVSSAAAYPYDVPDYGSHHHHHH |
| 253 (The CDRs are | TRH18 | QVQLQESGGGLVQAGGSLRLSCAASGRTF |
| marked in bold, SEQ ID | | SNVAMGWFRQAPGKEREFAAAISGSGGTT |
| Nos: 254-256) | | YYAASVKGRFTISRDNGKKMVYLQMNSLK |
| | | PEDTAVYYCSTYDGRRGQGTQVTVSSAAA |
| | | YPYDVPDYGSHHHHHH |
| 257 (The CDRs are | TRH20 | QVQLQESGGGLVQAGGSLRLSCAASGLTF |
| marked in bold, SEQ ID | | SRNAIAWFRQAPGKEREFVAAISWNAITT |
| Nos: 258-260) | | AYGDSVKGRFTIFRGNTKNTVYLQMNSLK |
| | | PEDTAVYYCAARYSSGSYYYARTYEYDYW |
| | | GQGTQVTVSSAAAYPYDVPDYGSHHHHHH |

TABLE 5
Amino acid sequences of anti-cytochrome p450 Nanobodies

| | Nanobody | |
|---|---|---|
| SEQ ID NO: | name | Amino Acid Sequence |
| 261 (The CDRs are | P45001A | QVQLQESGGGLVQTGGSLRLSCAASGRTS |
| marked in bold, SEQ ID | | RSYAMGWFRQAPGKERELVADIAWSDGRI |
| Nos: 262-264) | | YYSDSVKGRFTISRDNAKNTVYLEIDSLK |
| | | PGDTAVYYCASRARGTVLYNYWGQGTQVT |
| | | VSSAAAYPYDVPDYGSHHHHHH |
| 265 (The CDRs are | P45001B | QVQLQESGGGLVQPGGSLRLSCAASGRTS |
| marked in bold, SEQ ID | | RSYAMGWFRQAPGKEREHVADIAWSDGRI |
| Nos: 262-263, 266) | | YYSDSVKDRFTISRDNAKNTVYLEIDSLK |
| | | PGDTAVYYCASRARGTVSYNYWGQGTQVT |
| | | VSSAAAYPYDVPDYGSHHHHHH |
| 267 (The CDRs are | P45001C | QVQLQESGGGLVQAGGSLRLSCAASGRTS |
| marked in bold, SEQ ID | | RSYAMGWFRQAPGKERELVADIAWSDGRI |
| Nos: 262-264) | | YYSDSVKGRFTISRDNAKNTVYLEIDSLK |
| | | PGDTAVYYCASRARGTVLYNYWGQGTQVT |
| | | VSSAAAYPYDVPDYGSHHHHHH |
| 268 (The CDRs are | P45001D | QVQLQESGGGLVQAGGSLTLSCAASGRTS |
| marked in bold, SEQ ID | | RSYAMGWFRQAPGKERELVADIAWSDGRI |
| Nos: 262-263, 266) ’ | | YYSDSVKGRFTISRDNAKNTVYLEIDSLK |
| | | PGDTAVYYCASRARGTVSYNYWGQGTQVT |
| | | VSSAAAYPYDVPDYGSHHHHHH |
| 269 (The CDRs are | P45001E | QVQLQESGGGLVQAGDSLRLSCAASGRTS |
| marked in bold, SEQ ID | | RSYAMGWFRQAPGKERELVADISWSDGRI |
| Nos: 262, 270, 264) | | YYSDSVKGRFTISRDNAKNTVYLEIDSLK |
| | | PGDTAVYYCASRARGSVLYNYWGQGTQVT |
| | | VSSAAAYPYDVPDYGSHHHHHH |
| 271 (The CDRs are | P45001F | QVQLQESGGGSVQAGGSLRLSCAASGRTS |
| marked in bold, SEQ ID | | RSYAMGWFRQAPGKERELVADIAWSDGRI |
| Nos: 262-263, 266) | | YYSDSVKGRFTISRDNAKNTVYLEIDSLK |
| | | PGDTAVYYCASRARGTVSYNYWGQGTQVT |
| | | VSSAAAYPYDVPDYGSHHHHHH |
| 272 (The CDRs are | P45001G | QVQLQESGGGLVQAGGSLRLSCAASGRTS |
| marked in bold, SEQ ID | | RSYAMGWFRQAPGKERELVADIAWSDGRI |
| Nos: 262-263, 266) | | YYSDSVKGRFTISRDNAKNTVYLEIDSLK |
| | | PGDTAVYYCASRARGTVSYNYWGQGTQVT |
| | | VSSAAAYPYDVPDYGSHHHHHH |
| 273 (The CDRs are | P45002A | QVQLQESGGGLVQAGGSLRLSCVYSGNVF |
| marked in bold, SEQ ID | | SINLMGWYRQAPGKQRELLAGINRGGRTN |
| Nos: 274-276) | | YTDVVKGRFTISRENAKNTIYLQMNGLKP |
| | | EDTGVYYCAASRDPYTGYWGQGTQVTVSS |
| | | AAAYPYDVPDYGSHHHHHH |
| 277 (The CDRs are | P45002B | QVQLQESGGGLVQAGGSLRLSCVYSGNVF |
| marked in bold, SEQ ID | | SINLMGWYRQAPGKQRELLAGINRGGRTN |
| Nos: 274-276) | | YTDVVKGRFTISKENAKNTIYLQMNSLKP |
| | | EDTGVYYCAASRDPYTGYWGQGTQVTVSS |
| | | AAAYPYDVPDYGSHHHHHH |
| 278 (The CDRs are | P45002C | QVQLQESGGGLVQAGGSLRLSCVYSGNVF |
| marked in bold, SEQ ID | | SINLMGWYRQAPGKQRELLAGINRGGRTN |
| Nos: 274-276) | | YTDVVKGRFTISRENAKNTIYLQMNSLKP |
| | | EDTGVYYCAASRDPYTGYWGQGTQVTVSS |
| | | AAAYPYDVPDYGSHHHHHH |
| 279 (The CDRs are | P45002D | QVQLQESGGGLVQPGGSLRLSCVASGSVD |
| marked in bold, SEQ ID | | SINLMGWYRQAPGKQRELLAGINRGGRTN |
| Nos: 280, 275-276) ’ | | YTDVVKGRFTISRENAKNTIYLQMNSLKP |
| | | EDTGVYYCAASRDPYTGYWGQGTQVTVSS |
| | | AAAYPYDVPDYGSHHHHHH |
| 281 (The CDRs are | P45002E | QVQLQESGGGLVPSGGSLRLSCVYSGNVF |
| marked in bold, SEQ ID | | SINLMGWYRQAPGKQRELLAGINRGGRTN |
| Nos: 282, 275-276) | | YTDWKGRFTISRENAKNTIYLQMNGLKPE |
| | | DTGVYYCAASRDPYTGYWGQGTQVTVSSA |
| | | AAYPYDVPDYGSHHHHHH |
| 283 (The CDRs are | P45002F | QVQLQESGGGLVQAGGSLRLSCVYSGNVF |
| marked in bold, SEQ ID | | SINLMGWYRQAPGKQRELLAGITRGGRTN |
| Nos: 282, 275-276) | | YTDVVKGRFTISRESAKNTIYLQMNSLKP |
| | | EDTGVYYCAASRDPYTGYWGQGTQVTVSS |
| | | AAAYPYDVPDYGSHHHHHH |
| 284 (The CDRs are | P45002G | QVQLQESGGGLVQAGGSLRLSCVYSGNVF |
| marked in bold, SEQ ID | | SINLMGWYRQAPGKQRELLAGITRGGRTN |
| Nos: 282, 275, 285) | | YTDVVKGRFTISRENAKNTIYLQMNSLKP |
| | | EDTGVYYCAASRDPYTGYWGQGTQVTVSS |
| | | AAAYPYDVPDYGSHHHHHH |
| 286 (The CDRs are | P45003A | QVQLQESGGGLVQAGDSLRLSCVASKRSF |
| marked in bold, SEQ ID | | SSHAMGWFRQAPGKAREFVATISWNSGST |
| Nos: 287, 220, 289) | | FYSDSSRGRFTISRDNGKNTLYLQMNSLK |
| | | PEDTAVYYCAAASGRGITASDFRYDAWGQ |
| | | GTQVTVSSAAAYPYDVPDYGSHHHHHH |
| 290 (The CDRs are | P45003B | QVQLQESGGGVVQAGGSLRLSCVGSGRTF |
| marked in bold, SEQ ID | | SSHAMGWFRQAPGKEREFVATISWNSGST |
| Nos: 291,220, 289)’ | | FYADSVRGRFTISRDNAKNTLYLQMNSLK |
| | | AEDTAVYYCAAASGRGITASDFRYDAWGQ |
| | | GTQVTVSSAAAYPYDVPDYGSHHHHHH |
| 292 (The CDRs are | P45003C | QVQLQESGGGSVQAGGSLRLSCVASGRSF |
| marked in bold, SEQ ID | | SRHAMAWFRQAPGKEREFVATISWNAGST |
| Nos: 293-294,289) | | YYADSVKGRFTISRDNAKNTVTLQMNSLK |
| | | PEDTAVYYCAAASGRGITASDFRYDAWGQ |
| | | GTQVTVSSAAAYPYDVPDYGSHHHHHH |
| 295 (The CDRs are | P45004 | QVQLQESGGGLVQAGGSLRLSCAASGRTF |
| marked in bold, SEQ ID | | SRINMGWFRQAPGKEREFVAVISWNSIAD |
| Nos: 296-298) | | YADSVKARFTISRDNAKNTVYLQMNSLKP |
| | | EDTAVYYCAASAPFRSKNPTLYLYWGRGT |
| | | QVTVSSAAAYPYDVPDYGSHHHHHH |
| 299 (The CDRs are | P45005 | QVQLQESGGGLVQSGDSLRLSCSASGRTF |
| marked in bold, SEQ ID | | NPVAMAWFRXAPGKEREFVGTITWGIGST |
| Nos: 300-302) | | HYAVPVKGRFTISKENAKNTVYLQMNRLQ |
| | | PEDTAVYYCAARTSLLRRADEIPSVANYD |
| | | SWGQGTQVTVSSAAAYPYDVPDYGSHHHH |
| | | HH |
| 303 (The CDRs are | P45006 | QVXLQESGGGLVQAGDSLRLSCAASGRTF |
| marked in bold, SEQ ID | | SMYTMGWFRQAPGKEREFVAKISTSGRYT |
| Nos: 304-306) | | DYVDSVRGRFTLSRDNVKNTIYLQMNSLK |
| | | PDDTAVYYCAARLPRPDTWSQGKTDYWGQ |
| | | GTQVTVSSAAAYPYDVPDYGSHHHHHH |
| 307 (The CDRs are | P45007 | QVQLQESGGGLVQAGGSLRLSCAASGRTF |
| marked in bold, SEQ ID | | SNNPMGWFRXAPGKEREFVATKTGGNSGI |
| Nos: 308-310) | | TVYSNSVKGRFTISRDLAKNTVYLQMDGL |
| | | KLEDTAIYYCAASGTPLALRSEKNYDYWG |
| | | QGTQVTVSSAAAYPYDVPDYGSHHHHHH |
| 311 (The CDRs are | P45008 | QVXLQESGGGLVQPGGSLRLSCAASGSIF |
| marked in bold, SEQ ID | | NINTMGWYRQAPGNQRELVAAISSDGRPN |
| Nos: 312-314) | | HRDSVKGRFTISRDNAKNTVYLQMNSLKP |
| | | EDTAVYYCNTVPASRAGGYWGQGTQVTVS |
| | | SAAAYPYDVPDYGSHHHHHH |
| 315 (The CDRs are | P45009 | QVQLQESGGGLAQAGDSLRLSCVASGRTI |
| marked in bold, SEQ ID | | DNGAMGWFRQAPGKERESVAAINWSGSST |
| Nos: 316-318) | | YYADSVKGRFAISRDNVKHEVYLQMNRLR |
| | | REDTAVYYCAAAKSIGTYSSSSAYDYWGQ |
| | | GTQVTVSSAAAYPYDVPDYGSHHHHHH |

TABLE 6
Amino acid sequences of anti-chitin deacetylase Nanobodies

| | Nanobody | |
|---|---|---|
| SEQ ID NO: | name | Amino Acid Sequence |
| 319 (The CDRs are | CDA1a | QVQLQESGGGLVQAGGSLRLSCAASGRTF |
| marked in bold, SEQ ID | | NTYAMGWFRQAPGKEREFVAAMNRDGSTV |
| Nos: 320-322) | | LYADSVKGRFAISKDNAKNTGYLQMNSLK |
| | | PEDTAIYYCAANARYSDYTDGQSFVSWGQ |
| | | GTQVTVSSAAAYPYDVPDYGSHHHHHH |
| 323 (The CDRs are | CDA1b | QVQLQESGGGLVQAGGSLRLSCQASGRTF |
| marked in bold, SEQ ID | | NTYAMGWFRQAPGKEREFVAAMNRDGSTI |
| Nos: 320,324-325) | | LYGDSVKGRFAISRDNAKNTGYLQMNSLK |
| | | PEDTAIYYCAANARYSDYTHGQSFVSWGQ |
| | | GTQVTVSSAAAYPYDVPDYGSHHHHHH |
| 326 (The CDRs are | CDA1c | QVQLQESGGGLVQAGGSLRLSCQASGRTF |
| marked in bold, SEQ ID | | DAYAMGWFRQAPGKEREFVAAMNRDGSTV |
| Nos: 327,321, 328) | | LYADSVKGRFAISKDNAKNTGYLQMNSLK |
| | | PEDTAIYYCAANARYSDYTDGQSFISWGQ |
| | | GTQVTVSSAAAYPYDVPDYGSHHHHHH |
| 329 (The CDRs are | CDA1d | QVQLQESGGGLVQAGGSLRLSCQASGRTF |
| marked in bold, SEQ ID | | NTYAMGWFRQAPGKEREFVAAMNRDGSTV |
| Nos: 320-322) | | LYADSVKGRFAISKDNAKNTGYLQMNSLK |
| | | PEDTAIYYCAANARYSDYTDGQSFVSWGQ |
| | | GTQVTVSSAAAYPYDVPDYGSHHHHHH |
| 331 (The CDRs are | CDA1e | QVQLQESGGGLVQAGGSLRLSCVASGRTF |
| marked in bold, SEQ ID | | NTYAMGWFRQAPGKEREFVAAMNRDGSTV |
| Nos: 320-321, 332) | | LYADYVKGRFAISKDNAKNTGYLQMNSLK |
| | | PEDTAIYYCAANARYSDYTDEQSFVSWGQ |
| | | GTQVTVSSAAAYPYDVPDYGSHHHHHH |
| 333 (The CDRs are | CDA1f | QVQLQESGGGLVQAGGSLRLSCQASGRTF |
| marked in bold, SEQ ID | | NTYAMGWFRQAPGKEREFVAAMNRDGSTV |
| Nos: 320-322) | | LYADSVKGRFAISKDNAKSTGYLQMNSLK |
| | | PEDTAIYYCAANARYSDYTDGQSFVSWGQ |
| | | GTQVTVSSAAAYPYDVPDYGSHHHHHH |
| 334 (The CDRs are | CDA1g | QVQLQESGGGLVQAGGSLRLSCKASGRTF |
| marked in bold, SEQ ID | | STYAVGWFRQAPGKPREFVAAMNSRGSTI |
| Nos: 208, 336-337) | | NYADSVKGRFAISRDNAKNTGYLQMDSLK |
| | | PEDTAIYYCAADARYSDYTDGQSFKSWGQ |
| | | GTQVTVSSAAAYPYDVPDYGSHHHHHH |
| 338 (The CDRs are | CDA1h | QVQLQESGGGLVQAGGSLRLSCQASGRTF |
| marked in bold, SEQ ID | | NTYAMGWFRQAPGKEREFVAAMNRDGSTI |
| Nos: 320, 324, 339) | | LYADSVKGRFAISKDNAKNTGYLQMNSLK |
| | | PEDTAIYYCAANARYSDYTNGHSFVSWGQ |
| | | GTQVTVSSAAAYPYDVPDYGSHHHHHH |
| 340 (The CDRs are | CDA1i | QVQLQESGGGLVQPGGSLRLSCQASGRTF |
| marked in bold, SEQ ID | | NTYAMGWFRQAPGKEREFVAAMNRDGSTV |
| Nos: 320-322) | | LYADSVKGRFAISKDNARNTGYLQMNSLK |
| | | PEDTAIYYCAANARYSDYTDGQSFVSWGQ |
| | | GTQVTVSSAAAYPYDVPDYGSHHHHHH |
| 341 (The CDRs are | CDA1j | QVQLQESGGGLVQAGGSLRLSCQASGRTF |
| marked in bold, SEQ ID | | NTYAMGWFRQAPGKEREFVAAMNRDGSTV |
| Nos: 320-322) | | LYRDSVKGRFAISKDNARNTGYLQMNNLK |
| | | PEDTAIYYCAANARYSDYTDGQSFVSWGQ |
| | | GTQVTVSSAAAYPYDVPDYGSHHHHHH |
| 342 (The CDRs are | CDA1k | QVQLQESGGGLVQAGGSLRLSCQASERSF |
| marked in bold, SEQ ID | | STYAMGWFRQAPGKEREFVAAMNRNGNTI |
| Nos: 343-345) | | NYLDSVKGRFAISRDNAKSTGYLQMNSLK |
| | | PEDTATYYCSANARLSDYINPQSFVSWGQ |
| | | GTQVTVSSAAAYPYDVPDYGSHHHHHH |
| 346 (The CDRs are | CDA2a | QVQLQESGGGLVQAGGSLRLSCAASGRAF |
| marked in bold, SEQ ID | | SSYGMAWFRQAPGKEREFVAAINSNGRST |
| Nos: 347-349) | | YYADTVKGRFTISRDDGRNTLYLQMNSLK |
| | | PEDTAVYYCAADRQSMKGYEYGYWGQGTQ |
| | | VTVSSAAAYPYDVPDYGSHHHHHH |
| 350 (The CDRs are | CDA2b | QVQLQESGGGLVQAGGSLRLSCAASGRAF |
| marked in bold, SEQ ID | | SRYGMAWFRQAPGKEREFVAAITSNGRST |
| Nos: 351-353) | | YYADTVKGRFTISRDNGRNTLYLQMNTLK |
| | | PEDTAVYYCAADRKSMTGYEYGYWGQGTQ |
| | | VTVSSAAAYPYDVPDYGSHHHHHH |
| 354 (The CDRs are | CDA2c | QVQLQESGGGLVQPGGSLRLSCAASGRAF |
| marked in bold, SEQ ID | | SRYGMAWFRQAPGKEREFVAAITSNGRST |
| Nos: 351-353) | | YYADTVKGRFTISRDNGRNTLYLQMNTLK |
| | | PEDTAVYYCAADRKSMTGYEYGYWGQGTQ |
| | | VTVSSAAAYPYDVPDYGSHHHHHH |
| 355 (The CDRs are | CDA2d | QVQLQESGGGLVQAGGSLRLSCAASGRAF |
| marked in bold, SEQ ID | | SRYGMAWFRQAPGKEREFVGAINSNGRST |
| Nos: 351,347, 356) | | YYADTVKGRFTISRDNDRNTLYLQMNSLK |
| | | PEDTAVYYCAADRQSMTRYEYGYWGQGTQ |
| | | VTVSSAAAYPYDVPDYGSHHHHHH |
| 357 (The CDRs are | CDA3a | QVQLQESGGGLVQPGGSLRLSCAASGFTF |
| marked in bold, SEQ ID | | SNYAMGWFRQAPGKERDFVAAVSGIARRT |
| Nos: 358-360) | | YYADSVKGRSTISRDNGRNTLYLQMNNLK |
| | | PEDTAVYYCARATSRMTSVTTLNDYGYWG |
| | | QGTPVTVSSAAAYPYDVPDYGSHHHHHH |
| 361 (The CDRs are | CDA3b | QVQLQESGGGLVQPGESLRLSCAASGFTF |
| marked in bold, SEQ ID | | SNYAMGWFRQAPGKERDFVAAVSGIARRT |
| Nos: 358-359, 362) | | YYADSVKGRSTISRDNGRNTVYLQMNSLK |
| | | PEDTAVYYCARATSRMTSVTTLDDYGYWG |
| | | QGTQVTVSSAAAYPYDVPDYGSHHHHHH |
| 363 (The CDRs are | CDA3c | QVQLQESGGGLVQPGGSLRLSCAASGFTF |
| marked in bold, SEQ ID | | SNYAMGWFRQAPGKERDFVAAVSGIARRT |
| Nos: 358-360) | | YYADSVKGRSTISRDNGRNTVYLQMNSLK |
| | | PEDTAVYYCARATSRMTSVTTLNDYGYWG |
| | | QGTQVTVSSAAAYPYDVPDYGSHHHHHH |
| 364 (The CDRs are | CDA3d | QVQLQESGGGLVQPGGSLRLSCAASGFTF |
| marked in bold, SEQ ID | | SSYAMGWFRQAPGKEREFVAAVSGIARRT |
| Nos: 365, 359, 366) | | YYADSVKGRSTISRDNGRNTVYLQMNSLK |
| | | PEDTAVYYCARATSRMTSVTTLNDYAYWG |
| | | QGTQVTVSSAAAYPYDVPDYGSHHHHHH |
| 367 (The CDRs are | CDA4a | QVQLQESGGGSVQSGGSLTLSCSASGSLF |
| marked in bold, SEQ ID | | SINAMGWYRQAPRKQHELVATMMDGGSTS |
| Nos: 368-370) | | YADSVKGRFTISRDNDKKTVYLQMNSLKP |
| | | EDTGVYYCVADRLGSGRYAYGIDYWGQGT |
| | | QVTVSSAAAYPYDVPDYGSHHHHHH |
| 371 (The CDRs are | CDA4b | QVQLQESGGGLVQPGGSLRLACAASGSIF |
| marked in bold, SEQ ID | | SINAMGWYRQAPRKQRELVATMMDGGSTS |
| Nos: 372, 369-370) | | YADSVKGRFTISRDNDKKTVYLQMNSLKP |
| | | EDTGVYYCVADRLGSGRYAYGIDYWGQGT |
| | | QVTVSSAAAYPYDVPDYGSHHHHHH |
| 373 (The CDRs are | CDA4c | QVQLQESGGGLVQAGGSLRLSCAASGSIV |
| marked in bold, SEQ ID | | SINGMGWYRQAPRKQRELVATMMDGGSTS |
| Nos: 374, 369, 375) | | YADSVKGRFTISRDNDKKTVYLQMNSLKP |
| | | EDTGVYYCAADRLGSGRYAYAIDYWGQGT |
| | | QVTVSSAAAYPYDVPDYGSHHHHHH |
| 376 (The CDRs are | CDA5 | QVQLQESGGGLVQPGGSLRLSCTASGRTV |
| marked in bold, SEQ ID | | SNYGMAWFRQVAGKERAFVAAINNRGDSK |
| Nos: 377-379) | | YYAESVKGRFTIARDNAKNTVYLQMNVLK |
| | | PEDTAVYYCAADRRSLVRYEYNYWGQGTQ |
| | | VTVSSAAAYPYDVPDYGSHHHHHH |
| 380 (The CDRs are | CDA6a | QVQLQESGGGLVQAGGSLRLSCAASGRTF |
| marked in bold, SEQ ID | | SRYTMGWFRQAPGKEREFVAAIRWSSGNT |
| Nos: 381-383) | | YYTDAVKGRFTISRDNTENTVYLQMNSLK |
| | | PEDTAVYYCVADDGLSYASSSYLYWGQGT |
| | | QVTVSSAAAYPYDVPDYGSHHHHHH |
| 384 (The CDRs are | CDA6b | QVQLQESGGGLVQPGGSLRLSCAASGFTF |
| marked in bold, SEQ ID | | SRYTMGWFRQAPGKEREFVAAIRWSSGNT |
| Nos: 385, 382-383) | | YYTDAVKGRFTISRDNTENTVYLQMNSLK |
| | | PEDTAVYYCVADDGLSYASSSYLYWGQGT |
| | | QVTVSSAAAYPYDVPDYGSHHHHHH |
| 386 (The CDRs are | CDA7a | QVQLQESGGGLVQAGGSLRLSCVASGRTF |
| marked in bold, SEQ ID | | GNYGLAWFRQPPGKEREFVAAINNRGGNT |
| Nos: 387-389) | | YYADSVKGRFTISRDNAANTLYLQMSSLK |
| | | PEDTAVYYCAADRTSLHSYRYTYWGQGTQ |
| | | VTVSSAAAYPYDVPDYGSHHHHHH |
| 390 (The CDRs are | CDA7b | QVQLQESGGGLVQAGGSLRLSCVASGRTF |
| marked in bold, SEQ ID | | GNYGLAWFRQPPGKEREFVAAISNRGGNT |
| Nos: 387, 391, 389) | | YYADSVKGRFTISRDNAANTLYLQMSSLK |
| | | PEDTAVYYCAADRTSLHSYRYTYWGQGTQ |
| | | VTVSSAAAYPYDVPDYGSHHHHHH |
| 392 (The CDRs are | CDA7c | QVQLQESGGGLVQAGDSPKLSCVASGRTF |
| marked in bold, SEQ ID | | GNYGLAWFRQPPGKEREFVAAISNRGGNT |
| Nos: 387, 391, 389) | | YYADSVKGRFTISRDNAANTLYLQMSSLK |
| | | PEDTAVYYCAADRTSLHSYRYTYWGQGTQ |
| | | VTVSSAAAYPYDVPDYGSHHHHHH |
| 393 (The CDRs are | CDA8 | QVQLQESGGGLVQAGGSLRLSCAASERTF |
| marked in bold, SEQ ID | | STYNMAWFRQAPGKERESVSRINWNGGFT |
| Nos: 394-396) | | GYADSVKGRFTISRDNAKKAMYLQMNSLK |
| | | SEDTAVYYCAACGSAYPCRPEEYTYWGQG |
| | | TQVTVSSAAAYPYDVPDYGSHHHHHH |
| 397 (The CDRs are | CDA10 | QVQLQESGGGLVQTGESLRLSCAASGRTL |
| marked in bold, SEQ ID | | SSYTMGWFRQAPGKEREIVAASSWSGGRT |
| Nos: 398-400) | | YYADSVKGRFTMSRNNAENTVYLQMNSLN |
| | | PEDTAVYYCAADSSRAPRTYNYWGQGTQV |
| | | TVSSAAAYPYDVPDYGSHHHHHH |
| 401 (The CDRs are | CDA12 | QVQLQESGGGLVQAGGSLRLSCAASGRTF |
| marked in bold, SEQ ID | | STYNMSWFRQGPGKERIFVATISWSGRVT |
| Nos: 402-404) | | DYADSVRGRFTISRDNAKKMVYLQMNSLK |
| | | PEDTAVYYCAADSNSRRSRDYDYWGQGTQ |
| | | VTVSSAAAYPYDVPDYGSHHHHHH |
| 405 (The CDRs are | CDA14 | QVQLQESGGGLVQPGGSLRLSCAASGTTR |
| marked in bold, SEQ ID | | RINSMRWYRQTPGNERDLVAGITEGGFTA |
| Nos: 406-408) | | YVDSVKGRFTISRDNAKNTVYLQMNSLKP |
| | | EDTAVYYCYAAYLGAAYWGQGTQVTVSSA |
| | | AAYPYDVPDYGSHHHHHH |

TABLE 7
Amino acid sequences of anti-chitin
synthase Nanobodies

| | Nanobody | |
|---|---|---|
| SEQ ID NO: | name | Amino Acid Sequence |
| 411 (The CDRs | CHS1a | QVQLQESGGGLVQAGGSLRLSCSASTSIAS |
| are marked in | | INAVGWYRQAPGKQRELVAVIVNGSTTRYA |
| bold, SEQ ID | | DSVKGRFTISGDNAENTVPLLMSSLKPEDT |
| Nos: 412-414) | | AIYYCGARDLSGTYWGQGTQVTVSSAAAYP |
| | | YDVPDYGSHHHHHH |
| 415 (The CDRs | CHS1b | QVQLQESGGGLVQPGGSLRLSCSASTSIAS |
| are marked in | | INAVGWYRQAPGKQRELVAVIVNGSTTRYA |
| bold, SEQ ID | | DSVKGRFTISGDNAKNTVPLQMSSLKPEDT |
| Nos: 412-414) | | AIYYCGARDLSGTYWGQGTQVTVSSAAAYP |
| | | YDVPDYGSHHHHHH |
| 416 (The CDRs | CHS1c | QVQLQESGGGLVQAGGSLRLSCSASTSIAS |
| are marked in | | INAVGWYRQAPGKQRELVAVIVNGSTTRYA |
| bold, SEQ ID | | DSVKGRFTISGDNAKNTVPLQMSSLKPEDT |
| Nos: 412-414) | | AIYYCGARDLSGTYWGQGTQVTVSSAAAYP |
| | | YDVPDYGSHHHHHH |
| 417 (The CDRs | CHS2a | QVQLQESGGGLVQAGGSLRLSCAASRGTFS |
| are marked in | | RYVMGWFRQGPGKEREFVAGISWSGISTYY |
| bold, SEQ ID | | ADFVKGRFTISRDNAKNTVYLQMNSLKPED |
| Nos: 418-420) | | TAVYYCAADPGRGYDYWGQGTQVTVSSAAA |
| | | YPYDVPDYGSHHHHHH |
| 421 (The CDRs | CHS2b | QVQLQESGGGLVQAGGSLRLSCAASGRSFS |
| are marked in | | RSVMGWFRQAPGKEREFVAATSWSGGGTYY |
| bold, SEQ ID | | ADSVKGRFTISRDNAQNTVYLQMNSLKPED |
| Nos: 422-424) | | TAVYYCAADVGRGYHYWGQGTQVTVSSAAA |
| | | YPYDVPDYGSHHHHHH |
| 425 (The CDRs | CHS3a | QVQLQESGGGLVQAGGSLRLSCVASGGTFS |
| are marked in | | GLTMGWFRQAPQKEREFVAAISWTGRSTYY |
| bold, SEQ ID | | ADSVKGRFTISRDNVKNMVYLQMNSLKPED |
| Nos: 426-428) | | TGVYFCAADWASGTPYWGQGTQVTVSSAAA |
| | | YPYDVPDYGSHHHHHH |
| 429 (The CDRs | CHS3b | QVQLQESGGGLVQAGTSLRLSCAASGGTFS |
| are marked in | | GLTMGWFRQAPQKEREFVAAISWTGRSTYY |
| bold, SEQ ID | | ADSVKGRFTISRDNVKNMVYLQMVALNPED |
| Nos: 426-427 | | TAVYYCAADWASATPYWGQGTQVTVSSAAA |
| and 430 | | YPYDVPDYGSHHHHHH |
| 431 (The CDRs | CHS4 | QVQLQESGGGLVQSGESLTLACVISGITLE |
| are marked in | | RYTVGWFHQAPGKNPEGVSCIGKSNDETFY |
| bold, SEQ ID | | TDSVKGRFTISSDNAKNTVYLQMNSLTPSD |
| Nos: 432-434) | | AGVYYCAAAKAPVTAYDCSLYLYTWRSTYR |
| | | GQGTQVTVSSAAAYPYDVPDYGSHHHHHH |
| 435 (The CDRs | CHS5 | QVQLQESGGGLVQAGGSLTLSCAASARTFS |
| are marked in | | TYALAWFRQTPGKGREFVGAISRSGSSTRY |
| bold, SEQ ID | | ADSVKGRFAISRDNAQRAIYLQMNSLKPED |
| Nos: 436, 140 | | TAVYYCAAGSRSYYNIPYYDYWGQGTQVTV |
| and 437) | | SSAAAYPYDVPDYGSHHHHHH |
| 438 (The CDRs | CHS6 | QVQLQESGGGLVQAGASLRLSCAASVRTFS |
| are marked in | | SYAVGWFRQAPGKEREFVAGITWSGGSKYY |
| bold, SEQ ID | | RDAVKGRFTISRDNAKNAVYLQMNSLKPED |
| Nos: 439-441) | | TAVYSCAATSARYTSGALYYRDRQYNYWGQ |
| | | GTQVTVSSAAAYPYDVPDYGSHHHHHH |
| 442 (The CDRs | CHS7 | QVQLQESGGGLVQAGGSLRLSCAASTRTFG |
| are marked in | | NYAMGWFRQAPGKEREFVAAINRRGTTTYY |
| bold, SEQ ID | | ADSVKDRFTISTDYAKNTVYLEMISLKPED |
| Nos: 443-445) | | TAVYYCAVDRSTGWQPSTSRYDYASWGQGT |
| | | QVTVSSAAAYPYDVPDYGSHHHHHH |
| 446 (The CDRs | CHS8 | QVQLQESGGGLVQAGSSLRLSCAASGRTFR |
| are marked in | | YHAMGWFRQAPGKEREFVAGISTSGGMTYY |
| bold, SEQ ID | | PDSVKGRFTISRDNAKNTLYLQMNSLKPGD |
| Nos: 447-449) | | TAVYYCAKFHGDKGYGSSWYYDYWGQGTQV |
| | | TVSSAAAYPYDVPDYGSHHHHHH |
| 450 (The CDRs | CHS9 | QVQLQESGGGLVQPGGSLRLSCAASGFTFS |
| are marked in | | SYAMSWVRQAPGKGLERISIINSGGGSTSY |
| bold, SEQ ID | | ADSVKGRFTVSRDNNKNTLYLQMNSLQPED |
| Nos: 356 and | | TAVYYCGVRRNWGLGTHSGEYVYWGRGTQV |
| 451-452) | | TVSSAAAYPYDVPDYGSHHHHHH |
| 453 (The CDRs | CHS10 | QVQLQESGGGLVQAGGSLRLSCAASGFTFD |
| are marked in | | DYAIGWFRQAPGKVREGVSTIKSSDGSTYY |
| bold, SEQ ID | | ADSVKGRFTISLDNAKRTVSLQMNNLKPED |
| Nos: 454-456) | | TAVYYCAAGARRWPYDYIYWGRGTQVTVSS |
| | | AAAYPYDVPDYGSHHHHHH |
| 457 (The CDRs | CHS11 | QVQLQESGGGVVQAGASLRLSCAASERIFL |
| are marked in | | NYNMAWVRQAPGKEREFVAAITWSGSNIDY |
| bold, SEQ ID | | ADTVKGRFTISRDNAKNTVYLQMDSLKPED |
| Nos: 458-460) | | TAVYYCAADPSYWKIRTTLNGLDKWGQGTQ |
| | | VTVSSAAAYPYDVPDYGSHHHHHH |
| 461 (The CDRs | CHS12 | QVQLQESGGGFVQAGDSLRLSCAASGRTFS |
| are marked in | | RFPMGWFRQAPGKEREFVAAISWSGGSTLI |
| bold, SEQ ID | | TDSVKDRFTISRDNAKNTMYLQMNSLKPED |
| Nos: 462-464) | | TAVYYCAARRDSTTSYVTWGQGTQVTVSSA |
| | | AAYPYDVPDYGSHHHHHH |
| 465 (The CDRs | CHS13 | QVQLQESGGGLVQAGGSLRLSCTVSGGTFT |
| are marked in | | TYTMAWFRQAPGKEREFVAQISWSGGITAY |
| bold, SEQ ID | | LDSVKGRFTISRDNAKNTVYLQMNSLKPED |
| Nos: 466-468) | | TAVYYCARRGRKYEDDYWGQGTQVTVSSAA |
| | | AYPYDVPDYGSHHHHHH |
| 469 (The CDRs | CHS14 | QVQLQESGGGLVQAGGSLRLSCAASGRTFS |
| are marked in | | SYIMGWFRQAPGKVREFVASISWSGGFTYY |
| bold, SEQ ID | | ADSVKGRST1SRDNAKNTLYLQMNSLKSED |
| Nos: 470-472) | | TAVYYCAADPGQGYKYWGQGTQVTVSSAAA |
| | | YPYDVPDYGSHHHHHH |
| 473 (The CDRs | CHS15 | QVQLQESGGGLVQAGGSLRLSCVASGRSFR |
| are marked in | | SYTMGWLRQAPGKEREFVAAISMSGVVTYY |
| bold, SEQ ID | | ADSVKGRFTISRDNAAKTLYLQMNSLKPED |
| Nos: 474-476) | | TAVYYCAARPDRTGKADYSGQGTQVTVSSA |
| | | AAYPYDVPDYGSHHHHHH |
| 477 (The CDRs | CHS16 | QVQLQESGGGLVQPGGSLRLSCAASGGTFS |
| are marked in | | RSIMGWFRQAPGKERERVAAISWSGSLTFY |
| bold, SEQ ID | | ADSVKGRFTISRDNAKNSVYLQMDSLKPED |
| Nos: 478-480) | | TAVYYCAADKLGGTWDSWGPGTQVTVSSAA |
| | | AYPYDVPDYGSHHHHHH |
| 481 (The CDRs | CHS18 | QVQLQESGGGLVQAGGSLRLSCTASGRTFS |
| are marked in | | SYSMAWFRQASGKEREIVAAVSRFGKFKYY |
| bold, SEQ ID | | ADSVKGRFTISRDNAKNTLYLQMNSLKPED |
| Nos: 482, | | TAVYYCAKTDGSSWYLDYWGQGTQVTVSSA |
| 479 and 483) | | AAYPYDVPDYGSHHHHHH |
| 484 (The CDRs | CHS19 | QVQLQESGGGLVQAGDSLRLSCTASGRTFS |
| are marked in | | SYVMGWFRQAPGKEREFVAAISRSGGNTYF |
| bold, SEQ ID | | GDSAEARFTISRDNTKNTVYLQMSSLRPDD |
| Nos: 485-487) | | TAVYYCARYRLVAGSTSRYTYDQWGQGTQV |
| | | TVSSAAAYPYDVPDYGSHHHHHH |
| 488 (The CDRs | CHS20 | QVQLQESGGGLVQAGDSLRLTCAHSGRPFS |
| are marked in | | SSAMGWFRQAPGKEREFVAAISRGGLSKYY |
| bold, SEQ ID | | ADSVKGRFTIFRDNAKNTVYLQMNSLKPED |
| Nos: 489-491) | | TAIYYCAGSLRDRPTKDEYVVWGQGTQVTV |
| | | SSAAAYPYDVPDYGSHHHHHH |
| 492 (The CDRs | CHS21 | QVQLQESGGGSVQAGDSLKLSCVASGRSRY |
| are marked in | | ALGWFRQAPGKAREFVGAARGGAGNTYYHE |
| bold, SEQ ID | | SVKGRFTISRDNDKLTVYLQMNDLKPEDTA |
| Nos: 493-495) | | VYTCAAGKDFGTAVSWTSWGQGTQVTVSSA |
| | | AAYPYDVPDYGSHHHHHH |
| 496 (The CDRs | CHS22 | QVQLQESGGGLVQAGGSLRLSCAASGGTFS |
| are marked in | | GLTMGWFRQAPGKERDFVAAISWNGRFTY |
| bold, SEQ ID | | YKDSVKGRFAISRDNAKSTVYLQMTSLKPE |
| Nos: 426 and | | DTAVYYCGADVLSGRGYRYWGQGTQVTV |
| 497-498) | | SSAAAYPYDVPDYGSHHHHHH |
| 499 (The CDRs | CHS23 | QVQLQESGGGLVQAGGSLRLSCAVSGRAFS |
| are marked in | | TYGMGWFRQAPRKEREFITAINRNGDRTWY |
| bold, SEQ ID | | ADSVTGRFTISRDNDKNMVYLQMDSLKTED |
| Nos: 500-502) | | TGIYYCHTRRFGYDYWGQGTQVTVSSAAAY |
| | | PYDVPDYGSHHHHHH |
| 503 (The CDRs | CHS24 | QVQLQESGGGWVQPGGSLRLSCTISGLSRY |
| are marked in | | YAMGWFRQVPGKERESVATISLRGGRTYYA |
| bold, SEQ ID | | DSVEGRFTISRDNAKNTMYLQMNSLKPEDT |
| Nos: 504-506) | | AVYYCVADTTWGAPRSRYHYWGQGTQVTVS |
| | | SAAAYPYDVPDYGSHHHHHH |
| 507 (The CDRs | CHS26 | QVQLQESGGGLVQPGGSLRLSCAASGFTFR |
| are marked in | | SYAMSWVRQAPGQGLEWVSAINSGGGSTTY |
| bold, SEQ ID | | ADSVKGRFTISRDNAKNTLYLQMNSLKPED |
| Nos: 508-510) | | TAVYYCAKMGSSSRGNRYLEVWGQGTQVTV |
| | | SSAAAYPYDVPDYGSHHHHHH |

TABLE 8
Amino acid sequences of anti-NPC1 sterol
transporter Nanobodies

| | Nanobody | |
|---|---|---|
| SEQ ID NO: | name | Amino Acid Sequence |
| 511 (The CDRs | NPC1a | QVQLQESGGGLVQAGDSLRLSCEASARTFI |
| are marked in | | AYAVGWFRQAPGKEREFVAAISWNGGMTDY |
| bold, SEQ ID | | ADFVKGRFAISRDNAKIVSLQMNNLKPEDT |
| Nos: 512-514) | | ALYYCTAGPARRSYSYRDGYDYWGQGTQVT |
| | | VSSAAAYPYDVPDYGSHHHHHH |
| 515 (The CDRs | NPC1b | QVQLQESGGGLVQTGGSLRLSCEASARTFI |
| are marked in | | AYAVGWFRQAPGKEREFVAAISWNGGMTDY |
| bold, SEQ ID | | ADFVKGRFAISRDSAKTVSLQMNNLKPEDT |
| Nos: 512-514) | | ALYYCTAGPARRSYSYRDGYDYWGQGTQVT |
| | | VSSAAAYPYDVPDYGSHHHHHH |
| 516 (The CDRs | NPC1c | QVQLQESGGGLVQVGGSLRISCVASGRTGS |
| are marked in | | YYAMGWFRQAPGKEREFVAAISWNGGMTDY |
| bold, SEQ ID | | ADFVKGRFAISRDNAKTVSLQMNNLKPEDT |
| Nos: 517 and | | ALYYCTAGPARRSYSYRDGYDYWGQGTQVT |
| 513-514) | | VSSAAAYPYDVPDYGSHHHHHH |
| 518 (The CDRs | NPC1d | QVQLQESGGGLVQPGRSLRLSCEASARTFI |
| are marked in | | AYAVGWFRQAPGKEREFVAAISWNGGMTDY |
| bold, SEQ ID | | ADFVKGRFAISRDNAKTVSLQMNNLKPEDT |
| Nos: 512-514) | | ALYYCTAGPARRSYSYRDGYDYWGQGTQVT |
| | | VSSAAAYPYDVPDYGSHHHHHH |
| 519 (The CDRs | NPC1e | QVQLQESGGGLVQSGGSLTLSCAASARTFI |
| are marked in | | AYAVGWFRQAPGKEREFVAAISWNGGMTDY |
| bold, SEQ ID | | ADFVKGRFAISRDNAKTVSLQMNNLKPEDT |
| Nos: 512-514) | | ALYYCTAGPARRSYSYRDGYDYWGQGTQVT |
| | | VSSAAAYPYDVPDYGSHHHHHH |
| 520 (The CDRs | NPC2a | QVQLQESGGGLVQAGGSLRLSCAASGSIFS |
| are marked in | | TNAMGWYRQAPDKQREFLAVITPRGRTAYA |
| bold, SEQ ID | | DSAKGRFTISRDNAMNSVYLQMNSLKFEDT |
| Nos: 521-523) | | AVYYCYAGRYRSYDARFATDIWGQGTQVTV |
| | | SSAAAYPYDVPDYGSHHHHHH |
| 524 (The CDRs | NPC2b | QVQLQESGGGLVQPGGSLRLSCAASGSIFS |
| are marked in | | TNAMGWYRQAPDKQREFLAVITPRGRTAYA |
| bold, SEQ ID | | DSAKGRFTISRDNAMNSVYLQMNSLKFEDT |
| Nos: 521-523) | | AVYYCYAGRYRSYDARFATDIWGQGTQVTV |
| | | SSAAAYPYDVPDYGSHHHHHH |
| 525 (The CDRs | NPC2c | QVQLQESGGGLVQAGGSLRLSCAASGSIFS |
| are marked in | | TNAMGWYRQAPDKQREFLAVITPRGRTAYA |
| bold, SEQ ID | | DSAKGRFTISRDNAMNSVYLQMNSLKFEDT |
| Nos: 521-522 | | AVYYCYAGRYRSFDARFATDIWGQGTQVTV |
| and 526) | | SSAAAYPYDVPDYGSHHHHHH |
| 527 (The CDRs | NPC2d | QVQLQESGGGLVRAGGSLRLSCAASGSIFS |
| are marked in | | TNAMGWYRQAPDKQREFLAVITPRGRTAYA |
| bold, SEQ ID | | DSAKGRFTISRDNAMNSVYLQMNSLKFEDT |
| Nos: 521-522 | | AVYYCYAGRYRSFDARFATDIWGQGTQVTV |
| and 526) | | SSAAAYPYDVPDYGSHHHHHH |
| 528 (The CDRs | NPC2e | QVQLQESGGGLVQAGGSLRLSCAASGSIFS |
| are marked in | | TNAMGWYRQAPDKQREFLAVITPRGRTAYA |
| bold, SEQ ID | | DSAKGRFTISRDNAMNSVYLQMNSLKFEDT |
| Nos: 521-522 | | AVYYCYAGRYRSFDARFATDIWSQGTQVTV |
| and 526) | | SSAAAYPYDVPDYGSHHHHHH |
| 529 (The CDRs | NPC2f | QVQLQESGGGLVQAGGSLRLSCAASGSIFS |
| are marked in | | TNAMGWYRQAPDKQREFLAVITPRGRTAYA |
| bold, SEQ ID | | DSAKGRFTISRDNAVNSVYLQMNSLKFEDT |
| Nos: 521-522 | | AVYYCYAGRYRSFDARFATDIWGQGTQVTV |
| and 526) | | SSAAAYPYDVPDYGSHHHHHH |
| 530 (The CDRs | NPC3a | QVQLQESGGGLVHPGGSLRLSCVASGAFLT |
| are marked in | | GATVGWYRQAPGKLRELVAAIITGGTTTYA |
| bold, SEQ ID | | DSVKGRFSISRDYTKRALILQMDSLRPDDT |
| Nos: 531-533) | | AVYFCSIRGFYRQTQFREIWGQGTQVTVSS |
| | | AAAYPYDVPDYGSHHHHHH |
| 534 (The CDRs | NPC3b | QVQLQESGGGLVQPGESLRLSCVASGAFLT |
| are marked in | | GATVGWYRQAPGKLRELVAAIITGGTTTYA |
| bold, SEQ ID | | DSVKGRFSISRDYTKRALILQMDSLRPDDT |
| Nos: 531-533) | | AVYFCSIRGFYRQTQFREIWGQGTQVTVSS |
| | | AAAYPYDVPDYGSHHHHHH |
| 535 (The CDRs | NPC3c | QVQLQESGGGLVQAGGSLRLSCVASGAFLT |
| are marked in | | GATVGWYRQAPGKLRELVAAIITGGTTTYA |
| bold, SEQ ID | | DSVKGRFSISRDYTKRALILQMDSLRPDDT |
| Nos: 531-533) | | AVYFCSIRGFYRQTQFREIWGQGTQVTVSS |
| | | AAAYPYDVPDYGSHHHHHH |
| 536 (The CDRs | NPC3d | QVQLQESGGGEVQAGGSLRLSCVASGAFLT |
| are marked in | | GATVGWYRQAPGKLRELVAAIITGGTTTYA |
| bold, SEQ ID | | DSVKGRFS1SRDYTKRALILQMDSLRPDDT |
| Nos: 531-533) | | AVYFCSIRGFYRQTQFREIWGQGTQVTVSS |
| | | AAAYPYDVPDYGSHHHHHH |
| 537 (The CDRs | NPC3e | QVQLQESGGGEVQAGGSLRLSCVASGAFLT |
| are marked in | | GATVGWYRQTPGNLRELVAAIITGGTTTYA |
| bold, SEQ ID | | DSVKGRFSISRDYTKRALILQMDSLRPDDT |
| Nos: 531-533) | | AVYFCSIRGFYRQTQFREIWGQGTQVTVSS |
| | | AAAYPYDVPDYGSHHHHHH |
| 538 (The CDRs | NPC4a | QVQLQESGGGLVQTGGSLRLSCAASGRTFS |
| are marked in | | NYVMGWFRQAPGKERELIGAINRSSTRLYY |
| bold, SEQ ID | | ADSVKGRFSISRDNAKSTVYLQMNSLKPED |
| Nos: 539-541) | | TAVYYCAADLVSILGKGYRDVDYWGQGTQV |
| | | TVSSAAAYPYDVPDYGSHHHHHH |
| 542 (The CDRs | NPC4b | QVQLQESGGGSVQAGGSLRLSCAASGRTFS |
| are marked in | | NYVMGWFRQAPGKERELIGAINRSSTRLYY |
| bold, SEQ ID | | ADSVKGRFSISRDNAKSTVYLQMNSLKPED |
| Nos: 539-541) | | TAVYYCAADLVSILGKGYRDVDYWGQGTQV |
| | | TVSSAAAYPYDVPDYGSHHHHHH |
| 543 (The CDRs | NPC4c | QVQLQESGGGLVQAGGSLRLSCAASGRTFS |
| are marked in | | SYVMGWFRQAPGKERELIGAINRSSTRLYY |
| bold, SEQ ID | | ADSVKGRFSISRDNAKSTVYLQMNSLKPED |
| Nos: 485 and | | TAVYYCAADLVSILGKGYRDVDYWGQGTQV |
| 540-541) | | TVSSAAAYPYDVPDYGSHHHHHH |
| 544 (The CDRs | NPC4d | QVQLQESGGGLVQPGGSLRVSCAASGFSFS |
| are marked in | | NYVMGWFRQAPGKERELIGAINRSSTRLYY |
| bold, SEQ ID | | ADSVKGRFSISRDNAKSTVYLQMNSLKPED |
| Nos: 545 and | | TAVYYCAADLVSILGKGYRDVDYWGQGT |
| 540-541) | | QVTVSSAAAYPYDVPDYGSHHHHHH |
| 546 (The CDRs | NPC5a | QVQLQESGGGLVQAGGSLRLSCVASGRTFS |
| are marked in | | PYTMGWFRRAPGKEREFVAAISWGAGVKSY |
| bold, SEQ ID | | ADSVKGRFTISRDNAENTVYLQMNMLKPDD |
| Nos: 547-549) | | TALYYCAAKRPISGSYSNERDYAYWGQGTQ |
| | | VTVSSAAAYPYDVPDYGSHHHHHH |
| 550 (The CDRs | NPC5b | QVQLQESGGGLVQPGGSLRLSCVASGRTFS |
| are marked in | | PYTMGWFRRAPGKEREFVAAISWGAGVKSY |
| bold, SEQ ID | | ADSVKGRFTISRDNAENTVYLQMNMLKPDD |
| Nos: 547-549) | | TALYYCAAKRPISGSYSNERDYAYWGQGTQ |
| | | VTVSSAAAYPYDVPDYGSHHHHHH |
| 551 (The CDRs | NPC6 | QVQLQESGGGLVQTGESLRLSCAGTNFLSS |
| are marked in | | RFEMGWYRQIPGKQRELVARIFRDGNTDYV |
| bold, SEQ ID | | DSVKGRFTISRDTAKNTIDLQMNNLKPEDT |
| Nos: 552-554) | | AGYFCHVHILGRDYWGQGTQVTVSSAAAYP |
| | | YDVPDYGSHHHHHH |
| 555 (The CDRs | NPC7a | QVQLQESGGGLVQAGDSLRISCKASGRTFS |
| are marked in | | SYPIGWFRQAPGKEREFVAAISRSGGRTYY |
| bold, SEQ ID | | ADSVKGRLTISRDNAKNTVYLQMNSLKLED |
| Nos: 556-558) | | TAVYYCAAKTTMGLPVGGTYEYDYWGQGTQ |
| | | VTVSSAAAYPYDVPDYGSHHHHHH |
| 559 (The CDRs | NPC7b | QVQLQESGGGLVQAGDSLRISCKASGRTFS |
| are marked in | | SYPIGWFRQAPGKEREFVAAISRSGGRTYY |
| bold, SEQ ID | | ADSVKGRFTISRDNAKNTVYLQMNSLKLED |
| Nos: 556-558) | | TAVYYCAAKTTMGLPVGGTYEYDYWGQGTQ |
| | | VTVSSAAAYPYDVPDYGSHHHHHH |
| 560 (The CDRs | NPC8 | QVQLQESGGGLVQPGGSLRLSCAASGFTLD |
| are marked in | | DYAIGWFRQAPGKERSFVAAIDSNGSNTYY |
| bold, SEQ ID | | ADSLKGRFTIARDNAKSMVFLRMNNLEPED |
| Nos: 561-563) | | TAVYYCAAGQNFWTFTTTPPPYWGQGTQVT |
| | | VSSAAAYPYDVPDYGSHHHHHH |
| 564 (The CDRs | NPC9 | QVQLQESGGGLVQAGDSLRISCKASGRTFS |
| are marked in | | NYPIGWFRQAPGKEREFVAAISRSGGGTRY |
| bold, SEQ ID | | ADSVKGRFTISRDNAKNTVYLQMNSLKRDD |
| Nos: 565-567) | | TAVYYCAAYFGNLGGGVGRSSDYDYWGQGT |
| | | QVTVSSAAAYPYDVPDYGSHHHHHH |
| 568 (The CDRs | NPC10 | QVQLQESGGGSVQAGESLTLSCAASNVLVS |
| are marked in | | KFTVAWFRQAPGKQRELVADIARAGFTSYA |
| bold, SEQ ID | | DFVRGRFSISRDNAQNTVTLQMNSLTPEDT |
| Nos: 569-571) | | AVYYCNCHVLGRDYWGQGTQVTVSSAAAYP |
| | | YDVPDYGSHHHHHH |
| 572 (The CDRs | NPC11 | QVQLQESGGGSVQPGGSLRLSCAASIRTFS |
| are marked in | | TYAFAWYRQAPGKQRELVAGISSGSRTNYA |
| bold, SEQ ID | | DSVKGRFTISRDNAKKTVYLQMNNLQPEDT |
| Nos: 573-575) | | AVYYCTKGRTNIDYWGQGTQVTVSSAAAYP |
| | | YDVPDYGSHHHHHH |
| 576 (The CDRs | NPC12 | QVQLQESGGGVVQPGGSLRLSCAASGRTFS |
| are marked in | | SYVMGWFRQAPGKEREFVATISTGGGTTYY |
| bold, SEQ ID | | ADSVKGRFTISRDNAKNTVYLQMNGLKPED |
| Nos: 485 and | | TAVYYCAAKMATGTASIRTYEYAYWGQGTQ |
| 577-578) | | VTVSSAAAYPYDVPDYGSHHHHHH |
| 579 (The CDRs | NPC13 | QVQLQESGGGLVQPGGSLRLSCAASGFTFS |
| are marked in | | RYAMGWFRQTPGKEREFVAAILWGGVIADS |
| bold, SEQ ID | | VKGRFTISRDNAKNTVDLQMFSLKPEDTAV |
| Nos: 580-582) | | YYCARRRGGLNNHLGSVGNYDYWGQGTQVT |
| | | VSSAAAYPYDVPDYGSHHHHHH |
| 583 (The CDRs | NPC14 | QVQLQESGGGSVQPGGSLRLSCEIIGATVS |
| are marked in | | SSSMAWYRQAPGLQRELVAGITTPSNPHYA |
| bold, SEQ ID | | ASVRGRFTISRDGARNLNYLQIDSAKPEDT |
| Nos: 584-586) | | AVYYCHAAIRGSIYRGQGTQVTVSSAAAYP |
| | | YDVPDYGSHHHHHH |

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first, indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Protein Target Preparation for Immunization—

DNA sequences of the targets proteins from Helicoverpa armigera (see Table 1 hereinabove) were optimized and synthesized. The synthesized sequences were cloned with His tag into a pET-28a or pET-30a vector and expressed in E. coli. strain BL21 Star (DE3). A single colony was inoculated into LB medium containing kanamycin; and the culture was incubated at 37° C. at 200 rpm and then induced with Isopropyl β-D-1-thiogalactopyranoside (IPTG). SDS-PAGE analysis was used to monitor protein expression. Following, BL21 Star (DE3) stored in glycerol was inoculated into TB medium containing kanamycin and cultured at 37° C. When the OD600 reached 1.2, cell culture was induced with IPTG at 15° C. for 16 hours. Cells were harvested by centrifugation and pellets were resuspended with lysis buffer (20 mM NaPi pH7.4, 500 mM NaCl, 20 mM Imidazole, Protease Inhibitor cocktail—cOmplete™, EDTA-free Protease Inhibitor Cocktail-Roche cat #4693132001) followed by sonication and centrifugation. The precipitate was dissolved using urea; and the denatured supernatant was kept for future purification. Following, the protein was purified from the supernatant using a Ni-NTA column (GE-Healthcare, Cat No. 17-5318-02): The column was washed with 10 bed volumes of wash buffer (20 mM NaPi pH7.4, 500 mM NaCl, 50 mM Imidazole) and the protein was eluted with elution buffer (20 mM NaPi pH7.4, 500 mM NaCl, 500 mM Imidazole). Following the elution, the fractions were placed in MEGA tubes with membrane cutoff of 3.5 kDa (Tivan Biotech MEGA3-50) and dialyzed in 1 L PBS pH7.4 at 4° C. overnight, followed by a second round of dialysis under the same conditions. Some of the target proteins (e.g. the V-ATPase subunit C target protein) were also refolded and sterilized by 0.22 μm filter prior to storing in aliquots. The concentration was determined using a-Nano-drop. The protein purity and molecular weight were determined by a standard SDS-PAGE.

### Immunization—

A llama was subcutaneously injected on days 0, 7, 14, 21, 28 and 35, each time with about 125 or 150 μg target protein in combination with a Gerbu adjuvant P (Gerbu Biotech, #3111-0025). On day 40, about 100 ml anti-coagulated blood was collected from the llama for lymphocyte preparation.

### Construction of a VHH Library—

A VHH library was constructed from the llama lymphocytes to screen for the presence of antigen-specific nanobodies. To this end, total RNA from peripheral blood lymphocytes was used as a template for first strand cDNA synthesis with an oligo (dT) primer. Using this cDNA, the VHH encoding sequences were amplified by PCR, digested with PstI (#ER0611 from Thermofischer) and NotI (#11037668001 from Sigma), and cloned into the PstI & NotI sites of the phagemid vector pMECS. The VHH library comprised about 108 independent transformants, with about 80-92% of transformants harboring the vector with the right insert size.

### Isolation and Sequencing of Specific Target Nanobodies—

The library was panned for 3 rounds on solid-phase coated with the target antigen (100 μg/ml in 100 mM NaHCO3 pH 8.2). The enrichment for antigen-specific phages was assessed following each round of panning by comparing the number of phagemid particles eluted from antigen-coated wells with the number of phagemid particles eluted from negative control (uncoated blocked) wells. These experiments indicated that the phage population was enriched for antigen-specific phages by the 3rd round. The antigen used for panning & ELISA screening was the same as the one used for immunization, using uncoated blocked wells as a negative control. The amino acid sequences of the generated nanobodies are shown in Table 2A-6 hereinabove.

### Nanobodies Expression and Purification—

The nanobodies DNA sequence were optimized and synthesized by GenScript. The synthesized sequences were cloned with His tag into a pET-28b+ vector for protein expression in E. coli. Following, E. coli SHuffle T7 was transformed with the recombinant plasmids. A single colony was inoculated into LB medium containing kanamycin; and the culture was incubated at 37° C. at 220 rpm and then induced with 1M IPTG. SDS-PAGE analysis were used to monitor expression. Following, SHuffle T7 stored in glycerol was inoculated into TB medium containing kanamycin and cultured at 37° C. When the OD600 reached about 1.2, cell culture was induced with 1M IPTG at 30° C. for 20-22 hours. Cells were harvested by centrifugation and cell pellets were resuspended with lysis buffer (20 mM NaPi pH7.5, 500 mM NaCl, 20 mM imidazole) followed by sonication and centrifugation. The precipitate was dissolved using urea and the denatured supernatant was kept for future purification. The nanobodies were purified with a Ni-NTA column: The column was washed with 10 bed volumes of Wash Buffer (20 mM NaPi pH7.5, 500 mM NaCl, 50 mM imidazole) and the protein was eluted with elution buffer (20 mM NaPi pH7.5, 500 mM NaCl, 500 mM imidazole). Following, the elution fractions were dialyzed at 4° C. for overnight with dialyzed buffer (PBS pH 7.4). The concentration was determined by Nanodrop. Protein purity and molecular weight were determined by standard SDS-PAGE.

### Binding of the Generated Nanobodies to the Target Protein—

Maxisorb 96-wells plates were coated with 10 μg/ml of the target protein by an overnight incubation at 4° C. The coated plates were washed with PBS and blocked for 1 hour at 37° C. in blocking buffer containing PBS+0.5% tween+1% BSA, followed by 1 hour incubation at room temperature with 2 μg/ml of the tested nanobody. Following washes with PBS, the plates were incubation with MonoRab™ Rabbit Anti-Camelid VHH Antibody-HRP (Genscript, Cat no. A01860) in blocking buffer. Finally, the plates were washed with PBS, incubated for 30 minutes with TMB solution followed by addition of H₂SO₄ and absorbance was read at 450 nm.

### Stability Assays—

The stability of the generated nanobodies was tested in “field-like conditions” by incubating them in natural sunlight, natural UV radiation and high summer out door/filed temperatures. In addition, stability to enzymatic proteolytic activity and high pH was evaluated by incubation of the nanobodies with lumen juices harvested from H. armigera 4^{th} in start larvae guts [see e.g. Purcell, John P., John T. Greenplate, and R. Douglas Sammons. Insect Biochemistry and Molecular Biology 22.1 (1992): 41-47; Pang, A. S., & Gringorten, J. L. (1998). FEMS microbiology letters, 167(2), 281-285; and Ma, Gang, et al. Insect biochemistry and molecular biology 35.7(2005): 729-739]. Specifically, Maxisorb 96-wells plates were coated with 2 g/ml of the tested nanobody by an overnight incubation at 4° C. The coated plates were washed with PBS and blocked for 1 hour at 37° C. in blocking buffer containing PBS+0.5% tween+1% BSA and washed with PBS. The coated plates were incubated with PBS in “field-like condition” for 1-3 hours or in presence of lumen gut juices for 1 hour at room temperature. Following incubation, plates were washed with PBS and incubated with MonoRab™ Rabbit Anti-Camelid VHH Antibody-HRP in blocking buffer. Finally, the plates were washed with PBS, incubated for 30 min with TMB solution followed by addition of H₂SO₄ and absorbance was read at 450 nm.

### Insect Culture—

Cotton bollworms were collected from cotton fields in Israel and reared in the laboratory at 24° C. with 70% relative humidity on 10 hours day/14 hours night photoperiod. The collected larvae were kept on an artificial diet with Ward's diet powder Stonefly Heliothis Diet (Product No. 38-0600, Ward's Natural Science, Rochester, NY) supplemented with Vitamin mixture (Nicotinic acid 40 μM, Calcium pantothenate 5 μM, Riboflavin 5 μM, Thiamine hydrochloride 4 μM, Pyridoxine hydrochloride 5 μM, Folic acid 2.5 μM, D-biotin 4 μM and Cyanocobalamin 5 nM), Sorbic acid 0.2%, Methyl paraben 0.4%, Ascorbic acid 0.25%, Brewer Yeast 1%, white vinegar 4% and Linseed oil 0.5%.

### Feeding Bioassays—

0.5 gr of Heliothis artificial diet was added into each well of 24 wells plates and overlaid with 100 μl suspension of the tested nanobody in a concentration of 0.4 mg/ml or PBS as a negative control. To each well, one 24-48 hours old larva was added in 12 replications. The plates were coated with polyofin sealing foil (HJ-BIOANALITIC, Cat No. 900371) and incubated in the laboratory at 24° C. with 70% relative humidity in the dark. Each study for each nanobody was repeated 3-5 times. Following 7 days of incubation, the larvae were moved to larger petri dishes with a diameter of 5 cm with the same artificial food with no addition of the nanobody. The body length and weight of each larva was measured following 7 and 14 days of incubation and then followed by observation on the pupation and adult emerged timing.

### Cotton Growth—

Cotton cultivar Akalpi (Inter-specific hybrid (ISH) between G. hirsutum and G. barbadense; 1432 Intercott Hazera) was grown under a control temperature regime of 25/18° C. day/night. Temperatures were monitored and maintained by the SAS automation system. In addition, fertigation was provided with Sheffer7-7-7 plus calcium; keeping a leaching fraction of 30%. Pest control was conducted exclusively mechanically by using sticky-colored traps. Cropping cycle duration was between six to eight weeks. Following this period, plants were transferred to the laboratory serving as a plant model for the bioassay experiments.

### Leaf Bioassays—

1 ml sterilized agar was added to each well of 12 wells plates and a 2 cm Ø cotton leave disc was deposited over the agar in each well. Each leave disc was overlaid with 50 μl suspension of the tested nanobody in a concentration of 0.4, 1, 2 or 4 mg/ml, or PBS as a negative control. To each well, one 24-48 hours old larva was added in 12 replicates. The plates were sealed with polyofin sealing foil (HJ-BIOANALITIC, Cat No. 900371) and incubated in the laboratory at 24° C. and 70% relative humidity. Following 4 days of incubation, the larvae were moved to new plates with fresh cotton discs, treated with the same nanobody concentration or PBS, and on day 7 the larvae were moved once again to bigger wells (6 wells plates), with an addition of agar and fresh 2.8 cm Ø cotton leave discs treated with 100 μl of the same nanobody at the same concentration. The larvae mortality was evaluated on days 4, 7 and 10. Each study for each nanobody was repeated 2-3 times.

Immunofluorescence—Purified anti-CBD nanobody referred to herein as “CB20901” at a concentration of 1 mg/ml was conjugated to fluorescently-labeled Cy3 (ab188287-Cy3 Fast Conjugation kit Cambridge, UK) following manufacturer instructions. The feeding bioassay was carried as described hereinabove. Specifically, 12 larvae were fed with 100 μl Cy3-fluorescently-labeled nanobody and 12 larvae served as control and fed with 100 μl of PBS. Following 36 hours of incubation treated larvae were transferred to clean freshly prepared Heliothis artificial diet for another 12 hours, in order to eliminate false-positive fluorescent signals, following which the first imaging session took place. In the next step, larvae were returned to nanobody/PBS-food until they were six days old followed by 12 hours feeding on food without the nanobody, and finally a second imaging session of live larvae and extracted gut was carried. For valid imaging comparisons, set up was kept as fallow: Nikon Eclipse 80i microscope, NIS-element software, Camara DS-Ri2, lent 1×_zoom_2.0×_exposure 10 ms_gain_7.6× (for food imaging), lent 1×_zoom 1.4×_exposure 200 ms_gain_2.0× (for 48 hours old larvae) and lent 1×_zoom1.4× exposure 400 ms_gain_6.2× (for 6 days old live larvae and extracted gut).

### Example 1

### Anti-Chitin Binding Domain Nanobodies

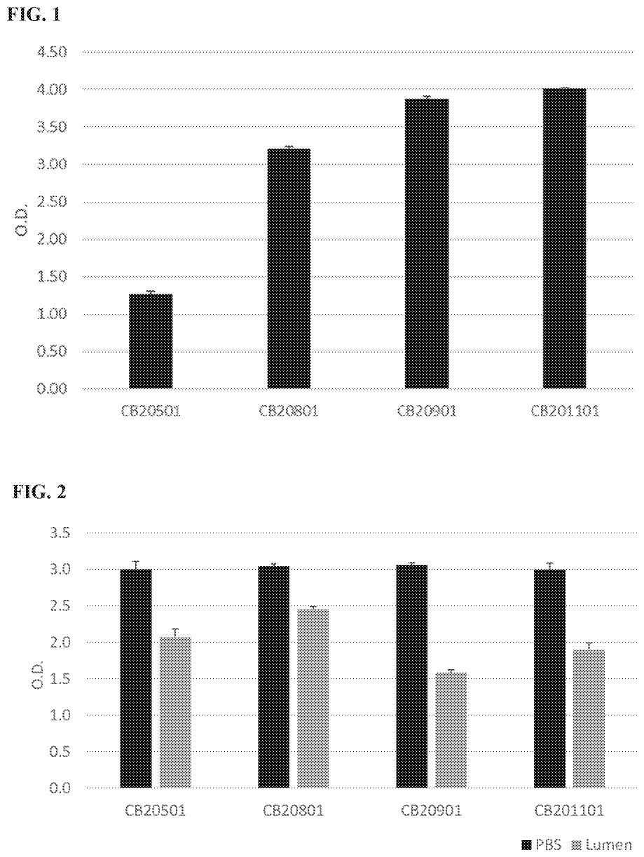

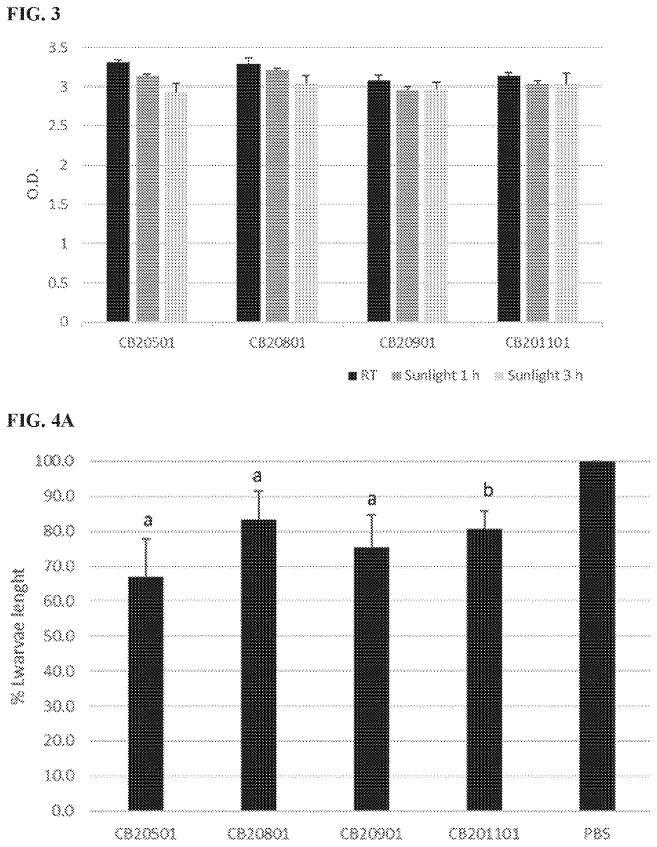

Several nanobodies were generated against the Helicoverpa armigera chitin binding domains (CBD) antigen, referred to herein as Ha-PMP5B1 and Ha-PMP5B2 (see Tables 2A-B hereinabove). The Ha-PMP5B1 protein contains 506 amino acid with five different CBDs from Helicoverpa armigera and the Ha-PMP5B2 protein is a partial protein of Ha-PMP5B1 and contains 192 amino acid with two CBDs. ELISA assays using plates coated with the target antigen verified that the generated nanobodies bind the target CBD antigen (FIG. 1). As specific embodiments disclose administration of the nanobodies to Helicoverpa armigera larvae by oral delivery, thereby the nanobodies are absorbed into the larvae gut lumen (which has a high pH and contains a high content of proteolytic enzymes) until reaching the target site in the peritrophic membrane of the larvae gut; the stability of the anti-CBD nanobodies in the lumen was studied. ELISA assays using plates coated with the generated anti-CBD nanobodies and incubated for 1 hour in the presence of lumen juices harvested from Helicoverpa armigera demonstrated that 40-70% of the nanobodies remained intact following an hour of incubation (FIG. 2), thus indicating the survival ability of the generated anti-CBD nanobodies in lumen conditions. In addition, stability of the generated anti-CBD nanobodies was also tested under conditions that mimic field conditions by incubation outside in the open air with natural solar radiation and at high external temperatures of 30° C. for 1-3 hours. The results demonstrated that following 3 hours of incubation in filed like condition of 30° C., 60-70% humidity and a UV index of 4-5, only a slight decrease in the nanobodies content was observed compared to control (FIG. 3).

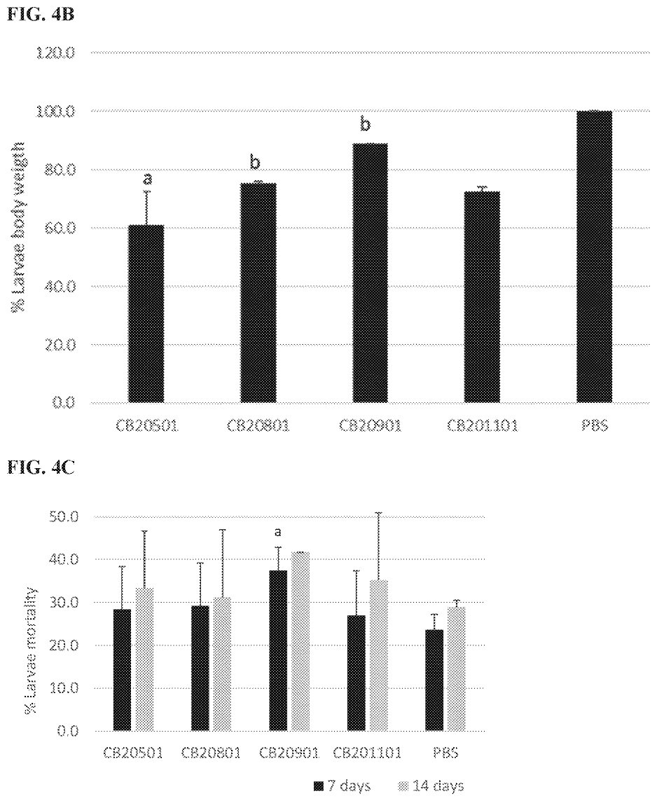

To assess the insecticidal activity of the generated nanobodies, young Helicoverpa armigera larvae at the age of no more than 48 hours were grown on artificial food supplemented with a single dose of the anti-CBD generated nanobodies. Observations were carried out through all of the insect's life cycle, including weight and length measurements of the larvae on day 7 and 14 and later on observation on the number of individuals transformed from pupa to adults. The results demonstrated that larvae fed 7 days with several anti-CBD nanobodies demonstrated loss in their length (FIG. 4A) and/or weight loss (FIG. 4B) and/or an increase in mortality (FIG. 4C), depending on the nanobody tested.

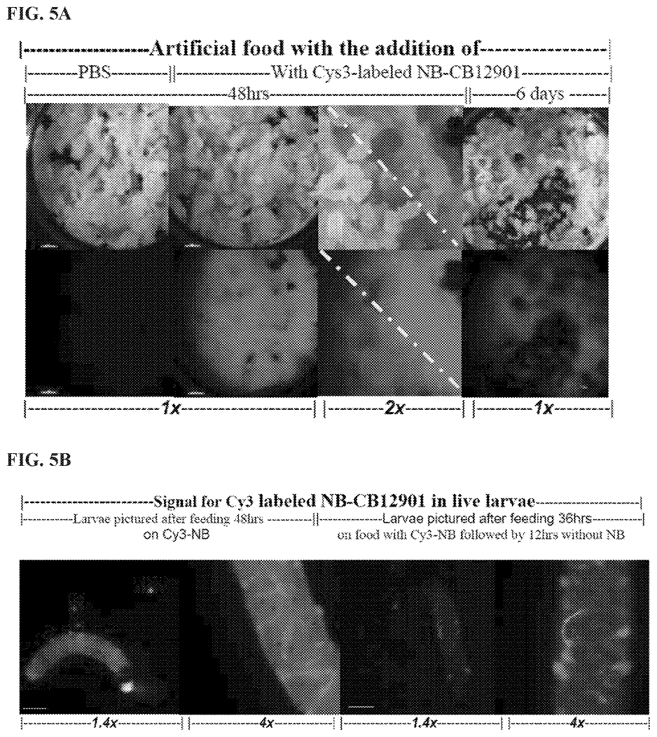

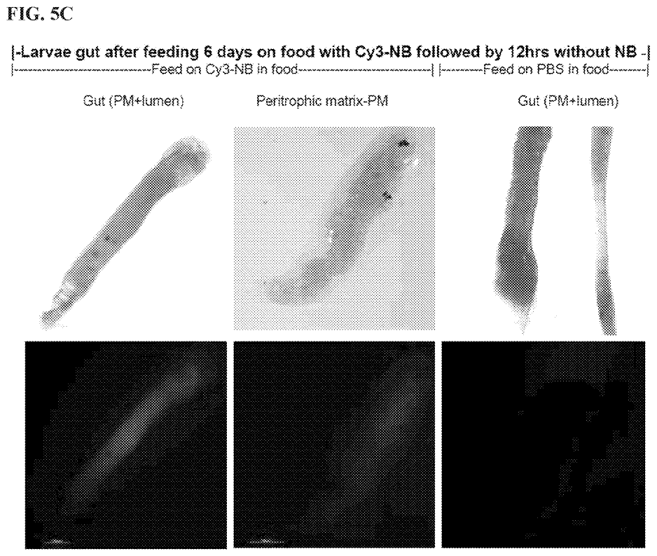

In order to track the nanobodies trajectory in larvae gut, Helicoverpa armigera larvae were grown on artificial food supplemented with a fluorescently-labeled Cy3 anti-CBD nanobody CB12901. As shown in FIG. 5A, the labeled nanobody was stable in the food for at least a week. In addition, a decrease in the fluorescence signal was observed between the food and larvae feces following feeding, suggesting uptake of the labeled nanobody by larvae gut (FIG. 5A). Moreover, the labeled nanobody was detected in larvae 12 hours following transfer of the larvae to food not containing the nanobody, indicating that the labeled nanobody is retained in larvae gut (FIG. 5B). Furthermore, the ability of the labeled nanobody to reach the peritrophic matrix was confirmed following 6 days of feeding: at this timing positive signal was observed from all gut and specifically from peritrophic matrix following separation between the peritrophic matrix and the lumen gut (F).

### Example 2

### ANTI-V-ATPase Subunit C Nanobodies

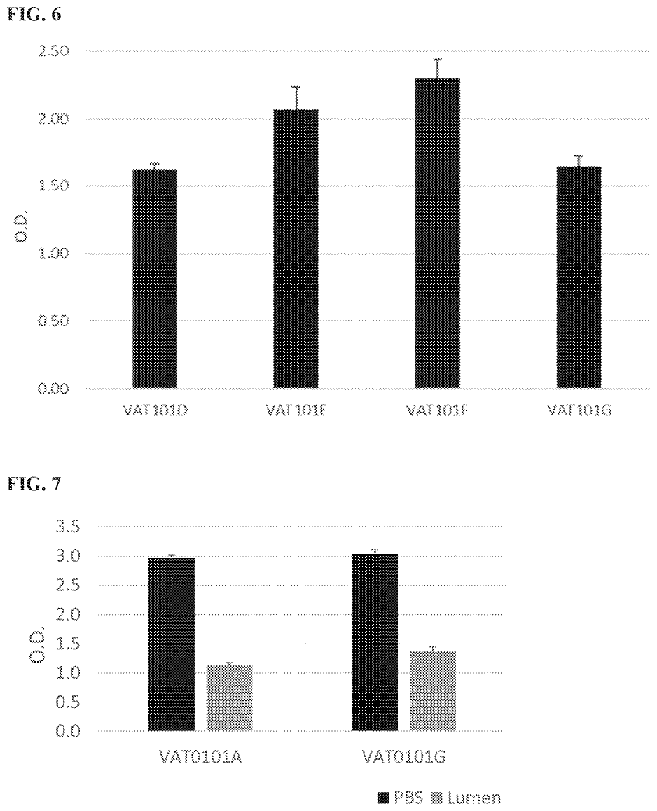

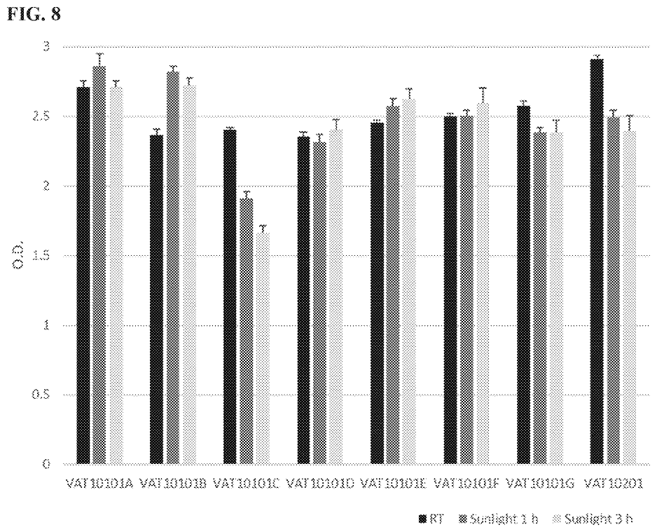

Several nanobodies were generated against the Helicoverpa armigera V-ATPases Subunit C (referred to herein as “VAT”) antigen (see Tables 3A-B hereinabove). ELISA assays using plates coated with the target antigen verified that the generated nanobodies bind the target VAT antigen (FIG. 6). Following, the stability of the anti-VAT nanobodies in lumen conditions and in field like conditions was studied (FIGS. 7-8). The results indicated that 40-70% of the nanobodies remained intact following an hour of incubation in the presence of lumen juices, thus indicating the survival ability of the generated anti-VAT nanobodies in lumen conditions. In addition, the results demonstrated that following 3 hours of incubation in filed like condition of 30° C., 60-70% humidity and a UV index of 4-5, only a slight decrease in the nanobodies content was observed compared to control.

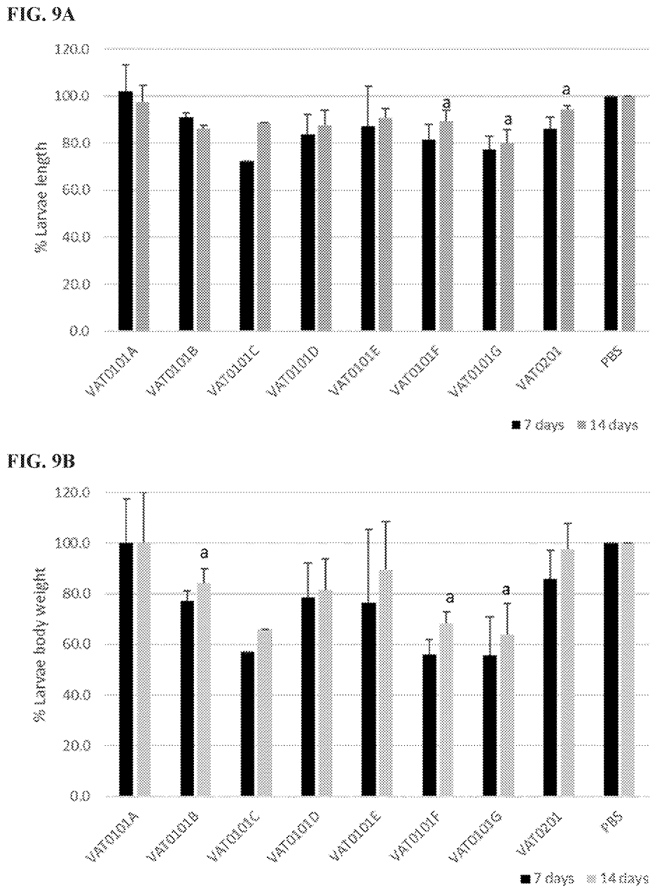

To assess the insecticidal activity of the generated nanobodies, young Helicoverpa armigera larvae at the age of no more than 48 hours were grown on artificial food supplemented with a single dose of the anti-VAT generated nanobodies. Observations were carried out through all of the insect's life cycle, including weight and length measurements of the larvae on day 7 and 14 and later on observation on the number of individuals transformed from pupa to adults. The results show that larvae fed 7 days with the generated anti-VAT nanobodies demonstrated loss in their length (FIG. 9A) and/or weight loss and/or an increase in mortality (FIG. 9C), depending on the nanobody tested.

In addition, the percentage of larvae that survived and completed a full life cycle from larvae to pupa stage and then turned to adult moths was determined. As shown in Table 9 hereinbelow, the larvae treated for 7 days with anti-VAT nanobodies had lower percentages of larvae, which completed a full life cycle to become adult moths. Importantly, though the larvae were exposed to the anti-VAT nanobodies only at the first 7 days out of 25-30 days of the experiment, the results showed that they had a long-term stable effect on the Helicoverpa armigera and influenced them also later development stages of pupation and adultness.

TABLE 9
Percentages of larvae, which completed a full life cycle to adult moths

| | % adult moths |
|---|---|
| VAT0101A | 58 |
| VAT0101B | 42 |
| VAT0101C | 33 |
| VAT0101D | 16 |
| VAT0101E | 33 |
| VAT0101F | 42 |
| VAT0101G | 58 |
| VAT0202 | 58 |
| PBS | 67 |

### Example 3

### Anti-Trehalase Nanobodies

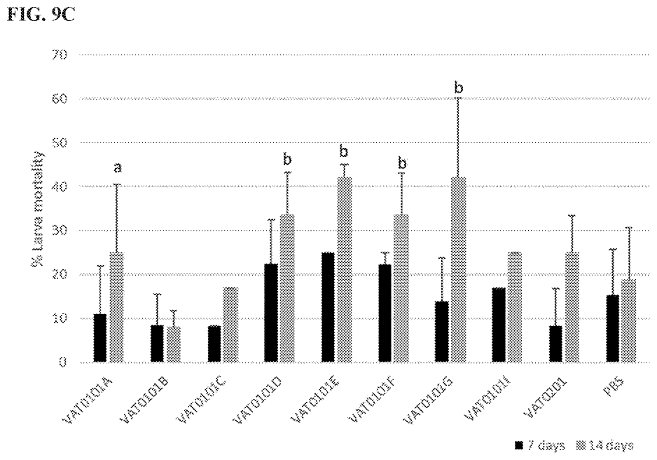

Several nanobodies were generated against the Helicoverpa armigera trehalase (referred to herein as “TRH”) antigen (see Table 4 hereinabove). ELISA assays using plates coated with the target antigen verified that the generated nanobodies bind the target trehalase antigen (FIG. 10). Following, the stability of the anti-TRH nanobodies in field-like conditions and in high temperature of 50° C. was studied (FIGS. 11A-B). The results demonstrated that overnight incubation in field like conditions of 30° C., 60-70% humidity and a UV index of 4-5 had no effect or induced only a slight decrease in the nanobodies content compared to control. Similar results were demonstrated following an overnight incubation at 50° C.

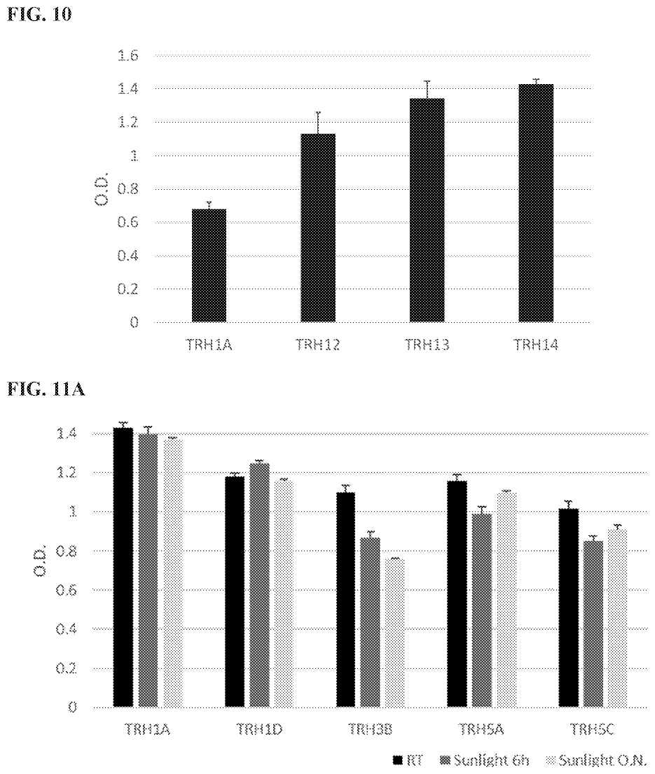

To assess the insecticidal activity of the generated nanobodies, young Helicoverpa armigera larvae at the age of no more than 48 hours were grown on artificial food supplemented with anti-TRH generated nanobodies; and mortality rate was measured on days 7 and 14. Results showed a significant increase in larvae mortality following treatment with the generated anti-TRH nanobodies (FIG. 12A). Following, young Helicoverpa armigera larvae at the age of no more than 48 hours were grown on cotton leave discs supplemented with the generated anti-TRH nanobodies administrated on days 0, 4 and 7, and mortality rate was measured on days 4, 7 and 10. Results showed a significant dose dependent increase in larvae mortality following treatment with the anti-TRH nanobodies (FIG. 12B).

In addition, observations were carried out through all of the insect's life cycle, including weight, length measurements of the larvae and later on observation on the number of individuals transformed from pupa to adults. In addition, the percentage of larvae that survive and complete a full cycle from larvae to pupa stage and then to adult moths was determined.

### Example 4

### Anti-Cytochrome P450 Monooxygenase Nanobodies

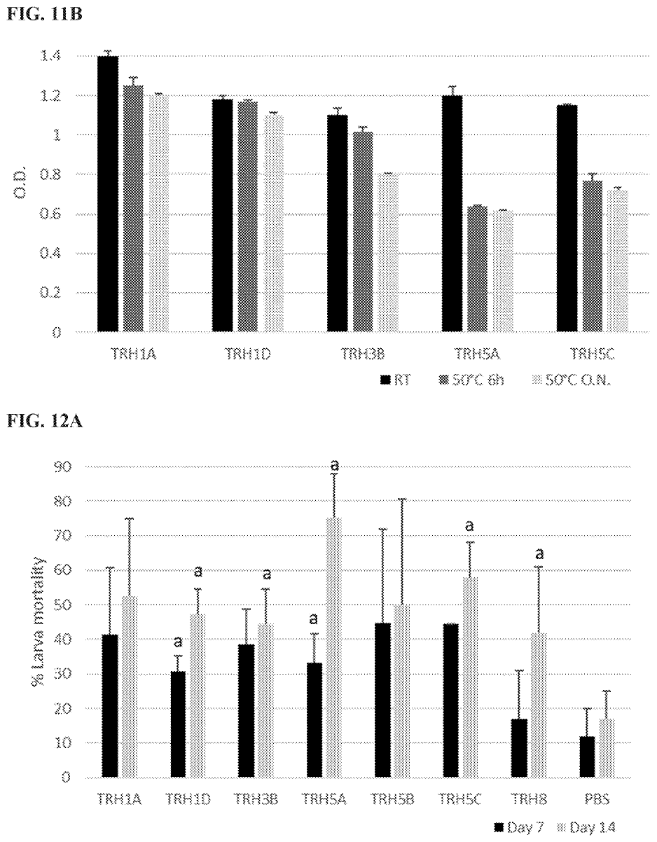

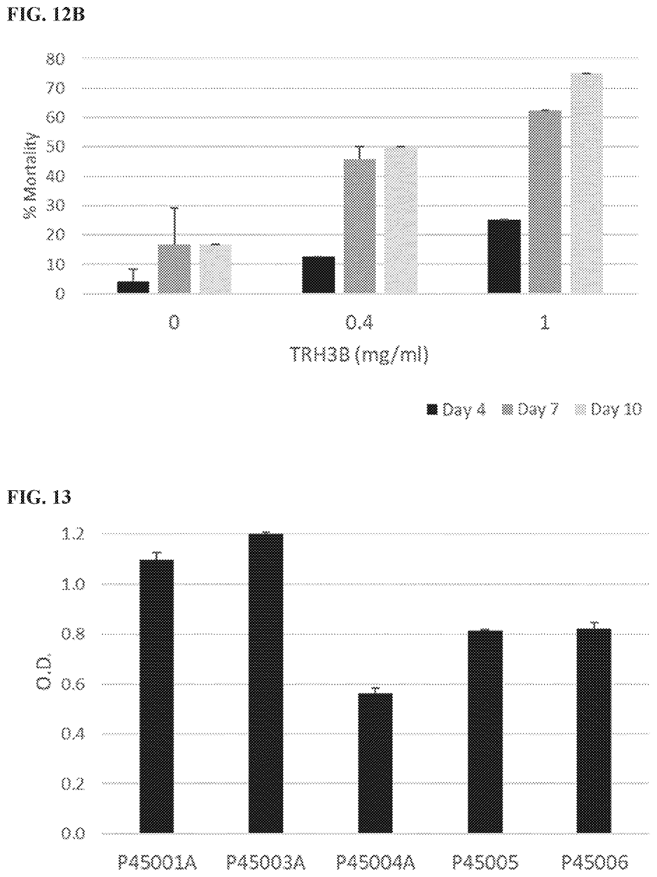

Several nanobodies were generated against the Helicoverpa armigera cytochrome p450 monooxygenase (referred to herein as “P450”) antigen (see Table 5 hereinabove). ELISA assays using plates coated with the target antigen verified that the generated nanobodies bind the target P450 antigen (FIG. 13). Following, the stability of the anti-P450 nanobodies in lumen conditions and in field like conditions is studied.

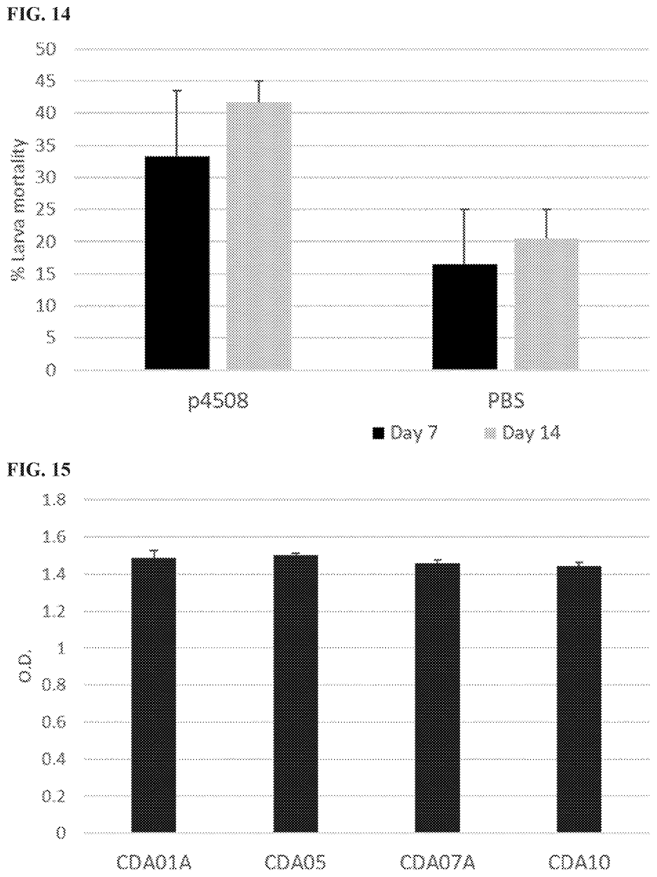

To assess the insecticidal activity of the generated nanobodies, young Helicoverpa armigera larvae at the age of no more than 48 hours were grown on artificial food supplemented with the anti-P450 generated nanobodies; and mortality rate was measured on days 7 and 14. Results showed a significant increase in larvae mortality following treatment with the anti-P450 nanobodies (FIG. 14).

In addition, observations were carried out through all of the insect's life cycle, including weight and length measurements of the larvae on day 7 and 14 and later on observation on the number of individuals transformed from pupa to adults. In addition, the percentage of larvae that survive and complete a full life cycle from larvae to pupa stage and then to adult moths was determined.

### Example 5

### Anti-Chitin Deacetylase Nanobodies

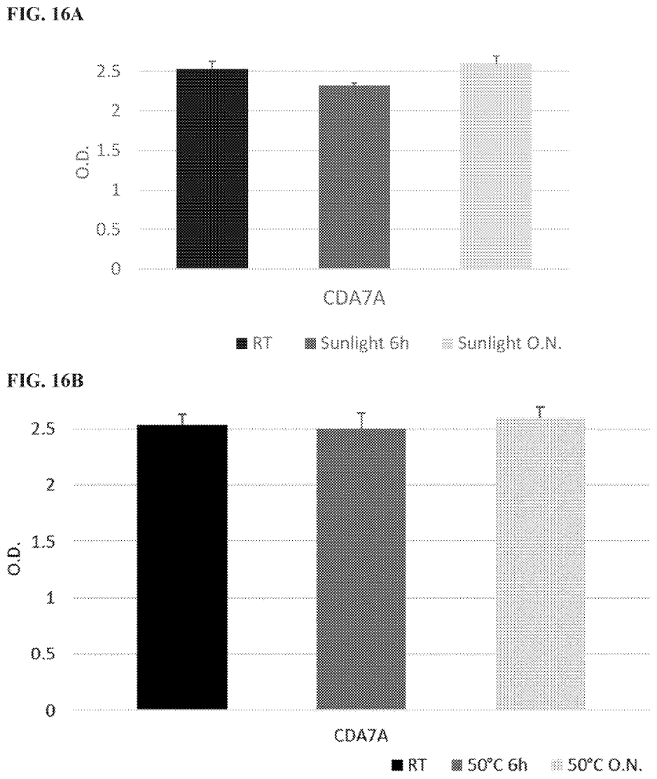

Several nanobodies were generated against the Helicoverpa armigera chitin deacetylase (referred to herein as “CDA”) antigen (see Table 6 hereinabove). ELISA assays using plates coated with the target antigen verified that the generated nanobodies bind the target CDA antigen (FIG. 15). Following, the stability of the anti-CDA nanobodies in field like conditions and in high temperature of 50° C. was studied (FIGS. 16A-B). The results demonstrated that overnight incubation in field-like conditions of 30° C., 60-70% humidity and a UV index of 4-5, resulted in no change in the nanobodies content compared to control. Similar results were demonstrated following an overnight incubation at 50° C.

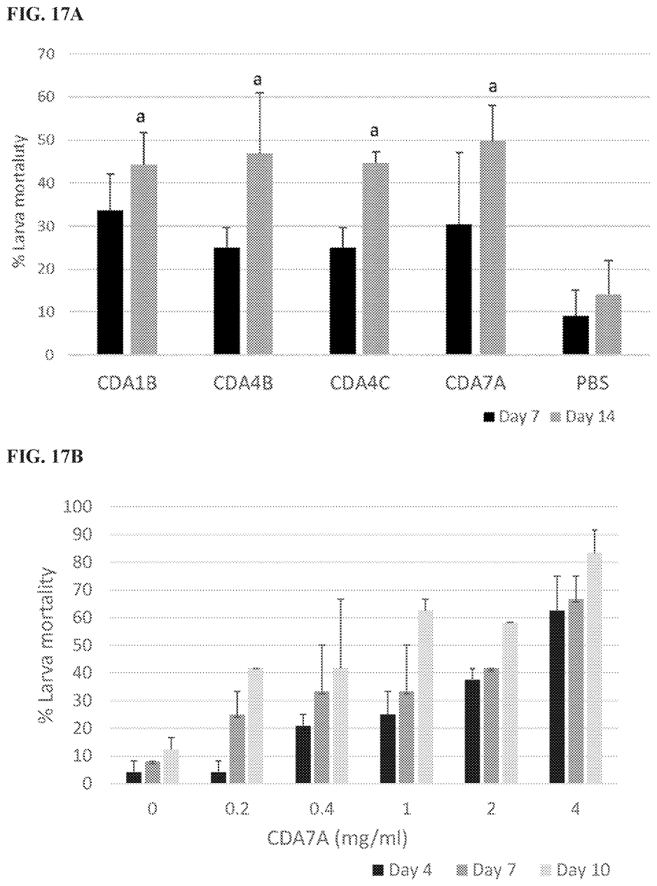

To assess the insecticidal activity of the generated nanobodies, young Helicoverpa armigera larvae at the age of no more than 48 hours were grown on artificial food supplemented with the generated anti-CDA nanobodies; and mortality rate was measured on days 7 and 14. Results showed a significant increase in larvae mortality following treatment with the anti-CDA nanobodies (FIG. 17A). Following, young Helicoverpa armigera larvae at the age of no more than 48 hours were grown on cotton leave discs supplemented with the anti-CDA nanobodies, administrated on days 0, 4 and 7, and mortality rate was measured on days 4, 7 and 10. Results showed a significant dose dependent increase in larvae mortality following treatment with the anti-CDA nanobodies (FIG. 17B).

In addition, observations were carried out through all of the insect's life cycle, including weight and length measurements of the larvae on day 7 and 14 and later on observation on the number of individuals transformed from pupa to adults. In addition, the percentage of larvae that survive and complete a full life cycle from larvae to pupa stage and then to adult moths was determined.

### Example 6

### Anti-Chitin Synthase Nanobodies

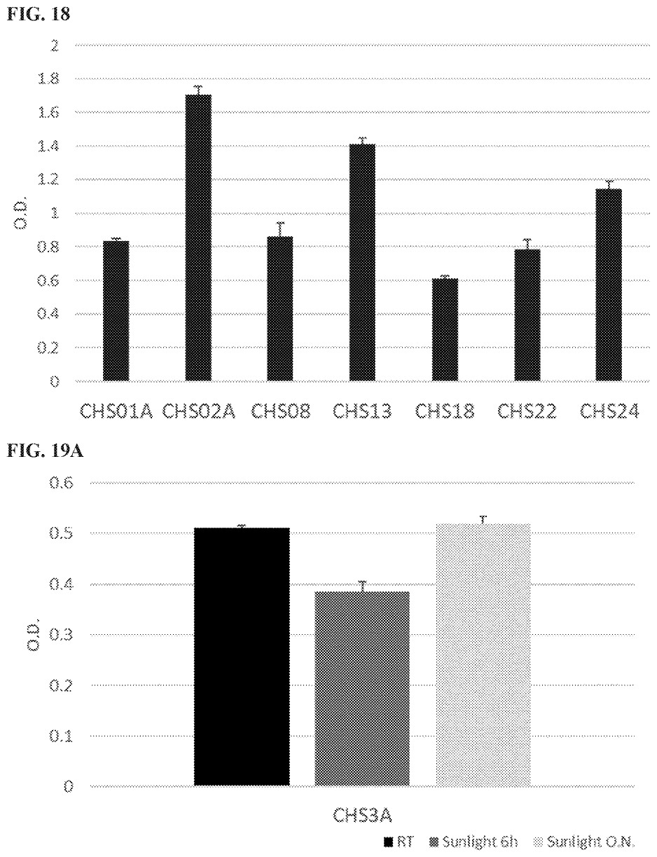

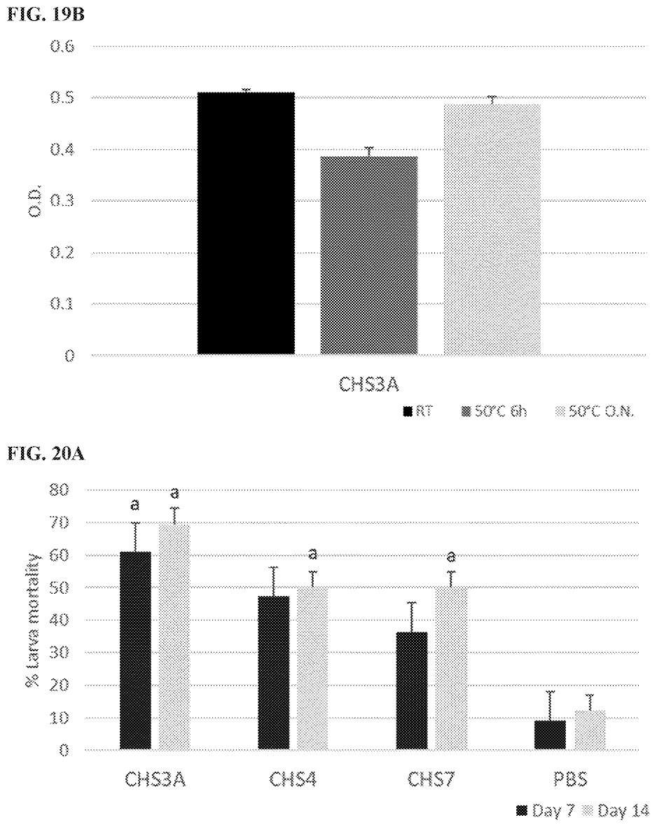

Several nanobodies were generated against the Helicoverpa armigera chitin synthase (referred to herein as “CHS) antigen (see Table 7 hereinabove). ELISA assays using plates coated with the target antigen verified that the generated nanobodies bind the target CHS antigen (FIG. 18). Following, the stability of the anti-CHS nanobodies in field-like conditions and in high temperature of 50° C. was studied (FIGS. 19A-B). The results demonstrated that overnight incubation in field-like conditions of 30° C., 60-70% humidity and a UV index of 4-5, generated only a slight decrease in the nanobodies content compared to control. Similar results were demonstrated following an overnight incubation at 50° C.

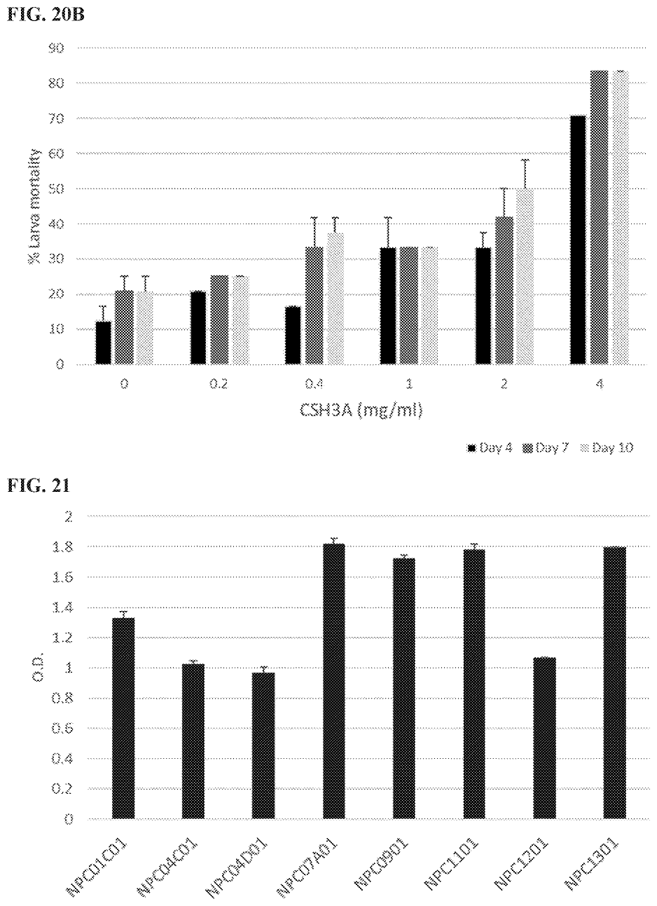

To assess the insecticidal activity of the generated nanobodies, young Helicoverpa armigera larvae at the age of no more than 48 hours were grown on artificial food supplemented with the generated anti-CHS nanobodies; and mortality rate was measured on days 7 and 14. Results showed a significant increase in larvae mortality following treatment with the anti-CHS nanobodies (FIG. 20A). Following, young Helicoverpa armigera larvae at the age of no more than 48 hours were grown on cotton leave discs supplemented with the anti-CHS nanobodies, administrated on days 0, 4 and 7, and mortality rate was measured on days 4, 7 and 10. Results showed a significant dose dependent increase in larvae mortality following treatment with the anti-CHS nanobodies (FIG. 20B).

In addition, observations were carried out through all of the insect's life cycle, including weight and length measurements of the larvae on day 7 and 14 and later on observation on the number of individuals transformed from pupa to adults. In addition, the percentage of larvae that survive and complete a full life cycle from larvae to pupa stage and then to adult moths was determined.

### Example 7

### ANTI-NPC1 Sterol Transporter Nanobodies

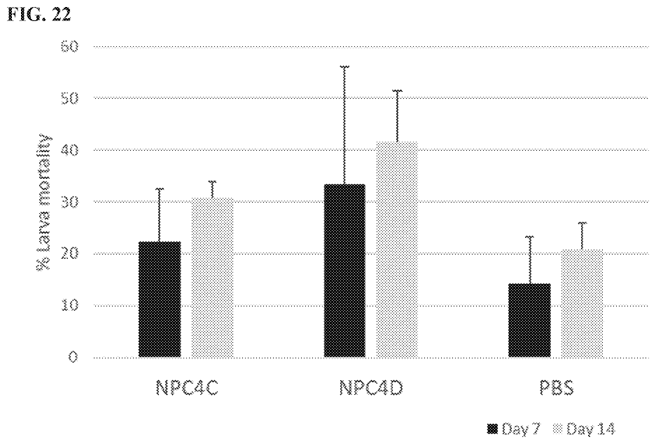

Several nanobodies were generated against the Helicoverpa armigera NPC1 sterol transporter (referred to herein as “NPC1”) antigen (see Table 8 hereinabove). ELISA assays using plates coated with the target antigen verified that the generated nanobodies bind the target NPC1 antigen (FIG. 21). Following, the stability of the anti-NPC1 sterol transporter nanobodies in lumen conditions and in field like conditions is studied.

To assess the insecticidal activity of the generated nanobodies, young Helicoverpa armigera larvae at the age of no more than 48 hours were grown on artificial food supplemented with the generated anti-NPC1 nanobodies; and mortality rate was measured on days 7 and 14. Results showed a significant increase in larvae mortality following treatment with the anti-NPC1 nanobodies (FIG. 22).

In addition, observations were carried out through all of the insect's life cycle, including weight and length measurements of the larvae on day 7 and 14 and later on observation on the number of individuals transformed from pupa to adults. In addition, the percentage of larvae that survive and complete a full life cycle from larvae to pupa stage and then to adult moths was determined.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A VHH antibody comprising complementarity determining regions (CDRs) as set forth in SEQ ID NOs: 15 and 28-29 arranged in a sequential order from N to C on said VHH antibody, wherein said VHH antibody is capable of binding chitin binding domain (CBD).

2. The VHH antibody of claim 1, further comprising a heterologous toxin moiety.

3. The VHH antibody of claim 1, wherein said VHH antibody amino acid sequence comprises SEQ ID NO: 26.

4. The VHH antibody of claim 1, being a synthetic recombinant VHH antibody.

5. The VHH antibody of claim 1, wherein said CBD is a Helicoverpa armigera CBD.

6. A polynucleotide encoding the VHH antibody of any one of claim 1.

7. A nucleic acid construct comprising the polynucleotide of claim 6 and a cis-acting regulatory element for directing expression of said polynucleotide.

8. The nucleic acid construct of claim 7, wherein said cis-acting regulatory element is heterologous to said polynucleotide.

9. A transformed host cell comprising the VHH antibody of claim 1 or a polynucleotide or a nucleic acid construct encoding same.

10. A method of producing an anti-chitin binding domain (CBD) VHH antibody, the method comprising culturing the cell of claim 9.

11. The method of claim 10, comprising isolating the VHH antibody.

12. A method of insect control, the method comprising contacting the insect with the VHH antibody of claim 1, a polynucleotide or a nucleic acid construct encoding same or a host cell expressing same.

13. The method of claim 12, wherein said contacting comprises applying the VHH antibody directly to the insect.

14. The method of claim 12, wherein said contacting comprises applying the VHH antibody to an organism or a surface, which may be in contact with said insect.

15. The method of claim 12, wherein said insect is selected from the group consisting of moth, stinkbug, hopper, beetle, aphid and honeybee.

16. The method of claim 12, wherein said insect is a moth.

17. The method of claim 16, wherein said moth is Helicoverpa armigera.

18. A plant comprising the VHH antibody of claim 1 or a polynucleotide or a nucleic acid construct encoding same.

19. The plant of claim 18, being a transgenic plant.

20. A commodity product comprising the VHH antibody of claim 1.


SEQUENCE LISTING (596 sequences)

SEQ ID NO: 1 - protein, 526 aa, synthetic construct
  REGION 1..526: note = A polypeptide comprising Chitin binding domain CBD - HaPMP5B1
MGSSHHHHHHSSGLVPRGSHMGDRGISEPGNDQGNDNDSNDNNSSNEQGGVCNCNPEEAPAICASPGSEGVLVAHENCEKYYICNHGRPVVASCSGNLLFNPYTNECGWPRDVDCGDRIEPGCTGCNDNNNNDDDDSDCDGDDPVPPPADNDDSESADIDDLPPPGDDASVRPPVDEGTCNCNPEQAPSICAEDDSDGVLVAHEDCNKFYKCHNGKPVALYCPGNLLYNPNTEQCDWPEKVDCGDRVIPDPEDNTVGGNNDGEDDSEGVLVAHENCNQFYKCSGGKPVALLCPGNLLFNPNTDQCDWPWEVDCGDRIIPDPDRTHCGSHCSTHCSTHCGSLLRLPLRLHCGSHCGSHCCTNTATNRRRNMQLQSWSTFHLCSRRLLIAHEDCNKFYICDHGKPVALSCPGNLLYNPYTEKCDWPENVECGDRAPDPDASQAPAICADSGSEGVLVAHENCDQYYICDGGRPVARPCQGGLLYNPLTQYCDGQEMSTAVTGLSLMTAPVIPEMRPDCAVSQTPKEAW

SEQ ID NO: 2 - protein, 220 aa, synthetic construct
  REGION 1..220: note = A polypeptide comprising Chitin binding domain CBD - HaPMP5B1
MGSSHHHHHHSSGLVPRGSHMDRVIPDPEDNTVGGNNDGEDDSEGVLVAHENCNQFYKCSGGKPVALLCPGNLLFNPNTDQCDWPWEVDCGDRIIPDPDRTHCGSHCSTHCSTHCGSLLRLPLRLHCGSHCGSHCCTNTATNRRRNMQLQSWSTFHLCSRRLLIAHEDCNKFYICDHGKPVALSCPGNLLYNPYTEKCDWPENVECGDRAPDPDASQAPA

SEQ ID NO: 3 - protein, 45 aa, synthetic construct
  REGION 1..45: note = Chitin binding domain (CBD)
SEGVLVAHENCEKYYICNHGRPVVASCSGNLLFNPYTNECGWPRD

SEQ ID NO: 4 - protein, 33 aa, synthetic construct
  REGION 1..33: note = Chitin binding domain (CBD)
CNKFYKCHNGKPVALYCPGNLLYNPNTEQCDWP

SEQ ID NO: 5 - protein, 45 aa, synthetic construct
  REGION 1..45: note = Chitin binding domain (CBD)
SEGVLVAHENCNQFYKCSGGKPVALLCPGNLLFNPNTDQCDWPWE

SEQ ID NO: 6 - protein, 37 aa, synthetic construct
  REGION 1..37: note = Chitin binding domain (CBD)
EDCNKFYICDHGKPVALSCPGNLLYNPYTEKCDWPEN

SEQ ID NO: 7 - protein, 41 aa, synthetic construct
  REGION 1..41: note = Chitin binding domain (CBD)
SEGVLVAHENCDQYYICDGGRPVARPCQGGLLYNPLTQYCD

SEQ ID NO: 8 - protein, 400 aa, synthetic construct
  REGION 1..400: note = V-ATPase sub unit c
MHHHHHHMSEYWVISAPGDKTCQQTWDTLNNATKSGNLSANYKFPIPDLKVGTLDQLVGLSDDLGKLDTFVESVTRKVAQYLGEVLEDQRDKLHENLMANNSDMPSYLTRFQWDMAKYPIKQSLRNIADIISKQVGQIDSDLKQKSAAYNALKGNLQNLEKKQTGSLLTRNLADLVKREHFILDSEYLTTLLVIVPKSMFNDWTANYEKITDMIVPRSSQLIHQDNDYGLFNVTLFKKVVEEFKHHARERKFVVREFSYNEADMAAARTRSPSSSPTRRSSSILRKFINFLGPLVRWLKVNFSECFCAWIHVKALRVFVESVLRYGLPVNFQAVVMVPSRKNTKKLREVLQTLYAHLDHSAHQHTSSAQDNAELAGLGFGSSEYFPYVFYKINVDMLDKN

SEQ ID NO: 9 - protein, 516 aa, synthetic construct
  REGION 1..516: note = Chitin deacetylase
METRVKRQEEDGGDEVNAEQLCDGRPADEYFRLTTEGDCRDVVRCTRSGLKQITCPSGLAFDLDKQTCDWKGKVTNCDKLEKPRKVLPILKTDEPICPEGKLACGSGDCIEKELFCNGKPDCKDESDENACTVDVDPNRAPDCDPNQCALPDCFCSADGTRIPGGIEVNQVPQMITITFNGAVNVDNIDLYEQIFNGNRHNPNGCQIRGTFFVSHKYTNYAAVQELHRKGHEISVFSITHKDDPQYWSSGSYDDWLAEMAGARLIVERFANITDSSIIGVRAPYLRVGGNKQFEMMADQYFVYDASITAPLGRVPIWPYTLYFRMPHKCNGNAHNCPSRSHPVWEMVMNELDRRDDPTFDESLPGCHVVDSCSNIQTGEQFARLLRHNSNRHYSTNRAPLGFHFHASWLKSKKEFRDELIKFIEEMLEKNDVYFTSLIQVIQWMQNPTELTSLRDFQEWKQDKCDVKGQPFCSLPNACPLTTRELPGETLRLFTCMECPNNYPWILDPTGEGFNVK

SEQ ID NO: 10 - protein, 548 aa, synthetic construct
  REGION 1..548: note = Trehalase
MDLPLTCTKPVYCNSNLLHQIQMARLYNDSKTFVDLQMNFDENKTLTDFETFFNLHNKNPTKEQLMEFVNEYFSNDNELEPWQPKDFSDNPAFLAKIKDDALREFGKGINNIWPLLARKVKAEVFQKPDQFSLVPLTHGFIIPGGRFKEIYYWDTFWIIEGLLISGMQETAKGMIENLIELLNLFGHIPNGSRGYYQQRSQPPMLNAMVATYYMYTKDLEFLRNNIAYLEKELDFWMDNRVVSVNRGGKNYTLLRYYAPSKGPRPESYYEDYSNTEGFSEEDSTNFCIDIKSAAESGWDFSTRWFLMPDGSNNGTLTDLHTRYIIPVDLNAIFAGAAQYVSNFHALLKNPQKAARYGQLAQTWRDNIQAVLWNDQDAMWYDFNIRDNLHRRYYYSSNAAPLWQNAVNPDFLKLNADRILKAITESGGVDFPGGVPTSLIRSGEQWDFPNVWPPEVSIEVAAIENIGTPEAITLAQEVAQTFVRSCHWGFQKYKQMFEKYDAETPGRFGGGGEYNVQFGFGWSNGVVLEFLNKYGSQLTADDSNNTNNS

SEQ ID NO: 11 - protein, 483 aa, synthetic construct
  REGION 1..483: note = cytochrome p450 monooxygenase
MRTFNYWKKRNVRGPEPVVFFGNLKDSALRKKNMGVVMEELYNMFPEEKVIGIYRMTSPCLLVRDLDVIKHIMIKDFEVFSDRGVEFSKEGLGSNLFHADGETWRALGNRFTPIFTSGKLKNMFYLMHEGADNFIDHVSAECEKNQEFEVHSLLQTYTMSTIAACAFGISYDSIGDKVKALDIVDKIISEPSYAIELDMMYPGLLSKLNLSIFPTVVKNFFKSLVDNIVAQRNGKPSGRNDFMDLILELRQLGEVTSNKYGSSASSLEITDEVICAQAFVFYIAGYETSATTMAYMIYQLALNPDIQNKLIAEVDEVLKANDGKVTYDTVKEMKYLNKAFDETLRMYSIVEPLQRKATRDYKIPGTDVVIEKDTIVLISPRGIHYDPKYYDNPKQFNPDRFDAEEVGKRHPCAYLPFGLGQRNCIGMRFGRLQSLLCITKILSKFRIEPSKNTDRNLQVEPHRGLIGPKGGIRVNAIPRKLVS

SEQ ID NO: 12 - protein, 415 aa, synthetic construct
  REGION 1..415: note = Chitin synthase
NCYFHGTVPDYLYFESPPVFTLSDFISRQMAWICRTFGLNEKLFVMPMYNGLLIDQSMALNRKRNDQRDVKTEDLAEIEKEKGDEYYETISVHTDNTGSSPKAIKSSDQITRIYACATMWHETKDEMMEFLKSILRLDEDQCARRVAQKYLRVVDPDYYEFETHIFLDDAFEISDHSDDDSQVNRFVKLLVDTIDEAASEVHQTNIRDVHVLPSRSSFNGTADILDRKEVMAENTYLLTLDGDIDFQPHAVRLLIDLMKKNKNLGAACGRIHPVGSGPMVWYQMFEYAIGHWLQKATEHMIGCVLCSPGCFSLFRGKALMDDNVMKKYTLRSDEARHYVHTIRGRSMVMYAITATWLSCRILSCLRCYTHCPEGFNEFYNRRRWVPSTIANIMDLLADCKHTIKINDNISSPYIA

SEQ ID NO: 13 - protein, 240 aa, synthetic construct
  REGION 1..240: note = 13 NPC1-sterol transporter
DSFQTKYFQYLNRYLNIGPPVYFVVTEGLNYSDMDTQNMICGTRFCRPDSLSMQLYAAYRNPNETYIAQPPNSWLDDYFDWSALPNCCKYFPSNSSFCPNDRGAPCKACGIALEGDEQRPNSTEFERYVPFFLQDIPDTSGSGCVKGGHAAYGQAVNYKMFNKTQAHVGATYYQGYHTVLKTSLDYYSALKGAREVAANLTETLNRNLKHQLNGTTINVFPYSVFYVFYEQYLTMWPDTL

SEQ ID NO: 14 - protein, 144 aa, synthetic construct
  REGION 1..144: note = nanobody amino acid sequence
MQVQLQESGGGLVQAGDSLRLSCAASGRSFSIYTMGWFRQAPGNEREFVTAISPSGGSTHYADFVKGRFTISRDNTKNTMYLQMNSLKPEDTAVYFCASTSSQHYEDTEESYKYWGQGTQVTVSSAAAYPYDVPDYGSHHHHHH

SEQ ID NO: 15 - protein, 8 aa, synthetic construct
  REGION 1..8: note = Complementarity-determining region amino acid sequence
GRSFSIYT

SEQ ID NO: 16 - protein, 8 aa, synthetic construct
  REGION 1..8: note = Complementarity-determining region amino acid sequence
ISPSGGST

SEQ ID NO: 17 - protein, 17 aa, synthetic construct
  REGION 1..17: note = Complementarity-determining region amino acid sequence
ASTSSQHYEDTEESYKY

SEQ ID NO: 18 - protein, 140 aa, synthetic construct
  REGION 1..140: note = nanobody amino acid sequence
MQVQLQESGGGLVEAAGSLRLSCAALGSNLRINTMGWYRQAPGKQRELVATITNGGRKNYADSVKGRFTIFRGNANTVYLQMNSLKPEDTAVYYCNAGLLDPPYSAPGDYWGEGTQVTVSSAAAYPYDVPDYGSHHHHHH

SEQ ID NO: 19 - protein, 9 aa, synthetic construct
  REGION 1..9: note = Complementarity-determining region amino acid sequence
LGSNLRINT

SEQ ID NO: 20 - protein, 10 aa, synthetic construct
  REGION 1..10: note = Complementarity-determining region amino acid sequence
TITNGGRKNY

SEQ ID NO: 21 - protein, 15 aa, synthetic construct
  REGION 1..15: note = Complementarity-determining region amino acid sequence
NAGLLDPPYSAPGDY

SEQ ID NO: 22 - protein, 144 aa, synthetic construct
  REGION 1..144: note = nanobody amino acid sequence
MQVQLQESGGGLVQAGGSLTLSCAASGRSFSTYAMGWSRQAPGKEREFLAGISRGGGTTVYADSVKGRFTISRDNVKNTVYLQMNSLKPEDAAVYYCAAALRPFDGSGERRYDYWGQGTQVTVSSAAAYPYDVPDYGSHHHHHH

SEQ ID NO: 23 - protein, 8 aa, synthetic construct
  REGION 1..8: note = Complementarity-determining region amino acid sequence
GRSFSTYA

SEQ ID NO: 24 - protein, 9 aa, synthetic construct
  REGION 1..9: note = Complementarity-determining region amino acid sequence
GISRGGGTT

SEQ ID NO: 25 - protein, 16 aa, synthetic construct
  REGION 1..16: note = Complementarity-determining region amino acid sequence
AALRPFDGSGERRYDY

SEQ ID NO: 26 - protein, 143 aa, synthetic construct
  REGION 1..143: note = nanobody amino acid sequence
MQVQLQESGGGLVQAGDSLRLSCAASGRSFSIYTMGWFRQAPGKEREFVAAISPSGVSTDYADSVRGRFTISRDNAKNTMYLQMNSLKPEDTAVYYCAAGGRHYTRHPYDYDYWGQGTQVTVSSAAAYPYDVPDYGSHHHHHH

SEQ ID NO: 27 - skipped

SEQ ID NO: 28 - protein, 10 aa, synthetic construct
  REGION 1..10: note = Complementarity-determining region amino acid sequence
AISPSGVSTD

SEQ ID NO: 29 - protein, 16 aa, synthetic construct
  REGION 1..16: note = Complementarity-determining region amino acid sequence
AAGGRHYTRHPYDYDY

SEQ ID NO: 30 - protein, 143 aa, synthetic construct
  REGION 1..143: note = nanobody amino acid sequence
MQVQLQESGGGLVQTGGSLRLSCAASGRSFSSYSMGWFRQGPGKGREWVADINESGSSTSYYDPVKGRFTISRDNSKNTVYLQMNDLKPEDTADYYCAALVTGGDTDLGEWDFWGPGTQVTVSSAAAYPYDVPDYGSHHHHHH

SEQ ID NO: 31 - protein, 8 aa, synthetic construct
  REGION 1..8: note = Complementarity-determining region amino acid sequence
GRSFSSYS

SEQ ID NO: 32 - protein, 8 aa, synthetic construct
  REGION 1..8: note = Complementarity-determining region amino acid sequence
INESGSST

SEQ ID NO: 33 - protein, 16 aa, synthetic construct
  REGION 1..16: note = Complementarity-determining region amino acid sequence
AALVTGGDTDLGEWDF

SEQ ID NO: 34 - protein, 144 aa, synthetic construct
  REGION 1..144: note = nanobody amino acid sequence
MQVQLQESGGGLVQAGDSLKLSCAASGGTFSSYVMGWFRQAPGKEREFVARIGVSEGYLYYADSVKDRFTISRDSAKNTGYLQMNALKPEDTAVYYCAAGPRRYWTREPDAYDYWGQGTQVTVSSAAAYPYDVPDYGSHHHHHH

SEQ ID NO: 35 - protein, 8 aa, synthetic construct
  REGION 1..8: note = Complementarity-determining region amino acid sequence
GGTFSSYV

SEQ ID NO: 36 - protein, 8 aa, synthetic construct
  REGION 1..8: note = Complementarity-determining region amino acid sequence
IGVSEGYL

SEQ ID NO: 37 - protein, 17 aa, synthetic construct
  REGION 1..17: note = Complementarity-determining region amino acid sequence
AAGPRRYWTREPDAYDY

SEQ ID NO: 38 - protein, 145 aa, synthetic construct
  REGION 1..145: note = nanobody amino acid sequence
MQVQLQESGGGLVQAGGSLKLSCGASGRTFSTNAMGWFRQAPGKEREFVATISAGGSLTYYADSVKGRFTISRDNAKNTVYLRMNSLKPDDTAVYYCAADQDSGRLPLINSGYEYWGQGTQVTVSSAAAYPYDVPDYGSHHHHHH

SEQ ID NO: 39 - protein, 8 aa, synthetic construct
  REGION 1..8: note = Complementarity-determining region amino acid sequence
GRTFSTNA

SEQ ID NO: 40 - protein, 8 aa, synthetic construct
  REGION 1..8: note = Complementarity-determining region amino acid sequence
ISAGGSLT

SEQ ID NO: 41 - protein, 18 aa, synthetic construct
  REGION 1..18: note = Complementarity-determining region amino acid sequence
AADQDSGRLPLINSGYEY

SEQ ID NO: 42 - protein, 145 aa, synthetic construct
  REGION 1..145: note = nanobody amino acid sequence
MQVQLQESGGGLVQAGASLRLSCAASGRTFSSYGTAMGWFRQAPGKEREFVAAILWTGSSSYDSVKGRFTISRDNAKNTVYLQMISLNPEDTAVYYCAARSRYTGSYYEESTYNYWGQGTQVTVSSAAAYPYDVPDYGSHHHHHH

SEQ ID NO: 43 - protein, 10 aa, synthetic construct
  REGION 1..10: note = Complementarity-determining region amino acid sequence
GRTFSSYGTA

SEQ ID NO: 44 - protein, 8 aa, synthetic construct
  REGION 1..8: note = Complementarity-determining region amino acid sequence
ILWTGSSS

SEQ ID NO: 45 - protein, 18 aa, synthetic construct
  REGION 1..18: note = Complementarity-determining region amino acid sequence
AARSRYTGSYYEESTYNY

SEQ ID NO: 46 - protein, 147 aa, synthetic construct
  REGION 1..147: note = nanobody amino acid sequence
MQVQLQESGGGVVQPGGSLRLSCAASGRDFSNYNMAWFRQAPGKERDVVATIRRSGDITSYTDSVKGRFTISRDNAKNTVYLQMNSLKPEDTAAYYCAARTGSFLTVLITTPGNYNYWGQGTQVTVSSAAAYPYDVPDYGSHHHHHH

SEQ ID NO: 47 - protein, 8 aa, synthetic construct
  REGION 1..8: note = Complementarity-determining region amino acid sequence
GRDFSNYN

SEQ ID NO: 48 - protein, 8 aa, synthetic construct
  REGION 1..8: note = Complementarity-determining region amino acid sequence
IRRSGDIT

SEQ ID NO: 49 - protein, 20 aa, synthetic construct
  REGION 1..20: note = Complementarity-determining region amino acid sequence
AARTGSFLTVLITTPGNYNY

SEQ ID NO: 50 - protein, 140 aa, synthetic construct
  REGION 1..140: note = nanobody amino acid sequence
MQVQLQESGGGLVQAGDSLRLSCAASGRTFSAFRMAWFRQAPGKERALVADISRLSTRTYYADSVKGRFTISRDNAKNTVYLQMNSLKPEDTAVYYCAADLEGVGPMWEYWVQGTQVTVSSAAAYPYDVPDYGSHHHHHH

SEQ ID NO: 51 - protein, 8 aa, synthetic construct
  REGION 1..8: note = Complementarity-determining region amino acid sequence
GRTFSAFR

SEQ ID NO: 52 - protein, 8 aa, synthetic construct
  REGION 1..8: note = Complementarity-determining region amino acid sequence
ISRLSTRT

SEQ ID NO: 53 - protein, 13 aa, synthetic construct
  REGION 1..13: note = Complementarity-determining region amino acid sequence
AADLEGVGPMWEY

SEQ ID NO: 54 - protein, 144 aa, synthetic construct
  REGION 1..144: note = nanobody amino acid sequence
MQVQLQESGGGSVQAGNSLRLSCTYSGRTFSTRAMGWFRQAPGKERELVAGIGWNGATQYYADSVKGRFTISRDSASNTVALQMNSLEPEDTAVYYCAAHGREYVTPSYNNYDYWGQGTQVTVSSAAAYPYDVPDYGSHHHHHH

SEQ ID NO: 55 - protein, 8 aa, synthetic construct
  REGION 1..8: note = Complementarity-determining region amino acid sequence
GRTFSTRA

SEQ ID NO: 56 - protein, 8 aa, synthetic construct
  REGION 1..8: note = Complementarity-determining region amino acid sequence
IGWNGATQ

SEQ ID NO: 57 - protein, 17 aa, synthetic construct
  REGION 1..17: note = Complementarity-determining region amino acid sequence
AAHGREYVTPSYNNYDY

SEQ ID NO: 58 - protein, 143 aa, synthetic construct
  REGION 1..143: note = nanobody amino acid sequence
MQVQLQESGGGLTQAGDSLRLSCAASGRSFVRYTTGWFRQAPGKEREFVASISWSRGSTYYADSVKGRFTISKDNAENTVYLQMNSPEPEDTAVYYCAGNSRGATTFAQYYDDWGQGTQVTVSSAAAYPYDVPDYGSHHHHHH

SEQ ID NO: 59 - protein, 8 aa, synthetic construct
  REGION 1..8: note = Complementarity-determining region amino acid sequence
GRSFVRYT

SEQ ID NO: 60 - protein, 8 aa, synthetic construct
  REGION 1..8: note = Complementarity-determining region amino acid sequence
ISWSRGST

SEQ ID NO: 61 - protein, 16 aa, synthetic construct
  REGION 1..16: note = Complementarity-determining region amino acid sequence
AGNSRGATTFAQYYDD

SEQ ID NO: 62 - protein, 137 aa, synthetic construct
  REGION 1..137: note = nanobody amino acid sequence
MQVQLQESGGGLVQAGGSLRLSCAASGRTFSLTRMGWFRQAPGKEREIVAHIMRSSDSTFYGDSVKGRFTISRDNAKNTVYLQMNRLNPEDTAVYYCAAAQWAGYDYWGQGTQVTVSSAAAYPYDVPDYGSHHHHHH

SEQ ID NO: 63 - protein, 8 aa, synthetic construct
  REGION 1..8: note = Complementarity-determining region amino acid sequence
GRTFSLTR

SEQ ID NO: 64 - protein, 8 aa, synthetic construct
  REGION 1..8: note = Complementarity-determining region amino acid sequence
IMRSSDST

SEQ ID NO: 65 - protein, 10 aa, synthetic construct
  REGION 1..10: note = Complementarity-determining region amino acid sequence
AAAQWAGYDY

SEQ ID NO: 66 - protein, 143 aa, synthetic construct
  REGION 1..143: note = nanobody amino acid sequence
MQVQLQESGGGLVPAGGSLRLSCAASGRTFTSSTMAWFRQVPGKEREFVAAISPRGLSQDYGHSVKGRFTISRDNAENTVYLQMNSLKSEDTALYYCAATSGSYSSSRNDYYYWGQGTQVTVSSAAAYPYDVPDYGSHHHHHH

SEQ ID NO: 67 - protein, 8 aa, synthetic construct
  REGION 1..8: note = Complementarity-determining region amino acid sequence
GRTFTSST

SEQ ID NO: 68 - protein, 8 aa, synthetic construct
  REGION 1..8: note = Complementarity-determining region amino acid sequence
ISPRGLSQ

SEQ ID NO: 69 - protein, 16 aa, synthetic construct
  REGION 1..16: note = Complementarity-determining region amino acid sequence
AATSGSYSSSRNDYYY

SEQ ID NO: 70 - protein, 137 aa, synthetic construct
  REGION 1..137: note = nanobody amino acid sequence
MQVQLQESGGGLVQPGGSLRLSCAASGFTLSNYRMAWFRQGLGKEREFVAHIMRNSDTTWYTESVKGRFTISRDNSKNTVYLQMNSLKPEDTAVYYCAASNAGTFDYWGQGTQVTVSSAAAYPYDVPDYGSHHHHHH

SEQ ID NO: 71 - protein, 8 aa, synthetic construct
  REGION 1..8: note = Complementarity-determining region amino acid sequence
GFTLSNYR

SEQ ID NO: 72 - protein, 8 aa, synthetic construct
  REGION 1..8: note = Complementarity-determining region amino acid sequence
IMRNSDTT

SEQ ID NO: 73 - protein, 10 aa, synthetic construct
  REGION 1..10: note = Complementarity-determining region amino acid sequence
AASNAGTFDY

SEQ ID NO: 74 - protein, 136 aa, synthetic construct
  REGION 1..136: note = nanobody amino acid sequence
MQVQLQESGGGLVQPGGSLRLSCASSGANIRLYGMAWYRQPPGEERELVASITVGGSITYAESVKARFTISRDNARDMVFLQMNSLKPEDTAVYYCNAMNPWYYWAWGQGTQVTVSSAAAYPYDVPDYGSHHHHHH

SEQ ID NO: 75 - protein, 8 aa, synthetic construct
  REGION 1..8: note = Complementarity-determining region amino acid sequence
GANIRLYG

SEQ ID NO: 76 - protein, 7 aa, synthetic construct
  REGION 1..7: note = Complementarity-determining region amino acid sequence
ITVGGSI

SEQ ID NO: 77 - protein, 10 aa, synthetic construct
  REGION 1..10: note = Complementarity-determining region amino acid sequence
NAMNPWYYWA

SEQ ID NO: 78 - protein, 141 aa, synthetic construct
  REGION 1..141: note = nanobody amino acid sequence
MQVQLQESGGGLVQAGGSLRLSCAASGGTLSSYDMGWFRQAPGKGRDFVAGIDWSGGSTNYERSVKGRFTITRDNAKNTVLLQMNSLKPEDTAVYYCAAARANSDLGIYDYWGQGTQVTVSSAAAYPYDVPDYGSHHHHHH

SEQ ID NO: 79 - protein, 8 aa, synthetic construct
  REGION 1..8: note = Complementarity-determining region amino acid sequence
GGTLSSYD

SEQ ID NO: 80 - protein, 8 aa, synthetic construct
  REGION 1..8: note = Complementarity-determining region amino acid sequence
IDWSGGST

SEQ ID NO: 81 - protein, 14 aa, synthetic construct
  REGION 1..14: note = Complementarity-determining region amino acid sequence
AAARANSDLGIYDY

SEQ ID NO: 82 - protein, 138 aa, synthetic construct
  REGION 1..138: note = nanobody amino acid sequence
MQVQLQESGGGLVEAGGSLGLACTTSGIIFSRNDMGWFRQAPGEQRTAVATITRSSSTNYAGSVKGRFTISRDNAENTVYLQMSNLKPEDTAVYYCATIPTATQPYVYWGQGTQVTVSSAAAYPYDVPDYGSHHHHHH

SEQ ID NO: 83 - protein, 8 aa, synthetic construct
  REGION 1..8: note = Complementarity-determining region amino acid sequence
GIIFSRND

SEQ ID NO: 84 - protein, 7 aa, synthetic construct
  REGION 1..7: note = Complementarity-determining region amino acid sequence
ITRSSST

SEQ ID NO: 85 - protein, 12 aa, synthetic construct
  REGION 1..12: note = Complementarity-determining region amino acid sequence
ATIPTATQPYVY

SEQ ID NO: 86 - protein, 144 aa, synthetic construct
  REGION 1..144: note = nanobody amino acid sequence
MQVQLQESGGGLVQTGGSLRLSCAASGRSFSSYSMGWFRQGPGKGREWVADINESGTTTNYWDPVKGRFIISRDNAQNMVYLQMNSLKPEDTAVYYCAAGPRTRWTREPDAYDYWGQGTQVTVSSAAAYPYDVPDYGSHHHHHH

SEQ ID NO: 87 - protein, 8 aa, synthetic construct
  REGION 1..8: note = Complementarity-determining region amino acid sequence
GRSFSSYS

SEQ ID NO: 88 - protein, 8 aa, synthetic construct
  REGION 1..8: note = Complementarity-determining region amino acid sequence
INESGTTT

SEQ ID NO: 89 - protein, 17 aa, synthetic construct
  REGION 1..17: note = Complementarity-determining region amino acid sequence
AAGPRTRWTREPDAYDY

SEQ ID NO: 90 - protein, 141 aa, synthetic construct
  REGION 1..141: note = nanobody amino acid sequence
MQVQLQESGGGLVQAGGSLRLSCVASGRTFSTYDMGWFRQAPGKGREFVAGIDWSGGSTNYVNFVKGRFTISRDNAKNTMYLQMNSLKPEDTAVYYCAAAVGDSEMATYDYWGQGTQVTVSSAAAYPYDVPDYGSHHHHHH

SEQ ID NO: 91 - protein, 8 aa, synthetic construct
  REGION 1..8: note = Complementarity-determining region amino acid sequence
GRTFSTYD

SEQ ID NO: 92 - skipped

SEQ ID NO: 93 - protein, 14 aa, synthetic construct
  REGION 1..14: note = Complementarity-determining region amino acid sequence
AAAVGDSEMATYDY

SEQ ID NO: 94 - protein, 137 aa, synthetic construct
  REGION 1..137: note = nanobody amino acid sequence
MQVQLQESGGGLVQVGGSLRLSCVASEPTFRPNRMGWFRQAPGKERELVAHIMWSSGSTWYGDSVKGRFTISRDNAKNTVILQMNSLNPEDTAVYYCAAAQRAGYDYWGQGTQVTVSSAAAYPYDVPDYGSHHHHHH

SEQ ID NO: 95 - protein, 8 aa, synthetic construct
  REGION 1..8: note = Complementarity-determining region amino acid sequence
EPTFRPNR

SEQ ID NO: 96 - protein, 9 aa, synthetic construct
  REGION 1..9: note = Complementarity-determining region amino acid sequence
IMWSSGSTW

SEQ ID NO: 97 - protein, 9 aa, synthetic construct
  REGION 1..9: note = Complementarity-determining region amino acid sequence
AAQRAGYDY

SEQ ID NO: 98 - protein, 143 aa, synthetic construct
  REGION 1..143: note = nanobody amino acid sequence
MQVQLQESGGGLVQAGGSLRLSCAASGRSLSSYSMGWIRQAPGKERDFVTAIRWSGGSTYYADSVKGRFTISRDIAKNAVYLQMNSLKTEDTAVYYCAARLGGRSWDAGDYQYWGQGTQVTVSSAAAYPYDVPDYGSHHHHHH

SEQ ID NO: 99 - protein, 8 aa, synthetic construct
  REGION 1..8: note = Complementarity-determining region amino acid sequence
GRSLSSYS

SEQ ID NO: 100 - protein, 8 aa, synthetic construct
  REGION 1..8: note = Complementarity-determining region amino acid sequence
IRWSGGST

SEQ ID NO: 101 - protein, 16 aa, synthetic construct
  REGION 1..16: note = Complementarity-determining region amino acid sequence
AARLGGRSWDAGDYQY

SEQ ID NO: 102 - protein, 135 aa, synthetic construct
  REGION 1..135: note = nanobody amino acid sequence
MQVQLQESGGGSVQSGGSLFLSCAASGFVFETSPMTWVRQAPGQGVRWVGSITTDGRRADYEDAVKGRFTISRDNVKNMLYLEMNNLKPEDTAMYFCRESRDLNAVTRGTQVTVSSAAAYPYDVPDYGSHHHHHH

SEQ ID NO: 103 - protein, 8 aa, synthetic construct
  REGION 1..8: note = Complementarity-determining region amino acid sequence
GFVFETSP

SEQ ID NO: 104 - protein, 8 aa, synthetic construct
  REGION 1..8: note = Complementarity-determining region amino acid sequence
ITTDGRRA

SEQ ID NO: 105 - protein, 8 aa, synthetic construct
  REGION 1..8: note = Complementarity-determining region amino acid sequence
RESRDLNA

SEQ ID NO: 106 - protein, 139 aa, synthetic construct
  REGION 1..139: note = nanobody amino acid sequence
MQVQLQESGGGLVQTGDSLRLSCAVSGRTGSINRMGWFRQAPGKEREIVSHIFWSNVGTWSAESVKGRFIISRDNAKNTVYLQMNSLKPEDTAVYSCAAATGSAYNYWVPSRGDPGHVSSAAAYPYDVPDYGSHHHHHH

SEQ ID NO: 107 - protein, 8 aa, synthetic construct
  REGION 1..8: note = Complementarity-determining region amino acid sequence
GRTGSINR

SEQ ID NO: 108 - protein, 8 aa, synthetic construct
  REGION 1..8: note = Complementarity-determining region amino acid sequence
IFWSNVGT

SEQ ID NO: 109 - protein, 10 aa, synthetic construct
  REGION 1..10: note = Complementarity-determining region amino acid sequence
AAATGSAYNY

SEQ ID NO: 110 - protein, 133 aa, synthetic construct
  REGION 1..133: note = nanobody amino acid sequence
MQVQLQESGGGQVQAGDSLRLSCVTSGMSFSTSAMGWYRQASGKQREWVAIIREDSTTNYSSFAKGRFTISRDNTNKTVYLLMNSLEPDDTAVYYCRTYTGGYWGQGTQVTVSSAAAYPYDVPDYGSHHHHHH

SEQ ID NO: 111 - protein, 8 aa, synthetic construct
  REGION 1..8: note = Complementarity-determining region amino acid sequence
GMSFSTSA

SEQ ID NO: 112 - protein, 7 aa, synthetic construct
  REGION 1..7: note = Complementarity-determining region amino acid sequence
IREDSTT

SEQ ID NO: 113 - protein, 7 aa, synthetic construct
  REGION 1..7: note = Complementarity-determining region amino acid sequence
RTYTGGY

SEQ ID NO: 114 - protein, 145 aa, synthetic construct
  REGION 1..145: note = nanobody amino acid sequence
MQVQLQESGGGLVQAGGSLRLSCAASGRTFSSYAMGWFRQAPGKEREFVAGISWSGRSTYYADSVKGRFTISRDDAKNTVYLQMNSLKPEDTAVYYCAAPDTAAQFTTPLYEYAYWGQGTQVTVSSAAAYPYDVPDYGSHHHHHH

SEQ ID NO: 115 - protein, 8 aa, synthetic construct
  REGION 1..8: note = Complementarity-determining region amino acid sequence
GRTFSSYA

SEQ ID NO: 116 - protein, 8 aa, synthetic construct
  REGION 1..8: note = Complementarity-determining region amino acid sequence
ISWSGRST

SEQ ID NO: 117 - protein, 16 aa, synthetic construct
  REGION 1..16: note = Complementarity-determining region amino acid sequence
AAPDTAAQFTTPLYEY

SEQ ID NO: 118 - protein, 143 aa, synthetic construct
  REGION 1..143: note = nanobody amino acid sequence
MQVQLQESGGGLVQAGDSLRLACASSSRTFSTYTMGWFRQTPGRERDFVAAISPSGATADYADSVKGRFTISRDNAKNTLYLQMDSLKPEDTAVYYCAARYLSWSRMNHEYPYWGQGTQVTVSSAAAYPYDVPDYGSHHHHHH

SEQ ID NO: 119 - protein, 8 aa, synthetic construct
  REGION 1..8: note = Complementarity-determining region amino acid sequence
SRTFSTYT

SEQ ID NO: 120 - protein, 8 aa, synthetic construct
  REGION 1..8: note = Complementarity-determining region amino acid sequence
ISPSGATA

SEQ ID NO: 121 - protein, 16 aa, synthetic construct
  REGION 1..16: note = Complementarity-determining region amino acid sequence
AARYLSWSRMNHEYPY

SEQ ID NO: 122 - protein, 146 aa, synthetic construct
  REGION 1..146: note = nanobody amino acid sequence
MQVQLQESGGGLVQAGDSLRLSCAASERTFSSYVMGWFRQAPRKEREFVAAMTWSGSSRIYYADSVKGRFTISRDNAKNTAYLQMNSLKPEDTAVYYCAAKDAYGGISFRPNTYHSWGQGTQVTVSSAAAYPYDVPDYGSHHHHHH

SEQ ID NO: 123 - protein, 8 aa, synthetic construct
  REGION 1..8: note = Complementarity-determining region amino acid sequence
ERTFSSYV

SEQ ID NO: 124 - protein, 9 aa, synthetic construct
  REGION 1..9: note = Complementarity-determining region amino acid sequence
MTWSGSSRI

SEQ ID NO: 125 - protein, 18 aa, synthetic construct
  REGION 1..18: note = Complementarity-determining region amino acid sequence
AAKDAYGGISFRPNTYHS

SEQ ID NO: 126 - protein, 130 aa, synthetic construct
  REGION 1..130: note = nanobody amino acid sequence
MQVQLQESGGGLVQPGGSLRLSCAALGNIVNINNMGWYRQAPGGQRELVATITRGAIKNYADSVKGRFTIFRGNANTVYLQMNSLKPEDTAVYYCVADSSWGQGTQVTVSSAAAYPYDVPDYGSHHHHHH

SEQ ID NO: 127 - protein, 8 aa, synthetic construct
  REGION 1..8: note = Complementarity-determining region amino acid sequence
GNIVNINN

SEQ ID NO: 128 - protein, 7 aa, synthetic construct
  REGION 1..7: note = Complementarity-determining region amino acid sequence
ITRGAIK

SEQ ID NO: 129 - protein, 5 aa, synthetic construct
  REGION 1..5: note = Complementarity-determining region amino acid sequence
VADSS

SEQ ID NO: 130 - protein, 143 aa, synthetic construct
  REGION 1..143: note = nanobody amino acid sequence
MQVQLQESGGGVVQAGGFLRLSCAASGLIFDDTAIGWFRQAPGKEREFVAAVSPSGVSTDYTDSVKGRFTISRDNAKKTVFLQMSRLKPEDTAVYYCAARLRHYSNDQHEYDSWGQGTQVTVSSAAAYPYDVPDYGSHHHHHH

SEQ ID NO: 131 - protein, 8 aa, synthetic construct
  REGION 1..8: note = Complementarity-determining region amino acid sequence
GLIFDDTA

SEQ ID NO: 132 - protein, 8 aa, synthetic construct
  REGION 1..8: note = Complementarity-determining region amino acid sequence
VSPSGVST

SEQ ID NO: 133 - protein, 16 aa, synthetic construct
  REGION 1..16: note = Complementarity-determining region amino acid sequence
AARLRHYSNDQHEYDS

SEQ ID NO: 134 - protein, 136 aa, synthetic construct
  REGION 1..136: note = nanobody amino acid sequence
MQVQLQESGGGLVQAGGSLRLSCAASGIVFSITTMGWYRQAPGKQRELVATIASGVRADYADSVKGRFTISRDNGKNTVYLQMNSLKPEDTAVYYCNANRFSLGNYWGQGTQVTVSSAAAYPYDVPDYGSHHHHHH

SEQ ID NO: 135 - protein, 8 aa, synthetic construct
  REGION 1..8: note = Complementarity-determining region amino acid sequence
GIVFSITT

SEQ ID NO: 136 - protein, 7 aa, synthetic construct
  REGION 1..7: note = Complementarity-determining region amino acid sequence
IASGVRA

SEQ ID NO: 137 - protein, 10 aa, synthetic construct
  REGION 1..10: note = Complementarity-determining region amino acid sequence
NANRFSLGNY

SEQ ID NO: 138 - protein, 144 aa, synthetic construct
  REGION 1..144: note = nanobody amino acid sequence
MQVQLQESGGGLVQTGDSLRLSCAASGGTFSAYTMGWFRQAPGKEREFVAAISRSGSSTHYANSVKGHFTISRDNAKKTVYLQMNSLKPEDTARYYCAGERTGHFTDLYYEYDYWGQGTQVTVSSAAAYPYDVPDYGSHHHHHH

SEQ ID NO: 139 - protein, 8 aa, synthetic construct
  REGION 1..8: note = Complementarity-determining region amino acid sequence
GGTFSAYT

SEQ ID NO: 140 - protein, 8 aa, synthetic construct
  REGION 1..8: note = Complementarity-determining region amino acid sequence
ISRSGSST

SEQ ID NO: 141 - protein, 17 aa, synthetic construct
  REGION 1..17: note = Complementarity-determining region amino acid sequence
AGERTGHFTDLYYEYDY

SEQ ID NO: 142 - protein, 140 aa, synthetic construct
  REGION 1..140: note = nanobody amino acid sequence
MQVQLQESGGGLVQAGGSLRLSCAAPGSNFRINTMGWYRQAPGKQRELVATIANSGRINSANSVKGRFTIFRGNANTVYLQMNSLKPEDTAVYYCNADVVESSNYQTLNYWGQGTQVTVSSAAAYPYDVPDYGSHHHHHH

SEQ ID NO: 143 - protein, 8 aa, synthetic construct
  REGION 1..8: note = Complementarity-determining region amino acid sequence
GSNFRINT

SEQ ID NO: 144 - protein, 7 aa, synthetic construct
  REGION 1..7: note = Complementarity-determining region amino acid sequence
IANSGRI

SEQ ID NO: 145 - protein, 15 aa, synthetic construct
  REGION 1..15: note = Complementarity-determining region amino acid sequence
NADVVESSNYQTLNY

SEQ ID NO: 146 - protein, 143 aa, synthetic construct
  REGION 1..143: note = nanobody amino acid sequence
MQVQLQESGGGLVQAGGSLRLSCVASRASGSIFGAQTMAWYRQASGERRELVATITSSGSTNYADSVKGRFTISRDNAKSTMFLQMNNLKPEDTAVYYCNVGFRSRYSYDSSVWGEGTQVTVSSAAAYPYDVPDYGSHHHHHH

SEQ ID NO: 147 - protein, 11 aa, synthetic construct
  REGION 1..11: note = Complementarity-determining region amino acid sequence
RASGSIFGAQT

SEQ ID NO: 148 - protein, 7 aa, synthetic construct
  REGION 1..7: note = Complementarity-determining region amino acid sequence
ITSSGST

SEQ ID NO: 149 - protein, 14 aa, synthetic construct
  REGION 1..14: note = Complementarity-determining region amino acid sequence
NVGFRSRYSYDSSV

SEQ ID NO: 150 - protein, 144 aa, synthetic construct
  REGION 1..144: note = nanobody amino acid sequence
MQVQLQESGGGLVQAGGSLRLSCAASGRTFSTSNMGWFRQAPGKEREFVGAISPSGRSTDYASSVEGRFTISRDNPKNTMYLQMNSLKPEDTAVYYCAARRSPSYTRVGDEYDYWGQGTQVTVSSAAAYPYDVPDYGSHHHHHH

SEQ ID NO: 151 - protein, 8 aa, synthetic construct
  REGION 1..8: note = Complementarity-determining region amino acid sequence
GRTFSTSN

SEQ ID NO: 152 - protein, 8 aa, synthetic construct
  REGION 1..8: note = Complementarity-determining region amino acid sequence
ISPSGRST

SEQ ID NO: 153 - protein, 17 aa, synthetic construct
  REGION 1..17: note = Complementarity-determining region amino acid sequence
AARRSPSYTRVGDEYDY

SEQ ID NO: 154 - protein, 148 aa, synthetic construct
  REGION 1..148: note = nanobody amino acid sequence
MQVQLQESGGGLVQAGDSLRLSCAASGRSFSIYTMGWFRQAPGKEREFVSLIMRSGGIIYADFVKGRFTISRDNAKNLVYLQMNSLKPEDTAVYYCAAGGSTNSRAYNYYKLSLAYDYWGQGTQVTVSSAAAYPYDVPDYGSHHHHHH

SEQ ID NO: 155 - skipped

SEQ ID NO: 156 - protein, 8 aa, synthetic construct
  REGION 1..8: note = Complementarity-determining region amino acid sequence
IMRSGGII

SEQ ID NO: 157 - protein, 22 aa, synthetic construct
  REGION 1..22: note = Complementarity-determining region amino acid sequence
AAGGSTNSRAYNYYKLSLAYDY

SEQ ID NO: 158 - protein, 144 aa, synthetic construct
  REGION 1..144: note = nanobody amino acid sequence
MQVQLQESGGGLVQAGDSLRLSCAASGGTFSTYTMGWFRQAPGKEREFVAAISRTGRGTDYADSVKGRFTISRDNVKNTVSLQMNSLKPEDTAVYYCAERSSVHYSGIAADYDYWSQGTQVTVSSAAAYPYDVPDYGSHHHHHH

SEQ ID NO: 159 - protein, 8 aa, synthetic construct
  REGION 1..8: note = Complementarity-determining region amino acid sequence
GGTFSTYT

SEQ ID NO: 160 - protein, 8 aa, synthetic construct
  REGION 1..8: note = Complementarity-determining region amino acid sequence
ISRTGRGT

SEQ ID NO: 161 - protein, 17 aa, synthetic construct
  REGION 1..17: note = Complementarity-determining region amino acid sequence
AERSSVHYSGIAADYDY

SEQ ID NO: 162 - protein, 144 aa, synthetic construct
  REGION 1..144: note = nanobody amino acid sequence
MQVQLQESGGGLVQPGGSLRLSCAASGRNLSRSAMGWFRQNPGEEREFVAAIDWWGDSTYYGDSVTGRFTISRDNAKNTLYLQMNSLKPEDTAVYYCAAGLRPFDGSWERRYDYWGQGTQVTVSSAAAYPYDVPDYGSHHHHHH

SEQ ID NO: 163 - protein, 8 aa, synthetic construct
  REGION 1..8: note = Complementarity-determining region amino acid sequence
GRNLSRSA

SEQ ID NO: 164 - protein, 8 aa, synthetic construct
  REGION 1..8: note = Complementarity-determining region amino acid sequence
IDWWGDST

SEQ ID NO: 165 - protein, 17 aa, synthetic construct
  REGION 1..17: note = Complementarity-determining region amino acid sequence
AAGLRPFDGSWERRYDY

SEQ ID NO: 166 - protein, 135 aa, synthetic construct
  REGION 1..135: note = nanobody amino acid sequence
QVQLQESGGGLVQAGGSLRLSCAASGSTFSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 167 - protein, 8 aa, synthetic construct
  REGION 1..8: note = Complementarity-determining region amino acid sequence
GSTFSGYI

SEQ ID NO: 168 - protein, 7 aa, synthetic construct
  REGION 1..7: note = Complementarity-determining region amino acid sequence
ITYVGST

SEQ ID NO: 169 - protein, 10 aa, synthetic construct
  REGION 1..10: note = Complementarity-determining region amino acid sequence
RAREGEQLDF

SEQ ID NO: 170 - protein, 135 aa, synthetic construct
  REGION 1..135: note = nanobody amino acid sequence
QVQLQESGGGLVQAGGSLRLSCAASGSTLSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 171 - protein, 8 aa, synthetic construct
  REGION 1..8: note = Complementarity-determining region amino acid sequence
SGSTFSGY

SEQ ID NO: 172 - protein, 135 aa, synthetic construct
  REGION 1..135: note = nanobody amino acid sequence
QVQLQESGGGLVQSGDSLRLSCAASGSTFSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 173 - protein, 135 aa, synthetic construct
  REGION 1..135: note = nanobody amino acid sequence
QVQLQESGGGLVQPGGSLRLSCVASGSTFSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 174 - protein, 12 aa, synthetic construct
  REGION 1..12: note = Complementarity-determining region amino acid sequence
ASGSTFSGYIMS

SEQ ID NO: 175 - protein, 135 aa, synthetic construct
  REGION 1..135: note = nanobody amino acid sequence
QVQLQESGGGLVQAGDSLRLSCAASGSTFSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 176 - protein, 135 aa, synthetic construct
  REGION 1..135: note = nanobody amino acid sequence
QVQLQESGGGTVQAGGSLRLSCAASGSTFSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 177 - protein, 135 aa, synthetic construct
  REGION 1..135: note = nanobody amino acid sequence
QVQLQESGGGLVQAGGSLRLSCAASGSTFSGYIMSWFRQPPGKERELVAAITYVGSTWYQDSVKGRFTISRDNAKNTVSLQMNSLKPEDTAVYYCRAREGEQLDFWGQGTQVTVSSAAAYPYDVPDYGSHHHHHH

SEQ ID NO: 178 - protein, 11 aa, synthetic construct
  REGION 1..11: note = Complementarity-determining region amino acid sequence
AITYVGSTWYQ

SEQ ID NO: 179 - protein, 142 aa, synthetic construct
  REGION 1..142: note = nanobody amino acid sequence
QVQLQESGGGTVQPGGSLKLSCAASGNLNYINVWSWYRQAPGKQRERVAGIATGGGRISYSESVKGRFIISRDDATNTVSLQMSGLTPEDTAVYYCNAFGSDPDFSDYKHDYWGQGTQVTVSSAAAYPYDVPDYGSHHHHHH

SEQ ID NO: 180 - protein, 8 aa, synthetic construct
  REGION 1..8: note = Complementarity-determining region amino acid sequence
GNLNYINV

SEQ ID NO: 181 - protein, 8 aa, synthetic construct
  REGION 1..8: note = Complementarity-determining region amino acid sequence
IATGGGRI

SEQ ID NO: 182 - protein, 16 aa, synthetic construct
  REGION 1..16: note = Complementarity-determining region amino acid sequence
NAFGSDPDFSDYKHDY

SEQ ID NO: 183 - protein, 135 aa, synthetic construct
  REGION 1..135: note = nanobody amino acid sequence
QVQLQESGGGLVQTGGSLRLSCAASGSTFSGYIMSWFRQPPGKERELVAAITYVGSTYVEDSVKGRFTISRDNAKNTVSLQMNSLKPEDTAVYYCRAREGEQLDFWGQGTQVTVSSAAAYPYDVPDYGSHHHHHH

SEQ ID NO: 184 - skipped

SEQ ID NO: 185 - protein, 135 aa, synthetic construct
  REGION 1..135: note = nanobody amino acid sequence
QVQLQESGGGLVQAGGSLRLSCAASGSTFSGYIMSWFRQPPGKERELVAAITYVGSTYVEDSVKGRFTISRDNVKNTVSLQMNSLKPEDTAVYYCRAREGEQLDFWGQGTQVTVSSAAAYPYDVPDYGSHHHHHH

SEQ ID NO: 186 - protein, 136 aa, synthetic construct
  REGION 1..136: note = nanobody amino acid sequence
QVQLQESGGGLVQPGGSLRLSCAASGTIFSAKALGWHRQAPGKQREVVAGITSGGSTNYADSVKGRFTISRDNAKNTVYLQMNSVKFEDTAVYYCVLYDLIKDRTYWGQGTQVTVSSAAAYPYDVPDYGSHHHHHH

SEQ ID NO: 187 - protein, 8 aa, synthetic construct
  REGION 1..8: note = Complementarity-determining region amino acid sequence
GTIFSAKA

SEQ ID NO: 188 - protein, 7 aa, synthetic construct
  REGION 1..7: note = Complementarity-determining region amino acid sequence
ITSGGST

SEQ ID NO: 189 - protein, 11 aa, synthetic construct
  REGION 1..11: note = Complementarity-determining region amino acid sequence
VLYDLIKDRTY

SEQ ID NO: 190 - protein, 137 aa, synthetic construct
  REGION 1..137: note = nanobody amino acid sequence
QVQLQESGGGLVQPGGSLRLSCATSTSIFSINVMDWYRQAPGKQRELVAGITSGDNTNYADSVKGRFTISRDNANNTVWLQMNSLKPEDTAVYYCRGRVYNGGWYDYWGQGTQVTVSSAAAYPYDVPDYGSHHHHHH

SEQ ID NO: 191 - protein, 7 aa, synthetic construct
  REGION 1..7: note = Complementarity-determining region amino acid sequence
TSIFSGT

SEQ ID NO: 192 - protein, 7 aa, synthetic construct
  REGION 1..7: note = Complementarity-determining region amino acid sequence
ITSGDNT

SEQ ID NO: 193 - protein, 12 aa, synthetic construct
  REGION 1..12: note = Complementarity-determining region amino acid sequence
RGRVYNGGWYDY

SEQ ID NO: 194 - protein, 141 aa, synthetic construct
  REGION 1..141: note = nanobody amino acid sequence
QVQLQESGGGLVQAGGSLRLACAASERTFSSYVMGWFRQGPGKEREFVAAISWSGGARYYADSVKGRFTISRDNAKNTVYLQMSSLKPEDTAVYVCAARRTYSPRTLEYDFWGQGTQVTVSSAAAYPYDVPDYGSHHHHHH

SEQ ID NO: 195 - protein, 8 aa, synthetic construct
  REGION 1..8: note = Complementarity-determining region amino acid sequence
ERTFSSYV

SEQ ID NO: 196 - protein, 8 aa, synthetic construct
  REGION 1..8: note = Complementarity-determining region amino acid sequence
ISWSGGAR

SEQ ID NO: 197 - protein, 15 aa, synthetic construct
  REGION 1..15: note = Complementarity-determining region amino acid sequence
AARRTYSPRTLEYDF

SEQ ID NO: 198 - protein, 141 aa, synthetic construct
  REGION 1..141: note = nanobody amino acid sequence
QVQLQESGGGLVQAGGSLRLSCAASGDTFSTYVMGWFRQGPGKEREFVAAISWSGGARYYADSVKGRFTISRDNAKNTVYLQMSSLKPEDTAVYVCAARRTYSPRTLEYDFWGQGTQVTVSSAAAYPYDVPDYGSHHHHHH

SEQ ID NO: 199 - protein, 8 aa, synthetic construct
  REGION 1..8: note = Complementarity-determining region amino acid sequence
GDTFSTYV

SEQ ID NO: 200 - protein, 140 aa, synthetic construct
  REGION 1..140: note = nanobody amino acid sequence
QVQLQESGGGLVQPGGSLRLSCAASGFTFSDYVMSWVRQAPGKGFEWVSIINTDGIGSRYADSVMGRFTISRDNEKKMMYLQMNSLKPEDTAMYYCARGNAALTVIRGRPWGQGTQVTVSSAAAYPYDVPDYGSHHHHHH

SEQ ID NO: 201 - protein, 8 aa, synthetic construct
  REGION 1..8: note = Complementarity-determining region amino acid sequence
GFTFSDYV

SEQ ID NO: 202 - protein, 8 aa, synthetic construct
  REGION 1..8: note = Complementarity-determining region amino acid sequence
INTDGIGS

SEQ ID NO: 203 - protein, 14 aa, synthetic construct
  REGION 1..14: note = Complementarity-determining region amino acid sequence
ARGNAALTVIRGRP

SEQ ID NO: 204 - protein, 140 aa, synthetic construct
  REGION 1..140: note = nanobody amino acid sequence
QVQLQESGGGLVQPGGSLRLSCAASGFTFSDYVMSWVRQAPGKGFEWVSIINTDGNGSRYADSVMGRFTISRDNDKKMMYLQMNSLKPEDTAMYYCAKGNAALSLIRGRPWGQGTQVTVSSAAAYPYDVPDYGSHHHHHH

SEQ ID NO: 205 - protein, 8 aa, synthetic construct
  REGION 1..8: note = Complementarity-determining region amino acid sequence
INTDGNGS

SEQ ID NO: 206 - protein, 14 aa, synthetic construct
  REGION 1..14: note = Complementarity-determining region amino acid sequence
AKGNAALSLIRGRP

SEQ ID NO: 207 - protein, 147 aa, synthetic construct
  REGION 1..147: note = nanobody amino acid sequence
QVQLQESGGGLVQPGGSLRLSCAASGRTFSTYAMGWFRQAHGKEREFVAAISWGRGNTYYGDSLKGRFTISRDNAKNTVYLQMDSLKPEDTAVYYCAADPGRAYVSGNYYSAATYDYWGQGTQVTVSSAAAYPYDVPDYGSHHHHHH

SEQ ID NO: 208 - protein, 8 aa, synthetic construct
  REGION 1..8: note = Complementarity-determining region amino acid sequence
GRTFSTYA

SEQ ID NO: 209 - protein, 8 aa, synthetic construct
  REGION 1..8: note = Complementarity-determining region amino acid sequence
ISWGRGNT

SEQ ID NO: 210 - protein, 21 aa, synthetic construct
  REGION 1..21: note = Complementarity-determining region amino acid sequence
AADPGRAYVSGNYYSAATYDY

SEQ ID NO: 211 - protein, 147 aa, synthetic construct
  REGION 1..147: note = nanobody amino acid sequence
QVQLQESGGGLVQAGGSLRLSCAASGRTFSSYAMGWFRRFPGKEREFVAAISWGRGNTYYGDSLKGRFTISRDNAKNTVYLQMDSLKPEDTAVYYCAADPGRAYVSGNYYSAATYDYWGQGTQVTVSSAAAYPYDVPDYGSHHHHHH

SEQ ID NO: 212 - protein, 8 aa, synthetic construct
  REGION 1..8: note = Complementarity-determining region amino acid sequence
GRTFSSYA

SEQ ID NO: 213 - protein, 147 aa, synthetic construct
  REGION 1..147: note = nanobody amino acid sequence
QVQLQESGGGLVQAGGSLRLSCAASGRTFSTYAMGWFRQAHGKEREFVAAISWGRGNTYYGDSLKGRFTISRDNAKNTVYLQMDSLKPEDTAVYYCAADPGRAYVSGNYYSAATYDYWGQGTQVTVSSAAAYPYDVPDYGSHHHHHH

SEQ ID NO: 214 - protein, 145 aa, synthetic construct
  REGION 1..145: note = nanobody amino acid sequence
QVQLQESGGGLVQAGGSLRLSCAASGGSFSTIPMGWFRQAPEMERGFVAAISYRGTYTYYTNSVKGRFTISRDNAKNTVYLQMNSLKPEDTAVYYCAAGSPGISAYWGDLSNWKNWGQGTQVTVSSAAAYPYDVPDYGSHHHHHH

SEQ ID NO: 215 - protein, 8 aa, synthetic construct
  REGION 1..8: note = Complementarity-determining region amino acid sequence
GGSFSTIP

SEQ ID NO: 216 - protein, 8 aa, synthetic construct
  REGION 1..8: note = Complementarity-determining region amino acid sequence
ISYRGTYT

SEQ ID NO: 217 - protein, 19 aa, synthetic construct
  REGION 1..19: note = Complementarity-determining region amino acid sequence
AAGSPGISAYWGDLSNWKN

SEQ ID NO: 218 - protein, 143 aa, synthetic construct
  REGION 1..143: note = nanobody amino acid sequence
QVQLQESGGGVVQAGGSLRLSCVASGRSFSSHAMGWFRQATGKEREFVATISWNSGSTFYADSLRGRFTISRDNAKNTLYLQMNSLTAEDTAVYYCAAASGRGITASAFRYDVWGQGTQVTVSSAAAYPYDVPDYGSHHHHHH

SEQ ID NO: 219 - protein, 8 aa, synthetic construct
  REGION 1..8: note = Complementarity-determining region amino acid sequence
GRSFSSHA

SEQ ID NO: 220 - protein, 8 aa, synthetic construct
  REGION 1..8: note = Complementarity-determining region amino acid sequence
ISWNSGST

SEQ ID NO: 221 - protein, 17 aa, synthetic construct
  REGION 1..17: note = Complementarity-determining region amino acid sequence
AAASGRGITASAFRYDV

SEQ ID NO: 222 - protein, 148 aa, synthetic construct
  REGION 1..148: note = nanobody amino acid sequence
QVQLQESGGGLVQAGDSLRLSCAASGRTFSSYTMGWFRQAPGKEREFVAAIRWSGSITYYSDSVKGRFTISRDNAKNTVYLQMNSLKPEDTAVYYCAADPGRIFGSGSYYGNRNTYDYWGQGTQVTVSSAAAYPYDVPDYGSHHHHHH

SEQ ID NO: 223 - protein, 8 aa, synthetic construct
  REGION 1..8: note = Complementarity-determining region amino acid sequence
GRTFSSYT

SEQ ID NO: 224 - protein, 8 aa, synthetic construct
  REGION 1..8: note = Complementarity-determining region amino acid sequence
IRWSGSIT

SEQ ID NO: 225 - protein, 22 aa, synthetic construct
  REGION 1..22: note = Complementarity-determining region amino acid sequence
AADPGRIFGSGSYYGNRNTYDY

SEQ ID NO: 226 - protein, 146 aa, synthetic construct
  REGION 1..146: note = nanobody amino acid sequence
QVQLQESGGGLVQAGGSLRLSCAVSERTFRTYTMAWFRQAPGKEREFVAAIRWNGDSTYYADSVKGRFTISRDNAKNTMYLQMNSLKPEDTAVYNCAARAPSGGYYYPNALSEYNYWGQGTQVTVSSAAAYPYDVPDYGSHHHHHH

SEQ ID NO: 227 - protein, 8 aa, synthetic construct
  REGION 1..8: note = Complementarity-determining region amino acid sequence
ERTFRTYT

SEQ ID NO: 228 - protein, 8 aa, synthetic construct
  REGION 1..8: note = Complementarity-determining region amino acid sequence
IRWNGDST

SEQ ID NO: 229 - protein, 20 aa, synthetic construct
  REGION 1..20: note = Complementarity-determining region amino acid sequence
AARAPSGGYYYPNALSEYNY

SEQ ID NO: 230 - protein, 146 aa, synthetic construct
  REGION 1..146: note = nanobody amino acid sequence
QVQLQESGGGLVQPGGSLRLSCAASERTFRTYTMGWFRQAPGKEREFVAAIRWNGDSTYYADSVKGRFTISRDNAKNTMYLQMNSLKPEDTAVYSCAARAYSIGYYYPNALSEYNYWGQGTQVTVSSAAAYPYDVPDYGSHHHHHH

SEQ ID NO: 231 - protein, 13 aa, synthetic construct
  REGION 1..13: note = Complementarity-determining region amino acid sequence
AASERTFRTYTMG

SEQ ID NO: 232 - protein, 20 aa, synthetic construct
  REGION 1..20: note = Complementarity-determining region amino acid sequence
AARAYSIGYYYPNALSEYNY

SEQ ID NO: 233 - protein, 145 aa, synthetic construct
  REGION 1..145: note = nanobody amino acid sequence
QVQLQESGGGLVQAGDSLRLSCAASGVTISRYTMGWFRQAPGKEREFVGLIRWSNGNTYYTDSVKGRFTISRDNAKNTVYLQMNSLKPEDTAVYYCATLRSGYVGSAYSQQAYDYWGQGTQVTVSSAAAYPYDVPDYGSHHHHHH

SEQ ID NO: 234 - protein, 8 aa, synthetic construct
  REGION 1..8: note = Complementarity-determining region amino acid sequence
GVTISRYT

SEQ ID NO: 235 - protein, 8 aa, synthetic construct
  REGION 1..8: note = Complementarity-determining region amino acid sequence
IRWSNGNT

SEQ ID NO: 236 - protein, 19 aa, synthetic construct
  REGION 1..19: note = Complementarity-determining region amino acid sequence
ATLRSGYVGSAYSQQAYDY

SEQ ID NO: 237 - protein, 138 aa, synthetic construct
  REGION 1..138: note = nanobody amino acid sequence
QVQLQESGGGLVQAGGSLRLSCAASTSTVSDYHMGWFRQGPGKEREFVAAISWSGDSTDYADSVKARFTISRDNAKKTMSLQMNSLKPEDTAAYYCAARRVFKATWDYWGQGTQVTVSSAAAYPYDVPDYGSHHHHHH

SEQ ID NO: 238 - protein, 8 aa, synthetic construct
  REGION 1..8: note = Complementarity-determining region amino acid sequence
TSTVSDYH

SEQ ID NO: 239 - protein, 8 aa, synthetic construct
  REGION 1..8: note = Complementarity-determining region amino acid sequence
ISWSGDST

SEQ ID NO: 240 - protein, 12 aa, synthetic construct
  REGION 1..12: note = Complementarity-determining region amino acid sequence
AARRVFKATWDY

SEQ ID NO: 241 - protein, 147 aa, synthetic construct
  REGION 1..147: note = nanobody amino acid sequence
QVQLQESGGGLVQAGGSLRLTCAATVRIFSSYTMGWFRQSPGKEREFVGAITWSGGNTYYADSVKGRFTISRDNAKNTVYLQMNSLKSEDTAVYYCAADPGRSYVLSRYYDQASYDYWGQGTQVTVSSAAAYPYDVPDYGSHHHHHH

SEQ ID NO: 242 - protein, 8 aa, synthetic construct
  REGION 1..8: note = Complementarity-determining region amino acid sequence
VRIFSSYT

SEQ ID NO: 243 - protein, 8 aa, synthetic construct
  REGION 1..8: note = Complementarity-determining region amino acid sequence
ITWSGGNT

SEQ ID NO: 244 - protein, 21 aa, synthetic construct
  REGION 1..21: note = Complementarity-determining region amino acid sequence
AADPGRSYVLSRYYDQASYDY

SEQ ID NO: 245 - protein, 148 aa, synthetic construct
  REGION 1..148: note = nanobody amino acid sequence
QVQLQESGGGLVQAGDSLRLSCAASGRPFSSYTMGWFRQAPGKEREFVAAISWSGGTTYYTDSVQGRFTISRDNAKNTVYLQMNRLKPEDTAVYYCATDVGRIYGGGSLYSSAFSYDYWGQGTQVTVSSAAAYPYDVPDYGSHHHHHH

SEQ ID NO: 246 - protein, 8 aa, synthetic construct
  REGION 1..8: note = Complementarity-determining region amino acid sequence
GRPFSSYT

SEQ ID NO: 247 - protein, 8 aa, synthetic construct
  REGION 1..8: note = Complementarity-determining region amino acid sequence
ISWSGGTT

SEQ ID NO: 248 - protein, 22 aa, synthetic construct
  REGION 1..22: note = Complementarity-determining region amino acid sequence
ATDVGRIYGGGSLYSSAFSYDY

SEQ ID NO: 249 - protein, 140 aa, synthetic construct
  REGION 1..140: note = nanobody amino acid sequence
QVQLQESGGGLVQAGASLRLSCAASGGTFSDYVMGWFRQPPGKEREFVASINYGGDKISYADSLEGRFTILRDNTKDTTSLQMNTLKPDDTAVYYCAAKWGYKTGPTYSYWGQGTQVTVSSAAAYPYDVPDYGSHHHHHH

SEQ ID NO: 250 - protein, 8 aa, synthetic construct
  REGION 1..8: note = Complementarity-determining region amino acid sequence
GGTFSDYV

SEQ ID NO: 251 - protein, 8 aa, synthetic construct
  REGION 1..8: note = Complementarity-determining region amino acid sequence
INYGGDKI

SEQ ID NO: 252 - protein, 14 aa, synthetic construct
  REGION 1..14: note = Complementarity-determining region amino acid sequence
AAKWGYKTGPTYSY

SEQ ID NO: 253 - protein, 132 aa, synthetic construct
  REGION 1..132: note = nanobody amino acid sequence
QVQLQESGGGLVQAGGSLRLSCAASGRTFSNVAMGWFRQAPGKEREFAAAISGSGGTTYYAASVKGRFTISRDNGKKMVYLQMNSLKPEDTAVYYCSTYDGRRGQGTQVTVSSAAAYPYDVPDYGSHHHHHH

SEQ ID NO: 254 - protein, 8 aa, synthetic construct
  REGION 1..8: note = Complementarity-determining region amino acid sequence
GRTFSNVA

SEQ ID NO: 255 - protein, 8 aa, synthetic construct
  REGION 1..8: note = Complementarity-determining region amino acid sequence
ISGSGGTT

SEQ ID NO: 256 - protein, 6 aa, synthetic construct
  REGION 1..6: note = Complementarity-determining region amino acid sequence
STYDGR

SEQ ID NO: 257 - protein, 145 aa, synthetic construct
  REGION 1..145: note = nanobody amino acid sequence
QVQLQESGGGLVQAGGSLRLSCAASGLTFSRNAIAWFRQAPGKEREFVAAISWNAITTAYGDSVKGRFTIFRGNTKNTVYLQMNSLKPEDTAVYYCAARYSSGSYYYARTYEYDYWGQGTQVTVSSAAAYPYDVPDYGSHHHHHH

SEQ ID NO: 258 - protein, 8 aa, synthetic construct
  REGION 1..8: note = Complementarity-determining region amino acid sequence
GLTFSRNA

SEQ ID NO: 259 - protein, 9 aa, synthetic construct
  REGION 1..9: note = Complementarity-determining region amino acid sequence
ISWNAITTA

SEQ ID NO: 260 - protein, 19 aa, synthetic construct
  REGION 1..19: note = Complementarity-determining region amino acid sequence
AARYSSGSYYYARTYEYDY

SEQ ID NO: 261 - protein, 138 aa, synthetic construct
  REGION 1..138: note = nanobody amino acid sequence
QVQLQESGGGLVQTGGSLRLSCAASGRTSRSYAMGWFRQAPGKERELVADIAWSDGRIYYSDSVKGRFTISRDNAKNTVYLEIDSLKPGDTAVYYCASRARGTVLYNYWGQGTQVTVSSAAAYPYDVPDYGSHHHHHH

SEQ ID NO: 262 - protein, 8 aa, synthetic construct
  REGION 1..8: note = Complementarity-determining region amino acid sequence
GRTSRSYA

SEQ ID NO: 263 - protein, 8 aa, synthetic construct
  REGION 1..8: note = Complementarity-determining region amino acid sequence
IAWSDGRI

SEQ ID NO: 264 - protein, 12 aa, synthetic construct
  REGION 1..12: note = Complementarity-determining region amino acid sequence
ASRARGTVLYNY

SEQ ID NO: 265 - protein, 138 aa, synthetic construct
  REGION 1..138: note = nanobody amino acid sequence
QVQLQESGGGLVQPGGSLRLSCAASGRTSRSYAMGWFRQAPGKEREHVADIAWSDGRIYYSDSVKDRFTISRDNAKNTVYLEIDSLKPGDTAVYYCASRARGTVSYNYWGQGTQVTVSSAAAYPYDVPDYGSHHHHHH

SEQ ID NO: 266 - protein, 12 aa, synthetic construct
  REGION 1..12: note = Complementarity-determining region amino acid sequence
ASRARGTVSYNY

SEQ ID NO: 267 - protein, 138 aa, synthetic construct
  REGION 1..138: note = nanobody amino acid sequence
QVQLQESGGGLVQAGGSLRLSCAASGRTSRSYAMGWFRQAPGKERELVADIAWSDGRIYYSDSVKGRFTISRDNAKNTVYLEIDSLKPGDTAVYYCASRARGTVLYNYWGQGTQVTVSSAAAYPYDVPDYGSHHHHHH

SEQ ID NO: 268 - protein, 138 aa, synthetic construct
  REGION 1..138: note = nanobody amino acid sequence
QVQLQESGGGLVQAGGSLTLSCAASGRTSRSYAMGWFRQAPGKERELVADIAWSDGRIYYSDSVKGRFTISRDNAKNTVYLEIDSLKPGDTAVYYCASRARGTVSYNYWGQGTQVTVSSAAAYPYDVPDYGSHHHHHH

SEQ ID NO: 269 - protein, 138 aa, synthetic construct
  REGION 1..138: note = nanobody amino acid sequence
QVQLQESGGGLVQAGDSLRLSCAASGRTSRSYAMGWFRQAPGKERELVADISWSDGRIYYSDSVKGRFTISRDNAKNTVYLEIDSLKPGDTAVYYCASRARGSVLYNYWGQGTQVTVSSAAAYPYDVPDYGSHHHHHH

SEQ ID NO: 270 - protein, 8 aa, synthetic construct
  REGION 1..8: note = Complementarity-determining region amino acid sequence
ISWSDGRI

SEQ ID NO: 271 - protein, 138 aa, synthetic construct
  REGION 1..138: note = nanobody amino acid sequence
QVQLQESGGGSVQAGGSLRLSCAASGRTSRSYAMGWFRQAPGKERELVADIAWSDGRIYYSDSVKGRFTISRDNAKNTVYLEIDSLKPGDTAVYYCASRARGTVSYNYWGQGTQVTVSSAAAYPYDVPDYGSHHHHHH

SEQ ID NO: 272 - protein, 138 aa, synthetic construct
  REGION 1..138: note = nanobody amino acid sequence
QVQLQESGGGLVQAGGSLRLSCAASGRTSRSYAMGWFRQAPGKERELVADIAWSDGRIYYSDSVKGRFTISRDNAKNTVYLEIDSLKPGDTAVYYCASRARGTVSYNYWGQGTQVTVSSAAAYPYDVPDYGSHHHHHH

SEQ ID NO: 273 - protein, 135 aa, synthetic construct
  REGION 1..135: note = nanobody amino acid sequence
QVQLQESGGGLVQAGGSLRLSCVYSGNVFSINLMGWYRQAPGKQRELLAGINRGGRTNYTDVVKGRFTISRENAKNTIYLQMNGLKPEDTGVYYCAASRDPYTGYWGQGTQVTVSSAAAYPYDVPDYGSHHHHHH

SEQ ID NO: 274 - protein, 8 aa, synthetic construct
  REGION 1..8: note = Complementarity-determining region amino acid sequence
GNVFSINL

SEQ ID NO: 275 - protein, 7 aa, synthetic construct
  REGION 1..7: note = Complementarity-determining region amino acid sequence
INRGGRT

SEQ ID NO: 276 - protein, 10 aa, synthetic construct
  REGION 1..10: note = Complementarity-determining region amino acid sequence
AASRDPYTGY

SEQ ID NO: 277 - protein, 135 aa, synthetic construct
  REGION 1..135: note = nanobody amino acid sequence
QVQLQESGGGLVQAGGSLRLSCVYSGNVFSINLMGWYRQAPGKQRELLAGINRGGRTNYTDVVKGRFTISKENAKNTIYLQMNSLKPEDTGVYYCAASRDPYTGYWGQGTQVTVSSAAAYPYDVPDYGSHHHHHH

SEQ ID NO: 278 - protein, 135 aa, synthetic construct
  REGION 1..135: note = nanobody amino acid sequence
QVQLQESGGGLVQAGGSLRLSCVYSGNVFSINLMGWYRQAPGKQRELLAGINRGGRTNYTDVVKGRFTISRENAKNTIYLQMNSLKPEDTGVYYCAASRDPYTGYWGQGTQVTVSSAAAYPYDVPDYGSHHHHHH

SEQ ID NO: 279 - protein, 135 aa, synthetic construct
  REGION 1..135: note = nanobody amino acid sequence
QVQLQESGGGLVQPGGSLRLSCVASGSVDSINLMGWYRQAPGKQRELLAGINRGGRTNYTDVVKGRFTISRENAKNTIYLQMNSLKPEDTGVYYCAASRDPYTGYWGQGTQVTVSSAAAYPYDVPDYGSHHHHHH

SEQ ID NO: 280 - protein, 8 aa, synthetic construct
  REGION 1..8: note = Complementarity-determining region amino acid sequence
GSVDSINL

SEQ ID NO: 281 - protein, 135 aa, synthetic construct
  REGION 1..135: note = nanobody amino acid sequence
QVQLQESGGGLVPSGGSLRLSCVYSGNVFSINLMGWYRQAPGKQRELLAGINRGGRTNYTDVVKGRFTISRENAKNTIYLQMNGLKPEDTGVYYCAASRDPYTGYWGQGTQVTVSSAAAYPYDVPDYGSHHHHHH

SEQ ID NO: 282 - protein, 7 aa, synthetic construct
  REGION 1..7: note = Complementarity-determining region amino acid sequence
NVFSINL

SEQ ID NO: 283 - protein, 135 aa, synthetic construct
  REGION 1..135: note = nanobody amino acid sequence
QVQLQESGGGLVQAGGSLRLSCVYSGNVFSINLMGWYRQAPGKQRELLAGITRGGRTNYTDVVKGRFTISRESAKNTIYLQMNSLKPEDTGVYYCAASRDPYTGYWGQGTQVTVSSAAAYPYDVPDYGSHHHHHH

SEQ ID NO: 284 - protein, 135 aa, synthetic construct
  REGION 1..135: note = nanobody amino acid sequence
QVQLQESGGGLVQAGGSLRLSCVYSGNVFSINLMGWYRQAPGKQRELLAGITRGGRTNYTDVVKGRFTISRENAKNTIYLQMNSLKPEDTGVYYCAASRDPYTGYWGQGTQVTVSSAAAYPYDVPDYGSHHHHHH

SEQ ID NO: 285 - protein, 11 aa, synthetic construct
  REGION 1..11: note = Complementarity-determining region amino acid sequence
CAASRDPYTGY

SEQ ID NO: 286 - protein, 143 aa, synthetic construct
  REGION 1..143: note = nanobody amino acid sequence
QVQLQESGGGLVQAGDSLRLSCVASKRSFSSHAMGWFRQAPGKAREFVATISWNSGSTFYSDSSRGRFTISRDNGKNTLYLQMNSLKPEDTAVYYCAAASGRGITASDFRYDAWGQGTQVTVSSAAAYPYDVPDYGSHHHHHH

SEQ ID NO: 287 - protein, 8 aa, synthetic construct
  REGION 1..8: note = Complementarity-determining region amino acid sequence
KRSFSSHA

SEQ ID NO: 288 - skipped

SEQ ID NO: 289 - protein, 17 aa, synthetic construct
  REGION 1..17: note = Complementarity-determining region amino acid sequence
AAASGRGITASDFRYDA

SEQ ID NO: 290 - protein, 143 aa, synthetic construct
  REGION 1..143: note = nanobody amino acid sequence
QVQLQESGGGVVQAGGSLRLSCVGSGRTFSSHAMGWFRQAPGKEREFVATISWNSGSTFYADSVRGRFTISRDNAKNTLYLQMNSLKAEDTAVYYCAAASGRGITASDFRYDAWGQGTQVTVSSAAAYPYDVPDYGSHHHHHH

SEQ ID NO: 291 - protein, 8 aa, synthetic construct
  REGION 1..8: note = Complementarity-determining region amino acid sequence
GRTFSSHA

SEQ ID NO: 292 - protein, 143 aa, synthetic construct
  REGION 1..143: note = nanobody amino acid sequence
QVQLQESGGGSVQAGGSLRLSCVASGRSFSRHAMAWFRQAPGKEREFVATISWNAGSTYYADSVKGRFTISRDNAKNTVTLQMNSLKPEDTAVYYCAAASGRGITASDFRYDAWGQGTQVTVSSAAAYPYDVPDYGSHHHHHH

SEQ ID NO: 293 - protein, 8 aa, synthetic construct
  REGION 1..8: note = Complementarity-determining region amino acid sequence
GRSFSRHA

SEQ ID NO: 294 - protein, 8 aa, synthetic construct
  REGION 1..8: note = Complementarity-determining region amino acid sequence
ISWNAGST

SEQ ID NO: 295 - protein, 141 aa, synthetic construct
  REGION 1..141: note = nanobody amino acid sequence
QVQLQESGGGLVQAGGSLRLSCAASGRTFSRINMGWFRQAPGKEREFVAVISWNSIADYADSVKARFTISRDNAKNTVYLQMNSLKPEDTAVYYCAASAPFRSKNPTLYLYWGRGTQVTVSSAAAYPYDVPDYGSHHHHHH

SEQ ID NO: 296 - protein, 8 aa, synthetic construct
  REGION 1..8: note = Complementarity-determining region amino acid sequence
GRTFSRIN

SEQ ID NO: 297 - protein, 7 aa, synthetic construct
  REGION 1..7: note = Complementarity-determining region amino acid sequence
ISWNSIA

SEQ ID NO: 298 - protein, 16 aa, synthetic construct
  REGION 1..16: note = Complementarity-determining region amino acid sequence
AASAPFRSKNPTLYLY

SEQ ID NO: 299 - protein, 147 aa, synthetic construct
  REGION 1..147: note = nanobody amino acid sequence
  VARIANT 39: note = Xaa can be any naturally occurring amino acid
QVQLQESGGGLVQSGDSLRLSCSASGRTFNPVAMAWFRXAPGKEREFVGTITWGIGSTHYAVPVKGRFTISKENAKNTVYLQMNRLQPEDTAVYYCAARTSLLRRADEIPSVANYDSWGQGTQVTVSSAAAYPYDVPDYGSHHHHHH

SEQ ID NO: 300 - protein, 8 aa, synthetic construct
  REGION 1..8: note = Complementarity-determining region amino acid sequence
GRTFNPVA

SEQ ID NO: 301 - protein, 8 aa, synthetic construct
  REGION 1..8: note = Complementarity-determining region amino acid sequence
ITWGIGST

SEQ ID NO: 302 - protein, 21 aa, synthetic construct
  REGION 1..21: note = Complementarity-determining region amino acid sequence
AARTSLLRRADEIPSVANYDS

SEQ ID NO: 303 - protein, 143 aa, synthetic construct
  REGION 1..143: note = nanobody amino acid sequence
  VARIANT 3: note = Xaa can be any naturally occurring amino acid
QVXLQESGGGLVQAGDSLRLSCAASGRTFSMYTMGWFRQAPGKEREFVAKISTSGRYTDYVDSVRGRFTLSRDNVKNTIYLQMNSLKPDDTAVYYCAARLPRPDTWSQGKTDYWGQGTQVTVSSAAAYPYDVPDYGSHHHHHH

SEQ ID NO: 304 - protein, 8 aa, synthetic construct
  REGION 1..8: note = Complementarity-determining region amino acid sequence
GRTFSMYT

SEQ ID NO: 305 - protein, 8 aa, synthetic construct
  REGION 1..8: note = Complementarity-determining region amino acid sequence
ISTSGRYT

SEQ ID NO: 306 - protein, 17 aa, synthetic construct
  REGION 1..17: note = Complementarity-determining region amino acid sequence
AARLPRPDTWSQGKTDY

SEQ ID NO: 307 - protein, 144 aa, synthetic construct
  REGION 1..144: note = nanobody amino acid sequence
  VARIANT 39: note = Xaa can be any naturally occurring amino acid
QVQLQESGGGLVQAGGSLRLSCAASGRTFSNNPMGWFRXAPGKEREFVATKTGGNSGITVYSNSVKGRFTISRDLAKNTVYLQMDGLKLEDTAIYYCAASGTPLALRSEKNYDYWGQGTQVTVSSAAAYPYDVPDYGSHHHHHH

SEQ ID NO: 308 - protein, 8 aa, synthetic construct
  REGION 1..8: note = Complementarity-determining region amino acid sequence
GRTFSNNP

SEQ ID NO: 309 - protein, 9 aa, synthetic construct
  REGION 1..9: note = Complementarity-determining region amino acid sequence
KTGGNSGIT

SEQ ID NO: 310 - protein, 17 aa, synthetic construct
  REGION 1..17: note = Complementarity-determining region amino acid sequence
AASGTPLALRSEKNYDY

SEQ ID NO: 311 - protein, 136 aa, synthetic construct
  REGION 1..136: note = nanobody amino acid sequence
  VARIANT 3: note = Xaa can be any naturally occurring amino acid
QVXLQESGGGLVQPGGSLRLSCAASGSIFNINTMGWYRQAPGNQRELVAAISSDGRPNHRDSVKGRFTISRDNAKNTVYLQMNSLKPEDTAVYYCNTVPASRAGGYWGQGTQVTVSSAAAYPYDVPDYGSHHHHHH

SEQ ID NO: 312 - protein, 8 aa, synthetic construct
  REGION 1..8: note = Complementarity-determining region amino acid sequence
GSIFNINT

SEQ ID NO: 313 - protein, 7 aa, synthetic construct
  REGION 1..7: note = Complementarity-determining region amino acid sequence
ISSDGRP

SEQ ID NO: 314 - protein, 11 aa, synthetic construct
  REGION 1..11: note = Complementarity-determining region amino acid sequence
NTVPASRAGGY

SEQ ID NO: 315 - protein, 143 aa, synthetic construct
  REGION 1..143: note = nanobody amino acid sequence
QVQLQESGGGLAQAGDSLRLSCVASGRTIDNGAMGWFRQAPGKERESVAAINWSGSSTYYADSVKGRFAISRDNVKHEVYLQMNRLRREDTAVYYCAAAKSIGTYSSSSAYDYWGQGTQVTVSSAAAYPYDVPDYGSHHHHHH

SEQ ID NO: 316 - protein, 8 aa, synthetic construct
  REGION 1..8: note = Complementarity-determining region amino acid sequence
GRTIDNGA

SEQ ID NO: 317 - protein, 8 aa, synthetic construct
  REGION 1..8: note = Complementarity-determining region amino acid sequence
INWSGSST

SEQ ID NO: 318 - protein, 17 aa, synthetic construct
  REGION 1..17: note = Complementarity-determining region amino acid sequence
AAAKSIGTYSSSSAYDY

SEQ ID NO: 319 - protein, 143 aa, synthetic construct
  REGION 1..143: note = nanobody amino acid sequence
QVQLQESGGGLVQAGGSLRLSCAASGRTFNTYAMGWFRQAPGKEREFVAAMNRDGSTVLYADSVKGRFAISKDNAKNTGYLQMNSLKPEDTAIYYCAANARYSDYTDGQSFVSWGQGTQVTVSSAAAYPYDVPDYGSHHHHHH

SEQ ID NO: 320 - protein, 8 aa, synthetic construct
  REGION 1..8: note = Complementarity-determining region amino acid sequence
GRTFNTYA

SEQ ID NO: 321 - protein, 8 aa, synthetic construct
  REGION 1..8: note = Complementarity-determining region amino acid sequence
MNRDGSTV

SEQ ID NO: 322 - protein, 17 aa, synthetic construct
  REGION 1..17: note = Complementarity-determining region amino acid sequence
AANARYSDYTDGQSFVS

SEQ ID NO: 323 - protein, 143 aa, synthetic construct
  REGION 1..143: note = nanobody amino acid sequence
QVQLQESGGGLVQAGGSLRLSCQASGRTFNTYAMGWFRQAPGKEREFVAAMNRDGSTILYGDSVKGRFAISRDNAKNTGYLQMNSLKPEDTAIYYCAANARYSDYTHGQSFVSWGQGTQVTVSSAAAYPYDVPDYGSHHHHHH

SEQ ID NO: 324 - protein, 8 aa, synthetic construct
  REGION 1..8: note = Complementarity-determining region amino acid sequence
MNRDGSTI

SEQ ID NO: 325 - protein, 17 aa, synthetic construct
  REGION 1..17: note = Complementarity-determining region amino acid sequence
AANARYSDYTHGQSFVS

SEQ ID NO: 326 - protein, 143 aa, synthetic construct
  REGION 1..143: note = nanobody amino acid sequence
QVQLQESGGGLVQAGGSLRLSCQASGRTFDAYAMGWFRQAPGKEREFVAAMNRDGSTVLYADSVKGRFAISKDNAKNTGYLQMNSLKPEDTAIYYCAANARYSDYTDGQSFISWGQGTQVTVSSAAAYPYDVPDYGSHHHHHH

SEQ ID NO: 327 - protein, 8 aa, synthetic construct
  REGION 1..8: note = Complementarity-determining region amino acid sequence
GRTFDAYA

SEQ ID NO: 328 - protein, 17 aa, synthetic construct
  REGION 1..17: note = Complementarity-determining region amino acid sequence
AANARYSDYTDGQSFIS

SEQ ID NO: 329 - protein, 143 aa, synthetic construct
  REGION 1..143: note = nanobody amino acid sequence
QVQLQESGGGLVQAGGSLRLSCQASGRTFNTYAMGWFRQAPGKEREFVAAMNRDGSTVLYADSVKGRFAISKDNAKNTGYLQMNSLKPEDTAIYYCAANARYSDYTDGQSFVSWGQGTQVTVSSAAAYPYDVPDYGSHHHHHH

SEQ ID NO: 330 - skipped

SEQ ID NO: 331 - protein, 143 aa, synthetic construct
  REGION 1..143: note = nanobody amino acid sequence
QVQLQESGGGLVQAGGSLRLSCVASGRTFNTYAMGWFRQAPGKEREFVAAMNRDGSTVLYADYVKGRFAISKDNAKNTGYLQMNSLKPEDTAIYYCAANARYSDYTDEQSFVSWGQGTQVTVSSAAAYPYDVPDYGSHHHHHH

SEQ ID NO: 332 - protein, 17 aa, synthetic construct
  REGION 1..17: note = Complementarity-determining region amino acid sequence
AANARYSDYTDEQSFVS

SEQ ID NO: 333 - protein, 143 aa, synthetic construct
  REGION 1..143: note = nanobody amino acid sequence
QVQLQESGGGLVQAGGSLRLSCQASGRTFNTYAMGWFRQAPGKEREFVAAMNRDGSTVLYADSVKGRFAISKDNAKSTGYLQMNSLKPEDTAIYYCAANARYSDYTDGQSFVSWGQGTQVTVSSAAAYPYDVPDYGSHHHHHH

SEQ ID NO: 334 - protein, 143 aa, synthetic construct
  REGION 1..143: note = nanobody amino acid sequence
QVQLQESGGGLVQAGGSLRLSCKASGRTFSTYAVGWFRQAPGKPREFVAAMNSRGSTINYADSVKGRFAISRDNAKNTGYLQMDSLKPEDTAIYYCAADARYSDYTDGQSFKSWGQGTQVTVSSAAAYPYDVPDYGSHHHHHH

SEQ ID NO: 335 - skipped

SEQ ID NO: 336 - protein, 8 aa, synthetic construct
  REGION 1..8: note = Complementarity-determining region amino acid sequence
MNSRGSTI

SEQ ID NO: 337 - protein, 17 aa, synthetic construct
  REGION 1..17: note = Complementarity-determining region amino acid sequence
AADARYSDYTDGQSFKS

SEQ ID NO: 338 - protein, 143 aa, synthetic construct
  REGION 1..143: note = nanobody amino acid sequence
QVQLQESGGGLVQAGGSLRLSCQASGRTFNTYAMGWFRQAPGKEREFVAAMNRDGSTILYADSVKGRFAISKDNAKNTGYLQMNSLKPEDTAIYYCAANARYSDYTNGHSFVSWGQGTQVTVSSAAAYPYDVPDYGSHHHHHH

SEQ ID NO: 339 - protein, 17 aa, synthetic construct
  REGION 1..17: note = Complementarity-determining region amino acid sequence
AANARYSDYTNGHSFVS

SEQ ID NO: 340 - protein, 143 aa, synthetic construct
  REGION 1..143: note = nanobody amino acid sequence
QVQLQESGGGLVQPGGSLRLSCQASGRTFNTYAMGWFRQAPGKEREFVAAMNRDGSTVLYADSVKGRFAISKDNARNTGYLQMNSLKPEDTAIYYCAANARYSDYTDGQSFVSWGQGTQVTVSSAAAYPYDVPDYGSHHHHHH

SEQ ID NO: 341 - protein, 143 aa, synthetic construct
  REGION 1..143: note = nanobody amino acid sequence
QVQLQESGGGLVQAGGSLRLSCQASGRTFNTYAMGWFRQAPGKEREFVAAMNRDGSTVLYRDSVKGRFAISKDNARNTGYLQMNNLKPEDTAIYYCAANARYSDYTDGQSFVSWGQGTQVTVSSAAAYPYDVPDYGSHHHHHH

SEQ ID NO: 342 - protein, 143 aa, synthetic construct
  REGION 1..143: note = nanobody amino acid sequence
QVQLQESGGGLVQAGGSLRLSCQASERSFSTYAMGWFRQAPGKEREFVAAMNRNGNTINYLDSVKGRFAISRDNAKSTGYLQMNSLKPEDTATYYCSANARLSDYTNPQSFVSWGQGTQVTVSSAAAYPYDVPDYGSHHHHHH

SEQ ID NO: 343 - protein, 8 aa, synthetic construct
  REGION 1..8: note = Complementarity-determining region amino acid sequence
ERSFSTYA

SEQ ID NO: 344 - protein, 8 aa, synthetic construct
  REGION 1..8: note = Complementarity-determining region amino acid sequence
MNRNGNTI

SEQ ID NO: 345 - protein, 17 aa, synthetic construct
  REGION 1..17: note = Complementarity-determining region amino acid sequence
SANARLSDYTNPQSFVS

SEQ ID NO: 346 - protein, 140 aa, synthetic construct
  REGION 1..140: note = nanobody amino acid sequence
QVQLQESGGGLVQAGGSLRLSCAASGRAFSSYGMAWFRQAPGKEREFVAAINSNGRSTYYADTVKGRFTISRDDGRNTLYLQMNSLKPEDTAVYYCAADRQSMKGYEYGYWGQGTQVTVSSAAAYPYDVPDYGSHHHHHH

SEQ ID NO: 347 - protein, 8 aa, synthetic construct
  REGION 1..8: note = Complementarity-determining region amino acid sequence
GRAFSSYG

SEQ ID NO: 348 - protein, 8 aa, synthetic construct
  REGION 1..8: note = Complementarity-determining region amino acid sequence
INSNGRST

SEQ ID NO: 349 - protein, 14 aa, synthetic construct
  REGION 1..14: note = Complementarity-determining region amino acid sequence
AADRQSMKGYEYGY

SEQ ID NO: 350 - protein, 140 aa, synthetic construct
  REGION 1..140: note = nanobody amino acid sequence
QVQLQESGGGLVQAGGSLRLSCAASGRAFSRYGMAWFRQAPGKEREFVAAITSNGRSTYYADTVKGRFTISRDNGRNTLYLQMNTLKPEDTAVYYCAADRKSMTGYEYGYWGQGTQVTVSSAAAYPYDVPDYGSHHHHHH

SEQ ID NO: 351 - protein, 8 aa, synthetic construct
  REGION 1..8: note = Complementarity-determining region amino acid sequence
GRAFSRYG

SEQ ID NO: 352 - protein, 8 aa, synthetic construct
  REGION 1..8: note = Complementarity-determining region amino acid sequence
ITSNGRST

SEQ ID NO: 353 - protein, 14 aa, synthetic construct
  REGION 1..14: note = Complementarity-determining region amino acid sequence
AADRKSMTGYEYGY

SEQ ID NO: 354 - protein, 140 aa, synthetic construct
  REGION 1..140: note = nanobody amino acid sequence
QVQLQESGGGLVQPGGSLRLSCAASGRAFSRYGMAWFRQAPGKEREFVAAITSNGRSTYYADTVKGRFTISRDNGRNTLYLQMNTLKPEDTAVYYCAADRKSMTGYEYGYWGQGTQVTVSSAAAYPYDVPDYGSHHHHHH

SEQ ID NO: 355 - protein, 140 aa, synthetic construct
  REGION 1..140: note = nanobody amino acid sequence
QVQLQESGGGLVQAGGSLRLSCAASGRAFSRYGMAWFRQAPGKEREFVGAINSNGRSTYYADTVKGRFTISRDNDRNTLYLQMNSLKPEDTAVYYCAADRQSMTRYEYGYWGQGTQVTVSSAAAYPYDVPDYGSHHHHHH

SEQ ID NO: 356 - protein, 14 aa, synthetic construct
  REGION 1..14: note = Complementarity-determining region amino acid sequence
AADRQSMTRYEYGY

SEQ ID NO: 357 - protein, 144 aa, synthetic construct
  REGION 1..144: note = nanobody amino acid sequence
QVQLQESGGGLVQPGGSLRLSCAASGFTFSNYAMGWFRQAPGKERDFVAAVSGIARRTYYADSVKGRSTISRDNGRNTLYLQMNNLKPEDTAVYYCARATSRMTSVTTLNDYGYWGQGTPVTVSSAAAYPYDVPDYGSHHHHHH

SEQ ID NO: 358 - protein, 8 aa, synthetic construct
  REGION 1..8: note = Complementarity-determining region amino acid sequence
GFTFSNYA

SEQ ID NO: 359 - protein, 8 aa, synthetic construct
  REGION 1..8: note = Complementarity-determining region amino acid sequence
VSGIARRT

SEQ ID NO: 360 - protein, 18 aa, synthetic construct
  REGION 1..18: note = Complementarity-determining region amino acid sequence
ARATSRMTSVTTLNDYGY

SEQ ID NO: 361 - protein, 144 aa, synthetic construct
  REGION 1..144: note = nanobody amino acid sequence
QVQLQESGGGLVQPGESLRLSCAASGFTFSNYAMGWFRQAPGKERDFVAAVSGIARRTYYADSVKGRSTISRDNGRNTVYLQMNSLKPEDTAVYYCARATSRMTSVTTLDDYGYWGQGTQVTVSSAAAYPYDVPDYGSHHHHHH

SEQ ID NO: 362 - protein, 18 aa, synthetic construct
  REGION 1..18: note = Complementarity-determining region amino acid sequence
ARATSRMTSVTTLDDYGY

SEQ ID NO: 363 - protein, 144 aa, synthetic construct
  REGION 1..144: note = nanobody amino acid sequence
QVQLQESGGGLVQPGGSLRLSCAASGFTFSNYAMGWFRQAPGKERDFVAAVSGIARRTYYADSVKGRSTISRDNGRNTVYLQMNSLKPEDTAVYYCARATSRMTSVTTLNDYGYWGQGTQVTVSSAAAYPYDVPDYGSHHHHHH

SEQ ID NO: 364 - protein, 144 aa, synthetic construct
  REGION 1..144: note = nanobody amino acid sequence
QVQLQESGGGLVQPGGSLRLSCAASGFTFSSYAMGWFRQAPGKEREFVAAVSGIARRTYYADSVKGRSTISRDNGRNTVYLQMNSLKPEDTAVYYCARATSRMTSVTTLNDYAYWGQGTQVTVSSAAAYPYDVPDYGSHHHHHH

SEQ ID NO: 365 - protein, 8 aa, synthetic construct
  REGION 1..8: note = Complementarity-determining region amino acid sequence
GFTFSSYA

SEQ ID NO: 366 - protein, 18 aa, synthetic construct
  REGION 1..18: note = Complementarity-determining region amino acid sequence
ARATSRMTSVTTLNDYAY

SEQ ID NO: 367 - protein, 141 aa, synthetic construct
  REGION 1..141: note = nanobody amino acid sequence
QVQLQESGGGSVQSGGSLTLSCSASGSLFSINAMGWYRQAPRKQHELVATMMDGGSTSYADSVKGRFTISRDNDKKTVYLQMNSLKPEDTGVYYCVADRLGSGRYAYGIDYWGQGTQVTVSSAAAYPYDVPDYGSHHHHHH

SEQ ID NO: 368 - protein, 8 aa, synthetic construct
  REGION 1..8: note = Complementarity-determining region amino acid sequence
GSLFSINA

SEQ ID NO: 369 - protein, 7 aa, synthetic construct
  REGION 1..7: note = Complementarity-determining region amino acid sequence
MMDGGST

SEQ ID NO: 370 - protein, 16 aa, synthetic construct
  REGION 1..16: note = Complementarity-determining region amino acid sequence
VADRLGSGRYAYGIDY

SEQ ID NO: 371 - protein, 141 aa, synthetic construct
  REGION 1..141: note = nanobody amino acid sequence
QVQLQESGGGLVQPGGSLRLACAASGSIFSINAMGWYRQAPRKQRELVATMMDGGSTSYADSVKGRFTISRDNDKKTVYLQMNSLKPEDTGVYYCVADRLGSGRYAYGIDYWGQGTQVTVSSAAAYPYDVPDYGSHHHHHH

SEQ ID NO: 372 - protein, 8 aa, synthetic construct
  REGION 1..8: note = Complementarity-determining region amino acid sequence
GSIFSINA

SEQ ID NO: 373 - protein, 141 aa, synthetic construct
  REGION 1..141: note = nanobody amino acid sequence
QVQLQESGGGLVQAGGSLRLSCAASGSIVSINGMGWYRQAPRKQRELVATMMDGGSTSYADSVKGRFTISRDNDKKTVYLQMNSLKPEDTGVYYCAADRLGSGRYAYAIDYWGQGTQVTVSSAAAYPYDVPDYGSHHHHHH

SEQ ID NO: 374 - protein, 8 aa, synthetic construct
  REGION 1..8: note = Complementarity-determining region amino acid sequence
GSIVSING

SEQ ID NO: 375 - protein, 16 aa, synthetic construct
  REGION 1..16: note = Complementarity-determining region amino acid sequence
AADRLGSGRYAYAIDY

SEQ ID NO: 376 - protein, 140 aa, synthetic construct
  REGION 1..140: note = nanobody amino acid sequence
QVQLQESGGGLVQPGGSLRLSCTASGRTVSNYGMAWFRQVAGKERAFVAAINNRGDSKYYAESVKGRFTIARDNAKNTVYLQMNVLKPEDTAVYYCAADRRSLVRYEYNYWGQGTQVTVSSAAAYPYDVPDYGSHHHHHH

SEQ ID NO: 377 - protein, 8 aa, synthetic construct
  REGION 1..8: note = Complementarity-determining region amino acid sequence
GRTVSNYG

SEQ ID NO: 378 - protein, 8 aa, synthetic construct
  REGION 1..8: note = Complementarity-determining region amino acid sequence
INNRGDSK

SEQ ID NO: 379 - protein, 14 aa, synthetic construct
  REGION 1..14: note = Complementarity-determining region amino acid sequence
AADRRSLVRYEYNY

SEQ ID NO: 380 - protein, 141 aa, synthetic construct
  REGION 1..141: note = nanobody amino acid sequence
QVQLQESGGGLVQAGGSLRLSCAASGRTFSRYTMGWFRQAPGKEREFVAAIRWSSGNTYYTDAVKGRFTISRDNTENTVYLQMNSLKPEDTAVYYCVADDGLSYASSSYLYWGQGTQVTVSSAAAYPYDVPDYGSHHHHHH

SEQ ID NO: 381 - protein, 8 aa, synthetic construct
  REGION 1..8: note = Complementarity-determining region amino acid sequence
GRTFSRYT

SEQ ID NO: 382 - protein, 8 aa, synthetic construct
  REGION 1..8: note = Complementarity-determining region amino acid sequence
IRWSSGNT

SEQ ID NO: 383 - protein, 15 aa, synthetic construct
  REGION 1..15: note = Complementarity-determining region amino acid sequence
VADDGLSYASSSYLY

SEQ ID NO: 384 - protein, 141 aa, synthetic construct
  REGION 1..141: note = nanobody amino acid sequence
QVQLQESGGGLVQPGGSLRLSCAASGFTFSRYTMGWFRQAPGKEREFVAAIRWSSGNTYYTDAVKGRFTISRDNTENTVYLQMNSLKPEDTAVYYCVADDGLSYASSSYLYWGQGTQVTVSSAAAYPYDVPDYGSHHHHHH

SEQ ID NO: 385 - protein, 8 aa, synthetic construct
  REGION 1..8: note = Complementarity-determining region amino acid sequence
GFTFSRYT

SEQ ID NO: 386 - protein, 140 aa, synthetic construct
  REGION 1..140: note = nanobody amino acid sequence
QVQLQESGGGLVQAGGSLRLSCVASGRTFGNYGLAWFRQPPGKEREFVAAINNRGGNTYYADSVKGRFTISRDNAANTLYLQMSSLKPEDTAVYYCAADRTSLHSYRYTYWGQGTQVTVSSAAAYPYDVPDYGSHHHHHH

SEQ ID NO: 387 - protein, 8 aa, synthetic construct
  REGION 1..8: note = Complementarity-determining region amino acid sequence
GRTFGNYG

SEQ ID NO: 388 - protein, 8 aa, synthetic construct
  REGION 1..8: note = Complementarity-determining region amino acid sequence
INNRGGNT

SEQ ID NO: 389 - protein, 14 aa, synthetic construct
  REGION 1..14: note = Complementarity-determining region amino acid sequence
AADRTSLHSYRYTY

SEQ ID NO: 390 - protein, 140 aa, synthetic construct
  REGION 1..140: note = nanobody amino acid sequence
QVQLQESGGGLVQAGGSLRLSCVASGRTFGNYGLAWFRQPPGKEREFVAAISNRGGNTYYADSVKGRFTISRDNAANTLYLQMSSLKPEDTAVYYCAADRTSLHSYRYTYWGQGTQVTVSSAAAYPYDVPDYGSHHHHHH

SEQ ID NO: 391 - protein, 8 aa, synthetic construct
  REGION 1..8: note = Complementarity-determining region amino acid sequence
ISNRGGNT

SEQ ID NO: 392 - protein, 140 aa, synthetic construct
  REGION 1..140: note = nanobody amino acid sequence
QVQLQESGGGLVQAGDSPKLSCVASGRTFGNYGLAWFRQPPGKEREFVAAISNRGGNTYYADSVKGRFTISRDNAANTLYLQMSSLKPEDTAVYYCAADRTSLHSYRYTYWGQGTQVTVSSAAAYPYDVPDYGSHHHHHH

SEQ ID NO: 393 - protein, 142 aa, synthetic construct
  REGION 1..142: note = nanobody amino acid sequence
QVQLQESGGGLVQAGGSLRLSCAASERTFSTYNMAWFRQAPGKERESVSRINWNGGFTGYADSVKGRFTISRDNAKKAMYLQMNSLKSEDTAVYYCAACGSAYPCRPEEYTYWGQGTQVTVSSAAAYPYDVPDYGSHHHHHH

SEQ ID NO: 394 - protein, 8 aa, synthetic construct
  REGION 1..8: note = Complementarity-determining region amino acid sequence
ERTFSTYN

SEQ ID NO: 395 - protein, 8 aa, synthetic construct
  REGION 1..8: note = Complementarity-determining region amino acid sequence
INWNGGFT

SEQ ID NO: 396 - protein, 14 aa, synthetic construct
  REGION 1..14: note = Complementarity-determining region amino acid sequence
AACGSAYPCRPEEY

SEQ ID NO: 397 - protein, 139 aa, synthetic construct
  REGION 1..139: note = nanobody amino acid sequence
QVQLQESGGGLVQTGESLRLSCAASGRTLSSYTMGWFRQAPGKEREIVAASSWSGGRTYYADSVKGRFTMSRNNAENTVYLQMNSLNPEDTAVYYCAADSSRAPRTYNYWGQGTQVTVSSAAAYPYDVPDYGSHHHHHH

SEQ ID NO: 398 - protein, 8 aa, synthetic construct
  REGION 1..8: note = Complementarity-determining region amino acid sequence
GRTLSSYT

SEQ ID NO: 399 - protein, 8 aa, synthetic construct
  REGION 1..8: note = Complementarity-determining region amino acid sequence
SSWSGGRT

SEQ ID NO: 400 - protein, 13 aa, synthetic construct
  REGION 1..13: note = Complementarity-determining region amino acid sequence
AADSSRAPRTYNY

SEQ ID NO: 401 - protein, 140 aa, synthetic construct
  REGION 1..140: note = nanobody amino acid sequence
QVQLQESGGGLVQAGGSLRLSCAASGRTFSTYNMSWFRQGPGKERIFVATISWSGRVTDYADSVRGRFTISRDNAKKMVYLQMNSLKPEDTAVYYCAADSNSRRSRDYDYWGQGTQVTVSSAAAYPYDVPDYGSHHHHHH

SEQ ID NO: 402 - protein, 8 aa, synthetic construct
  REGION 1..8: note = Complementarity-determining region amino acid sequence
GRTFSTYN

SEQ ID NO: 403 - protein, 8 aa, synthetic construct
  REGION 1..8: note = Complementarity-determining region amino acid sequence
ISWSGRVT

SEQ ID NO: 404 - protein, 14 aa, synthetic construct
  REGION 1..14: note = Complementarity-determining region amino acid sequence
AADSNSRRSRDYDY

SEQ ID NO: 405 - protein, 134 aa, synthetic construct
  REGION 1..134: note = nanobody amino acid sequence
QVQLQESGGGLVQPGGSLRLSCAASGTTRRINSMRWYRQTPGNERDLVAGITEGGFTAYVDSVKGRFTISRDNAKNTVYLQMNSLKPEDTAVYYCYAAYLGAAYWGQGTQVTVSSAAAYPYDVPDYGSHHHHHH

SEQ ID NO: 406 - protein, 8 aa, synthetic construct
  REGION 1..8: note = Complementarity-determining region amino acid sequence
GTTRRINS

SEQ ID NO: 407 - protein, 7 aa, synthetic construct
  REGION 1..7: note = Complementarity-determining region amino acid sequence
ITEGGFT

SEQ ID NO: 408 - protein, 6 aa, synthetic construct
  REGION 1..6: note = Complementarity-determining region amino acid sequence
YLGAAY

SEQ ID NO: 409 - protein, 133 aa, synthetic construct
  REGION 1..133: note = type 2 chitin-binding domain (ChtBD2)
  VARIANT 2..12: note = Xaa can be any naturally occurring amino acid
  VARIANT 43..47: note = Xaa can be any naturally occurring amino acid
  VARIANT 54..62: note = Xaa can be any naturally occurring amino acid
  VARIANT 87..98: note = Xaa can be any naturally occurring amino acid
  VARIANT 116..121: note = Xaa can be any naturally occurring amino acid
CXXXXXXXXXXXCCCCCCCCCCCCCCCCCCCCCCCCCCCCCCXXXXXCCCCCCXXXXXXXXXCCCCCCCCCCCCCCCCCCCCCCCCXXXXXXXXXXXXCCCCCCCCCCCCCCCCCXXXXXXCCCCCCCCCCCC

SEQ ID NO: 410 - protein, 1137 aa, synthetic construct
  REGION 1..1137: note = type 2 chitin-binding domain (ChtBD2)
MKDAVILLLCAIALADGFNHELLSNSRSCNCNPSEAQQICEANYGKDDVLIAHENCDKFYKCANGKPFPYKCPNNLLYDPYKEECEWPWLVDCGDRPISEGSDQEDGGVDNDNNDSNDNNGVDDGWTCNCNPSEAPSICAAEGSSGVLVAHENCDQFYMCDAGRPVTFDCPPTLMYDPHREECYWPHEVDCGDRGISEPGNDQGNDNDGNDNNSSNEQGGVCNCNPEEAPAICASPGSEGVLVAHENCEKYYICNHGRPVVASCSGNLLFNPYTNECGWPRDVDCGDRIEPGCTGCNDNNNNDDNDSDCDGDDPVPPPADNDDSESADIDDLPPPGDDASVRPPVDEGTCNCNPEQAPSICAEDDSDGVLVAHEDCNKFYKCHNGKPVALYCPGNLLYNPNTEQCDWPEKVDCGDRVIPDPEDNNNNDSSGGNNDGGNDGGNGGGGNCDPSEAPAICAEDDSEGVLVAHENCNQFYKCSGGKPVALLCPGNLLFNPNTDQCDWPWEVDCGDRIIPDPDQKPDPEDSSDDSSADIDDLPPPGDDVTTRPPGTCNCNPEEAPSICAQDGSNGTLIAHEDCNKFYICDHGKPVALSCPGNLLYNPYTEKCDWPENVECGDSDGILIAHEDCNKFYICDHGKPVVLSCPGNLFYNPYTEQCDWPVNVECGDRVTPDPDATPAPTAAPTSTTTVAPTAAPSTTTTVAPTAAPTTTTTVAPTTTTTVAPTTTTTVAPTAAPTTTTTVAPTAAPTTTTTVAPTAAPTTTTTVASTAAPTAAPTAAPTAASTPDDSDCDDDNNGGDDTCNCNPDEAASICSVGNSDGIHVAHENCNWFYKCDNGRPVPFRCPSGLMYNPYTQICDWPWDVECGDRVIADDDDSSEEDNDNDNDSGVVGPCNCNPEEAPAICAAEGSNGVHVAHQNCNQYYMCDNGRPVAFTCNGFLLYNPYTQQCDWPHLVECGDRVIPEPGDEDDEDCDDDDDNSNNVINDDPSQAPAICADSGSEGVLVAHENCDQYYICDGGRPVARPCQGGLLYNPLTQYCDWPGNVNCGDRIIPDDCACNPRNAPRLCSKPDSEGSLVAHENCNQFYICAHSVPVEHFCPVGLYYNIELELCDWAQNVNCENRNLPSLNKHWESRQTLRK

SEQ ID NO: 411 - protein, 134 aa, synthetic construct
  REGION 1..134: note = nanobody amino acid sequence
QVQLQESGGGLVQAGGSLRLSCSASTSIASINAVGWYRQAPGKQRELVAVIVNGSTTRYADSVKGRFTISGDNAENTVPLLMSSLKPEDTAIYYCGARDLSGTYWGQGTQVTVSSAAAYPYDVPDYGSHHHHHH

SEQ ID NO: 412 - protein, 8 aa, synthetic construct
  REGION 1..8: note = Complementarity-determining region amino acid sequence
TSIASINA

SEQ ID NO: 413 - protein, 7 aa, synthetic construct
  REGION 1..7: note = Complementarity-determining region amino acid sequence
IVNGSTT

SEQ ID NO: 414 - protein, 9 aa, synthetic construct
  REGION 1..9: note = Complementarity-determining region amino acid sequence
GARDLSGTY

SEQ ID NO: 415 - protein, 134 aa, synthetic construct
  REGION 1..134: note = nanobody amino acid sequence
QVQLQESGGGLVQPGGSLRLSCSASTSIASINAVGWYRQAPGKQRELVAVIVNGSTTRYADSVKGRFTISGDNAKNTVPLQMSSLKPEDTAIYYCGARDLSGTYWGQGTQVTVSSAAAYPYDVPDYGSHHHHHH

SEQ ID NO: 416 - protein, 134 aa, synthetic construct
  REGION 1..134: note = nanobody amino acid sequence
QVQLQESGGGLVQAGGSLRLSCSASTSIASINAVGWYRQAPGKQRELVAVIVNGSTTRYADSVKGRFTISGDNAKNTVPLQMSSLKPEDTAIYYCGARDLSGTYWGQGTQVTVSSAAAYPYDVPDYGSHHHHHH

SEQ ID NO: 417 - protein, 136 aa, synthetic construct
  REGION 1..136: note = nanobody amino acid sequence
QVQLQESGGGLVQAGGSLRLSCAASRGTFSRYVMGWFRQGPGKEREFVAGISWSGISTYYADFVKGRFTISRDNAKNTVYLQMNSLKPEDTAVYYCAADPGRGYDYWGQGTQVTVSSAAAYPYDVPDYGSHHHHHH

SEQ ID NO: 418 - protein, 8 aa, synthetic construct
  REGION 1..8: note = Complementarity-determining region amino acid sequence
RGTFSRYV

SEQ ID NO: 419 - protein, 8 aa, synthetic construct
  REGION 1..8: note = Complementarity-determining region amino acid sequence
ISWSGIST

SEQ ID NO: 420 - protein, 10 aa, synthetic construct
  REGION 1..10: note = Complementarity-determining region amino acid sequence
AADPGRGYDY

SEQ ID NO: 421 - protein, 136 aa, synthetic construct
  REGION 1..136: note = nanobody amino acid sequence
QVQLQESGGGLVQAGGSLRLSCAASGRSFSRSVMGWFRQAPGKEREFVAATSWSGGGTYYADSVKGRFTISRDNAQNTVYLQMNSLKPEDTAVYYCAADVGRGYHYWGQGTQVTVSSAAAYPYDVPDYGSHHHHHH

SEQ ID NO: 422 - protein, 8 aa, synthetic construct
  REGION 1..8: note = Complementarity-determining region amino acid sequence
GRSFSRSV

SEQ ID NO: 423 - protein, 8 aa, synthetic construct
  REGION 1..8: note = Complementarity-determining region amino acid sequence
TSWSGGGT

SEQ ID NO: 424 - protein, 10 aa, synthetic construct
  REGION 1..10: note = Complementarity-determining region amino acid sequence
AADVGRGYHY

SEQ ID NO: 425 - protein, 136 aa, synthetic construct
  REGION 1..136: note = nanobody amino acid sequence
QVQLQESGGGLVQAGGSLRLSCVASGGTFSGLTMGWFRQAPQKEREFVAAISWTGRSTYYADSVKGRFTISRDNVKNMVYLQMNSLKPEDTGVYFCAADWASGTPYWGQGTQVTVSSAAAYPYDVPDYGSHHHHHH

SEQ ID NO: 426 - protein, 8 aa, synthetic construct
  REGION 1..8: note = Complementarity-determining region amino acid sequence
GGTFSGLT

SEQ ID NO: 427 - protein, 8 aa, synthetic construct
  REGION 1..8: note = Complementarity-determining region amino acid sequence
ISWTGRST

SEQ ID NO: 428 - protein, 10 aa, synthetic construct
  REGION 1..10: note = Complementarity-determining region amino acid sequence
AADWASGTPY

SEQ ID NO: 429 - protein, 136 aa, synthetic construct
  REGION 1..136: note = nanobody amino acid sequence
QVQLQESGGGLVQAGTSLRLSCAASGGTFSGLTMGWFRQAPQKEREFVAAISWTGRSTYYADSVKGRFTISRDNVKNMVYLQMVALNPEDTAVYYCAADWASATPYWGQGTQVTVSSAAAYPYDVPDYGSHHHHHH

SEQ ID NO: 430 - protein, 10 aa, synthetic construct
  REGION 1..10: note = Complementarity-determining region amino acid sequence
AADWASATPY

SEQ ID NO: 431 - protein, 149 aa, synthetic construct
  REGION 1..149: note = nanobody amino acid sequence
QVQLQESGGGLVQSGESLTLACVISGITLERYTVGWFHQAPGKNPEGVSCIGKSNDETFYTDSVKGRFTISSDNAKNTVYLQMNSLTPSDAGVYYCAAAKAPVTAYDCSLYLYTWRSTYRGQGTQVTVSSAAAYPYDVPDYGSHHHHHH

SEQ ID NO: 432 - protein, 8 aa, synthetic construct
  REGION 1..8: note = Complementarity-determining region amino acid sequence
GITLERYT

SEQ ID NO: 433 - protein, 8 aa, synthetic construct
  REGION 1..8: note = Complementarity-determining region amino acid sequence
IGKSNDET

SEQ ID NO: 434 - protein, 23 aa, synthetic construct
  REGION 1..23: note = Complementarity-determining region amino acid sequence
AAAKAPVTAYDCSLYLYTWRSTY

SEQ ID NO: 435 - protein, 141 aa, synthetic construct
  REGION 1..141: note = nanobody amino acid sequence
QVQLQESGGGLVQAGGSLTLSCAASARTFSTYALAWFRQTPGKGREFVGAISRSGSSTRYADSVKGRFAISRDNAQRAIYLQMNSLKPEDTAVYYCAAGSRSYYNIPYYDYWGQGTQVTVSSAAAYPYDVPDYGSHHHHHH

SEQ ID NO: 436 - protein, 8 aa, synthetic construct
  REGION 1..8: note = Complementarity-determining region amino acid sequence
ARTFSTYA

SEQ ID NO: 437 - protein, 15 aa, synthetic construct
  REGION 1..15: note = Complementarity-determining region amino acid sequence
AAGSRSYYNIPYYDY

SEQ ID NO: 438 - protein, 147 aa, synthetic construct
  REGION 1..147: note = nanobody amino acid sequence
QVQLQESGGGLVQAGASLRLSCAASVRTFSSYAVGWFRQAPGKEREFVAGITWSGGSKYYRDAVKGRFTISRDNAKNAVYLQMNSLKPEDTAVYSCAATSARYTSGALYYRDRQYNYWGQGTQVTVSSAAAYPYDVPDYGSHHHHHH

SEQ ID NO: 439 - protein, 8 aa, synthetic construct
  REGION 1..8: note = Complementarity-determining region amino acid sequence
VRTFSSYA

SEQ ID NO: 440 - protein, 8 aa, synthetic construct
  REGION 1..8: note = Complementarity-determining region amino acid sequence
ITWSGGSK

SEQ ID NO: 441 - protein, 21 aa, synthetic construct
  REGION 1..21: note = Complementarity-determining region amino acid sequence
AATSARYTSGALYYRDRQYNY

SEQ ID NO: 442 - protein, 145 aa, synthetic construct
  REGION 1..145: note = nanobody amino acid sequence
QVQLQESGGGLVQAGGSLRLSCAASTRTFGNYAMGWFRQAPGKEREFVAAINRRGTTTYYADSVKDRFTISTDYAKNTVYLEMISLKPEDTAVYYCAVDRSTGWQPSTSRYDYASWGQGTQVTVSSAAAYPYDVPDYGSHHHHHH

SEQ ID NO: 443 - protein, 8 aa, synthetic construct
  REGION 1..8: note = Complementarity-determining region amino acid sequence
TRTFGNYA

SEQ ID NO: 444 - protein, 8 aa, synthetic construct
  REGION 1..8: note = Complementarity-determining region amino acid sequence
INRRGTTT

SEQ ID NO: 445 - protein, 19 aa, synthetic construct
  REGION 1..19: note = Complementarity-determining region amino acid sequence
AVDRSTGWQPSTSRYDYAS

SEQ ID NO: 446 - protein, 143 aa, synthetic construct
  REGION 1..143: note = nanobody amino acid sequence
QVQLQESGGGLVQAGSSLRLSCAASGRTFRYHAMGWFRQAPGKEREFVAGISTSGGMTYYPDSVKGRFTISRDNAKNTLYLQMNSLKPGDTAVYYCAKFHGDKGYGSSWYYDYWGQGTQVTVSSAAAYPYDVPDYGSHHHHHH

SEQ ID NO: 447 - protein, 8 aa, synthetic construct
  REGION 1..8: note = Complementarity-determining region amino acid sequence
GRTFRYHA

SEQ ID NO: 448 - protein, 8 aa, synthetic construct
  REGION 1..8: note = Complementarity-determining region amino acid sequence
ISTSGGMT

SEQ ID NO: 449 - protein, 17 aa, synthetic construct
  REGION 1..17: note = Complementarity-determining region amino acid sequence
AKFHGDKGYGSSWYYDY

SEQ ID NO: 450 - protein, 143 aa, synthetic construct
  REGION 1..143: note = nanobody amino acid sequence
QVQLQESGGGLVQPGGSLRLSCAASGFTFSSYAMSWVRQAPGKGLERISIINSGGGSTSYADSVKGRFTVSRDNNKNTLYLQMNSLQPEDTAVYYCGVRRNWGLGTHSGEYVYWGRGTQVTVSSAAAYPYDVPDYGSHHHHHH

SEQ ID NO: 451 - protein, 8 aa, synthetic construct
  REGION 1..8: note = Complementarity-determining region amino acid sequence
INSGGGST

SEQ ID NO: 452 - protein, 17 aa, synthetic construct
  REGION 1..17: note = Complementarity-determining region amino acid sequence
GVRRNWGLGTHSGEYVY

SEQ ID NO: 453 - protein, 139 aa, synthetic construct
  REGION 1..139: note = nanobody amino acid sequence
QVQLQESGGGLVQAGGSLRLSCAASGFTFDDYAIGWFRQAPGKVREGVSTIKSSDGSTYYADSVKGRFTISLDNAKRTVSLQMNNLKPEDTAVYYCAAGARRWPYDYIYWGRGTQVTVSSAAAYPYDVPDYGSHHHHHH

SEQ ID NO: 454 - protein, 8 aa, synthetic construct
  REGION 1..8: note = Complementarity-determining region amino acid sequence
GFTFDDYA

SEQ ID NO: 455 - protein, 8 aa, synthetic construct
  REGION 1..8: note = Complementarity-determining region amino acid sequence
IKSSDGST

SEQ ID NO: 456 - protein, 13 aa, synthetic construct
  REGION 1..13: note = Complementarity-determining region amino acid sequence
AAGARRWPYDYIY

SEQ ID NO: 457 - protein, 144 aa, synthetic construct
  REGION 1..144: note = nanobody amino acid sequence
QVQLQESGGGVVQAGASLRLSCAASERIFLNYNMAWVRQAPGKEREFVAAITWSGSNIDYADTVKGRFTISRDNAKNTVYLQMDSLKPEDTAVYYCAADPSYWKIRTTLNGLDKWGQGTQVTVSSAAAYPYDVPDYGSHHHHHH

SEQ ID NO: 458 - protein, 8 aa, synthetic construct
  REGION 1..8: note = Complementarity-determining region amino acid sequence
ERIFLNYN

SEQ ID NO: 459 - protein, 8 aa, synthetic construct
  REGION 1..8: note = Complementarity-determining region amino acid sequence
ITWSGSNI

SEQ ID NO: 460 - protein, 18 aa, synthetic construct
  REGION 1..18: note = Complementarity-determining region amino acid sequence
AADPSYWKIRTTLNGLDK

SEQ ID NO: 461 - protein, 138 aa, synthetic construct
  REGION 1..138: note = nanobody amino acid sequence
QVQLQESGGGFVQAGDSLRLSCAASGRTFSRFPMGWFRQAPGKEREFVAAISWSGGSTLITDSVKDRFTISRDNAKNTMYLQMNSLKPEDTAVYYCAARRDSTTSYVTWGQGTQVTVSSAAAYPYDVPDYGSHHHHHH

SEQ ID NO: 462 - protein, 8 aa, synthetic construct
  REGION 1..8: note = Complementarity-determining region amino acid sequence
GRTFSRFP

SEQ ID NO: 463 - protein, 8 aa, synthetic construct
  REGION 1..8: note = Complementarity-determining region amino acid sequence
ISWSGGST

SEQ ID NO: 464 - protein, 11 aa, synthetic construct
  REGION 1..11: note = Complementarity-determining region amino acid sequence
ARRDSTTSYVT

SEQ ID NO: 465 - protein, 137 aa, synthetic construct
  REGION 1..137: note = nanobody amino acid sequence
QVQLQESGGGLVQAGGSLRLSCTVSGGTFTTYTMAWFRQAPGKEREFVAQISWSGGITAYLDSVKGRFTISRDNAKNTVYLQMNSLKPEDTAVYYCARRGRKYEDDYWGQGTQVTVSSAAAYPYDVPDYGSHHHHHH

SEQ ID NO: 466 - protein, 8 aa, synthetic construct
  REGION 1..8: note = Complementarity-determining region amino acid sequence
GGTFTTYT

SEQ ID NO: 467 - protein, 8 aa, synthetic construct
  REGION 1..8: note = Complementarity-determining region amino acid sequence
ISWSGGIT

SEQ ID NO: 468 - protein, 11 aa, synthetic construct
  REGION 1..11: note = Complementarity-determining region amino acid sequence
ARRGRKYEDDY

SEQ ID NO: 469 - protein, 136 aa, synthetic construct
  REGION 1..136: note = nanobody amino acid sequence
QVQLQESGGGLVQAGGSLRLSCAASGRTFSSYIMGWFRQAPGKVREFVASISWSGGFTYYADSVKGRSTISRDNAKNTLYLQMNSLKSEDTAVYYCAADPGQGYKYWGQGTQVTVSSAAAYPYDVPDYGSHHHHHH

SEQ ID NO: 470 - protein, 8 aa, synthetic construct
  REGION 1..8: note = Complementarity-determining region amino acid sequence
GRTFSSYI

SEQ ID NO: 471 - protein, 8 aa, synthetic construct
  REGION 1..8: note = Complementarity-determining region amino acid sequence
ISWSGGFT

SEQ ID NO: 472 - protein, 10 aa, synthetic construct
  REGION 1..10: note = Complementarity-determining region amino acid sequence
AADPGQGYKY

SEQ ID NO: 473 - protein, 138 aa, synthetic construct
  REGION 1..138: note = nanobody amino acid sequence
QVQLQESGGGLVQAGGSLRLSCVASGRSFRSYTMGWLRQAPGKEREFVAAISMSGVVTYYADSVKGRFTISRDNAAKTLYLQMNSLKPEDTAVYYCAARPDRTGKADYSGQGTQVTVSSAAAYPYDVPDYGSHHHHHH

SEQ ID NO: 474 - protein, 8 aa, synthetic construct
  REGION 1..8: note = Complementarity-determining region amino acid sequence
GRSFRSYT

SEQ ID NO: 475 - protein, 8 aa, synthetic construct
  REGION 1..8: note = Complementarity-determining region amino acid sequence
ISMSGVVT

SEQ ID NO: 476 - protein, 12 aa, synthetic construct
  REGION 1..12: note = Complementarity-determining region amino acid sequence
AARPDRTGKADY

SEQ ID NO: 477 - protein, 137 aa, synthetic construct
  REGION 1..137: note = nanobody amino acid sequence
QVQLQESGGGLVQPGGSLRLSCAASGGTFSRSIMGWFRQAPGKERERVAAISWSGSLTFYADSVKGRFTISRDNAKNSVYLQMDSLKPEDTAVYYCAADKLGGTWDSWGPGTQVTVSSAAAYPYDVPDYGSHHHHHH

SEQ ID NO: 478 - protein, 8 aa, synthetic construct
  REGION 1..8: note = Complementarity-determining region amino acid sequence
GGTFSRSI

SEQ ID NO: 479 - protein, 8 aa, synthetic construct
  REGION 1..8: note = Complementarity-determining region amino acid sequence
ISWSGSLT

SEQ ID NO: 480 - protein, 11 aa, synthetic construct
  REGION 1..11: note = Complementarity-determining region amino acid sequence
AADKLGGTWDS

SEQ ID NO: 481 - protein, 138 aa, synthetic construct
  REGION 1..138: note = nanobody amino acid sequence
QVQLQESGGGLVQAGGSLRLSCTASGRTFSSYSMAWFRQASGKEREIVAAVSRFGKFKYYADSVKGRFTISRDNAKNTLYLQMNSLKPEDTAVYYCAKTDGSSWYLDYWGQGTQVTVSSAAAYPYDVPDYGSHHHHHH

SEQ ID NO: 482 - protein, 8 aa, synthetic construct
  REGION 1..8: note = Complementarity-determining region amino acid sequence
GRTFSSYS

SEQ ID NO: 483 - protein, 12 aa, synthetic construct
  REGION 1..12: note = Complementarity-determining region amino acid sequence
AKTDGSSWYLDY

SEQ ID NO: 484 - protein, 143 aa, synthetic construct
  REGION 1..143: note = nanobody amino acid sequence
QVQLQESGGGLVQAGDSLRLSCTASGRTFSSYVMGWFRQAPGKEREFVAAISRSGGNTYFGDSAEARFTISRDNTKNTVYLQMSSLRPDDTAVYYCARYRLVAGSTSRYTYDQWGQGTQVTVSSAAAYPYDVPDYGSHHHHHH

SEQ ID NO: 485 - protein, 8 aa, synthetic construct
  REGION 1..8: note = Complementarity-determining region amino acid sequence
GRTFSSYV

SEQ ID NO: 486 - protein, 8 aa, synthetic construct
  REGION 1..8: note = Complementarity-determining region amino acid sequence
ISRSGGNT

SEQ ID NO: 487 - protein, 17 aa, synthetic construct
  REGION 1..17: note = Complementarity-determining region amino acid sequence
ARYRLVAGSTSRYTYDQ

SEQ ID NO: 488 - protein, 141 aa, synthetic construct
  REGION 1..141: note = nanobody amino acid sequence
QVQLQESGGGLVQAGDSLRLTCAHSGRPFSSSAMGWFRQAPGKEREFVAAISRGGLSKYYADSVKGRFTIFRDNAKNTVYLQMNSLKPEDTAIYYCAGSLRDRPTKDEYVVWGQGTQVTVSSAAAYPYDVPDYGSHHHHHH

SEQ ID NO: 489 - protein, 8 aa, synthetic construct
  REGION 1..8: note = Complementarity-determining region amino acid sequence
GRPFSSSA

SEQ ID NO: 490 - protein, 8 aa, synthetic construct
  REGION 1..8: note = Complementarity-determining region amino acid sequence
ISRGGLSK

SEQ ID NO: 491 - protein, 15 aa, synthetic construct
  REGION 1..15: note = Complementarity-determining region amino acid sequence
AGSLRDRPTKDEYVV

SEQ ID NO: 492 - protein, 138 aa, synthetic construct
  REGION 1..138: note = nanobody amino acid sequence
QVQLQESGGGSVQAGDSLKLSCVASGRSRYALGWFRQAPGKAREFVGAARGGAGNTYYHESVKGRFTISRDNDKLTVYLQMNDLKPEDTAVYTCAAGKDFGTAVSWTSWGQGTQVTVSSAAAYPYDVPDYGSHHHHHH

SEQ ID NO: 493 - protein, 6 aa, synthetic construct
  REGION 1..6: note = Complementarity-determining region amino acid sequence
GRSRYA

SEQ ID NO: 494 - protein, 8 aa, synthetic construct
  REGION 1..8: note = Complementarity-determining region amino acid sequence
ARGGAGNT

SEQ ID NO: 495 - protein, 14 aa, synthetic construct
  REGION 1..14: note = Complementarity-determining region amino acid sequence
AAGKDFGTAVSWTS

SEQ ID NO: 496 - protein, 138 aa, synthetic construct
  REGION 1..138: note = nanobody amino acid sequence
QVQLQESGGGLVQAGGSLRLSCAASGGTFSGLTMGWFRQAPGKERDFVAAISWNGRFTYYKDSVKGRFAISRDNAKSTVYLQMTSLKPEDTAVYYCGADVLSGRGYRYWGQGTQVTVSSAAAYPYDVPDYGSHHHHHH

SEQ ID NO: 497 - protein, 8 aa, synthetic construct
  REGION 1..8: note = Complementarity-determining region amino acid sequence
ISWNGRFT

SEQ ID NO: 498 - protein, 12 aa, synthetic construct
  REGION 1..12: note = Complementarity-determining region amino acid sequence
GADVLSGRGYRY

SEQ ID NO: 499 - protein, 135 aa, synthetic construct
  REGION 1..135: note = nanobody amino acid sequence
QVQLQESGGGLVQAGGSLRLSCAVSGRAFSTYGMGWFRQAPRKEREFITAINRNGDRTWYADSVTGRFTISRDNDKNMVYLQMDSLKTEDTGIYYCHTRRFGYDYWGQGTQVTVSSAAAYPYDVPDYGSHHHHHH

SEQ ID NO: 500 - protein, 8 aa, synthetic construct
  REGION 1..8: note = Complementarity-determining region amino acid sequence
GRAFSTYG

SEQ ID NO: 501 - protein, 8 aa, synthetic construct
  REGION 1..8: note = Complementarity-determining region amino acid sequence
INRNGDRT

SEQ ID NO: 502 - protein, 9 aa, synthetic construct
  REGION 1..9: note = Complementarity-determining region amino acid sequence
HTRRFGYDY

SEQ ID NO: 503 - protein, 140 aa, synthetic construct
  REGION 1..140: note = nanobody amino acid sequence
QVQLQESGGGWVQPGGSLRLSCTISGLSRYYAMGWFRQVPGKERESVATISLRGGRTYYADSVEGRFTISRDNAKNTMYLQMNSLKPEDTAVYYCVADTTWGAPRSRYHYWGQGTQVTVSSAAAYPYDVPDYGSHHHHHH

SEQ ID NO: 504 - protein, 7 aa, synthetic construct
  REGION 1..7: note = Complementarity-determining region amino acid sequence
GLSRYYA

SEQ ID NO: 505 - protein, 8 aa, synthetic construct
  REGION 1..8: note = Complementarity-determining region amino acid sequence
ISLRGGRT

SEQ ID NO: 506 - protein, 15 aa, synthetic construct
  REGION 1..15: note = Complementarity-determining region amino acid sequence
VADTTWGAPRSRYHY

SEQ ID NO: 507 - protein, 141 aa, synthetic construct
  REGION 1..141: note = nanobody amino acid sequence
QVQLQESGGGLVQPGGSLRLSCAASGFTFRSYAMSWVRQAPGQGLEWVSAINSGGGSTTYADSVKGRFTISRDNAKNTLYLQMNSLKPEDTAVYYCAKMGSSSRGNRYLEVWGQGTQVTVSSAAAYPYDVPDYGSHHHHHH

SEQ ID NO: 508 - protein, 15 aa, synthetic construct
  REGION 1..15: note = Complementarity-determining region amino acid sequence
AKMGSSSRGNRYLEV

SEQ ID NO: 509 - protein, 8 aa, synthetic construct
  REGION 1..8: note = Complementarity-determining region amino acid sequence
INSGGGST

SEQ ID NO: 510 - protein, 15 aa, synthetic construct
  REGION 1..15: note = Complementarity-determining region amino acid sequence
AKMGSSSRGNRYLEV

SEQ ID NO: 511 - protein, 142 aa, synthetic construct
  REGION 1..142: note = nanobody amino acid sequence
QVQLQESGGGLVQAGDSLRLSCEASARTFIAYAVGWFRQAPGKEREFVAAISWNGGMTDYADFVKGRFAISRDNAKTVSLQMNNLKPEDTALYYCTAGPARRSYSYRDGYDYWGQGTQVTVSSAAAYPYDVPDYGSHHHHHH

SEQ ID NO: 512 - protein, 8 aa, synthetic construct
  REGION 1..8: note = Complementarity-determining region amino acid sequence
ARTFIAYA

SEQ ID NO: 513 - protein, 8 aa, synthetic construct
  REGION 1..8: note = Complementarity-determining region amino acid sequence
ISWNGGMT

SEQ ID NO: 514 - protein, 17 aa, synthetic construct
  REGION 1..17: note = Complementarity-determining region amino acid sequence
TAGPARRSYSYRDGYDY

SEQ ID NO: 515 - protein, 142 aa, synthetic construct
  REGION 1..142: note = nanobody amino acid sequence
QVQLQESGGGLVQTGGSLRLSCEASARTFIAYAVGWFRQAPGKEREFVAAISWNGGMTDYADFVKGRFAISRDSAKTVSLQMNNLKPEDTALYYCTAGPARRSYSYRDGYDYWGQGTQVTVSSAAAYPYDVPDYGSHHHHHH

SEQ ID NO: 516 - protein, 142 aa, synthetic construct
  REGION 1..142: note = nanobody amino acid sequence
QVQLQESGGGLVQVGGSLRISCVASGRTGSYYAMGWFRQAPGKEREFVAAISWNGGMTDYADFVKGRFAISRDNAKTVSLQMNNLKPEDTALYYCTAGPARRSYSYRDGYDYWGQGTQVTVSSAAAYPYDVPDYGSHHHHHH

SEQ ID NO: 517 - protein, 8 aa, synthetic construct
  REGION 1..8: note = Complementarity-determining region amino acid sequence
GRTGSYYA

SEQ ID NO: 518 - protein, 142 aa, synthetic construct
  REGION 1..142: note = nanobody amino acid sequence
QVQLQESGGGLVQPGRSLRLSCEASARTFIAYAVGWFRQAPGKEREFVAAISWNGGMTDYADFVKGRFAISRDNAKTVSLQMNNLKPEDTALYYCTAGPARRSYSYRDGYDYWGQGTQVTVSSAAAYPYDVPDYGSHHHHHH

SEQ ID NO: 519 - protein, 142 aa, synthetic construct
  REGION 1..142: note = nanobody amino acid sequence
QVQLQESGGGLVQSGGSLTLSCAASARTFIAYAVGWFRQAPGKEREFVAAISWNGGMTDYADFVKGRFAISRDNAKTVSLQMNNLKPEDTALYYCTAGPARRSYSYRDGYDYWGQGTQVTVSSAAAYPYDVPDYGSHHHHHH

SEQ ID NO: 520 - protein, 141 aa, synthetic construct
  REGION 1..141: note = nanobody amino acid sequence
QVQLQESGGGLVQAGGSLRLSCAASGSIFSTNAMGWYRQAPDKQREFLAVITPRGRTAYADSAKGRFTISRDNAMNSVYLQMNSLKFEDTAVYYCYAGRYRSYDARFATDIWGQGTQVTVSSAAAYPYDVPDYGSHHHHHH

SEQ ID NO: 521 - protein, 8 aa, synthetic construct
  REGION 1..8: note = Complementarity-determining region amino acid sequence
GSIFSTNA

SEQ ID NO: 522 - protein, 7 aa, synthetic construct
  REGION 1..7: note = Complementarity-determining region amino acid sequence
ITPRGRT

SEQ ID NO: 523 - protein, 16 aa, synthetic construct
  REGION 1..16: note = Complementarity-determining region amino acid sequence
YAGRYRSYDARFATDI

SEQ ID NO: 524 - protein, 141 aa, synthetic construct
  REGION 1..141: note = nanobody amino acid sequence
QVQLQESGGGLVQPGGSLRLSCAASGSIFSTNAMGWYRQAPDKQREFLAVITPRGRTAYADSAKGRFTISRDNAMNSVYLQMNSLKFEDTAVYYCYAGRYRSYDARFATDIWGQGTQVTVSSAAAYPYDVPDYGSHHHHHH

SEQ ID NO: 525 - protein, 141 aa, synthetic construct
  REGION 1..141: note = nanobody amino acid sequence
QVQLQESGGGLVQAGGSLRLSCAASGSIFSTNAMGWYRQAPDKQREFLAVITPRGRTAYADSAKGRFTISRDNAMNSVYLQMNSLKFEDTAVYYCYAGRYRSFDARFATDIWGQGTQVTVSSAAAYPYDVPDYGSHHHHHH

SEQ ID NO: 526 - protein, 16 aa, synthetic construct
  REGION 1..16: note = Complementarity-determining region amino acid sequence
YAGRYRSFDARFATDI

SEQ ID NO: 527 - protein, 141 aa, synthetic construct
  REGION 1..141: note = nanobody amino acid sequence
QVQLQESGGGLVRAGGSLRLSCAASGSIFSTNAMGWYRQAPDKQREFLAVITPRGRTAYADSAKGRFTISRDNAMNSVYLQMNSLKFEDTAVYYCYAGRYRSFDARFATDIWGQGTQVTVSSAAAYPYDVPDYGSHHHHHH

SEQ ID NO: 528 - protein, 141 aa, synthetic construct
  REGION 1..141: note = nanobody amino acid sequence
QVQLQESGGGLVQAGGSLRLSCAASGSIFSTNAMGWYRQAPDKQREFLAVITPRGRTAYADSAKGRFTISRDNAMNSVYLQMNSLKFEDTAVYYCYAGRYRSFDARFATDIWSQGTQVTVSSAAAYPYDVPDYGSHHHHHH

SEQ ID NO: 529 - protein, 141 aa, synthetic construct
  REGION 1..141: note = nanobody amino acid sequence
QVQLQESGGGLVQAGGSLRLSCAASGSIFSTNAMGWYRQAPDKQREFLAVITPRGRTAYADSAKGRFTISRDNAVNSVYLQMNSLKFEDTAVYYCYAGRYRSFDARFATDIWGQGTQVTVSSAAAYPYDVPDYGSHHHHHH

SEQ ID NO: 530 - protein, 139 aa, synthetic construct
  REGION 1..139: note = nanobody amino acid sequence
QVQLQESGGGLVHPGGSLRLSCVASGAFLTGATVGWYRQAPGKLRELVAAIITGGTTTYADSVKGRFSISRDYTKRALILQMDSLRPDDTAVYFCSIRGFYRQTQFREIWGQGTQVTVSSAAAYPYDVPDYGSHHHHHH

SEQ ID NO: 531 - protein, 8 aa, synthetic construct
  REGION 1..8: note = Complementarity-determining region amino acid sequence
GAFLTGAT

SEQ ID NO: 532 - protein, 7 aa, synthetic construct
  REGION 1..7: note = Complementarity-determining region amino acid sequence
IITGGTT

SEQ ID NO: 533 - protein, 14 aa, synthetic construct
  REGION 1..14: note = Complementarity-determining region amino acid sequence
SIRGFYRQTQFREI

SEQ ID NO: 534 - protein, 139 aa, synthetic construct
  REGION 1..139: note = nanobody amino acid sequence
QVQLQESGGGLVQPGESLRLSCVASGAFLTGATVGWYRQAPGKLRELVAAIITGGTTTYADSVKGRFSISRDYTKRALILQMDSLRPDDTAVYFCSIRGFYRQTQFREIWGQGTQVTVSSAAAYPYDVPDYGSHHHHHH

SEQ ID NO: 535 - protein, 139 aa, synthetic construct
  REGION 1..139: note = nanobody amino acid sequence
QVQLQESGGGLVQAGGSLRLSCVASGAFLTGATVGWYRQAPGKLRELVAAIITGGTTTYADSVKGRFSISRDYTKRALILQMDSLRPDDTAVYFCSIRGFYRQTQFREIWGQGTQVTVSSAAAYPYDVPDYGSHHHHHH

SEQ ID NO: 536 - protein, 139 aa, synthetic construct
  REGION 1..139: note = nanobody amino acid sequence
QVQLQESGGGEVQAGGSLRLSCVASGAFLTGATVGWYRQAPGKLRELVAAIITGGTTTYADSVKGRFSISRDYTKRALILQMDSLRPDDTAVYFCSIRGFYRQTQFREIWGQGTQVTVSSAAAYPYDVPDYGSHHHHHH

SEQ ID NO: 537 - protein, 139 aa, synthetic construct
  REGION 1..139: note = nanobody amino acid sequence
QVQLQESGGGEVQAGGSLRLSCVASGAFLTGATVGWYRQTPGNLRELVAAIITGGTTTYADSVKGRFSISRDYTKRALILQMDSLRPDDTAVYFCSIRGFYRQTQFREIWGQGTQVTVSSAAAYPYDVPDYGSHHHHHH

SEQ ID NO: 538 - protein, 143 aa, synthetic construct
  REGION 1..143: note = nanobody amino acid sequence
QVQLQESGGGLVQTGGSLRLSCAASGRTFSNYVMGWFRQAPGKERELIGAINRSSTRLYYADSVKGRFSISRDNAKSTVYLQMNSLKPEDTAVYYCAADLVSILGKGYRDVDYWGQGTQVTVSSAAAYPYDVPDYGSHHHHHH

SEQ ID NO: 539 - protein, 8 aa, synthetic construct
  REGION 1..8: note = Complementarity-determining region amino acid sequence
GRTFSNYV

SEQ ID NO: 540 - protein, 8 aa, synthetic construct
  REGION 1..8: note = Complementarity-determining region amino acid sequence
INRSSTRL

SEQ ID NO: 541 - protein, 17 aa, synthetic construct
  REGION 1..17: note = Complementarity-determining region amino acid sequence
AADLVSILGKGYRDVDY

SEQ ID NO: 542 - protein, 143 aa, synthetic construct
  REGION 1..143: note = nanobody amino acid sequence
QVQLQESGGGSVQAGGSLRLSCAASGRTFSNYVMGWFRQAPGKERELIGAINRSSTRLYYADSVKGRFSISRDNAKSTVYLQMNSLKPEDTAVYYCAADLVSILGKGYRDVDYWGQGTQVTVSSAAAYPYDVPDYGSHHHHHH

SEQ ID NO: 543 - protein, 143 aa, synthetic construct
  REGION 1..143: note = nanobody amino acid sequence
QVQLQESGGGLVQAGGSLRLSCAASGRTFSSYVMGWFRQAPGKERELIGAINRSSTRLYYADSVKGRFSISRDNAKSTVYLQMNSLKPEDTAVYYCAADLVSILGKGYRDVDYWGQGTQVTVSSAAAYPYDVPDYGSHHHHHH

SEQ ID NO: 544 - protein, 143 aa, synthetic construct
  REGION 1..143: note = nanobody amino acid sequence
QVQLQESGGGLVQPGGSLRVSCAASGFSFSNYVMGWFRQAPGKERELIGAINRSSTRLYYADSVKGRFSISRDNAKSTVYLQMNSLKPEDTAVYYCAADLVSILGKGYRDVDYWGQGTQVTVSSAAAYPYDVPDYGSHHHHHH

SEQ ID NO: 545 - protein, 8 aa, synthetic construct
  REGION 1..8: note = Complementarity-determining region amino acid sequence
GFSFSNYV

SEQ ID NO: 546 - protein, 144 aa, synthetic construct
  REGION 1..144: note = nanobody amino acid sequence
QVQLQESGGGLVQAGGSLRLSCVASGRTFSPYTMGWFRRAPGKEREFVAAISWGAGVKSYADSVKGRFTISRDNAENTVYLQMNMLKPDDTALYYCAAKRPISGSYSNERDYAYWGQGTQVTVSSAAAYPYDVPDYGSHHHHHH

SEQ ID NO: 547 - protein, 8 aa, synthetic construct
  REGION 1..8: note = Complementarity-determining region amino acid sequence
GRTFSPYT

SEQ ID NO: 548 - protein, 8 aa, synthetic construct
  REGION 1..8: note = Complementarity-determining region amino acid sequence
ISWGAGVK

SEQ ID NO: 549 - protein, 18 aa, synthetic construct
  REGION 1..18: note = Complementarity-determining region amino acid sequence
AAKRPISGSYSNERDYAY

SEQ ID NO: 550 - protein, 144 aa, synthetic construct
  REGION 1..144: note = nanobody amino acid sequence
QVQLQESGGGLVQPGGSLRLSCVASGRTFSPYTMGWFRRAPGKEREFVAAISWGAGVKSYADSVKGRFTISRDNAENTVYLQMNMLKPDDTALYYCAAKRPISGSYSNERDYAYWGQGTQVTVSSAAAYPYDVPDYGSHHHHHH

SEQ ID NO: 551 - protein, 134 aa, synthetic construct
  REGION 1..134: note = nanobody amino acid sequence
QVQLQESGGGLVQTGESLRLSCAGTNFLSSRFEMGWYRQIPGKQRELVARIFRDGNTDYVDSVKGRFTISRDTAKNTIDLQMNNLKPEDTAGYFCHVHILGRDYWGQGTQVTVSSAAAYPYDVPDYGSHHHHHH

SEQ ID NO: 552 - protein, 8 aa, synthetic construct
  REGION 1..8: note = Complementarity-determining region amino acid sequence
NFLSSRFE

SEQ ID NO: 553 - protein, 7 aa, synthetic construct
  REGION 1..7: note = Complementarity-determining region amino acid sequence
IFRDGNT

SEQ ID NO: 554 - protein, 9 aa, synthetic construct
  REGION 1..9: note = Complementarity-determining region amino acid sequence
HVHILGRDY

SEQ ID NO: 555 - protein, 144 aa, synthetic construct
  REGION 1..144: note = nanobody amino acid sequence
QVQLQESGGGLVQAGDSLRISCKASGRTFSSYPIGWFRQAPGKEREFVAAISRSGGRTYYADSVKGRLTISRDNAKNTVYLQMNSLKLEDTAVYYCAAKTTMGLPVGGTYEYDYWGQGTQVTVSSAAAYPYDVPDYGSHHHHHH

SEQ ID NO: 556 - protein, 8 aa, synthetic construct
  REGION 1..8: note = Complementarity-determining region amino acid sequence
GRTFSSYP

SEQ ID NO: 557 - protein, 8 aa, synthetic construct
  REGION 1..8: note = Complementarity-determining region amino acid sequence
ISRSGGRT

SEQ ID NO: 558 - protein, 18 aa, synthetic construct
  REGION 1..18: note = Complementarity-determining region amino acid sequence
AAKTTMGLPVGGTYEYDY

SEQ ID NO: 559 - protein, 144 aa, synthetic construct
  REGION 1..144: note = nanobody amino acid sequence
QVQLQESGGGLVQAGDSLRISCKASGRTFSSYPIGWFRQAPGKEREFVAAISRSGGRTYYADSVKGRFTISRDNAKNTVYLQMNSLKLEDTAVYYCAAKTTMGLPVGGTYEYDYWGQGTQVTVSSAAAYPYDVPDYGSHHHHHH

SEQ ID NO: 560 - protein, 142 aa, synthetic construct
  REGION 1..142: note = nanobody amino acid sequence
QVQLQESGGGLVQPGGSLRLSCAASGFTLDDYAIGWFRQAPGKERSFVAAIDSNGSNTYYADSLKGRFTIARDNAKSMVFLRMNNLEPEDTAVYYCAAGQNFWTFTTTPPPYWGQGTQVTVSSAAAYPYDVPDYGSHHHHHH

SEQ ID NO: 561 - protein, 8 aa, synthetic construct
  REGION 1..8: note = Complementarity-determining region amino acid sequence
GFTLDDYA

SEQ ID NO: 562 - protein, 8 aa, synthetic construct
  REGION 1..8: note = Complementarity-determining region amino acid sequence
IDSNGSNT

SEQ ID NO: 563 - protein, 16 aa, synthetic construct
  REGION 1..16: note = Complementarity-determining region amino acid sequence
AAGQNFWTFTTTPPPY

SEQ ID NO: 564 - protein, 145 aa, synthetic construct
  REGION 1..145: note = nanobody amino acid sequence
QVQLQESGGGLVQAGDSLRISCKASGRTFSNYPIGWFRQAPGKEREFVAAISRSGGGTRYADSVKGRFTISRDNAKNTVYLQMNSLKRDDTAVYYCAAYFGNLGGGVGRSSDYDYWGQGTQVTVSSAAAYPYDVPDYGSHHHHHH

SEQ ID NO: 565 - protein, 8 aa, synthetic construct
  REGION 1..8: note = Complementarity-determining region amino acid sequence
GRTFSNYP

SEQ ID NO: 566 - protein, 8 aa, synthetic construct
  REGION 1..8: note = Complementarity-determining region amino acid sequence
ISRSGGGT

SEQ ID NO: 567 - protein, 19 aa, synthetic construct
  REGION 1..19: note = Complementarity-determining region amino acid sequence
AAYFGNLGGGVGRSSDYDY

SEQ ID NO: 568 - protein, 134 aa, synthetic construct
  REGION 1..134: note = nanobody amino acid sequence
QVQLQESGGGSVQAGESLTLSCAASNVLVSKFTVAWFRQAPGKQRELVADIARAGFTSYADFVRGRFSISRDNAQNTVTLQMNSLTPEDTAVYYCNCHVLGRDYWGQGTQVTVSSAAAYPYDVPDYGSHHHHHH

SEQ ID NO: 569 - protein, 8 aa, synthetic construct
  REGION 1..8: note = Complementarity-determining region amino acid sequence
NVLVSKFT

SEQ ID NO: 570 - protein, 7 aa, synthetic construct
  REGION 1..7: note = Complementarity-determining region amino acid sequence
IARAGFT

SEQ ID NO: 571 - protein, 9 aa, synthetic construct
  REGION 1..9: note = Complementarity-determining region amino acid sequence
NCHVLGRDY

SEQ ID NO: 572 - protein, 134 aa, synthetic construct
  REGION 1..134: note = nanobody amino acid sequence
QVQLQESGGGSVQPGGSLRLSCAASIRTFSTYAFAWYRQAPGKQRELVAGISSGSRTNYADSVKGRFTISRDNAKKTVYLQMNNLQPEDTAVYYCTKGRTNIDYWGQGTQVTVSSAAAYPYDVPDYGSHHHHHH

SEQ ID NO: 573 - protein, 8 aa, synthetic construct
  REGION 1..8: note = Complementarity-determining region amino acid sequence
IRTFSTYA

SEQ ID NO: 574 - protein, 7 aa, synthetic construct
  REGION 1..7: note = Complementarity-determining region amino acid sequence
ISSGSRT

SEQ ID NO: 575 - protein, 9 aa, synthetic construct
  REGION 1..9: note = Complementarity-determining region amino acid sequence
TKGRTNIDY

SEQ ID NO: 576 - protein, 144 aa, synthetic construct
  REGION 1..144: note = nanobody amino acid sequence
QVQLQESGGGVVQPGGSLRLSCAASGRTFSSYVMGWFRQAPGKEREFVATISTGGGTTYYADSVKGRFTISRDNAKNTVYLQMNGLKPEDTAVYYCAAKMATGTASIRTYEYAYWGQGTQVTVSSAAAYPYDVPDYGSHHHHHH

SEQ ID NO: 577 - protein, 8 aa, synthetic construct
  REGION 1..8: note = Complementarity-determining region amino acid sequence
ISTGGGTT

SEQ ID NO: 578 - protein, 18 aa, synthetic construct
  REGION 1..18: note = Complementarity-determining region amino acid sequence
AAKMATGTASIRTYEYAY

SEQ ID NO: 579 - protein, 142 aa, synthetic construct
  REGION 1..142: note = nanobody amino acid sequence
QVQLQESGGGLVQPGGSLRLSCAASGFTFSRYAMGWFRQTPGKEREFVAAILWGGVIADSVKGRFTISRDNAKNTVDLQMFSLKPEDTAVYYCARRRGGLNNHLGSVGNYDYWGQGTQVTVSSAAAYPYDVPDYGSHHHHHH

SEQ ID NO: 580 - protein, 8 aa, synthetic construct
  REGION 1..8: note = Complementarity-determining region amino acid sequence
GFTFSRYA

SEQ ID NO: 581 - protein, 6 aa, synthetic construct
  REGION 1..6: note = Complementarity-determining region amino acid sequence
ILWGGV

SEQ ID NO: 582 - protein, 19 aa, synthetic construct
  REGION 1..19: note = Complementarity-determining region amino acid sequence
ARRRGGLNNHLGSVGNYDY

SEQ ID NO: 583 - protein, 134 aa, synthetic construct
  REGION 1..134: note = nanobody amino acid sequence
QVQLQESGGGSVQPGGSLRLSCEIIGATVSSSSMAWYRQAPGLQRELVAGITTPSNPHYAASVRGRFTISRDGARNLNYLQIDSAKPEDTAVYYCHAAIRGSIYRGQGTQVTVSSAAAYPYDVPDYGSHHHHHH

SEQ ID NO: 584 - protein, 8 aa, synthetic construct
  REGION 1..8: note = Complementarity-determining region amino acid sequence
GATVSSSS

SEQ ID NO: 585 - protein, 7 aa, synthetic construct
  REGION 1..7: note = Complementarity-determining region amino acid sequence
ITTPSNP

SEQ ID NO: 586 - protein, 9 aa, synthetic construct
  REGION 1..9: note = Complementarity-determining region amino acid sequence
HAAIRGSIY

SEQ ID NO: 587 - protein, 138 aa, synthetic construct
  REGION 1..138: note = nanobody amino acid sequence
QVQLQESGGGLVQAGGSLRLSCATSGRAFTNYHMGWFRQTPGKEREFVALISWSGDSTRYADSVKGRFTISRDNAKRTVALQMDSLKPEDSAVYYCASRLVGQSQYEVWGQGTQVTVSSAAAYPYDVPDYGSHHHHHH

SEQ ID NO: 588 - protein, 8 aa, synthetic construct
  REGION 1..8: note = Complementarity-determining region amino acid sequence
GRAFTNYH

SEQ ID NO: 589 - protein, 8 aa, synthetic construct
  REGION 1..8: note = Complementarity-determining region amino acid sequence
ISWSGDST

SEQ ID NO: 590 - protein, 12 aa, synthetic construct
  REGION 1..12: note = Complementarity-determining region amino acid sequence
ASRLVGQSQYEV

SEQ ID NO: 591 - protein, 138 aa, synthetic construct
  REGION 1..138: note = nanobody amino acid sequence
QVQLQESGGGLVQAGGSLRLSCAASGRAFTNYHMGWFRQTPGKEREFVALISWSGDSTRYADSVKGRFTISRDNAKRTVALQMDSLKPEDSAVYYCASRLVGQSQYEIWGQGTQVTVSSAAAYPYDVPDYGSHHHHHH

SEQ ID NO: 592 - protein, 12 aa, synthetic construct
  REGION 1..12: note = Complementarity-determining region amino acid sequence
ASRLVGQSQYEI

SEQ ID NO: 593 - protein, 138 aa, synthetic construct
  REGION 1..138: note = nanobody amino acid sequence
QVQLQESGGGLVQAGGSLRLSCAASGRAFTNYHMGWFRQTPGKEREFVALISWSGDSTRYADSVKGRFTISRDNAKRTVALQMDSLKPEDSAVYYCASRLVGQSQYEVWGQGTQVTVSSAAAYPYDVPDYGSHHHHHH

SEQ ID NO: 594 - protein, 138 aa, synthetic construct
  REGION 1..138: note = nanobody amino acid sequence
QVQLQESGGGLVQAGGSLRLSCAASTRAFTNYHMGWFRQTPGKEREFVALISWSGDSTRYADSVKGRFTISRDNAKRTVALQMDSLKPEDSAVYYCASRLVGQSQYEIWGQGTQVTVSSAAAYPYDVPDYGSHHHHHH

SEQ ID NO: 595 - protein, 8 aa, synthetic construct
  REGION 1..8: note = Complementarity-determining region amino acid sequence
TRAFTNYH

SEQ ID NO: 596 - protein, 138 aa, synthetic construct
  REGION 1..138: note = nanobody amino acid sequence
QVQLQESGGGLVQTGGSLRLSCAASGRAFTNYHMGWFRQTPGKEREFVALISWSGDSTRYADSVKGRFTISRDNAKRTVALQMDSLKPEDSAVYYCASRLVGQSQYEVWGQGTQVTVSSAAAYPYDVPDYGSHHHHHH